(19) United States
(12) Patent Application Publication
(10) Pub. No.: US 2025/0098679 A1
(43) Pub. Date: Mar. 27, 2025

(54) HERBICIDAL PYRIDONE DERIVATIVES

(71) Applicant: SYNGENTA CROP PROTECTION AG, Basel (CH)

(72) Inventors: Louisa WHALLEY, Bracknell, Berkshire (GB); James Alan MORRIS, Bracknell, Berkshire (GB); Christopher James MARTIN, Bracknell, Berkshire (GB); Gordon Richard MUNNS, Bracknell, Berkshire (GB)

(73) Assignee: SYNGENTA CROP PROTECTION AG, Basel (CH)

(21) Appl. No.: 18/720,820

(22) PCT Filed: Dec. 9, 2022
(86) PCT No.: PCT/EP2022/085139

§ 371 Date: Jun. 17, 2024

(30) Foreign Application Priority Data
Dec. 17, 2021 (EP) 21215564.2

(51) Int. Cl. A01N 43/56; A01N 43/50; A01N 43/647; A01N 43/653; A01N 43/90; A01P 13/00; C07D 401/06; C07D 471/04; C07D 491/044

(52) U.S. Cl. CPC A01N 43/56; A01N 43/50; A01N 43/647; A01N 43/653; A01N 43/90; A01P 13/00; C07D 401/06; C07D 471/04; C07D 491/044

(57) ABSTRACT

Compounds of Formula (I) wherein the substituents are as defined in claim 1. The invention further relates to herbicidal compositions which comprise a compound of Formula (I) and to the use of compounds of Formula (I) for controlling weeds, in particular in crops of useful plants.

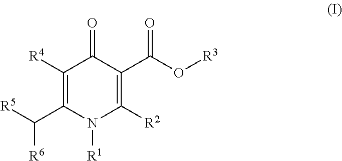

The present invention relates to herbicidal pyridone derivatives, e.g., as active ingredients, which have herbicidal activity. The invention also relates to agrochemical compositions which comprise at least one of the pyridone derivatives, to processes of preparation of these compounds and to uses of the pyridone derivatives or compositions in agriculture or horticulture for controlling weeds, in particular in crops of useful plants.

EP0239391, EP0127313, EP0040082, CN108617661, and GB2182931 describe pyridone derivatives as herbicidal agents.

According to the present invention, there is provided a compound of Formula (I):

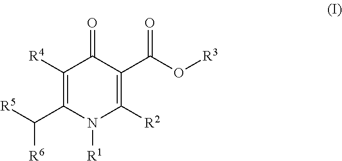

wherein
R¹ is C₁-C₆alkyl, C₁-C₆alkoxy, C₂-C₆alkenyl, C₂-C₆alkynyl, or C₁-C₆alkoxyC₁-C₆alkyl;
R² is phenyl or heteroaryl, wherein the heteroaryl moiety is a 5- or 6-membered aromatic ring which comprises 1, 2, 3 or 4 heteroatoms individually selected from N, O and S, and wherein each phenyl and heteroaryl moiety may be optionally substituted with 1, 2, 3, or 4 groups, which may be the same or different, represented by R⁷;
R³ is hydrogen or C₁-C₆alkyl;
R⁴ is hydrogen, halogen, C₁-C₆alkyl, C₁-C₆haloalkyl, or phenyl, and wherein the phenyl moieties may each be optionally substituted with 1, 2, 3, or 4 groups, which may be the same or different, represented by R⁸;
R⁵ is heteroaryl wherein the heteroaryl moiety is a 5-membered aromatic monocyclic ring comprising 1, 2, or 3 nitrogen atoms, and wherein the heteroaryl moieties are attached to the rest of the molecule through a nitrogen atom in the heteroaryl ring, and wherein the heteroaryl moieties may each be optionally substituted with 1, 2, 3, or 4 groups, which may be the same or different, represented by R⁹;
R⁶ is hydrogen, C₁-C₃alkyl, or C₁-C₆alkoxy;
R⁷ is cyano, nitro, amino, halogen, C₁-C₆alkyl, C₁-C₆alkoxy, C₁-C₆haloalkyl, C₁-C₆haloalkoxy, C₁-C₆alkoxyC₁-C₆alkyl, C₁-C₆alkylsulfanyl, C₁-C₆alkylsulfinyl, C₁-C₆alkylsulfonyl, C₁-C₆alkylsulfonamido, C₁-C₆alkylcarbonyl, C₁-C₆alkoxycarbonyl, C₁-C₆alkylaminocarbonyl, C₃-C₆cycloalkyl, C₃-C₆cycloalkylaminocarbonyl, or N,N-di(C₁-C₄alkyl)aminocarbonyl;
R⁸ is halogen, C₁-C₃alkyl, or C₁-C₃alkoxy;
R⁹ is formyl, acetyl, cyano, amino, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkyl, cyanoC₁-C₆alkoxy, C₁-C₆alkoxyC₁-C₆alkyl, C₁-C₆alkoxyC₁-C₆alkoxy, C₁-C₆alkoxycarbonylC₁-C₆alkoxy, C₁-C₆alkoxycarbonyl, C₁-C₆alkylsulfanyl, C₁-C₆alkylsulfinyl, C₁-C₆alkylsulfonyl, C₁-C₆alkylsulfonamido, hydroxycarbonyl, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₆alkoxyiminoC₁-C₆alkyl, C₁-C₆alkoxyC₁-C₆alkylcarbonylamino, C₂-C₆alkenylcarbonylamino, C₃-C₆cycloalkyl, C₃-C₆cycloalkylaminocarbonyl, N,N-di(C₁-C₄alkyl)amino, N,N-di(C₁-C₄alkyl)aminocarbonyl, phenyl, benzyloxy, or heteroaryl, wherein the heteroaryl moiety is a 5- or 6-membered aromatic ring which comprises 1, 2, or 3 heteroatoms individually selected from N, O and S, and wherein the phenyl and heteroaryl moieties may be optionally substituted with 1, 2, 3 or 4 groups, which may be the same or different, represented by R¹⁰; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, 3 or 4 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, 3 or 4 heteroatoms individually selected from N, O and S, and wherein the heteroaryl moiety may be optionally substituted with 1, 2, 3 or 4 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, 3 or 4 groups, which may be the same or different, represented by R¹⁰;
R¹⁰ is halogen, nitro, C₁-C₃alkyl, or C₁-C₃alkoxy;
or a salt or an N-oxide thereof.

Surprisingly, it has been found that the novel compounds of Formula (I) have, for practical purposes, a very advantageous level of herbicidal activity.

According to a second aspect of the invention, there is provided an agrochemical composition comprising a herbicidally effective amount of a compound of Formula (I) according to the present invention. Such an agricultural composition may further comprise at least one additional active ingredient and/or an agrochemically-acceptable diluent or carrier.

According to a third aspect of the invention, there is provided a method of controlling weeds at a locus comprising applying to the locus a weed controlling amount of a composition comprising a compound of Formula (I).

According to a fourth aspect of the invention, there is provided the use of a compound of Formula (I) as a herbicide.

Where substituents are indicated as being “optionally substituted”, this means that they may or may not carry one or more identical or different substituents, e.g., one, two or three R⁷ substituents. For example, C₁-C₆alkyl substituted by 1, 2 or 3 halogens, may include, but not be limited to, —CH₂Cl, —CHCl₂, —CCl₃, —CH₂F, —CHF₂, —CF₃, —CH₂CF₃ or —CF₂CH₃ groups. As another example, C₁-C₆alkoxy substituted by 1, 2 or 3 halogens, may include, but not limited to, CH₂ClO—, CHCl₂O—, CCl₃O—, CH₂FO—, CHF₂O—, CF₃O—, CF₃CH₂O— or CH₃CF₂O— groups.

As used herein, the term “cyano” means a —CN group.

As used herein, the term “hydroxy” or “hydroxyl” refers to an —OH group.

As used herein, the term “halogen” refers to fluorine (fluoro), chlorine (chloro), bromine (bromo) or iodine (iodo).

As used herein, the term “nitro” means an —NO₂ group.

As used herein, the term “formyl” means a —C(O)H group.

As used herein, the term “acetyl” means a —C(O)CH₃ group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. “C₁-C₄alkyl” and “C₁-C₃alkyl” are to be construed accordingly. Examples of C₁-C₆alkyl include, but are not limited to, methyl, ethyl, n-propyl, and the isomers thereof, for example, iso-propyl. A “C₁-C₆alkylene” group refers to the corresponding definition of C₁-C₆alkyl, except that such radical is attached to the rest of the molecule by two single bonds. The term “C₁-C₂alkylene” is to be construed accordingly. Examples of C₁-C₆alkylene, include, but are not limited to, —CH₂—, —CH₂CH₂— and —(CH₂)₃—.

As used herein, the term “C₁-C₆haloalkyl” refers a C₁-C₆alkyl radical as generally defined above substituted by one or more of the same or different halogen atoms. The terms “C₁-C₄haloalkyl” and “C₁-C₃haloalkyl”, are to be construed accordingly. Examples of C₁-C₆haloalkyl include, but are not limited to trifluoromethyl.

As used herein, the term “C₁-C₆alkoxy” refers to a radical of the formula —ORₐ where Rₐ is a C₁-C₆alkyl radical as generally defined above. The terms “C₁-C₄alkoxy” and “C₁-C₃alkoxy” are to be construed accordingly. Examples of C₁-C₆alkoxy include, but are not limited to, methoxy, ethoxy, 1-methylethoxy (iso-propoxy), and propoxy.

As used herein, the term “C₁-C₆haloalkoxy” refers to a C₁-C₆alkoxy radical as generally defined above substituted by one or more of the same or different halogen atoms. The terms “C₁-C₄haloalkoxy” and “C₁-C₃haloalkoxy”, are to be construed accordingly. Examples of C₁-C₆haloalkoxy include, but are not limited to trifluoromethoxy.

As used herein, the term “cyanoC₁-C₆alkoxy” refers to a C₁-C₆alkoxy radical as generally defined above substituted by one or more cyano groups. The terms “cyanoC₁-C₄alkoxy” and “cyanoC₁-C₃alkoxy”, are to be construed accordingly. Examples of cyanoC₁-C₆alkoxy include, but are not limited to cyanoethoxy.

As used herein, the term “cyanoC₁-C₆alkyl” refers to a C₁-C₆alkyl radical as generally defined above substituted by one or more cyano groups. The terms “cyanoC₁-C₄alkyl”, “cyanoC₁-C₃alkyl”, and “cyanoC₁-C₂alkyl”, are to be construed accordingly. Examples of cyanoC₁-C₆alkyl include, but are not limited to cyanomethyl.

As used herein, the term “C₂-C₆alkenyl” refers to a straight or branched hydrocarbon chain radical group consisting solely of carbon and hydrogen atoms, containing at least one double bond that can be of either the (E)- or (2)-configuration, having from two to six carbon atoms, which is attached to the rest of the molecule by a single bond. The term “C₂-C₃alkenyl” is to be construed accordingly. Examples of C₂-C₆alkenyl include, but are not limited to, ethenyl (vinyl), prop-1-enyl, prop-2-enyl (allyl), but-1-enyl.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. The term “C₂-C₃alkynyl” is to be construed accordingly. Examples of C₂-C₆alkynyl include, but are not limited to, ethynyl, prop-1-ynyl, but-1-ynyl.

As used herein, the term “C₂-C₆alkenyloxy” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule through an oxygen linker.

As used herein, the term “C₁-C₆alkoxyC₁-C₆alkyl” refers to a radical of the formula R_{b}OR^{a}— wherein R_{b} is a C₁-C₆alkyl radical as generally defined above, and Rₐ is a C₁-C₆alkylene radical as generally defined above. The term “C₁-C₄alkoxyC₁-C₄alkyl” is to be construed accordingly.

As used herein, the term “C₁-C₆alkoxyC₁-C₆alkoxy” refers to a radical of the formula R_{b}ORₐO— wherein Rₐ and R_{b} are both C₁-C₆alkylene radicals as generally defined above. The term “C₁-C₄alkoxyC₁-C₄alkoxy” is to be construed accordingly.

As used herein, the term “C₁-C₆alkoxycarbonylC₁-C₆alkoxy” refers to a radical of the formula RbOC(O)RaO—, wherein Rₐ and R_{b} are both C₁-C₆alkylene radicals as generally defined above.

As used herein, the term “C₃-C₆cycloalkyl” refers to a radical which is a monocyclic saturated ring system, and which contains 3 to 6 carbon atoms. The terms “C₃-C₅cycloalkyl” and “C₃-C₄cycloalkyl” are to be construed accordingly. Examples of C₃-C₆cycloalkyl include, but are not limited to, cyclopropyl, cyclobutyl, cyclopentyl, and cyclohexyl.

As used herein, the term “C₃-C₆cycloalkylaminocarbonyl” refers to a C₃-C₆cycloalkyl ring attached to the rest of the molecule through an —NHC(O)— linker.

As used herein, the term “benzyloxy” refers to a benzyl ring attached to the rest of the molecule through an oxygen atom.

As used herein, the term “heteroaryl” refers to a 5- or 6-membered aromatic monocyclic ring radical which comprises 1, 2, 3 or 4 heteroatoms individually selected from nitrogen, oxygen and sulfur.

In some instances, the heteroaryl moieties may be attached to the rest of the molecule through a nitrogen atom in the heteroaryl ring. In other instances, the heteroaryl moieties may be attached to the rest of the molecule through a carbon atom in the heteroaryl ring. Examples of heteroaryl include, but are not limited to, furanyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl or pyridyl.

As used herein, the term “heterocyclyl” refers to a stable 4-, 5- or 6-membered (and in the present case, preferably a 5- or 6-membered), non-aromatic monocyclic ring which comprises 1, 2 or 3 heteroatoms, wherein the heteroatoms are individually selected from nitrogen, oxygen and sulfur. The heterocyclyl radical may be bonded to the rest of the molecule via a carbon atom or heteroatom.

Examples of heterocyclyl include, but are not limited to, aziridinyl, azetidinyl, oxetanyl, thietanyl, tetrahydrofuryl, pyrrolidinyl, pyrazolidinyl, imidazolidnyl, piperidinyl, piperazinyl, morpholinyl, dioxolanyl, dithiolanyl and thiazolidinyl.

As used herein, the term “C₁-C₆alkylcarbonyl” refers to a radical of the formula —C(O)Rₐ, where Rₐ is a C₁-C₆alkyl radical as generally defined above. Examples of C₁-C₆alkylcarbonyl include, but are not limited to, acetyl.

As used herein, the term “C₁-C₆alkoxycarbonyl” refers to a radical of the formula —C(O)OR^{a}, where Rₐ is a C₁-C₆alkyl radical as generally defined above.

As used herein, the term “C₁-C₆alkylamino” refers to a radical of the formula RₐNH— wherein Rₐ is a C₁-C₆alkyl radical as generally defined above.

As used herein, the term “C₁-C₆alkylaminocarbonyl” refers to a radical of the formula —C(O)NHRa, wherein Rₐ is a C₁-C₆alkyl radical as generally defined above.

As used herein, the term “C₁-C₆alkylcarbonylamino” refers to a radical of the formula RₐC(O)NH— wherein Rₐ is a C₁-C₆alkyl radical as generally defined above.

As used herein, the term “C₁-C₆alkoxyC₁-C₆alkylaminocarbonyl” refers to a radical of the formula RₐOR_{b}NHC(O)—, wherein Rₐ is a C₁-C₆alkyl radical, Rₐ is a C₁-C₆alkylene radical as generally defined above.

As used herein, the term “C₁-C₆alkoxyC₁-C₆alkylcarbonylamino” refers to a radical of the formula RₐOR_{b}C(O)NH—, wherein Rₐ is a C₁-C₆alkyl radical, Rₐ is a C₁-C₆alkylene radical as generally defined above.

As used herein, the term “C₂-C₆alkenylcarbonylamino” refers to a radical of the formula RₐOC(O)NH—, wherein Rₐ is a C₁-C₆alkylene radical as generally defined above.

As used herein, the term “N,N-di(C₁-C₄alkyl)amino” refers to a radical of the formula (Rₐ)(R_{b})NH— wherein Rₐ and R_{b} are each individually a C₁-C₄alkyl radical as generally defined above.

As used herein, the term “N,N-di(C₁-C₄alkyl)aminocarbonyl” refers to a radical of the formula —C(O)N(Rₐ)(R_{b}), wherein Rₐ and R_{b} are each individually a C₁-C₄alkyl radical as generally defined above. The term “N,N-di(C₁-C₃alkyl)aminocarbonyl” is to be construed accordingly.

As used herein, the term “C₁-C₆alkylsulfanyl” refers to a radical of the formula —SRₐ, where Rₐ is a C₁-C₆alkyl radical as generally defined above. The terms “C₁-C₄alkylsulfanyl” and “C₁-C₃alkylsulfanyl”, are to be construed accordingly. Examples of C₁-C₆alkylsulfanyl include, but are not limited to methylsulfanyl.

As used herein, the term “C₁-C₆alkylsulfinyl” refers to a radical of the formula —S(O)Rₐ, where Rₐ is a C₁-C₆alkyl radical as generally defined above. The terms “C₁-C₄alkylsulfinyl” and “C₁-C₃alkylsulfinyl”, are to be construed accordingly. Examples of C₁-C₆alkylsulfinyl include, but are not limited to methylsulfinyl.

As used herein, the term “C₁-C₆alkylsulfonyl” refers to a radical of the formula —S(O)₂Rₐ, where Rₐ is a C₁-C₆alkyl radical as generally defined above. The terms “C₁-C₄alkylsulfonyl” and “C₁-C₃alkylsulfonyl”, are to be construed accordingly. Examples of C₁-C₆alkylsolfanyl include, but are not limited to methylsulfonyl.

As used herein, the term “C₁-C₆alkylsulfonamido” refers to a radical of the formula —NHS(O)₂Rₐ, wherein Rₐ is a C₁-C₆alkyl radical as generally defined above.

As used herein, the term “hydroxycarbonyl” or “carboxy;” refers to a radical of the formula —COOH.

As used herein, the term “C₁-C₆alkoxyiminoC₁-C₆alkyl” refers to a radical of the formula Rₐ═NR_{b}— wherein Rₐ is a C₁-C₆alkoxy radical as generally defined above, and R_{b} is a C₁-C₆alkylene radical as generally defined above.

The presence of one or more possible stereogenic elements in a compound of formula (I) means that the compounds may occur in optically isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms for a compound of formula (I).

In each case, the compounds of formula (I) according to the invention are in free form, in oxidized form as an N-oxide, or in salt form, e.g., an agronomically usable salt form. Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

N-oxides are oxidized forms of tertiary amines or oxidized forms of nitrogen-containing heteroaromatic compounds. They are described for instance in the book “Heterocyclic N-oxides” by A. Albini and S. Pietra, CRC Press, Boca Raton (1991).

The following list provides definitions, including preferred definitions, for substituents R¹, R², R³, R⁴, R⁵, R⁶, R¹, R⁸, and R⁹ with reference to compounds of Formula (I). For any one of these substituents, any of the definitions given below may be combined with any definition of any other substituent given below or elsewhere in this document.

R¹ is C₁-C₆alkyl, C₁-C₆alkoxy, C₂-C₆alkenyl, C₂-C₆alkynyl, or C₁-C₆alkoxyC₁-C₆alkyl. Preferably, R¹ is C₁-C₄alkyl, C₁-C₄alkoxy, C₂-C₄alkenyl, C₂-C₄alkynyl, or C₁-C₄alkoxyC₁-C₄alkyl. More preferably, R¹ is C₁-C₃alkyl, C₁-C₃alkoxy, C₂-C₃alkenyl, C₂-C₃alkynyl, or C₁-C₃alkoxyC₁-C₃alkyl. More preferably still, R¹ is methyl, ethyl, n-propyl, methoxy, 2-methoxyethyl, ally, and prop-2-ynyl. Even more preferably, R¹ is methyl or ethyl. In one set of embodiments, R¹ is ethyl.

R² is phenyl or heteroaryl, wherein the heteroaryl moiety is a 5- or 6-membered aromatic ring which comprises 1, 2, 3 or 4 heteroatoms individually selected from N, O and S, and wherein each phenyl and heteroaryl moiety may be optionally substituted with 1, 2, 3, or 4 groups, which may be the same or different, represented by R⁷.

Preferably, R² is phenyl or heteroaryl, wherein the heteroaryl moiety is a 5- or 6-membered aromatic ring which comprises 1, 2, or 3 heteroatoms individually selected from N, O and S, and wherein each phenyl and heteroaryl moiety may be optionally substituted with 1, 2, or 3 groups, which may be the same or different, represented by R⁷.

More preferably, R² is phenyl or heteroaryl, wherein the heteroaryl moiety is a 5- or 6-membered aromatic ring which comprises 1 or 2 heteroatoms individually selected from N and O, and wherein each phenyl and heteroaryl moiety may be optionally substituted with 1, 2, or 3 groups, which may be the same or different, represented by R⁷.

More preferably still, R² is phenyl or heteroaryl, wherein the heteroaryl moiety is a 5- or 6-membered aromatic ring which comprises 1 or 2 heteroatoms individually selected from N and O, and wherein each phenyl and heteroaryl moiety may be optionally substituted with 1 or 2 groups, which may be the same or different, represented by R⁷.

Even more preferably, R² is phenyl optionally substituted with 1 or 2 groups, which may be the same or different, represented by R⁷. In one set of embodiments, R² is 3,4-dichlorophenyl, 4-chloro-3-cyanophenyl, 4-chlorophenyl, 2-chloro-4-methylsulfonylphenyl, or 4-amino-3-chlorophenyl. In a further set of embodiments, R² is 3,4-dichlorophenyl.

R³ is hydrogen or C₁-C₆alkyl. Preferably, R³ is hydrogen or C₁-C₄alkyl. More preferably, R³ is hydrogen, methyl, ethyl, or t-butyl. More preferably still, R³ is hydrogen, methyl, or ethyl.

R⁴ is hydrogen, halogen, C₁-C₆alkyl, C₁-C₆haloalkyl, or phenyl, and wherein the phenyl moieties may each be optionally substituted with 1, 2, 3, or 4 groups, which may be the same or different, represented by R⁸. Preferably, R⁴ is hydrogen, halogen, C₁-C₄alkyl, C₁-C₄haloalkyl, or phenyl, and wherein the phenyl moieties may be optionally substituted with 1, 2, or 3 groups, which may be the same or different, represented by R⁸. More preferably, R⁴ is hydrogen, halogen, C₁-C₃alkyl, C₁-C₃haloalkyl, or phenyl, and wherein the phenyl moieties may be optionally substituted with 1 or 2 groups, which may be the same or different, represented by R⁸. Even more preferably, R⁴ is hydrogen, halogen, C₁-C₃alkyl, or phenyl, wherein the phenyl moieties may be optionally substituted with a single R⁸ group. More preferably still, R⁴ is hydrogen, halogen, methyl, or 4-chlorophenyl. Even more preferably still, R⁴ is hydrogen, bromo, methyl, or 4-chlorophenyl. In one set of embodiments, R⁴ is hydrogen, bromo, or 4-chlorophenyl. In another set of embodiments, R⁴ is hydrogen.

R⁵ is heteroaryl wherein the heteroaryl moiety is a 5-membered aromatic monocyclic ring comprising 1, 2, or 3 nitrogen atoms, and wherein the heteroaryl moieties are attached to the rest of the molecule through a nitrogen atom in the heteroaryl ring, and wherein the heteroaryl moieties may each be optionally substituted with 1, 2, 3, or 4 groups, which may be the same or different, represented by R⁹.

Preferably, R⁵ is heteroaryl wherein the heteroaryl moiety is a 5-membered aromatic monocyclic ring comprising 1, 2, or 3 nitrogen atoms, and wherein the heteroaryl moieties are attached to the rest of the molecule through a nitrogen atom in the heteroaryl ring, and wherein the heteroaryl moieties may each be optionally substituted with 1, 2, or 3 groups, which may be the same or different, represented by R⁹.

More preferably, R⁵ is heteroaryl wherein the heteroaryl moiety is a 5-membered aromatic monocyclic ring comprising 2 or 3 nitrogen atoms, and wherein the heteroaryl moieties are attached to the rest of the molecule through a nitrogen atom in the heteroaryl ring, and wherein the heteroaryl moieties may each be optionally substituted with 1, 2, or 3 groups, which may be the same or different, represented by R⁹.

More preferably still, R⁵ is pyrazolyl, imidazolyl, or triazolyl, wherein any of the pyrazolyl, imidazolyl, and triazolyl moieties may each be optionally substituted with 1, 2, or 3 groups, which may be the same or different, represented by R⁹.

In one set of embodiments, R⁵ is benzotriazol-2-ylmethyl, indazol-1-yl, indazol-2-yl, 4,5,6,7-tetrahydroindazol-2-yl, imidazo[4,5-b]pyridin-1-yl, 6-chloropyrazolo[4,3-c]pyridin-1-yl, 3-(difluoromethyl)-6,7-dihydro-4H-pyrano[4,3-c]pyrazol-1-yl, 3-(difluoromethyl)-6,7-dihydro-4H-pyrano[4,3-c]pyrazol-2-yl, 4,6-dihydrofuro[3,4-c]pyrazol-2-yl, or 3-(trifluoromethyl)-6,7-dihydro-4H-pyrano[4,3-c]pyrazol-1-yl.

R⁶ is hydrogen, C₁-C₃alkyl, or C₁-C₆alkoxy. Preferably, R⁶ is hydrogen or C₁-C₃alkyl. More preferably, R⁶ is hydrogen or methyl. More preferably still, R⁶ is hydrogen.

R⁷ is cyano, nitro, amino, halogen, C₁-C₆alkyl, C₁-C₆alkoxy, C₁-C₆haloalkyl, C₁-C₆haloalkoxy, C₁-C₆alkoxyC₁-C₆alkyl, C₁-C₆alkylsulfanyl, C₁-C₆alkylsulfinyl, C₁-C₆alkylsulfonyl, C₁-C₆alkylsulfonamido, C₁-C₆alkylcarbonyl, C₁-C₆alkoxycarbonyl, C₁-C₆alkylaminocarbonyl, C₃-C₆cycloalkyl, C₃-C₆cycloalkylaminocarbonyl, or N,N-di(C₁-C₄alkyl)aminocarbonyl.

Preferably, R⁷ is cyano, nitro, amino, halogen, C₁-C₄alkyl, C₁-C₄alkoxy, C₁-C₄haloalkyl, C₁-C₄haloalkoxy, C₁-C₄alkoxyC₁-C₃alkyl, C₁-C₄alkylsulfanyl, C₁-C₄alkylsulfinyl, C₁-C₄alkylsulfonyl, C₁-C₄alkylsulfonamido, C₁-C₄alkylcarbonyl, C₁-C₄alkoxycarbonyl, C₁-C₄alkylaminocarbonyl, C₃-C₆cycloalkyl, C₃-C₆cycloalkylaminocarbonyl, or N,N-di(C₁-C₃alkyl)aminocarbonyl.

More preferably, R⁷ is cyano, nitro, amino, halogen, C₁-C₃alkyl, C₁-C₃alkoxy, C₁-C₃haloalkyl, C₁-C₃haloalkoxy, C₁-C₃alkoxyC₁-C₃alkyl, C₁-C₃alkylsulfanyl, C₁-C₃alkylsulfinyl, C₁-C₃alkylsulfonyl, C₁-C₃alkylsulfonamido, C₁-C₃alkylcarbonyl, C₁-C₃alkoxycarbonyl, C₁-C₃alkylaminocarbonyl, C₃-C₆cycloalkyl, C₃-C₆cycloalkylaminocarbonyl, or N,N-di(C₁-C₃alkyl)aminocarbonyl.

More preferably still, R⁷ is cyano, nitro, amino, halogen, and C₁-C₃alkylsulfonyl, even more preferably, R⁷ is cyano, nitro, amino, chloro, fluoro, and methylsulfonyl.

In one set of embodiments, R⁷ is cyano, nitro, halogen, C₁-C₆alkyl, C₁-C₆alkoxy, C₁-C₆haloalkyl, C₁-C₆haloalkoxy, C₁-C₆alkoxyC₁-C₆alkyl, C₁-C₆alkylsulfanyl, C₁-C₆alkylsulfinyl, C₁-C₆alkylsulfonyl, C₁-C₆alkylsulfonamido, C₁-C₆alkylcarbonyl, C₁-C₆alkoxycarbonyl, C₁-C₆alkylaminocarbonyl, C₃-C₆cycloalkyl, C₃-C₆cycloalkylaminocarbonyl, or N,N-di(C₁-C₄alkyl)aminocarbonyl.

Preferably, R⁷ is cyano, nitro, halogen, C₁-C₄alkyl, C₁-C₄alkoxy, C₁-C₄haloalkyl, C₁-C₄haloalkoxy, C₁-C₄alkoxyC₁-C₃alkyl, C₁-C₄alkylsulfanyl, C₁-C₄alkylsulfinyl, C₁-C₄alkylsulfonyl, C₁-C₄alkylsulfonamido, C₁-C₄alkylcarbonyl, C₁-C₄alkoxycarbonyl, C₁-C₄alkylaminocarbonyl, C₃-C₆cycloalkyl, C₃-C₆cycloalkylaminocarbonyl, or N,N-di(C₁-C₃alkyl)aminocarbonyl.

More preferably, R⁷ is cyano, nitro, halogen, C₁-C₃alkyl, C₁-C₃alkoxy, C₁-C₃haloalkyl, C₁-C₃haloalkoxy, C₁-C₃alkoxyC₁-C₃alkyl, C₁-C₃alkylsulfanyl, C₁-C₃alkylsulfinyl, C₁-C₃alkylsulfonyl, C₁-C₃alkylsulfonamido, C₁-C₃alkylcarbonyl, C₁-C₃alkoxycarbonyl, C₁-C₃alkylaminocarbonyl, C₃-C₆cycloalkyl, C₃-C₆cycloalkylaminocarbonyl, or N,N-di(C₁-C₃alkyl)aminocarbonyl.

More preferably still, R⁷ is cyano, nitro, halogen, or C₁-C₃alkylsulfonyl, even more preferably, R⁷ is cyano, nitro, chloro, fluoro, and methylsulfonyl.

R⁸ is halogen, C₁-C₃alkyl, or C₁-C₃alkoxy. Preferably, R⁸ is halogen, methyl, ethyl, methoxy, or ethoxy. More preferably, R⁸ is chloro, bromo, fluoro, methyl, or methoxy. More preferably still, R⁸ is chloro.

R⁹ is formyl, acetyl, cyano, amino, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkyl, cyanoC₁-C₆alkoxy, C₁-C₆alkoxyC₁-C₆alkyl, C₁-C₆alkoxyC₁-C₆alkoxy, C₁-C₆alkoxycarbonylC₁-C₆alkoxy, C₁-C₆alkoxycarbonyl, C₁-C₆alkylsulfanyl, C₁-C₆alkylsulfinyl, C₁-C₆alkylsulfonyl, C₁-C₆alkylsulfonamido, hydroxycarbonyl, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₆alkoxyiminoC₁-C₆alkyl, C₁-C₆alkoxyC₁-C₆alkylcarbonylamino, C₂-C₆alkenylcarbonylamino, C₃-C₆cycloalkyl, C₃-C₆cycloalkylaminocarbonyl, N,N-di(C₁-C₄alkyl)amino, N,N-di(C₁-C₄alkyl)aminocarbonyl, phenyl, benzyloxy, or heteroaryl, wherein the heteroaryl moiety is a 5- or 6-membered aromatic ring which comprises 1, 2, or 3 heteroatoms individually selected from N, O and S, and wherein the phenyl and heteroaryl moieties may be optionally substituted with 1, 2, 3 or 4 groups, which may be the same or different, represented by R¹⁰; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, 3 or 4 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, 3 or 4 heteroatoms individually selected from N, O and S, and wherein the heteroaryl moiety may be optionally substituted with 1, 2, 3 or 4 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, 3 or 4 groups, which may be the same or different, represented by R¹⁰.

Preferably, R⁹ is formyl, acetyl, cyano, amino, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkyl, C₁-C₆alkoxyC₁-C₆alkyl, C₁-C₆alkoxyC₁-C₆alkoxy, C₁-C₆alkoxycarbonyl, C₁-C₆alkylsulfanyl, C₁-C₆alkylsulfonyl, hydroxycarbonyl, C₁-C₆alkylcarbonyl, C₁-C₆alkylcarbonylamino, C₁-C₆alkoxyiminoC₁-C₆alkyl, C₁-C₆alkoxyC₁-C₆alkylcarbonylamino, C₂-C₆alkenylcarbonylamino, C₃-C₆cycloalkyl, phenyl, or heteroaryl, wherein the heteroaryl moiety is a 5- or 6-membered aromatic ring which comprises 1, 2, or 3 heteroatoms individually selected from N, O and S, and wherein the phenyl and heteroaryl moieties may be optionally substituted with 1, 2, or 3 groups, which may be the same or different, represented by R¹⁰; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, or 3 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, or 3 heteroatoms individually selected from N, O and S, and wherein the heteroaryl moiety may be optionally substituted with 1, 2, or 3 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, or 3 groups, which may be the same or different, represented by R¹⁰.

More preferably, R⁹ is acetyl, cyano, nitro, hydroxy, halogen, C₁-C₄alkyl, C₁-C₄alkoxy, C₁-C₄haloalkyl, C₁-C₄haloalkoxy, C₂-C₄alkenyloxy, cyanoC₁-C₄alkyl, C₁-C₄alkoxyC₁-C₄alkyl, C₁-C₄alkoxyC₁-C₄alkoxy, C₁-C₄alkoxycarbonyl, C₁-C₄alkylsulfanyl, C₁-C₄alkylsulfonyl, C₁-C₄alkylcarbonyl, C₁-C₄alkylcarbonylamino, C₁-C₄alkoxyiminoC₁-C₄alkyl, C₁-C₄alkoxyC₁-C₄alkylcarbonylamino, C₂-C₄alkenylcarbonylamino, C₃-C₄cycloalkyl, phenyl, or heteroaryl, wherein the heteroaryl moiety is a 5- or 6-membered aromatic ring which comprises 1 or 2 heteroatoms individually selected from N, O and S, and wherein the phenyl and heteroaryl moieties may be optionally substituted with 1 or 2 groups, which may be the same or different, represented by R¹⁰; or
any two adjacent R⁹ groups together with the carbon atoms to which they are attached, may form a C₅-C₆cycloalkyl ring or phenyl ring, wherein the C₅-C₆cycloalkyl and phenyl moieties may be optionally substituted with 1 or 2 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1 or 2 heteroatoms individually selected from N, O and S, and wherein the heteroaryl moiety may be optionally substituted with 1 or 2 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1 or 2 groups, which may be the same or different, represented by R¹⁰.

Even more preferably, R⁹ is acetyl, cyano, nitro, hydroxy, halogen, C₁-C₄alkyl, C₁-C₄alkoxy, C₁-C₃haloalkyl, C₁-C₃haloalkoxy, C₂-C₃alkenyloxy, cyanoC₁-C₂alkyl, C₁-C₃alkoxyC₁-C₃alkyl, C₁-C₃alkoxyC₁-C₃alkoxy, C₁-C₂alkoxycarbonyl, C₁-C₂alkylsulfanyl, C₁-C₂alkylsulfonyl, C₁-C₃alkylcarbonyl, C₁-C₃alkylcarbonylamino, C₁-C₂alkoxyiminoC₁-C₂alkyl, C₂-C₃alkenylcarbonylamino, cyclopropyl, phenyl, or heteroaryl, wherein the heteroaryl moiety is a 5- or 6-membered aromatic ring which comprises 1 or 2 heteroatoms individually selected from N, O and S, and wherein the phenyl and heteroaryl moieties may be optionally substituted with a single R¹⁰ group; or
any two adjacent R⁹ groups together with the carbon atoms to which they are attached, may form a cyclohexyl or phenyl ring, wherein the phenyl moieties may be optionally substituted with 1 or 2 groups, which may be the same or different, represented by R¹⁰; or
any two adjacent R⁹ groups together with the carbon atoms to which they are attached, may form a heteroaryl ring, wherein the heteroaryl moiety is a 6-membered aromatic ring which comprises a single nitrogen atom, and wherein the heteroaryl moiety may be optionally substituted with a single R¹⁰ group; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1 a single R¹⁰ group.

In one set of embodiments, R⁹ is formyl, cyano, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkoxy, C₁-C₆alkoxyC₁-C₆alkyl, C₁-C₆alkoxyC₁-C₆alkoxy, C₁-C₆alkoxycarbonylC₁-C₆alkoxy, C₁-C₆alkoxycarbonyl, C₁-C₆alkylsulfanyl, C₁-C₆alkylsulfinyl, C₁-C₆alkylsulfonyl, C₁-C₆alkylsulfonamido, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₆alkoxyC₁-C₆alkylcarbonylamino, C₂-C₆alkenylcarbonylamino, C₃-C₆cycloalkyl, C₃-C₆cycloalkylaminocarbonyl, N,N-di(C₁-C₄alkyl)amino, N,N-di(C₁-C₄alkyl)aminocarbonyl, or benzyloxy; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, 3 or 4 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, 3 or 4 heteroatoms individually selected from N, O and S, and wherein the heteroaryl moiety may be optionally substituted with 1, 2, 3 or 4 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, 3 or 4 groups, which may be the same or different, represented by R¹⁰;

Preferably, R⁹ is formyl, cyano, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₄alkoxy, C₁-C₄alkoxyC₁-C₄alkyl, C₁-C₆alkoxyC₁-C₆alkoxy, C₁-C₄alkoxycarbonylC₁-C₄alkoxy, C₁-C₆alkoxycarbonyl, C₁-C₄alkylsulfanyl, C₁-C₄alkylsulfinyl, C₁-C₄alkylsulfonyl, C₁-C₄alkylsulfonamido, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₄alkoxyC₁-C₄alkylcarbonylamino, C₂-C₆alkenylcarbonylamino, C₃-C₆cycloalkyl, C₃-C₆cycloalkylaminocarbonyl, N,N-di(C₁-C₃alkyl)amino, N,N-di(C₁-C₃alkyl)aminocarbonyl, or benzyloxy; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, or 3 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, or 3 heteroatoms individually selected from N, O and S, and wherein the heteroaryl moiety may be optionally substituted with 1, 2, or 3 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, or 3 groups, which may be the same or different, represented by R¹⁰; More preferably, R⁹ is formyl, cyano, nitro, hydroxy, halogen, C₁-C₄alkyl, C₁-C₄alkoxy, C₁-C₄haloalkyl, C₁-C₄haloalkoxy, C₂-C₄alkenyloxy, cyanoC₁-C₃alkoxy, C₁-C₃alkoxyC₁-C₃alkyl, C₁-C₃alkoxyC₁-C₃alkoxy, C₁-C₃alkoxycarbonylC₁-C₃alkoxy, C₁-C₃alkoxycarbonyl, C₁-C₃alkylsulfanyl, C₁-C₃alkylsulfinyl, C₁-C₃alkylsulfonyl, C₁-C₃alkylsulfonamido, C₁-C₃alkylcarbonyl, C₁-C₃alkylaminocarbonyl, C₁-C₃alkylcarbonylamino, C₁-C₃alkylamino, C₁-C₃alkoxyC₁-C₃alkylaminocarbonyl, C₁-C₃alkoxyC₁-C₃alkylcarbonylamino, C₂-C₄alkenylcarbonylamino, C₃-C₆cycloalkyl, C₃-C₆cycloalkylaminocarbonyl, N,N-di(C₁-C₃alkyl)amino, N,N-di(C₁-C₃alkyl)aminocarbonyl, or benzyloxy; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1 or 2 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, or 3 heteroatoms individually selected from N, O and S, and wherein the heteroaryl moiety may be optionally substituted with 1 or 2 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1 or 2 groups, which may be the same or different, represented by R¹⁰;
Even more preferably, R⁹ is formyl, cyano, nitro, halogen, C₁-C₄alkyl, C₁-C₃alkoxy, C₁-C₃haloalkyl, C₁-C₂haloalkoxy, C₂-C₃alkenyloxy, cyanoC₁-C₂alkoxy, C₁-C₃alkoxyC₁-C₃alkyl, C₁-C₃alkoxyC₁-C₃alkoxy, C₁-C₃alkoxycarbonylC₁-C₃alkoxy, C₁-C₃alkoxycarbonyl, C₁-C₃alkylsulfanyl, C₁-C₃alkylsulfonyl, C₁-C₃alkylcarbonyl, C₁-C₃alkylaminocarbonyl, C₁-C₃alkylcarbonylamino, C₁-C₃alkylamino, C₁-C₃alkoxyC₁-C₃alkylaminocarbonyl, C₁-C₂alkoxyC₁-C₂alkylcarbonylamino, C₃-C₄alkenylcarbonylamino, C₃-C₄cycloalkyl, or N,N-di(C₁-C₃alkyl)amino; or
any two adjacent R⁹ groups together with the carbon atoms to which they are attached, may form a C₅-C₆cycloalkyl ring or phenyl ring, wherein the C₅-C₆cycloalkyl and phenyl moieties may be optionally substituted with a single R¹⁰ group; or
any two adjacent R⁹ groups together with the carbon atoms to which they are attached, may form a 6-membered heteroaryl ring, wherein the heteroaryl moiety is a 6-membered aromatic ring which comprises a single nitrogen atom, and wherein the heteroaryl moiety may be optionally substituted with a single R¹⁰ group; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with a single R¹⁰ group.

In a particularly preferred set of embodiments, R⁹ is acetyl, cyano, nitro, hydroxy, chloro, fluoro, bromo, methyl, ethyl, methoxy, ethoxy, isopropoxy, tertbutoxy, difluoromethyl, trifluoromethyl, difluoromethoxy, trifluoromethoxy, 2,2,2-trifluoroethoxy, allyloxy, cyanomethyl, methoxymethyl, ethoxymethyl, methoxymethoxy, methoxyethoxy, methoxycarbonyl, methylsulfanyl, methylsulfonyl, methylcarbonylamino (acetamino), propanoylamino, methoxyiminoethyl, (but-2-enoyl)amino, cyclopropyl, phenyl, pyridyl, thienyl, furanyl, thiazolyl, or pyrazinyl, and wherein the phenyl and heteroaryl moieties may be optionally substituted with a single R¹⁰ group.

R¹⁰ is halogen, nitro, C₁-C₃alkyl, or C₁-C₃alkoxy. Preferably, R¹⁰ is halogen, nitro, methyl or methoxy. More preferably, R¹⁰ is halogen or methyl. Even more preferably, R¹⁰ is chloro, fluoro, or methyl. More preferably still, R¹⁰ is chloro or fluoro, and especially preferred is when R¹⁰ is chloro.

In one set of embodiments, R¹⁰ is halogen, C₁-C₃alkyl, or C₁-C₃alkoxy. Preferably, R¹⁰ is halogen, methyl or methoxy. More preferably, R¹⁰ is halogen. Even more preferably, R¹⁰ is chloro or fluoro. More preferably still, R¹⁰ is chloro.

In a compound of formula (I) according to the present invention, preferably:
R¹ is methyl or ethyl;
R² is phenyl optionally substituted with 1 or 2 groups, which may be the same or different, represented by R⁷;
R³ is hydrogen, methyl, or ethyl;
R⁴ is hydrogen, bromo, methyl, or 4-chlorophenyl;
R⁵ is pyrazolyl, imidazolyl, or triazolyl, wherein any of the pyrazolyl, imidazolyl, and triazolyl moieties may each be optionally substituted with 1, 2, or 3 groups, which may be the same or different, represented by R⁹; or
R⁵ is benzotriazol-2-ylmethyl, indazol-1-yl, indazol-2-yl, 4,5,6,7-tetrahydroindazol-2-yl, imidazo[4,5-b]pyridin-1-yl, 6-chloropyrazolo[4,3-c]pyridin-1-yl, 3-(difluoromethyl)-6,7-dihydro-4H-pyrano[4,3-c]pyrazol-1-yl, 3-(difluoromethyl)-6,7-dihydro-4H-pyrano[4,3-c]pyrazol-2-yl, 4,6-dihydrofuro[3,4-c]pyrazol-2-yl, or 3-(trifluoromethyl)-6,7-dihydro-4H-pyrano[4,3-c]pyrazol-1-yl;
R⁶ is hydrogen or methyl;
R⁷ is cyano, nitro, halogen, or C₁-C₃alkylsulfonyl; and
R⁹ is formyl, cyano, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkoxy, C₁-C₆alkoxyC₁-C₆alkyl, C₁-C₆alkoxyC₁-C₆alkoxy, C₁-C₆alkoxycarbonylC₁-C₆alkoxy, C₁-C₆alkoxycarbonyl, C₁-C₆alkylsulfanyl, C₁-C₆alkylsulfinyl, C₁-C₆alkylsulfonyl, C₁-C₆alkylsulfonamido, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₆alkoxyC₁-C₆alkylcarbonylamino, C₂-C₆alkenylcarbonylamino, C₃-C₆cycloalkyl, C₃-C₆cycloalkylaminocarbonyl, N,N-di(C₁-C₄alkyl)amino, N,N-di(C₁-C₄alkyl)aminocarbonyl, or benzyloxy.

In one embodiment, in a compound of formula (I) according to the present invention, preferably:
R¹ is methyl or ethyl;
R² is 3,4-dichlorophenyl;
R³ is hydrogen or C₁-C₄alkyl;
R⁴ is hydrogen, bromo, or 4-chlorophenyl;
R⁵ is heteroaryl wherein the heteroaryl moiety is a 5-membered aromatic monocyclic ring comprising 2 or 3 nitrogen atoms, and wherein the heteroaryl moieties are attached to the rest of the molecule through a nitrogen atom in the heteroaryl ring, and wherein the heteroaryl moieties may each be optionally substituted with 1, 2, or 3 groups, which may be the same or different, represented by R⁹;
R⁶ is hydrogen;
R⁹ is formyl, cyano, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₄alkoxy, C₁-C₄alkoxyC₁-C₄alkyl, C₁-C₆alkoxyC₁-C₆alkoxy, C₁-C₄alkoxycarbonylC₁-C₄alkoxy, C₁-C₆alkoxycarbonyl, C₁-C₄alkylsulfanyl, C₁-C₄alkylsulfinyl, C₁-C₄alkylsulfonyl, C₁-C₄alkylsulfonamido, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₄alkoxyC₁-C₄alkylcarbonylamino, C₂-C₆alkenylcarbonylamino, C₃-C₆cycloalkyl, C₃-C₆cycloalkylaminocarbonyl, N,N-di(C₁-C₃alkyl)amino, N,N-di(C₁-C₃alkyl)aminocarbonyl, or benzyloxy; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, or 3 groups, which may be the same or different, represented by
R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, or 3 heteroatoms individually selected from N, O and S, and wherein the heteroaryl moiety may be optionally substituted with 1, 2, or 3 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, or 3 groups, which may be the same or different, represented by
R¹⁰;
R¹⁰ is halogen.

In another embodiment, in a compound of formula (I) according to the present invention, preferably:
R¹ is methyl or ethyl;
R² is 3,4-dichlorophenyl;
R³ is hydrogen, methyl, or ethyl;
R⁴ is hydrogen, bromo, methyl, or 4-chlorophenyl;
R⁵ is heteroaryl wherein the heteroaryl moiety is a 5-membered aromatic monocyclic ring comprising 1, 2, or 3 nitrogen atoms, and wherein the heteroaryl moieties are attached to the rest of the molecule through a nitrogen atom in the heteroaryl ring, and wherein the heteroaryl moieties may each be optionally substituted with 1, 2, or 3 groups, which may be the same or different, represented by R⁹;
R⁶ is hydrogen or methyl;
R⁷ is cyano, nitro, halogen, or C₁-C₃alkylsulfonyl; and
R⁹ is formyl, cyano, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkoxy, C₁-C₆alkoxyC₁-C₆alkyl, C₁-C₆alkoxyC₁-C₆alkoxy, C₁-C₆alkoxycarbonylC₁-C₆alkoxy, C₁-C₆alkoxycarbonyl, C₁-C₆alkylsulfanyl, C₁-C₆alkylsulfinyl, C₁-C₆alkylsulfonyl, C₁-C₆alkylsulfonamido, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₆alkoxyC₁-C₆alkylcarbonylamino, C₂-C₆alkenylcarbonylamino, C₃-C₆cycloalkyl, C₃-C₆cycloalkylaminocarbonyl, N,N-di(C₁-C₄alkyl)amino, N,N-di(C₁-C₄alkyl)aminocarbonyl, or benzyloxy; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, 3 or 4 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, 3 or 4 heteroatoms individually selected from N, O and S, and wherein the heteroaryl moiety may be optionally substituted with 1, 2, 3 or 4 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, 3 or 4 groups, which may be the same or different, represented by R¹⁰; and
R¹⁰ is chloro or fluoro.

Compounds of the invention can be made as shown in the following schemes, in which, unless otherwise stated, the definition of each variable is as defined above for a compound of Formula (I). General methods for the production of compounds of Formula (I) are described below. Unless otherwise stated in the text, R¹, R², R³, R⁴, R⁵ and R⁶, are as defined hereinbefore. The starting materials used for the preparation of the compounds of the invention may be purchased from usual commercial suppliers or may be prepared by known methods. The starting materials as well as the intermediates may be purified before use in the next step by state of the art methodologies such as chromatography, crystallisation, distillation and filtration.

Compounds of Formula (I) according to the present invention may be prepared from compounds of Formula (I) as shown below in Scheme 1.

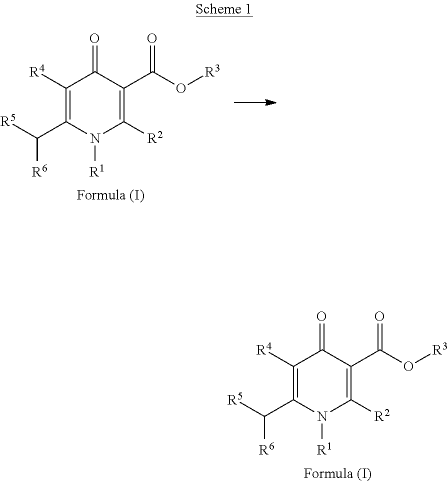

Compounds of Formula (I) wherein R³ is hydrogen may be prepared by hydrolysis of a compound of Formula (I) wherein R³ is not hydrogen, but any other R³ group as defined above, with a suitable base (such as sodium hydroxide or lithium hydroxide) or with a suitable acid (such as trifluoroacetic acid, hydrochloric acid, formic acid or sulfuric acid) in a suitable solvent (such as methanol, ethanol, dichloromethane, chloroform, ethyl acetate or tetrahydrofuran) with an optional co-solvent (such as water). In the cases where a base was used, the product was obtained following acidification with a suitable acid (such as hydrochloric acid).

Alternatively, compounds of Formula (I) wherein R³ is hydrogen may be prepared by hydrolysis of a compound of Formula (I) wherein R³ is not hydrogen, but any other R³ group as defined above in the presence of a water stable Lewis acid such as scandium(III) trifluoromethanesulfonate, in a suitable solvent (such as tetrahydrofuran) in the presence of water.

Compounds of Formula (I) may additionally be prepared by methods as described below.

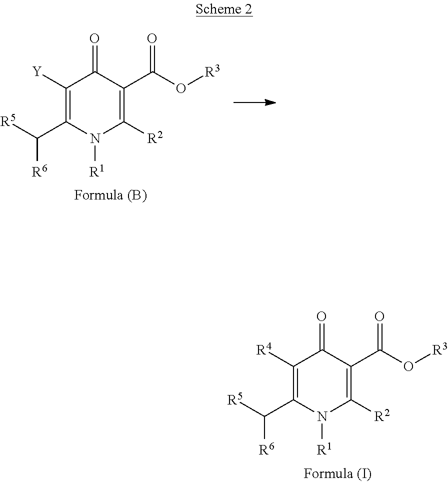

Compound of Formula (B) wherein Y═Br, Cl or I may be converted to a compound of Formula (I) wherein R⁴ is hydrogen by reduction using hydrogen gas in the presence of a suitable catalyst (such as palladium or platinum on carbon) in a suitable organic solvent (such as methanol, ethanol or ethyl acetate) optionally in the presence of an organic acid (such as acetic acid).

Alternately, compounds of Formula (B) wherein Y is Br may be converted to a compound of Formula (I) wherein R⁴ is hydrogen under transfer hydrogenation conditions using a suitable hydrogen source (such as ammonium formate) in the presence of a suitable catalyst (such as dichlorobis(triphenylphosphine)palladium(II)) in an organic solvent (such as acetonitrile) at elevated temperature.

In another transformation, compounds of Formula (I) wherein R⁴ is C₁-C₆alkyl may be obtained from reaction of a compound of Formula (B) where Y is I or Br by reaction with an alkyl boronic acid (such as methyl boronic acid) under Suzuki-Miyaura cross-coupling conditions in analogy to literature conditions.

Typically the reaction is performed by reaction of a compound of Formula (B) with R⁴-boronic acid or boroxine in the presence of a suitable catalyst (such as dichlorobis(triphenylphosphine)palladium(II), tetrakis(triphenylphosphine)palladium), tris(dibenzylideneacetone)dipalladium, or dichloro(1,1′-bis(diphenylphosphanyl)ferrocene) palladium(II) dichloromethane adduct) or palladium diacetate optionally with a ligand (such as 2-dicyclohexylphosphino-2′,6′-dimethoxybiphenyl) in the presence of a base (such as potassium or cesium carbonate or tripotassium phosphate) in a suitable organic solvent (such as 1,4-dioxane, toluene or tetrahydrofuran) optionally in the presence of water at elevated temperature.

In another transformation, compounds of Formula (I) wherein R⁴ is phenyl may be obtained from reaction of a compound of Formula (B) where Y is I or Br by reaction with an phenyl boronic acid or boroxine under Suzuki-Miyaura cross-coupling conditions in analogy to literature conditions. This is shown above in Scheme 2.

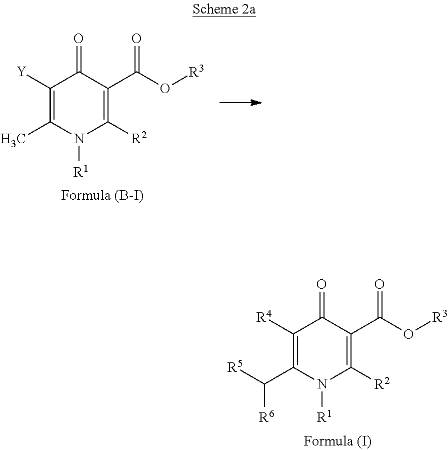

In an alternative transformation, compounds of Formula (I) wherein R⁴ and R⁶ are hydrogen and R³ is hydrogen or any other R³ group as defined above, may be obtained from reaction of a compound of Formula (B-1) where Y is I or Brand by reaction with a compound of formula R⁵—H (wherein the compound of formula R⁵—H is an azole such as a pyrazole, imidazole or triazole) in the presence of stoichiometric copper (such as copper (I) iodide), with a suitable ligand (such as 1,10-phenanthroline) and a suitable base (such as potassium carbonate) in an organic solvent (such as N,N-dimethylacetamide) at elevated temperature. Azoles are commercially available or may be prepared by methods reported in the literature (Tetrahedron 2012, 68, 3165-3171). This is shown above in Scheme 2a.

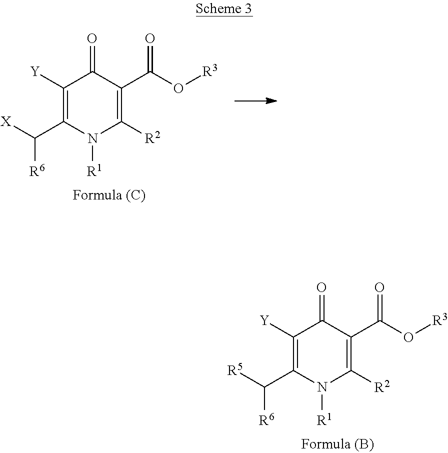

Compounds of Formula (C) wherein Y is Br, C₁, I or H, and X is Br may be converted to a compound of Formula (B) by a nucleophilic substitution reaction with nucleophile R⁵—H (such as a pyrazole, imidazole, or triazole) in the presence of a base (such as potassium carbonate or sodium hydride) in a suitable solvent (such as acetonitrile, N,N-dimethylformamide or tetrahydrofuran) at ambient temperature or elevated temperature. Azoles are commercially available or may be prepared by methods familiar to persons skilled in the art. This is shown above in Scheme 3.

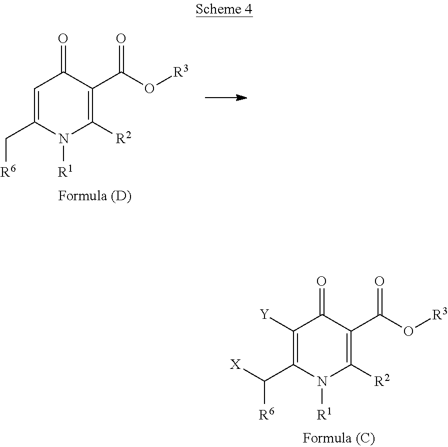

Compounds of Formula (C) wherein Y and X are the same and X is Br, Cl or I may be prepared by treatment of compounds of Formula (D) with a suitable halogenating agent (such as N-bromo-, N-chloro- or N-iodo-succinimide) in a suitable solvent (such as acetonitrile) optionally with an additional acid (such as trifluoroacetic acid) at ambient temperature or elevated temperature. This is shown above in Scheme 4.

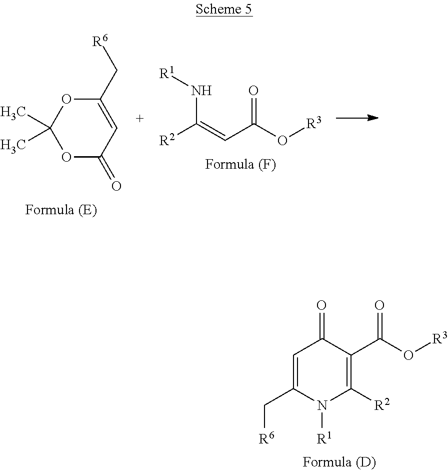

Compounds of Formula (D) may be prepared by reacting a compound of Formula (E) with a compound of Formula (F) optionally in the presence of a solvent (such as dioxane) at an elevated temperature (for example 120° C.). Compounds of Formula (E) are commercially available or may be prepared by methods familiar to persons skilled in the art. This is shown above in Scheme 5.

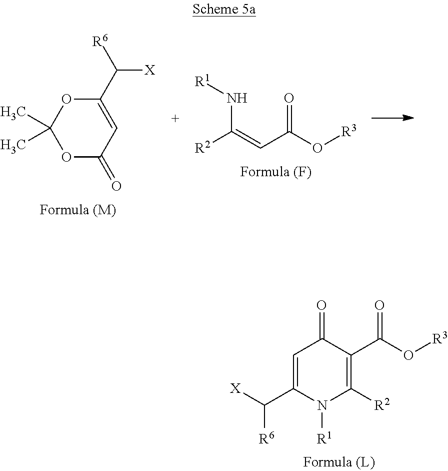

In a different transformation, compounds of Formula (L) where X is Br may be prepared by reacting a compound of Formula (M) with a compound of Formula (F) optionally in the presence of a solvent (such as dioxane) at an elevated temperature (for example 120° C.). Compounds of Formula (M) are commercially available or may be prepared by methods familiar to persons skilled in the art. This is shown above in Scheme 5a.

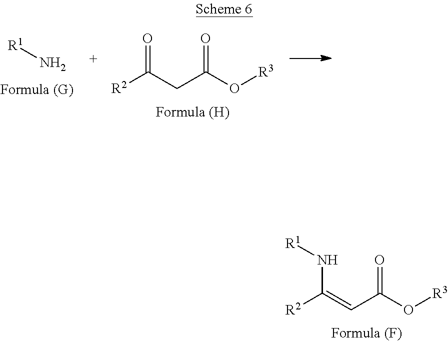

Compounds of Formula (F) maybe be prepared from reaction of β-keto esters of Formula (G) with an amine salt. The amine salts can be prepared in situ by acidification of amines of Formula (H) with a suitable acid (such as acetic acid). These amine salts may then be reacted with compounds of Formula G in a suitable solvent (such as toluene) in the presence of an acid (such as acetic acid) and a drying agent (such as 4 Å molecular sieves). Compounds of Formula (G) are commercially available or may be prepared using conditions described below. Compounds of Formula (H) are commercially available or may be prepared by methods reported in the literature. This is shown above in Scheme 6.

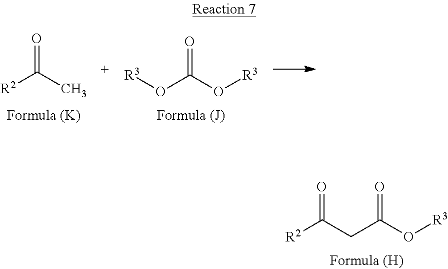

Compounds of Formula (H) may be prepared by treatment of ketones of Formula (K) with a base (such as sodium hydride) in the presence of dialkyl carbonates of Formula (J) (such as dimethyl carbonate). Compounds of Formula (K) and Formula (J) are commercially available or may be prepared by methods familiar to persons skilled in the art. This is shown above in Scheme 7.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising useful (crop) plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much improved selectivity compared to know, structurally similar compounds. Generally, the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I).

The rates of application of compounds of Formula (I) may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

The term “useful plants” is to be understood as also including useful plants that have been rendered tolerant to herbicides like bromoxynil or classes of herbicides such as, for example, 4-Hydroxyphenylpyruvate dioxygenase (HPPD) inhibitors, ALS inhibitors, for example primisulfuron, prosulfuron and trifloxysulfuron, 5-enol-pyrovyl-shikimate-3-phosphate-synthase (EPSPS) inhibitors, glutamine synthetase (GS) inhibitors or protoporphyrinogen-oxidase (PPO) inhibitors as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 corn borer (CB) trait), Agrisure® RW (corn rootworm trait) and Protecta®.

Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compounds of Formula (I) (or compositions comprising such) can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

Compounds of Formula (I) may be used in unmodified form or, preferably, together with the adjuvants conventionally employed in the art of formulation to provide herbicidal compositions, using formulation adjuvants, such as carriers, solvents and surface-active agents (SAA). The invention therefore further provides a herbicidal composition, comprising at least one compound Formula (I) and an agriculturally acceptable carrier and optionally an adjuvant. An agricultural acceptable carrier is for example a carrier that is suitable for agricultural use. Agricultural carriers are well known in the art.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates. Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols. Suitable suspending agents include hydrophilic colloids (such as polysaccharides, polyvinylpyrrolidone or sodium carboxymethylcellulose) and swelling clays (such as bentonite or attapulgite).

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, fomesafen flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, [(E)-[2-(trifluoromethyl)phenyl]methyleneamino]2,6-bis[(4,6-dimethoxypyrimidin-2-yl)oxy]benzoate, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester,4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl-2-[[3-[[3-chloro-5-fluoro-6-[3-methyl-2,6-dioxo-4-(trifluoromethyl)pyrimidin-1-yl]-2-pyridyl]oxy]acetate,6-chloro-4-(2,7-dimethyl-1-naphthyl)-5-hydroxy-2-methyl-pyridazin-3-one, tetrahydrofuran-2-ylmethyl (2R)-2-[(4-amino-3,5-dichloro-6-fluoro-2-pyridyl)oxy]propanoate, (2R)-2-[(4-amino-3,5-dichloro-6-fluoro-2-pyridyl)oxy]propanoic acid, tetrahydrofuran-2-ylmethyl 2-[(4-amino-3,5-dichloro-6-fluoro-2-pyridyl)oxy]propanoate, 2-[(4-amino-3,5-dichloro-6-fluoro-2-pyridyl)oxy]propanoic acid, 2-fluoro-N-(5-methyl-1,3,4-oxadiazol-2-yl)-3-[(R)-propylsulfinyl]-4-(trifluoromethyl)benzamide, 2-fluoro-N-(5-methyl-1,3,4-oxadiazol-2-yl)-3-propylsulfinyl-4-(trifluoromethyl)benzamide, (2-fluorophenyl)methyl 6-amino-5-chloro-2-(4-chloro-2-fluoro-3-methoxy-phenyl)pyrimidine-4-carboxylate, 6-amino-5-chloro-2-(4-chloro-2-fluoro-3-methoxy-phenyl)pyrimidine-4-carboxylic acid, 3-(3-chlorophenyl)-6-(5-hydroxy-1,3-dimethyl-pyrazole-4-carbonyl)-1,5-dimethyl-quinazoline-2,4-dione and [4-[3-(3-chlorophenyl)-1,5-dimethyl-2,4-dioxo-quinazoline-6-carbonyl]-2,5-dimethyl-pyrazol-3-yl]N,N-diethylcarbamate, methyl 2-[(E)-[2-chloro-4-fluoro-5-[3-methyl-2,6-dioxo-4-(trifluoromethyl)pyrimidin-1-yl]phenyl]methyleneamino]oxypropanoate and methyl (2R)-2-[(E)-[2-chloro-4-fluoro-5-[3-methyl-2,6-dioxo-4-(trifluoromethyl)pyrimidin-1-yl]phenyl]methyleneamino]oxypropanoate.

The mixing partners of the compound of Formula (I) may also be in the form of esters or salts, as mentioned e.g. in The Pesticide Manual, Sixteenth Edition, British Crop Protection Council, 2012. The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil. Particularly preferred are mixtures of a compound of Formula (I) with cyprosulfamide, isoxadifen-ethyl, cloquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The compounds of Formula (I) are normally used in the form of agrochemical compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants. Germinated plants and young plants which are to be transplanted after germination or after emergence from the soil, may also be mentioned. These young plants may be protected before transplantation by a total or partial treatment by immersion. Preferably “plant propagation material” is understood to denote seeds.

Pesticidal agents referred to herein using their common name are known, for example, from “The Pesticide Manual”, 15th Ed., British Crop Protection Council 2009.

The compounds of formula (I) may be used in unmodified form or, preferably, together with the adjuvants conventionally employed in the art of formulation. To this end, they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g., for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

The compounds of Formula (I) are normally used in the form of compositions and can be applied to the crop area or plant to be treated, simultaneously or in succession with further compounds. These further compounds can be, e.g., fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compound of Formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) together with component (B) and (C), and optionally other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

The tables below illustrate examples of individual compounds of Formula (I) according to the invention:

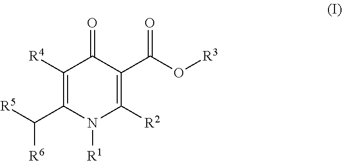

TABLE 1
Individual compounds of Formula (I) according to the invention

| Cpd | | | | |
|---|---|---|---|---|
| No. | R¹ | R² | R⁵ | R⁶ |
| 001 | Et | (3-chloro-4-methylsulfonyl- | [3-(trifluoromethyl)pyrazol-1-yl] | H |
| | | phenyl) | | |
| 002 | Et | (4-chloro-3-nitro-phenyl) | [3-(trifluoromethyl)pyrazol-1-yl] | H |
| 003 | Et | (4-chloro-3-cyano-phenyl) | [3-(trifluoromethyl)pyrazol-1-yl] | H |
| 004 | Et | (3-chloro-4-methylsulfonyl- | [3-(trifluoromethyl)pyrazol-1-yl] | H |
| | | phenyl) | | |
| 005 | Et | (3-chloro-4-nitro-phenyl) | [3-(trifluoromethyl)pyrazol-1-yl] | H |
| 006 | Et | (3-chloro-4-cyano-phenyl) | [3-(trifluoromethyl)pyrazol-1-yl] | H |
| 007 | Et | (3-chloro-4-methylsulfonyl- | (3-chloropyrazol-1-yl) | H |
| | | phenyl) | | |
| 008 | Et | (4-chloro-3-nitro-phenyl) | (3-chloropyrazol-1-yl) | H |
| 009 | Et | (4-chloro-3-cyano-phenyl) | (3-chloropyrazol-1-yl) | H |
| 010 | Et | (3-chloro-4-methylsulfonyl- | (3-chloropyrazol-1-yl) | H |
| | | phenyl) | | |
| 011 | Et | (3-chloro-4-nitro-phenyl) | (3-chloropyrazol-1-yl) | H |
| 012 | Et | (3-chloro-4-cyano-phenyl) | (3-chloropyrazol-1-yl) | H |
| 013 | Me | (3-chloro-4-methylsulfonyl- | [3-(trifluoromethyl)pyrazol-1-yl] | H |
| | | phenyl) | | |
| 014 | Me | (4-chloro-3-nitro-phenyl) | [3-(trifluoromethyl)pyrazol-1-yl] | H |
| 015 | Me | (4-chloro-3-cyano-phenyl) | [3-(trifluoromethyl)pyrazol-1-yl] | H |
| 016 | Me | (3-chloro-4-methylsulfonyl- | [3-(trifluoromethyl)pyrazol-1-yl] | H |
| | | phenyl) | | |
| 017 | Me | (3-chloro-4-nitro-phenyl) | [3-(trifluoromethyl)pyrazol-1-yl] | H |
| 018 | Me | (3-chloro-4-cyano-phenyl) | [3-(trifluoromethyl)pyrazol-1-yl] | H |
| 019 | Me | (3-chloro-4-methylsulfonyl- | (3-chloropyrazol-1-yl) | H |
| | | phenyl) | | |
| 020 | Me | (4-chloro-3-nitro-phenyl) | (3-chloropyrazol-1-yl) | H |
| 021 | Me | (4-chloro-3-cyano-phenyl) | (3-chloropyrazol-1-yl) | H |
| 022 | Me | (3-chloro-4-methylsulfonyl- | (3-chloropyrazol-1-yl) | H |
| | | phenyl) | | |
| 023 | Me | (3-chloro-4-nitro-phenyl) | (3-chloropyrazol-1-yl) | H |
| 024 | Me | (3-chloro-4-cyano-phenyl) | (3-chloropyrazol-1-yl) | H |
| 025 | Et | (3,4-dichlorophenyl) | (3-propoxypyrazol-1-yl) | H |
| 026 | Et | (3,4-dichlorophenyl) | (3-ethoxypyrazol-1-yl) | H |
| 027 | Et | (3,4-dichlorophenyl) | [3-(2-methoxyethoxy)pyrazol-1-yl] | H |
| 028 | Et | (3,4-dichlorophenyl) | (3-allyloxypyrazol-1-yl) | H |
| 029 | Et | (3,4-dichlorophenyl) | [3-(2-cyanoethoxy)pyrazol-1-yl] | H |
| 030 | Et | (3,4-dichlorophenyl) | (3-allyloxypyrazol-1-yl) | H |
| 031 | Et | (3,4-dichlorophenyl) | [3-(2-methoxy-2-oxo-ethoxy)pyrazol-1-yl] | H |
| 032 | Et | (3,4-dichlorophenyl) | (3-acetamidopyrazol-1-yl) | H |
| 033 | Et | (3,4-dichlorophenyl) | [3-[(2-methoxyacetyl)amino]pyrazol-1-yl] | H |
| 034 | Et | (3,4-dichlorophenyl) | [3-(but-3-enoylamino)pyrazol-1-yl] | H |
| 035 | Et | (3,4-dichlorophenyl) | [3-(ethylcarbamoyl)pyrazol-1-yl] | H |
| 036 | Et | (3,4-dichlorophenyl) | [3-(2-methoxyethylcarbamoyl)pyrazol-1- | H |
| | | | yl] | |
| 037 | Et | (3,4-dichlorophenyl) | [5-(2-methoxyethoxy)-3-methyl-pyrazol- | H |
| | | | 1-yl] | |
| 038 | Et | (3,4-dichlorophenyl) | [5-(2-methoxyethoxy)-3- | H |
| | | | (trifluoromethyl)pyrazol-1-yl] | |
| 039 | Et | (3,4-dichlorophenyl) | (5-ethoxy-3-methyl-pyrazol-1-yl) | H |
| 040 | Et | (3,4-dichlorophenyl) | [5-ethoxy-3-(trifluoromethyl)pyrazol-1-yl] | H |
| 041 | Et | (3,4-dichlorophenyl) | [3-(propylamino)pyrazol-1-yl] | H |
| 042 | Et | (3,4-dichlorophenyl) | [3-[methyl(propyl)amino]pyrazol-1-yl] | H |
| 043 | ⁿPr | (3,4-dichlorophenyl) | [3-(trifluoromethyl)pyrazol-1-yl] | H |
| 044 | allyl | (3,4-dichlorophenyl) | [3-(trifluoromethyl)pyrazol-1-yl] | H |
| 045 | OMe | (3,4-dichlorophenyl) | [3-(trifluoromethyl)pyrazol-1-yl] | H |
| 046 | prop-2-ynyl | (3,4-dichlorophenyl) | [3-(trifluoromethyl)pyrazol-1-yl] | H |
| 047 | 2-methoxyethyl | (3,4-dichlorophenyl) | [3-(trifluoromethyl)pyrazol-1-yl] | H |
| 048 | ⁿPr | (3,4-dichlorophenyl) | (3-chloropyrazol-1-yl) | H |
| 049 | allyl | (3,4-dichlorophenyl) | (3-chloropyrazol-1-yl) | H |
| 050 | OMe | (3,4-dichlorophenyl) | (3-chloropyrazol-1-yl) | H |
| 051 | prop-2-ynyl | (3,4-dichlorophenyl) | (3-chloropyrazol-1-yl) | H |
| 052 | 2-methoxyethyl | (3,4-dichlorophenyl) | (3-chloropyrazol-1-yl) | H |
| 053 | Et | (3,4-dichlorophenyl) | [3-(trifluoromethyl)pyrazol-1-yl] | Me |
| 054 | Et | (3,4-dichlorophenyl) | (3-chloropyrazol-1-yl) | Me |

Table A-1 provides 54 compounds A-1.001 to A.1.054 of Formula (I) wherein R³ and R⁴ are both hydrogen, and R¹, R², R⁵, and R⁶ are as defined in Table 1.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

| Emulsifiable concentrate | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

| Extruder granules | |
|---|---|
| Active ingredient [compound of formula (I)] | 15% |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

| Coated granules | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

Suspension concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution | 0.5% |
| in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinyl alcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

### EXAMPLES

The following non-limiting examples provide specific synthesis methods for representative compounds of the present invention, as referred to in Table 2 below.

### List of Abbreviations

brd=broad doublet, brm=broad multiplet, brs=broad singlet, ° C.=degrees Celsius, d=doublet, dd=doublet of doublets, DMSO=dimethyl sulfoxide, HPLC=high performance liquid chromatography, LCMS=liquid chromatography mass spectrometry, M=molar, m=multiplet, MHz=megahertz, q=quartet, s=singlet, t=triplet.

### Example 1: Synthesis of 6-[(3-chloropyrazol-1-yl)methyl]-2-(3,4-dichlorophenyl)-1-ethyl-4-oxo-pyridine-3-carboxylic acid (Compound 4)

### Step 1: Synthesis of methyl 3-(3,4-dichlorophenyl)-3-oxo-propanoate

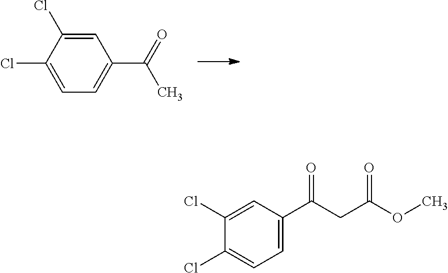

To a stirred solution of 1-(3,4-dichlorophenyl)ethanone (5.00 g, 26.5 mmol) and dimethyl carbonate (40 mL, 466 mmol) under nitrogen and cooled to 0° C. was added portionwise sodium hydride (3.17 g, 79.5 mmol, 60 mass %). The reaction mixture was allowed to warm to room temperature and stirred for 16 hours. Overnight the reaction mixture became a solid paste which was not possible to stir. More dimethyl carbonate (10 mL) was added in an attempt to create a mobile slurry for quenching. The reaction mixture was cooled to 0° C. and quenched by addition of water (25 mL) under nitrogen. The reaction mixture was acidified to pH3 by addition of 2 M aqueous hydrochloric acid and then extracted with ethyl acetate. The organic extract was dried over magnesium sulfate and evaporated to dryness under reduced pressure. The crude residue was purified by flash chromatography on silica gel using a gradient of 0-15% ethyl acetate in isohexane as eluent to give methyl 3-(3,4-dichlorophenyl)-3-oxo-propanoate (mixture of tautomers) as a colourless liquid.

Enol: ¹H NMR (400 MHz, chloroform) δ=12.47 (s, 1H), 7.87 (d, 1H), 7.59 (m, 3H), 7.49 (d, 1H), 5.65 (s, 1H), 3.82 (s, 3H)

Keto: ¹H NMR (400 MHz, chloroform) δ=8.03 (d, 1H), 7.77 (m, 1H), 7.58 (d, 2H), 3.97 (s, 2H), 3.76 (s, 3H)

### Step 2: Synthesis of ethyl (Z)-3-(3,4-dichlorophenyl)-3-(ethylamino)prop-2-enoate

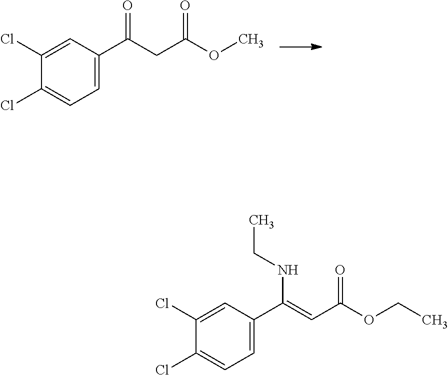 [SMILES: COC(=O)CC(=O)c1ccc(Cl)c(Cl)c1 ; CCN/C(=C\C(=O)OCC)c1ccc(Cl)c(Cl)c1]

To a stirred solution of ethyl 3-(3,4-dichlorophenyl)-3-oxo-propanoate (1.65 g, 6.319 mmol) in toluene (11 mL) was added ethylammonium;acetate (19.0 mmol) and acetic acid (6.32 mmol). The orange reaction mixture was heated at reflux for 6 hours. The cooled reaction mixture was diluted with ethyl acetate and was washed with saturated aqueous sodium bicarbonate solution. The phases were separated and the aqueous was extracted with ethyl acetate (×3). The organic extract was dried over magnesium sulfate and evaporated to dryness under reduced pressure. The crude residue was purified by flash chromatography on silica gel using a gradient of 0-10% ethyl acetate in cyclohexane as eluent to give ethyl (Z)-3-(3,4-dichlorophenyl)-3-(ethylamino)prop-2-enoate as a colourless oil.

¹H NMR (400 MHz, chloroform) δ=7.49-7.43 (m, 2H), 7.23-7.15 (m, 1H), 4.58-4.50 (m, 1H), 4.17-4.08 (m, 2H), 3.12-2.91 (m, 2H), 1.31-1.22 (m, 3H), 1.15-1.06 (m, 3H)

### Step 3: Synthesis of ethyl 2-(3,4-dichlorophenyl)-1-ethyl-6-methyl-4-oxo-pyridine-3-carboxylate

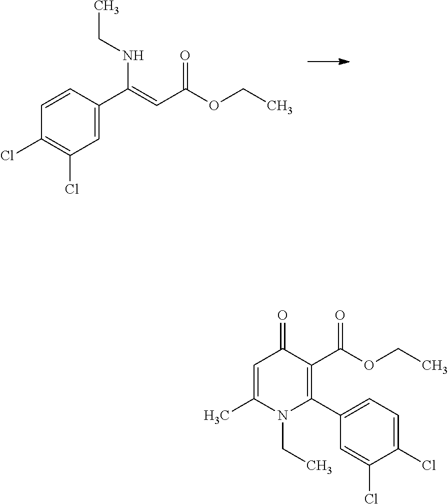

A stirred mixture of ethyl (Z)-3-(3,4-dichlorophenyl)-3-(ethylamino)prop-2-enoate (0.583 g, 2.02 mmol) and 2,2,6-trimethyl-1,3-dioxin-4-one (4.05 mmol) under nitrogen were heated at 120° C. for 3 hours. The cooled reaction mixture was diluted with ethyl acetate and evaporated to dryness under reduced pressure. The crude residue was purified by flash chromatography on silica gel using a gradient of 0-10% methanol in dichloromethane as eluent to give ethyl 2-(3,4-dichlorophenyl)-1-ethyl-6-methyl-4-oxo-pyridine-3-carboxylate as a brown solid.

¹H NMR (400 MHz, chloroform) δ=7.59 (d, 1H), 7.53 (d, 1H), 7.29 (dd, 1H), 6.39 (s, 1H), 4.04-3.95 (m, 2H), 3.77 (q, 2H), 2.44 (s, 3H), 1.14 (t, 3H), 0.97 (t, 3H)

### Step 4: Synthesis of ethyl 2-(3,4-dichlorophenyl)-1-ethyl-5-iodo-6-methyl-4-oxo-pyridine-3-carboxylate

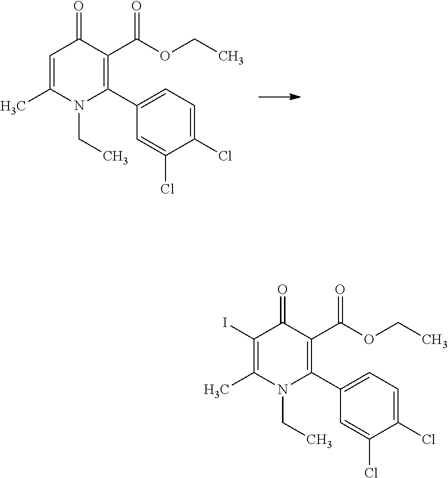 [SMILES: CCOC(=O)c1c(-c2ccc(Cl)c(Cl)c2)n(CC)c(C)cc1=O ; CCOC(=O)c1c(-c2ccc(Cl)c(Cl)c2)n(CC)c(C)c(I)c1=O]

To a solution of ethyl 2-(3,4-dichlorophenyl)-1-ethyl-6-methyl-4-oxo-pyridine-3-carboxylate (80 g, 226 mmol) in acetonitrile (800 mL) at 80° C. was added 2,2,2-trifluoroacetic acid (5.2 mL, 67.8 mmol) followed by portionwise addition of 1-iodopyrrolidine-2,5-dione (224 mmol). The reaction mixture was stirred at room temperature for 3 hours. The cooled reaction mixture was quenched by addition of saturated aqueous sodium hydrogen carbonate solution and extracted with dichloromethane (×3). The combined organic extracts were washed with saturated sodium thiosulfate solution then brine, dried over magnesium sulfate, filtered and evaporated under reduced pressure. The crude residue was purified by flash chromatography on silica gel using a gradient of 40-45% ethyl acetate in cyclohexane as eluent to give ethyl 2-(3,4-dichlorophenyl)-1-ethyl-5-iodo-6-methyl-4-oxo-pyridine-3-carboxylate as a white solid.

¹H NMR (400 MHz, chloroform) δ=7.57 (d, J=8.3 Hz, 1H), 7.50 (d, J=2.0 Hz, 1H), 7.24 (dd, J=2.1, 8.2 Hz, 1H), 4.10-3.97 (m, 2H), 3.89 (q, J=7.1 Hz, 2H), 2.87 (s, 3H), 1.17 (t, J=7.2 Hz, 3H), 1.03 (t, J=7.2 Hz, 3H)

### Step 5: Synthesis of ethyl 6-[(3-chloropyrazol-1-yl)methyl]-2-(3,4-dichlorophenyl)-1-ethyl-4-oxo-pyridine-3-carboxylate

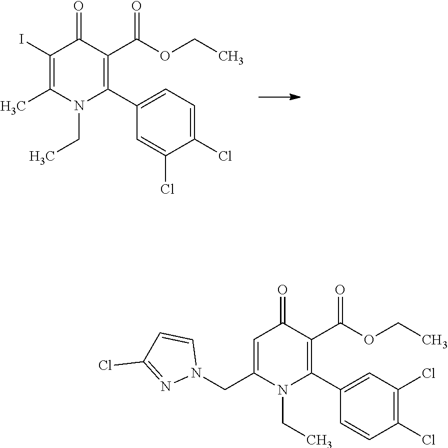 [SMILES: CCOC(=O)c1c(-c2ccc(Cl)c(Cl)c2)n(CC)c(C)c(I)c1=O ; CCOC(=O)c1c(-c2ccc(Cl)c(Cl)c2)n(CC)c(Cn2ccc(Cl)n2)cc1=O]

A mixture of ethyl 2-(3,4-dichlorophenyl)-1-ethyl-5-iodo-6-methyl-4-oxo-pyridine-3-carboxylate (0.200 g, 0.417 mmol), 3-chloro-1H-pyrazole (0.154 g, 1.50 mmol), copper(I) iodide (0.0801 g, 0.417 mmol), 1,10-phenanthroline (0.155 g, 0.833 mmol) and potassium carbonate (0.116 g, 0.833 mmol) were added to a pressure release screw capped vial. The contents were purged with nitrogen (×3) before addition of degassed N,N-dimethylacetamide (2.8 mL). The reaction mixture was heated at 120° C. for 18 hours overnight. The cooled reaction mixture was diluted with water and acidified to pH 1 by addition of 2M aqueous hydrochloric acid before being extracted with dichloromethane. The crude residue was purified by mass-directed reverse phase HPLC to give ethyl 6-[(3-chloropyrazol-1-yl)methyl]-2-(3,4-dichlorophenyl)-1-ethyl-4-oxo-pyridine-3-carboxylate as an orange gum.

¹H NMR (400 MHz, chloroform) δ=7.59 (d, 1H), 7.56-7.52 (m, 2H), 7.29 (d, 1H), 6.58 (brs, 1H), 6.36-6.25 (m, 1H), 5.31 (s, 2H), 4.08-3.93 (m, 4H), 1.09 (t, 3H), 0.99 (t, 3H)

### Step 6: Synthesis of 6-[(3-chloropyrazol-1-yl)methyl]-2-(3,4-dichlorophenyl)-1-ethyl-4-oxo-pyridine-3-carboxylic acid (Compound 4)

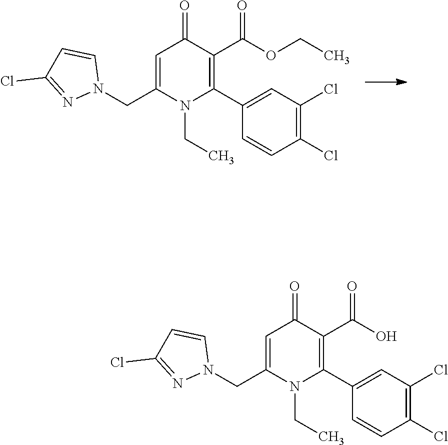 [SMILES: CCOC(=O)c1c(-c2ccc(Cl)c(Cl)c2)n(CC)c(Cn2ccc(Cl)n2)cc1=O ; CCn1c(Cn2ccc(Cl)n2)cc(=O)c(C(=O)O)c1-c1ccc(Cl)c(Cl)c1]

To a solution of ethyl 6-[(3-chloropyrazol-1-yl)methyl]-2-(3,4-dichlorophenyl)-1-ethyl-4-oxo-pyridine-3-carboxylate (0.064 g, 0.14 mmol) in a mixture of methanol (1.15 mL) and water (0.32 mL) was added lithium hydroxide (0.013 g, 0.56 mmol). The resultant solution was heated at reflux 1 hour. The cooled reaction mixture was diluted with dichloromethane and water and acidified to pH 1 by addition of 2M aqueous hydrochloric acid before extraction into dichloromethane. The organic extracts were evaporated under reduced pressure to give 6-[(3-chloropyrazol-1-yl)methyl]-2-(3,4-dichlorophenyl)-1-ethyl-4-oxo-pyridine-3-carboxylic acid as an orange gum.

¹H NMR (400 MHz, chloroform) δ=7.61 (d, 1H), 7.56 (d, 1H), 7.42 (d, 1H), 7.17 (dd, 1H), 6.32 (d, 1H), 6.24 (s, 1H), 5.36 (s, 2H), 3.96 (q, 2H), 1.21 (t, 3H)

### Example 2: Synthesis of 2-(3,4-dichlorophenyl)-1-ethyl-4-oxo-6-[[4-(trifluoromethyl)imidazol-1-yl]methyl]pyridine-3-carboxylic acid (Compound 10)

### Step 1: Synthesis of ethyl 2-(3,4-dichlorophenyl)-1-ethyl-4-oxo-6-[[4-(trifluoromethyl)imidazol-1-yl]methyl]pyridine-3-carboxylate and ethyl 2-(3,4-dichlorophenyl)-1-ethyl-4-oxo-6-[[5-(trifluoromethyl)imidazol-1-yl]methyl]pyridine-3-carboxylate

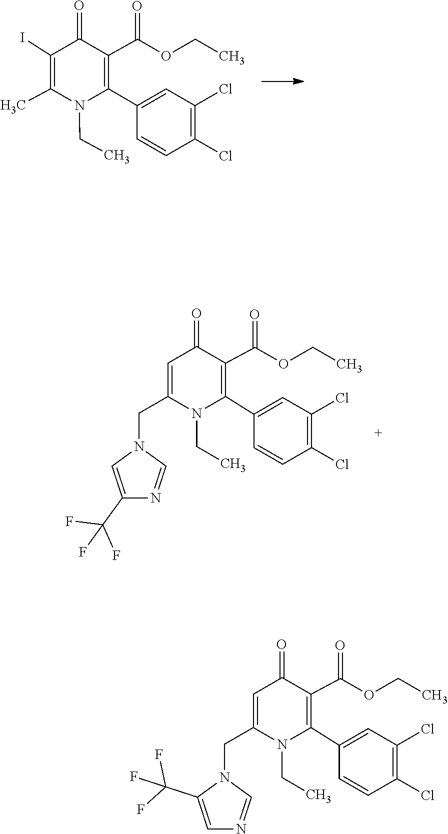 [SMILES: CCOC(=O)c1c(-c2ccc(Cl)c(Cl)c2)n(CC)c(C)c(I)c1=O ; CCOC(=O)c1c(-c2ccc(Cl)c(Cl)c2)n(CC)c(Cn2cnc(C(F)(F)F)c2)cc1=O ; CCOC(=O)c1c(-c2ccc(Cl)c(Cl)c2)n(CC)c(Cn2cncc2C(F)(F)F)cc1=O]

A mixture of ethyl 2-(3,4-dichlorophenyl)-1-ethyl-5-iodo-6-methyl-4-oxo-pyridine-3-carboxylate (0.200 g, 0.417 mmol), 5-(trifluoromethyl)-1H-imidazole (0.204 g, 1.50 mmol), copper(I) iodide (0.0801 g, 0.417 mmol), 1,10-phenanthroline (0.155 g, 0.833 mmol) and potassium carbonate (0.116 g, 0.833 mmol) were added to a pressure release screw capped vial followed by N,N-dimethylacetamide (2.8 mL). The reaction mixture was heated at 120° C. for 18 hours overnight. The cooled reaction mixture was poured into saturated aqueous ammonium chloride solution and diluted with ethyl acetate. Water was added to dissolve the solid precipitates in the aqueous phase. The mixture was filtered through diatomaceous earth and the organic phase was washed sequentially with saturated aqueous ammonium chloride, water then brine, then dried over magnesium sulfate and concentrated under reduced pressure. The crude residue was purified by mass-directed reverse phase HPLC to give ethyl 2-(3,4-dichlorophenyl)-1-ethyl-4-oxo-6-[[4-(trifluoromethyl)imidazol-1-yl]methyl]pyridine-3-carboxylate (0.017 g, 0.035 mmol, 8%) and ethyl 2-(3,4-dichlorophenyl)-1-ethyl-4-oxo-6-[[5-(trifluoromethyl)imidazol-1-yl]methyl]pyridine-3-carboxylate.

Isomer 1: ¹H NMR (400 MHz, chloroform) δ=7.70 (brs, 1H), 7.61 (d, 1H), 7.55 (d, 1H), 7.41 (brs, 1H), 7.29 (dd, 1H), 6.24 (brs, 1H), 5.27 (brs, 2H), 4.06-3.94 (m, 2H), 3.77 (brd, 2H), 1.08 (brt, 3H), 0.97 (t, 3H)

Isomer 2: ¹H NMR (400 MHz, chloroform) δ=8.05 (brm, 1H), 7.63-7.56 (m, 3H), 7.32 (dd, 1H), 6.01 (s, 1H), 5.38 (s, 2H), 4.09-3.98 (m, 2H), 3.79 (q, 2H), 1.26-1.11 (m, 3H), 0.99 (t, 3H)

### Step 2: Synthesis of 2-(3,4-dichlorophenyl)-1-ethyl-4-oxo-6-[[4-(trifluoromethyl)imidazol-1-yl]methyl]pyridine-3-carboxylic acid

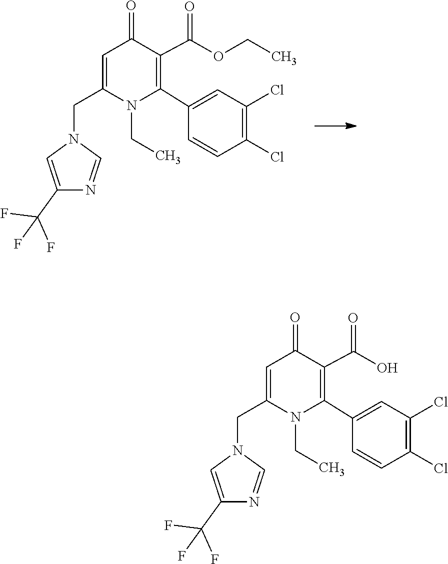 [SMILES: CCOC(=O)c1c(-c2ccc(Cl)c(Cl)c2)n(CC)c(Cn2cnc(C(F)(F)F)c2)cc1=O ; CCn1c(Cn2cnc(C(F)(F)F)c2)cc(=O)c(C(=O)O)c1-c1ccc(Cl)c(Cl)c1]

To a solution of ethyl 2-(3,4-dichlorophenyl)-1-ethyl-4-oxo-6-[[4-(trifluoromethyl)imidazol-1-yl]methyl]pyridine-3-carboxylate (0.015 g, 0.031 mmol) in a mixture of methanol (0.27 mL) and water (0.075 mL) was added lithium hydroxide (0.0029 g, 0.12 mmol). The resultant solution was heated at reflux for 1.2 hours. The cooled reaction mixture was diluted with dichloromethane and water and acidified to pH 1 by addition of 2 M aqueous hydrochloric acid before extraction into dichloromethane. The organic extracts were concentrated under reduced pressure to give 2-(3,4-dichlorophenyl)-1-ethyl-4-oxo-6-[[4-(trifluoromethyl)imidazol-1-yl]methyl]pyridine-3-carboxylic acid as a white solid.

¹H NMR (400 MHz, methanol) 6=8.07-8.00 (m, 1H), 7.89-7.84 (m, 1H), 7.77-7.71 (m, 1H), 7.68-7.65 (m, 1H), 7.39-7.33 (m, 1H), 6.10 (s, 1H), 5.67 (s, 2H), 4.02-3.94 (m, 2H), 1.22 (t, 3H)

### Example 3: Synthesis of 2-(3,4-dichlorophenyl)-1-ethyl-4-oxo-6-[[5-(trifluoromethyl)imidazol-1-yl]methyl]pyridine-3-carboxylic acid (Compound 16)

### Step 1: Synthesis of 2-(3,4-dichlorophenyl)-1-ethyl-4-oxo-6-[[5-(trifluoromethyl)imidazol-1-yl]methyl]pyridine-3-carboxylic acid

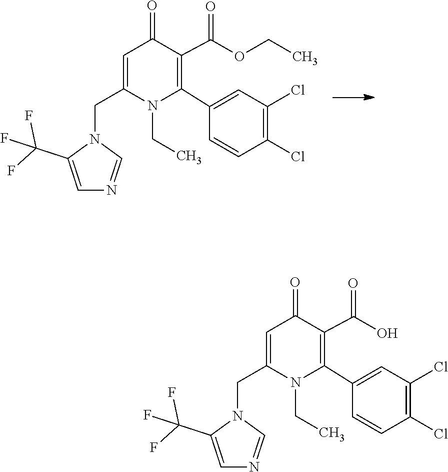 [SMILES: CCOC(=O)c1c(-c2ccc(Cl)c(Cl)c2)n(CC)c(Cn2cncc2C(F)(F)F)cc1=O ; CCn1c(Cn2cncc2C(F)(F)F)cc(=O)c(C(=O)O)c1-c1ccc(Cl)c(Cl)c1]

To a solution of ethyl 2-(3,4-dichlorophenyl)-1-ethyl-4-oxo-6-[[5-(trifluoromethyl)imidazol-1-yl]methyl]pyridine-3-carboxylate (0.023 g, 0.047 mmol) in a mixture of methanol (0.41 mL) and water (0.11 mL) was added lithium hydroxide (0.005 g, 0.19 mmol). The resultant solution was heated at reflux for 1.2 hours and then stood at room temperature for 18 hours. The cooled reaction mixture was diluted with dichloromethane and water and acidified to pH 1 by addition of 2 M aqueous hydrochloric acid before extraction into dichloromethane. The organic extracts were concentrated under reduced pressure to give 2-(3,4-dichlorophenyl)-1-ethyl-4-oxo-6-[[5-(trifluoromethyl)imidazol-1-yl]methyl]pyridine-3-carboxylic acid as a white solid.

¹H NMR (400 MHz, chloroform) δ=7.78 (s, 1H), 7.66-7.60 (m, 2H), 7.41 (d, 1H), 7.17 (dd, 1H), 5.99 (s, 1H), 5.37 (s, 2H), 3.84 (q, 2H), 1.33-1.17 (m, 3H)

### Example 4: Synthesis of 2-(3,4-dichlorophenyl)-1-ethyl-4-oxo-6-[[3-(trifluoromethyl)pyrazol-1-yl]methyl]pyridine-3-carboxylic acid (Compound 2)

### Step 1: Synthesis of ethyl 5-bromo-6-(bromomethyl)-2-(3,4-dichlorophenyl)-1-ethyl-4-oxo-pyridine-3-carboxylate

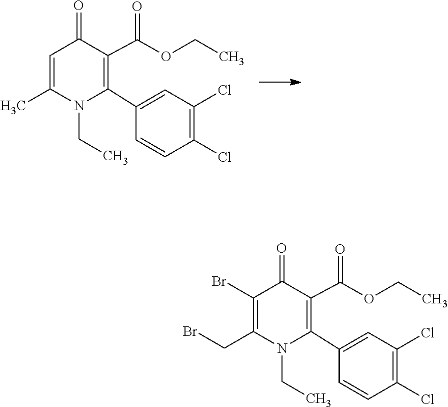 [SMILES: CCOC(=O)c1c(-c2ccc(Cl)c(Cl)c2)n(CC)c(C)cc1=O ; CCOC(=O)c1c(-c2ccc(Cl)c(Cl)c2)n(CC)c(CBr)c(Br)c1=O]

To a suspension of ethyl 2-(3,4-dichlorophenyl)-1-ethyl-6-methyl-4-oxo-pyridine-3-carboxylate (10.05 g, 28.37 mmol) in acetonitrile (100 mL) was added N-bromosuccinimide (NBS, 5.56 g, 31.2 mmol) portionwise over 3 minutes. On completion of addition, the reaction mixture was heated at 40° C. for 3 hours. After this time, the reaction mixture was diluted with acetonitrile (100 mL) and trifluoroacetic acid (3.93 g, 2.66 mL, 34 mmol) was added followed by portionwise addition of NBS (5.05 g, 28.4 mmol) over 5 minutes. The resultant reaction mixture was heated at 80° C. for 4 hours and then stood at room temperature for 18 hours. The acetonitrile was removed under reduced pressure and the residue was partitioned between dichloromethane and water. The organic phase was washed with a saturated aqueous solution of sodium hydrogen carbonate then brine, then dried over magnesium sulfate, filtered and concentrated under reduced pressure. The crude residue was purified by flash chromatography on silica gel using a gradient of 0-100% ethyl acetate in cyclohexane as eluent to give ethyl 5-bromo-6-(bromomethyl)-2-(3,4-dichlorophenyl)-1-ethyl-4-oxo-pyridine-3-carboxylate as an off-white solid.

¹H NMR (500 MHz, chloroform) δ=7.59 (d, 1H), 7.53 (d, 1H), 7.28 (d, 1H), 4.72 (brs, 2H), 4.10-3.95 (m, 4H), 1.25 (t, 3H), 1.04 (t, 3H)

### Step 2: Synthesis of ethyl 5-bromo-2-(3,4-dichlorophenyl)-1-ethyl-4-oxo-6-[[3-(trifluoromethyl)pyrazol-1-yl]methyl]pyridine-3-carboxylate

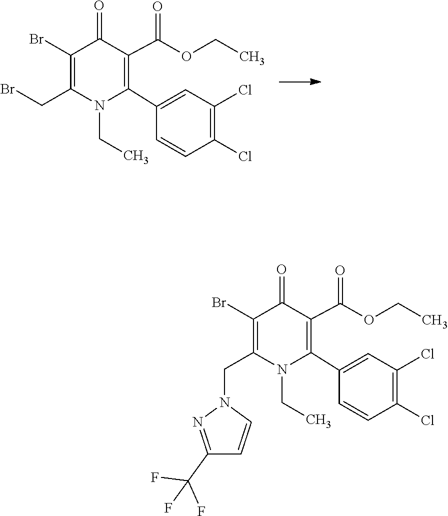 [SMILES: CCOC(=O)c1c(-c2ccc(Cl)c(Cl)c2)n(CC)c(CBr)c(Br)c1=O ; CCOC(=O)c1c(-c2ccc(Cl)c(Cl)c2)n(CC)c(Cn2ccc(C(F)(F)F)n2)c(Br)c1=O]

To a solution of ethyl 5-bromo-6-(bromomethyl)-2-(3,4-dichlorophenyl)-1-ethyl-4-oxo-pyridine-3-carboxylate (5.2 g, 10.1 mmol) in acetonitrile (100 mL) was added 3-(trifluoromethyl)-1H-pyrazole (1.52 g, 11.2 mmol) followed by portionwise addition of potassium carbonate (2.83 g, 20.3 mmol) over 2 minutes. The reaction mixture was stirred at room temperature for 1.7 hours. The acetonitrile was removed under reduced pressure and the residue was dissolved in a mixture of dichloromethane (100 mL) and water (100 mL). The aqueous phase was extracted with dichloromethane (×2) and the combined organic extracts were washed with brine, dried over magnesium sulfate, filtered and concentrated under reduced pressure to give ethyl 5-bromo-2-(3,4-dichlorophenyl)-1-ethyl-4-oxo-6-[[3-(trifluoromethyl)pyrazol-1-yl]methyl]pyridine-3-carboxylate as a white foam.

¹H NMR (400 MHz, chloroform) δ=7.80 (dd, 1H), 7.58 (d, 1H), 7.50 (d, 1H), 7.23 (dd, 1H), 6.64-6.61 (m, 1H), 5.81 (s, 2H), 4.23 (brd, 2H), 4.11-3.99 (m, 2H), 1.09-1.01 (m, 6H)

### Step 3: Synthesis of ethyl 2-(3,4-dichlorophenyl)-1-ethyl-4-oxo-6-[[3-(trifluoromethyl)pyrazol-1-yl]methyl]pyridine-3-carboxylate

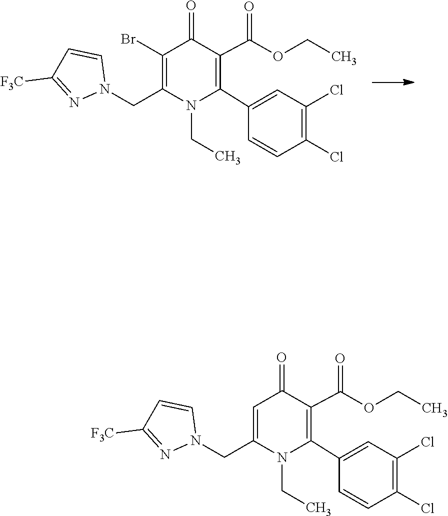 [SMILES: CCOC(=O)c1c(-c2ccc(Cl)c(Cl)c2)n(CC)c(Cn2ccc(C(F)(F)F)n2)c(Br)c1=O ; CCOC(=O)c1c(-c2ccc(Cl)c(Cl)c2)n(CC)c(Cn2ccc(C(F)(F)F)n2)cc1=O]

A hydrogenation vessel was loaded with 5% palladium on carbon (50% wet) (1.3 g), ethyl 5-bromo-2-(3,4-dichlorophenyl)-1-ethyl-4-oxo-6-[[3-(trifluoromethyl)pyrazol-1-yl]methyl]pyridine-3-carboxylate (5.0 g, 8.8 mmol), ethanol (130 mL) and acetic acid (25 mL). The mixture was stirred under an atmosphere of hydrogen (2 bar) for 5.5 hours. After this time, more 5% palladium on carbon (50% wet) (0.5 g) was added and the mixture was stirred under an atmosphere of hydrogen (2 bar) for 2.3 hours. The reaction mixture was filtered through diatomaceous earth, washing with dichloromethane and the filtrate was evaporated under reduced pressure. The crude residue was purified by flash chromatography on C-18 silica gel using a gradient of 40-80% acetonitrile (+0.1% formic acid) in water (+0.1% formic acid) as eluent to give ethyl 2-(3,4-dichlorophenyl)-1-ethyl-4-oxo-6-[[3-(trifluoromethyl)pyrazol-1-yl]methyl]pyridine-3-carboxylate as a white solid.

¹H NMR (400 MHz, chloroform) δ=7.65-7.62 (m, 1H), 7.57 (d, 1H), 7.51 (d, 1H), 7.26-7.23 (m, 1H), 6.65 (d, 1H), 6.34 (s, 1H), 5.35 (s, 2H), 4.09-3.97 (m, 2H), 3.87 (q, 2H), 1.04-0.95 (m, 6H)

### Step 4: Synthesis of 2-(3,4-dichlorophenyl)-1-ethyl-4-oxo-6-[[3-(trifluoromethyl)pyrazol-1-yl]methyl]pyridine-3-carboxylic acid

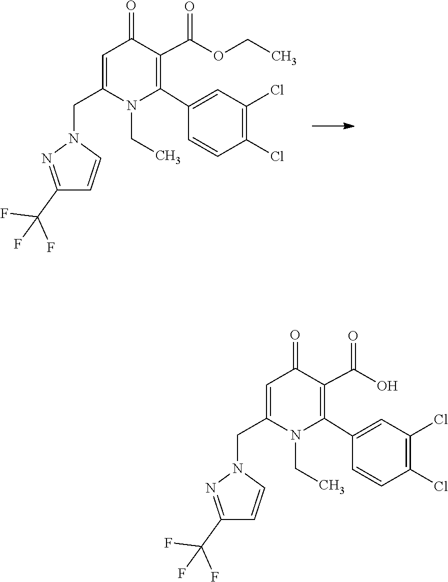 [SMILES: CCOC(=O)c1c(-c2ccc(Cl)c(Cl)c2)n(CC)c(Cn2ccc(C(F)(F)F)n2)cc1=O ; CCn1c(Cn2ccc(C(F)(F)F)n2)cc(=O)c(C(=O)O)c1-c1ccc(Cl)c(Cl)c1]

To a solution of ethyl 2-(3,4-dichlorophenyl)-1-ethyl-4-oxo-6-[[3-(trifluoromethyl)pyrazol-1-yl]methyl]pyridine-3-carboxylate (3.08 g, 6.31 mmol) in a mixture of methanol (55 mL) and water (15 mL) was added lithium hydroxide (0.604 g, 25.2 mmol). The resultant solution was heated at reflux for 2 hours. The reaction mixture was allowed to cool to 40° C. before being diluted with water (50 mL) and acidified to pH 1 by addition of 2 M aqueous hydrochloric acid (20 mL) resulting in precipitation of a white solid. The methanol was removed under reduced pressure and the resultant aqueous mixture was cooled in an ice bath. The resultant white solid was isolated by filtration, washed with water and air-dried to give 2-(3,4-dichlorophenyl)-1-ethyl-4-oxo-6-[[3-(trifluoromethyl)pyrazol-1-yl]methyl]pyridine-3-carboxylic acid as a white solid.

¹H NMR (400 MHz, chloroform) δ=7.73-7.68 (m, 1H), 7.61 (d, 1H), 7.43 (d, 1H), 7.17 (dd, J=2.0, 8.3 Hz, 1H), 6.67 (d, 1H), 6.20 (s, 1H), 5.47 (s, 2H), 4.02-3.91 (m, 2H), 1.20 (t, 3H)

### Example 5: Synthesis of 2-(3,4-dichlorophenyl)-1-ethyl-6-[(4-methoxycarbonylpyrazol-1-yl)methyl]-4-oxo-pyridine-3-carboxylic acid (Compound 40)

### Step 1: Synthesis of 2-(3,4-dichlorophenyl)-1-ethyl-5-iodo-6-methyl-4-oxo-pyridine-3-carboxylic acid

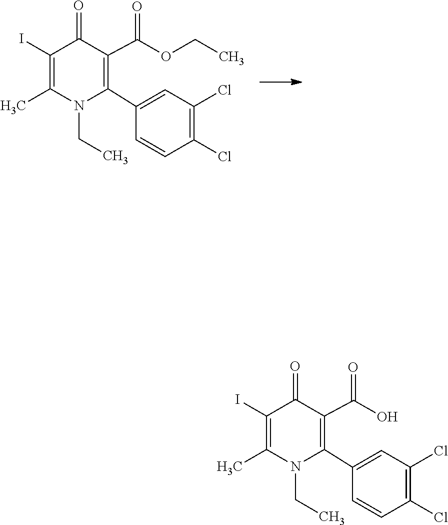 [SMILES: CCOC(=O)c1c(-c2ccc(Cl)c(Cl)c2)n(CC)c(C)c(I)c1=O ; CCn1c(C)c(I)c(=O)c(C(=O)O)c1-c1ccc(Cl)c(Cl)c1]

Ethyl 2-(3,4-dichlorophenyl)-1-ethyl-5-iodo-6-methyl-4-oxo-pyridine-3-carboxylate (6.0 g, 12.5 mmol) was dissolved in a mixture of methanol (54 mL) and water (24 mL) and lithium hydroxide (1.32 g, 31.5 mmol) was added. The reaction mixture was heated at 80° C. for 2 hours. The cooled reaction mixture was evaporated to dryness under reduced pressure and the residue was diluted with cold water and acidified by addition of 2 M aqueous hydrochloric acid. The resultant precipitate was filtered, washed with water and dried under reduced pressure to give 2-(3,4-dichlorophenyl)-1-ethyl-5-iodo-6-methyl-4-oxo-pyridine-3-carboxylic acid as a white solid.

¹H NMR (400 MHz, chloroform) δ=7.60 (d, 1H), 7.35 (d, 1H), 7.11 (dd, 1H), 4.13-3.92 (q, 2H), 3.00 (s, 3H), 1.23 (t, 3H).

### Step 2: Synthesis of tert-butyl 2-(3,4-dichlorophenyl)-1-ethyl-5-iodo-6-methyl-4-oxo-pyridine-3-carboxylate

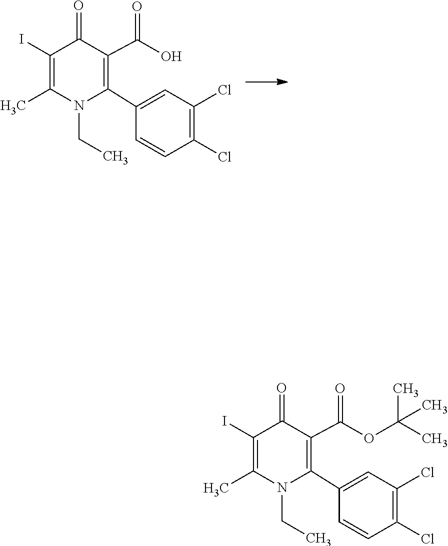 [SMILES: CCn1c(C)c(I)c(=O)c(C(=O)O)c1-c1ccc(Cl)c(Cl)c1 ; CCn1c(C)c(I)c(=O)c(C(=O)OC(C)(C)C)c1-c1ccc(Cl)c(Cl)c1]

To a suspension of 2-(3,4-dichlorophenyl)-1-ethyl-5-iodo-6-methyl-4-oxo-pyridine-3-carboxylic acid (4.7 g, 10 mmol) in 2-methylpropan-2-ol (3 mL/mmol) at room temperature was added sequentially N,N-dimethylpyridin-4-amine (5.2 mmol) and tert-butoxycarbonyl tert-butyl carbonate (21 mmol). The reaction mixture was stirred at room temperature for 18 hours. The reaction mixture was diluted with saturated aqueous sodium hydrogen carbonate solution and extracted into ethyl acetate (×3). The combined extracts were washed once with saturated aqueous sodium hydrogen carbonate solution then dried over magnesium sulfate. The residue was purified by flash chromatography on silica gel using ethyl acetate in cyclohexane as eluent to give tert-butyl 2-(3,4-dichlorophenyl)-1-ethyl-5-iodo-6-methyl-4-oxo-pyridine-3-carboxylate.

¹H NMR (400 MHz, chloroform) δ=7.64-7.45 (m, 1H), 7.56-7.46 (m, 1H), 7.26-7.24 (m, 1H), 3.97-3.85 (q, 2H), 2.87 (s, 3H), 1.25 (s, 9H), 1.20-1.15 (m, 3H).

### Step 3: Synthesis of tert-butyl 2-(3,4-dichlorophenyl)-1-ethyl-6-[(4-methoxycarbonylpyrazol-1-yl)methyl]-4-oxo-pyridine-3-carboxylate

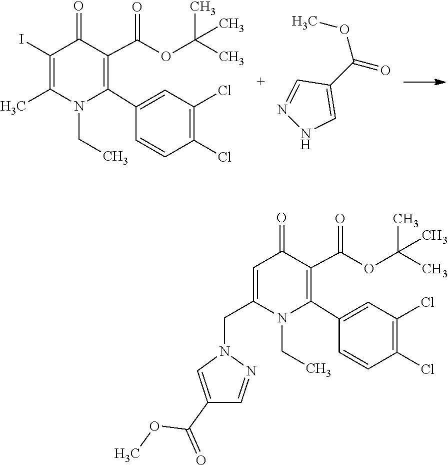

To a mixture of tert-butyl 2-(3,4-dichlorophenyl)-1-ethyl-5-iodo-6-methyl-4-oxo-pyridine-3-carboxylate (1.0 g, 1.97 mmol), methyl 1H-pyrazole-4-carboxylate (0.89 g, 7.1 mmol), copper(I) iodide (0.379 g, 1.97 mmol), 1,10-phenanthroline (0.731 g, 3.94 mmol) and potassium carbonate (0.55 g, 3.94 mmol) under nitrogen was added degassed N,N-dimethylacetamide (13 mL). The mixture was heated with stirring under microwave irradiation at 120° C. for 2 hours. The cooled reaction mixture was poured into saturated aqueous ammonium chloride solution and ethyl acetate and water were added. The mixture was filtered through diatomaceous earth and the organic phase was washed sequentially with saturated aqueous ammonium chloride, water then brine, then dried over magnesium sulfate. The crude residue was purified by HPLC to give tert-butyl 2-(3,4-dichlorophenyl)-1-ethyl-6-[(4-methoxycarbonylpyrazol-1-yl)methyl]-4-oxo-pyridine-3-carboxylate as white solid.

1H NMR (400 MHz, chloroform) δ=8.16-8.12 (m, 1H), 7.99-7.97 (m, 1H), 7.64-7.59 (m, 1H), 7.58-7.55 (m, 1H), 7.33-7.29 (m, 1H), 6.85-6.44 (m, 1H), 5.56-5.27 (m, 2H), 4.01-3.93 (m, 2H), 3.89-3.86 (m, 3H), 1.26-1.20 (m, 9H), 1.14-1.05 (m, 3H)

### Step 4: Synthesis of 2-(3,4-dichlorophenyl)-1-ethyl-6-[(4-methoxycarbonylpyrazol-1-yl)methyl]-4-oxo-pyridine-3-carboxylic acid

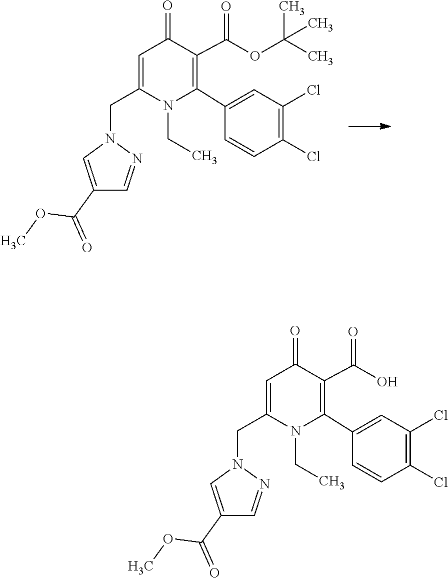

To a solution of tert-butyl 2-(3,4-dichlorophenyl)-1-ethyl-6-[(4-methoxycarbonylpyrazol-1-yl)methyl]-4-oxo-pyridine-3-carboxylate (100 mg, 0.20 mmol) in trifluoromethylbenzene (10 mL/g) and at 0° C. was added 2,2,2-trifluoroacetic acid (15 equiv., 2.962 mmol). The reaction mixture was warmed to room temperature and stirred for 24 hours. The reaction mixture evaporated to dryness under reduced pressure and the residue was washed with It was then allowed to stir at rt for 24 h. The reaction was monitored by LCMS. It was showed completion. Then the reaction mixture was concentrated under reduced pressure and residue was washed with methyl tert-butyl ether to give 2-(3,4-dichlorophenyl)-1-ethyl-6-[(4-methoxycarbonylpyrazol-1-yl)methyl]-4-oxo-pyridine-3-carboxylic acid.

1H NMR (400 MHz, DMSO-d6) δ=8.66 (s, 1H), 8.07 (s, 1H), 7.86-7.73 (m, 2H), 7.44 (dd, 1H), 6.18 (s, 1H), 5.73 (s, 2H), 3.94-3.89 (m, 2H), 3.78 (s, 3H), 1.05 (t, 3H)

### Example 6: Synthesis 2-(3,4-dichlorophenyl)-1-ethyl-6-[(3-methoxy-1,2,4-triazol-1-yl)methyl]-4-oxo-pyridine-3-carboxylic acid (Compound 44)

### Step 1: Synthesis of ethyl 2-(3,4-dichlorophenyl)-1-ethyl-6-[(3-nitro-1,2,4-triazol-1-yl)methyl]-4-oxo-pyridine-3-carboxylate

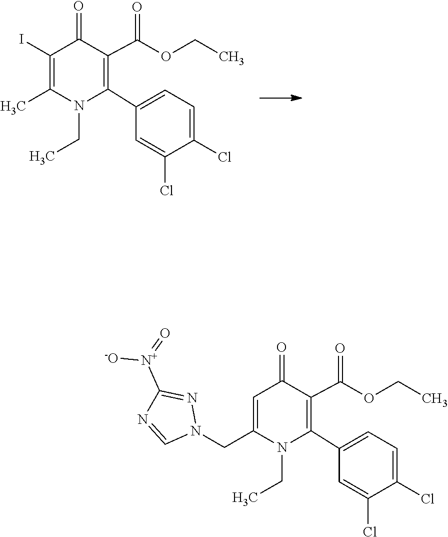

To a mixture of ethyl 2-(3,4-dichlorophenyl)-1-ethyl-5-iodo-6-methyl-4-oxo-pyridine-3-carboxylate (1.00 g, 2.08 mmol), 3-nitro-1H-1,2,4-triazole (0.855 g, 7.50 mmol), copper(I) iodide (0.401 g, 2.08 mmol), 1,10-phenanthroline (0.774 g, 4.17 mmol) and potassium carbonate (0.582 g, 4.17 mmol) under nitrogen was added degassed N,N-dimethylacetamide (14 mL). The reaction mixture was heated with stirring under microwave irradiation at 150° C. for 1 hour. The cooled reaction mixture was poured into saturated aqueous ammonium chloride solution and ethyl acetate and water were added. The mixture was filtered through diatomaceous earth and the organic phase was washed sequentially with saturated aqueous ammonium chloride, water then brine, then dried over magnesium sulfate. The crude residue was purified by HPLC to give ethyl 2-(3,4-dichlorophenyl)-1-ethyl-6-[(3-nitro-1,2,4-triazol-1-yl)methyl]-4-oxo-pyridine-3-carboxylate.

1H NMR (400 MHz, DMSO-d6) δ=9.10 (s, 1H), 7.85 (d, 1H), 7.82 (d, 1H), 7.49 (dd, 1H), 6.17 (s, 1H), 5.78 (s, 2H), 3.91-3.76 (m, 4H), 1.03 (t, 3H), 0.82 (t, 3H)

### Step 2: Synthesis of 2-(3,4-dichlorophenyl)-1-ethyl-6-[(3-methoxy-1,2,4-triazol-1-yl)methyl]-4-oxo-pyridine-3-carboxylic acid

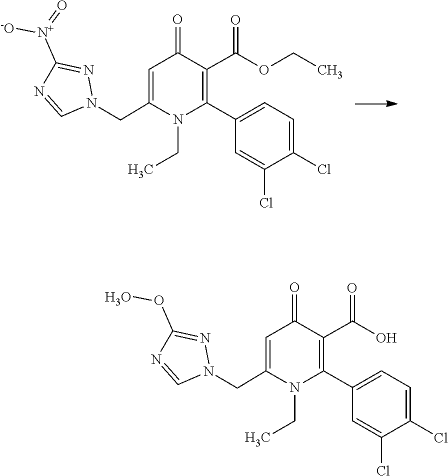

To a solution of ethyl 2-(3,4-dichlorophenyl)-1-ethyl-6-[(3-nitro-1,2,4-triazol-1-yl)methyl]-4-oxo-pyridine-3-carboxylate (40 mg, 0.086 mmol) in methanol (10 mL) was added a suspension of lithium hydroxide (0.014 g, 0.34 mmol) in water (0.04 mL). The reaction mixture was stirred at 80° C. for 1 hour. The cooled reaction mixture was acidified by addition of 2 M aqueous hydrochloric acid and diluted with additional water (10 mL), then extracted into ethyl acetate (2×10 mL). The combined organic extracts were washed with brine, dried over sodium sulfate, filtered and concentrated under reduced pressure. The crude material was triturated with tert-butyl methyl ether, filtered and the solid filtrate was dried under reduced pressure to give 2-(3,4-dichlorophenyl)-1-ethyl-6-[(3-methoxy-1,2,4-triazol-1-yl)methyl]-4-oxo-pyridine-3-carboxylic acid.

1H NMR (400 MHz, DMSO-d6) δ=8.48 (s, 1H), 7.84-7.80 (m, 1H), 7.78 (d, 1H), 7.44 (dd, 1H), 6.27 (s, 1H), 5.62 (s, 2H), 3.92-3.86 (m, 5H), 1.07 (t, 3H)

### Example 7: Synthesis of 6-[(3-bromopyrazol-1-yl)methyl]-2-(3,4-dichlorophenyl)-1-ethyl-4-oxo-pyridine-3-carboxylic acid (Compound 177)

### Step 1: Synthesis of 6-(bromomethyl)-2,2-dimethyl-1,3-dioxin-4-one

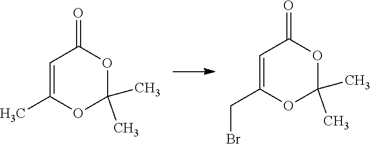

A stirred solution of 2,2,6-trimethyl-1,3-dioxin-4-one (4.88 g, 34.3 mmol) and N-bromosuccinimide (7.94 g, 44.6 mmol) in dichloromethane (120 mL) was irradiated with 450 nm light for 12 hours at room temperature. The orange reaction mixture was washed with a 50:50 mixture of saturated aqueous sodium bicarbonate solution and saturated aqueous sodium thiosulfate solution. The organic phase was dried over magnesium sulfate, filtered and evaporated to dryness under reduced pressure. The crude residue was purified by flash chromatography on C-18 silica gel using a gradient of 30-60% acetonitrile (+0.1% formic acid) in water (+0.1% formic acid) as eluent to give 6-(bromomethyl)-2,2-dimethyl-1,3-dioxin-4-one as a yellow oil.

¹H NMR (400 MHz, chloroform) δ=5.54 (s, 1H), 3.90 (s, 2H), 1.73 (s, 6H)

### Step 2: Synthesis of ethyl 6-(bromomethyl)-2-(3,4-dichlorophenyl)-1-ethyl-4-oxo-pyridine-3-carboxylate

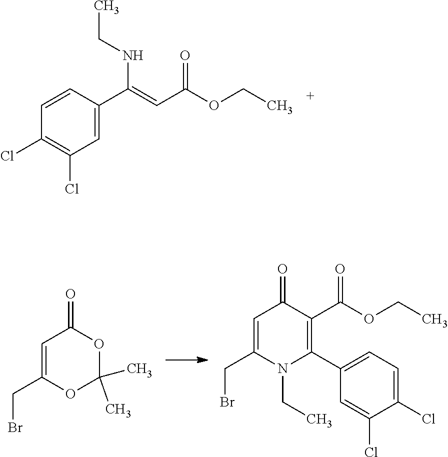 [SMILES: CCN/C(=C\C(=O)OCC)c1ccc(Cl)c(Cl)c1 ; CC1(C)OC(=O)C=C(CBr)O1 ; CCOC(=O)c1c(-c2ccc(Cl)c(Cl)c2)n(CC)c(CBr)cc1=O]

To a stirred solution of ethyl (Z)-3-(3,4-dichlorophenyl)-3-(ethylamino)prop-2-enoate (2.00 g, 6.94 mmol) in xylene (20.0 mL) was added 6-(bromomethyl)-2,2-dimethyl-1,3-dioxin-4-one (2.30 g, 10.4 mmol). The resultant reaction mixture was heated with stirring at 140° C. for 5 minutes. The cooled reaction mixture was diluted with ethyl acetate and the organic solution was washed with brine, dried over magnesium sulfate, filtered and evaporated to dryness under reduced pressure to 6-(bromomethyl)-2-(3,4-dichlorophenyl)-1-ethyl-4-oxo-pyridine-3-carboxylate as brown solid.

¹H NMR (400 MHz, DMSO-d6) δ=7.89 (s, 1H), 7.79 (d, 1H), 7.49 (d, 1H), 6.59 (s, 1H), 4.72 (s, 2H), 3.82-3.77 (q, 4H), 1.11 (t, 3H), 0.84 (t, 3H).

### Step 3: Synthesis of ethyl 6-[(3-bromopyrazol-1-yl)methyl]-2-(3,4-dichlorophenyl)-1-ethyl-4-oxo-pyridine-3-carboxylate

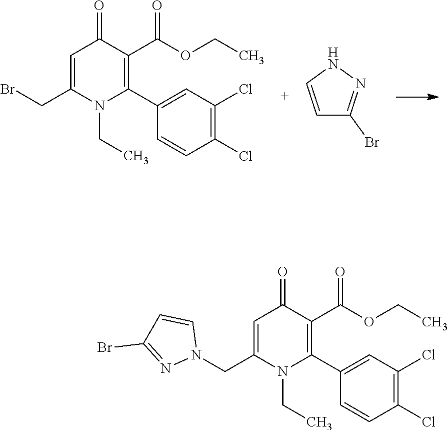 [SMILES: CCOC(=O)c1c(-c2ccc(Cl)c(Cl)c2)n(CC)c(CBr)cc1=O ; Brc1cc[nH]n1 ; CCOC(=O)c1c(-c2ccc(Cl)c(Cl)c2)n(CC)c(Cn2ccc(Br)n2)cc1=O]

To a solution of ethyl 6-(bromomethyl)-2-(3,4-dichlorophenyl)-1-ethyl-4-oxo-pyridine-3-carboxylate (0.500 g, 1.15 mmol) in acetonitrile (10 mL) was added 3-bromo-1H-pyrazole (0.19 g, 1.27 mmol) followed by portion-wise addition of potassium carbonate (0.32 g, 2.31 mmol) over 2 minutes. The reaction mixture was stirred at room temperature for 16 hours. The reaction mixture was diluted with water and ethyl acetate and the phases were separated. The organic extract was dried over anhydrous magnesium sulfate, filtered and evaporated to dryness under reduced pressure. The crude residue was purified by flash chromatography on reverse-phase C-18 silica gel using a gradient of 20 to 80% acetonitrile (+0.1% formic acid) in water (+0.1% formic acid) as eluent to give ethyl 6-[(3-bromopyrazol-1-yl)methyl]-2-(3,4-dichlorophenyl)-1-ethyl-4-oxo-pyridine-3-carboxylate as a white solid foam.

1H NMR (400 MHz, chloroform) δ=7.56 (d, 1H), 7.50 (d, 1H), 7.42 (d, 1H), 7.24 (d, 1H), 6.40 (d, 1H), 6.38 (s, 1H), 5.25 (s, 2H), 4.09-3.97 (m, 2H), 3.86 (q, 2H), 1.04 (t, 3H), 0.98 (t, 3H)

### Step 4: Synthesis of 6-[(3-bromopyrazol-1-yl)methyl]-2-(3,4-dichlorophenyl)-1-ethyl-4-oxo-pyridine-3-carboxylic acid

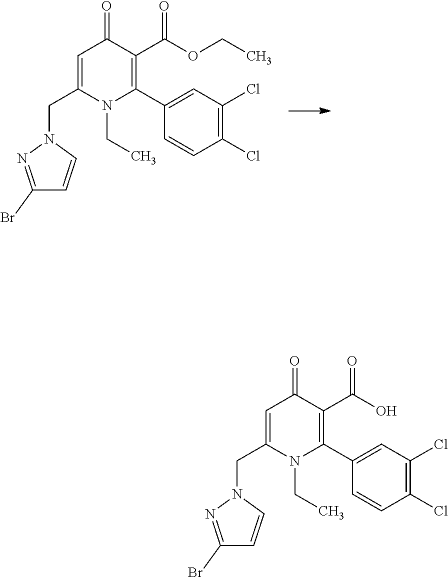 [SMILES: CCOC(=O)c1c(-c2ccc(Cl)c(Cl)c2)n(CC)c(Cn2ccc(Br)n2)cc1=O ; CCn1c(Cn2ccc(Br)n2)cc(=O)c(C(=O)O)c1-c1ccc(Cl)c(Cl)c1]

To a solution of ethyl 6-[(3-bromopyrazol-1-yl)methyl]-2-(3,4-dichlorophenyl)-1-ethyl-4-oxo-pyridine-3 carboxylate (0.139 g, 0.278 mmol) in a mixture of methanol (1.25 mL) and water (0.35 mL) was added lithium hydroxide (0.048 g, 1.11 mmol). The resultant reaction mixture was heated at 80° C. for 3 hours. The cooled reaction mixture was diluted with dichloromethane and water and acidified to pH 1 by addition of 2 M aqueous hydrochloric acid. The phases were separated, and the organic phase was evaporated to dryness under reduced pressure to give 6-[(3-bromopyrazol-1-yl)methyl]-2-(3,4-dichlorophenyl)-1-ethyl-4-oxo-pyridine-3-carboxylic acid as a white solid foam.

1H NMR (400 MHz, chloroform) δ=7.61 (d, 1H), 7.49 (d, 1H), 7.39 (d, 1H), 7.14 (dd, 1H), 6.43 (d, 1H), 6.34 (s, 1H), 5.37 (s, 2H), 3.96 (q, 2H), 1.20 (t, 3H)

### Example 8: Synthesis of 2-(3,4-dichlorophenyl)-1-ethyl-5-fluoro-4-oxo-6-[[3-(trifluoromethyl)pyrazol-1-yl]methyl]pyridine-3-carboxylic acid (Compound 206)

### Step 1: Synthesis of ethyl 2-(3,4-dichlorophenyl)-1-ethyl-5-fluoro-6-methyl-4-oxo-pyridine-3-carboxylate

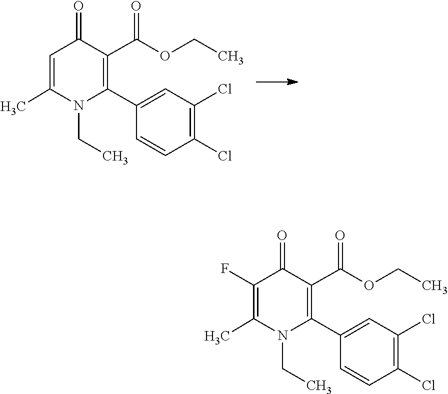 [SMILES: CCOC(=O)c1c(-c2ccc(Cl)c(Cl)c2)n(CC)c(C)cc1=O ; CCOC(=O)c1c(-c2ccc(Cl)c(Cl)c2)n(CC)c(C)c(F)c1=O]

To a stirred solution of ethyl 2-(3,4-dichlorophenyl)-1-ethyl-6-methyl-4-oxo-pyridine-3-carboxylate (2.50 g, 7.06 mmol) in ethanol (100 mL) at room temperature was added 1-(chloromethyl)-4-fluoro-1,4-diazoniabicyclo[2.2.2]octane; ditetrafluoroborate (7.50 g, 21.2 mmol). The resultant reaction mixture was heated with stirring at 80° C. for 48 hours. The cooled reaction mixture was diluted with water (100 mL) and extracted into dichloromethane (2×100 mL). The combined organic extracts were dried over anhydrous sodium sulfate, filtered and evaporated to dryness under reduced pressure. The crude residue was purified by flash chromatography on reverse-phase C-18 silica gel using a gradient of 0 to 65% acetonitrile (+0.1% formic acid) in water (+0.1% formic acid) as eluent to give 2-(3,4-dichlorophenyl)-1-ethyl-5-fluoro-6-methyl-4-oxo-pyridine-3-carboxylate as white solid.

### Step 2: Synthesis of ethyl 6-(bromomethyl)-2-(3,4-dichlorophenyl)-1-ethyl-5-fluoro-4-oxo-pyridine-3-carboxylate

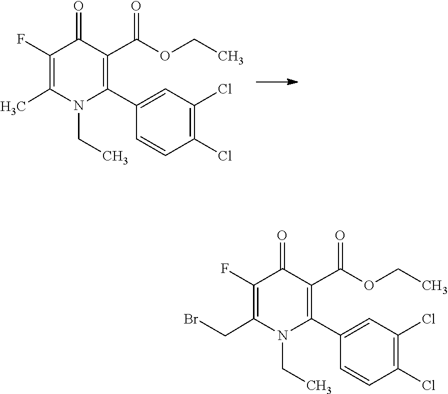 [SMILES: CCOC(=O)c1c(-c2ccc(Cl)c(Cl)c2)n(CC)c(C)c(F)c1=O ; CCOC(=O)c1c(-c2ccc(Cl)c(Cl)c2)n(CC)c(CBr)c(F)c1=O]

To a stirred solution of ethyl 2-(3,4-dichlorophenyl)-1-ethyl-5-fluoro-6-methyl-4-oxo-pyridine-3-carboxylate (500 mg, 1.01 mmol) in acetonitrile (25 mL) and at room temperature was added 1-bromopyrrolidine-2,5-dione (0.269 g, 1.51 mmol) and trifluoroacetic acid (0.293 g, 1.21 mmol).

The resultant reaction mixture was heated with stirring at 80° C. for 2 hours. The cooled reaction mixture was diluted with water (50 mL) and extracted into ethyl acetate (×2). The combined organic extracts were washed with brine (20 mL), dried over anhydrous sodium sulfate, filtered and evaporated to dryness under reduced pressure. The crude residue was purified by flash chromatography on silica gel using a gradient of 10 to 50% ethyl acetate in hexanes as eluent to give ethyl 6-(bromomethyl)-2-(3,4-dichlorophenyl)-1-ethyl-5-fluoro-4-oxo-pyridine-3-carboxylate as white solid.

1H NMR (400 MHz, DMSO-d6) δ=7.92-7.81 (m, 2H), 7.56-7.43 (m, 1H), 3.90-3.81 (m, 4H), 2.70 (s, 2H), 1.11 (t, 3H), 0.93 (t, 3H)

### Step 3: Synthesis of ethyl 2-(3,4-dichlorophenyl)-1-ethyl-5-fluoro-4-oxo-6-[[3-(trifluoromethyl)pyrazol-1-yl]methyl]pyridine-3-carboxylate

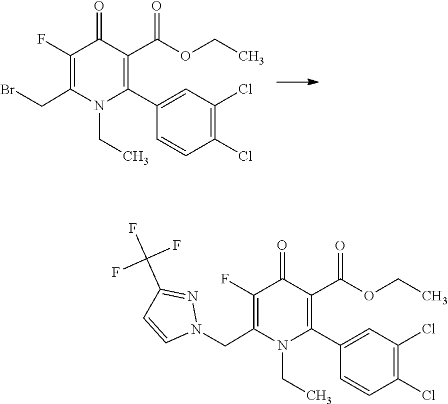 [SMILES: CCOC(=O)c1c(-c2ccc(Cl)c(Cl)c2)n(CC)c(CBr)c(F)c1=O ; CCOC(=O)c1c(-c2ccc(Cl)c(Cl)c2)n(CC)c(Cn2ccc(C(F)(F)F)n2)c(F)c1=O]

To a stirred solution of ethyl 6-(bromomethyl)-2-(3,4-dichlorophenyl)-1-ethyl-5-fluoro-4-oxo-pyridine-3-carboxylate (400 mg, 0.84 mmol) in acetonitrile (10 mL) was added 3-(trifluoromethyl)-1H-pyrazole (126 mg, 0.93 mmol) and potassium carbonate (232 mg, 1.68 mmol). The resultant reaction mixture was stirred at room temperature for 16 hours. The reaction mixture was diluted with water (50 mL) and extracted into ethyl acetate (×2). The combined organic extracts were dried over anhydrous sodium sulfate, filtered and evaporated to dryness under reduced pressure. The residue was purified by flash chromatography on silica gel using a gradient of 10 to 50% ethyl acetate in hexanes as eluent to give ethyl 2-(3,4-dichlorophenyl)-1-ethyl-5-fluoro-4-oxo-6-[[3-(trifluoromethyl)pyrazol-1-yl]methyl]pyridine-3-carboxylate as white solid.

### Step 4: Synthesis of 2-(3,4-dichlorophenyl)-1-ethyl-5-fluoro-4-oxo-6-[[3-(trifluoromethyl)pyrazol-1-yl]methyl]pyridine-3-carboxylic acid

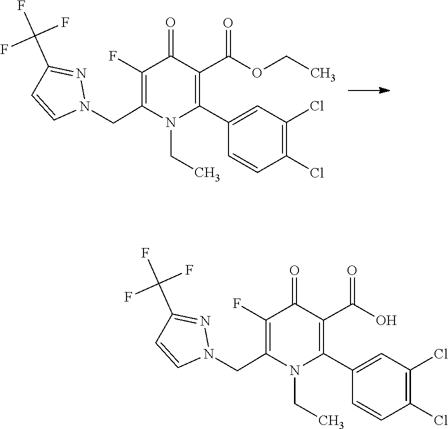 [SMILES: CCOC(=O)c1c(-c2ccc(Cl)c(Cl)c2)n(CC)c(Cn2ccc(C(F)(F)F)n2)c(F)c1=O ; CCn1c(Cn2ccc(C(F)(F)F)n2)c(F)c(=O)c(C(=O)O)c1-c1ccc(Cl)c(Cl)c1]

To a stirred solution of ethyl 2-(3,4-dichlorophenyl)-1-ethyl-5-fluoro-4-oxo-6-[[3-(trifluoromethyl)pyrazol-1-yl]methyl]pyridine-3-carboxylate (190 mg, 0.38 mmol) in a mixture of acetonitrile (7.5 mL) and water (2.5 mL) at room temperature was added lithium hydroxide hydrate (0.063 g, 1.5 mmol) at ambient temperature. The resultant reaction mixture was heated with stirring at 80° C. for 4 hours. The cooled reaction mixture was diluted with water (20 mL) and neutralised by addition of 2 M aqueous hydrogen chloride solution. The precipitated solids were filtered and dried by evaporation of solvents under reduced pressure to give 2-(3,4-dichlorophenyl)-1-ethyl-5-fluoro-4-oxo-6-[[3-(trifluoromethyl)pyrazol-1-yl]methyl]pyridine-3-carboxylic acid as a light brown solid.

1H NMR (400 MHz, DMSO-d6) δ=8.24 (d, 1H), 7.81 (dd, 2H), 7.46 (dd, 1H), 6.86 (d, 1H), 5.80 (d, 2H), 3.96 (q, 2H), 0.59 (t, 3H)

### Example 9: Synthesis of 2-(3,4-dichlorophenyl)-1-ethyl-5-methyl-4-oxo-6-[[3-(trifluoromethyl)pyrazol-1-yl]methyl]pyridine-3-carboxylic acid (Compound 20)

### Step 1: Synthesis of ethyl 2-(3,4-dichlorophenyl)-1-ethyl-5-methyl-4-oxo-6-[[3-(trifluoromethyl)pyrazol-1-yl]methyl]pyridine-3-carboxylate

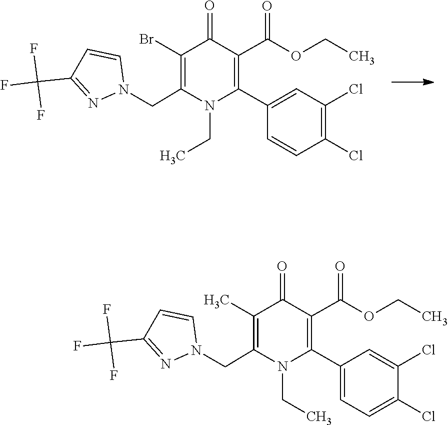 [SMILES: CCOC(=O)c1c(-c2ccc(Cl)c(Cl)c2)n(CC)c(Cn2ccc(C(F)(F)F)n2)c(Br)c1=O ; CCOC(=O)c1c(-c2ccc(Cl)c(Cl)c2)n(CC)c(Cn2ccc(C(F)(F)F)n2)c(C)c1=O]

To a degassed mixture of acetonitrile (8 mL) and water (2 mL) under nitrogen was added ethyl 5-bromo-2-(3,4-dichlorophenyl)-1-ethyl-4-oxo-6-[[3-(trifluoromethyl)pyrazol-1-yl]methyl]pyridine-3-carboxylate (200 mg, 0.35 mmol), methylboronic acid (40 mg, 0.67 mmol) and bis(triphenylphosphine)palladium(II)) dichloride (60 mg, 0.083 mmol) followed by potassium carbonate (200 mg, 1.4 mmol). The resulting suspension was blanketed with nitrogen and heated under microwave irradiation at 100° C. for 0.5 hours. The cooled reaction mixture was filtered through diatomaceous earth, washing with dichloromethane. The filtrate was washed with brine, then dried over anhydrous magnesium sulfate, filtered and evaporated to dryness under reduced pressure. The crude residue was purified by flash chromatography on silica gel using a gradient of ethyl acetate in cyclohexane as eluent to give ethyl 2-(3,4-dichlorophenyl)-1-ethyl-5-methyl-4-oxo-6-[[3-(trifluoromethyl)pyrazol-1-yl]methyl]pyridine-3-carboxylate (170 mg, 0.34 mmol) as a cream solid.

1H NMR (400 MHz, chloroform) δ=7.56 (d, 1H), 7.48 (d, 1H), 7.45 (d, 1H), 7.22 (dd, 1H), 6.63 (d, 1H), 5.51 (s, 2H), 4.11-3.98 (m, 2H), 3.92 (q, 2H), 2.31 (s, 3H), 1.06-0.98 (m, 6H)

### Step 2: Synthesis of 2-(3,4-dichlorophenyl)-1-ethyl-5-methyl-4-oxo-6-[[3-(trifluoromethyl)pyrazol-1-yl]methyl]pyridine-3-carboxylic acid

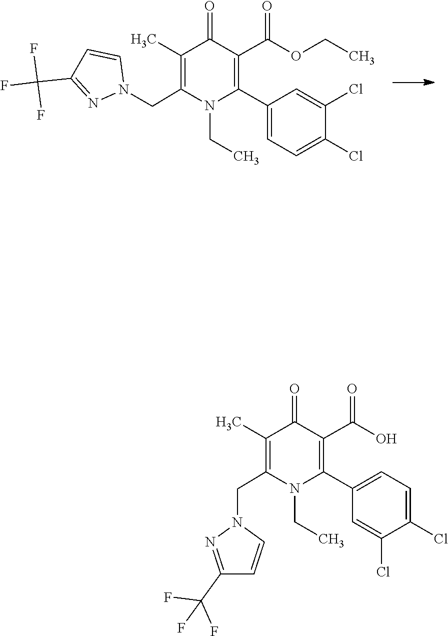 [SMILES: CCOC(=O)c1c(-c2ccc(Cl)c(Cl)c2)n(CC)c(Cn2ccc(C(F)(F)F)n2)c(C)c1=O ; CCn1c(Cn2ccc(C(F)(F)F)n2)c(C)c(=O)c(C(=O)O)c1-c1ccc(Cl)c(Cl)c1]

To a solution of ethyl 2-(3,4-dichlorophenyl)-1-ethyl-5-methyl-4-oxo-6-[[3-(trifluoromethyl)pyrazol-1-yl]methyl]pyridine-3-carboxylate (170 mg, 0.34 mmol) in methanol (5.0 mL) was added a solution of lithium hydroxide hydrate (75 mg, 1.8 mmol) in water (1 mL). The resultant reaction mixture was heated with stirring at 80° C. for 1.5 hours. The cooled reaction mixture was poured into water, acidified with 2M aqueous hydrogen chloride solution and extracted into dichloromethane. The crude material was purified by flash chromatography on reverse-phase C-18 silica gel using a gradient of acetonitrile (+0.1% formic acid) in water (+0.1% formic acid) as eluent to give 2-(3,4-dichlorophenyl)-1-ethyl-5-methyl-4-oxo-6-[[3-(trifluoromethyl)pyrazol-1-yl]methyl]pyridine-3-carboxylic acid as a cream coloured solid.

1H NMR (400 MHz, methanol) 6=7.64 (d, 1H), 7.61 (d, 1H), 7.40 (d, 1H), 7.14 (dd, 1H), 6.65 (d, 1H), 5.61 (s, 2H), 4.19-4.08 (m, 2H), 2.36 (s, 3H), 1.14 (t, 3H)

### Example 10: Synthesis of 5-(4-chlorophenyl)-2-(3,4-dichlorophenyl)-1-ethyl-4-oxo-6-[[3-(trifluoromethyl)pyrazol-1-yl]methyl]pyridine-3-carboxylic acid (Compound 21)

### Step 1: Synthesis of ethyl 5-(4-chlorophenyl)-2-(3,4-dichlorophenyl)-1-ethyl-4-oxo-6-[[3-(trifluoromethyl)pyrazol-1-yl]methyl]pyridine-3-carboxylate

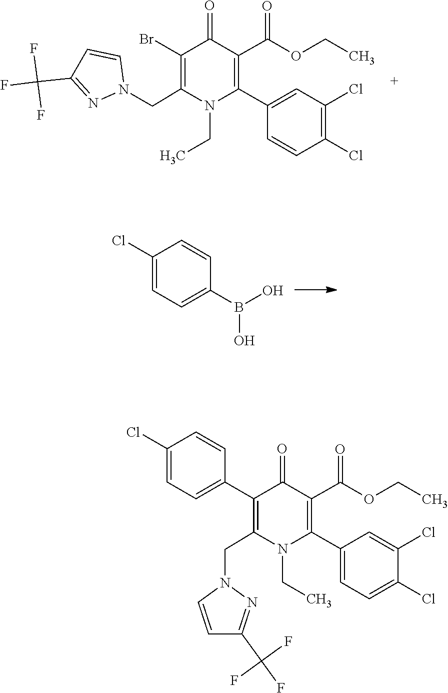 [SMILES: CCOC(=O)c1c(-c2ccc(Cl)c(Cl)c2)n(CC)c(Cn2ccc(C(F)(F)F)n2)c(Br)c1=O ; OB(O)c1ccc(Cl)cc1 ; CCOC(=O)c1c(-c2ccc(Cl)c(Cl)c2)n(CC)c(Cn2ccc(C(F)(F)F)n2)c(-c2ccc(Cl)cc2)c1=O]

To a degassed mixture of acetonitrile (8 mL) and water (2 mL) under nitrogen was added ethyl 5-bromo-2-(3,4-dichlorophenyl)-1-ethyl-4-oxo-6-[[3-(trifluoromethyl)pyrazol-1-yl]methyl]pyridine-3-carboxylate (200 mg, 0.35 mmol), (4-chlorophenyl)boronic acid (70 mg, 0.45 mmol) and bis(triphenylphosphine)palladium(II)) dichloride (50 mg, 0.069 mmol) followed by potassium carbonate (150 mg, 1.1 mmol). The resultant suspension was blanketed with nitrogen and heated under microwave irradiation at 100° C. for 0.5 hours. The cooled reaction mixture was filtered through diatomaceous earth, washing with dichloromethane. The filtrate was washed with brine, then dried over anhydrous magnesium sulfate, filtered and evaporated to dryness under reduced pressure. The crude residue was purified by flash chromatography on silica gel using a gradient of ethyl acetate in cyclohexane as eluent to give ethyl 5-(4-chlorophenyl)-2-(3,4-dichlorophenyl)-1-ethyl-4-oxo-6-[[3-(trifluoromethyl)pyrazol-1-yl]methyl]pyridine-3-carboxylate as a colourless gum.

1H NMR (500 MHz, chloroform) δ=7.59 (d, 1H), 7.56 (d, 1H), 7.43 (d, 2H), 7.29 (dd, 1H), 7.15 (d, 2H), 7.10 (d, 1H), 6.54 (d, 1H), 5.29 (s, 2H), 4.14-4.00 (m, 4H), 1.11-1.00 (m, 6H)

### Step 2: Synthesis of 5-(4-chlorophenyl)-2-(3,4-dichlorophenyl)-1-ethyl-4-oxo-6-[[3-(trifluoromethyl)pyrazol-1-yl]methyl]pyridine-3-carboxylic acid

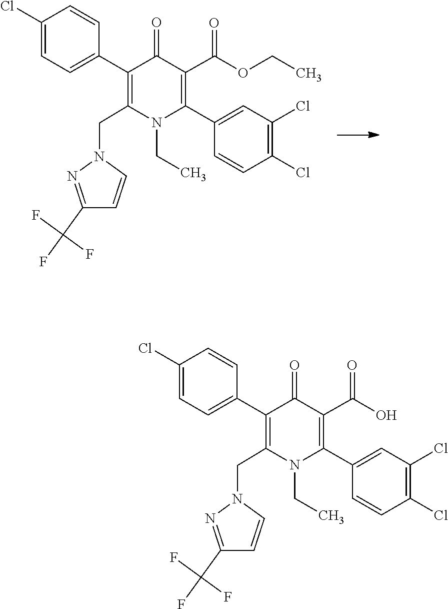 [SMILES: CCOC(=O)c1c(-c2ccc(Cl)c(Cl)c2)n(CC)c(Cn2ccc(C(F)(F)F)n2)c(-c2ccc(Cl)cc2)c1=O ; CCn1c(Cn2ccc(C(F)(F)F)n2)c(-c2ccc(Cl)cc2)c(=O)c(C(=O)O)c1-c1ccc(Cl)c(Cl)c1]

To a solution of ethyl 5-(4-chlorophenyl)-2-(3,4-dichlorophenyl)-1-ethyl-4-oxo-6-[[3-(trifluoromethyl)pyrazol-1-yl]methyl]pyridine-3-carboxylate (160 mg, 0.27 mmol) in a mixture of methanol (10 mL) and water (1 mL) was added lithium hydroxide hydrate (35 mg, 0.83 mmol). The resultant reaction mixture was heated with stirring at 80° C. for 4 hours. The cooled reaction mixture was poured into a 2 M aqueous solution of hydrogen chloride and extracted into dichloromethane. The organic extract was evaporated to dryness under reduced pressure to give 5-(4-chlorophenyl)-2-(3,4-dichlorophenyl)-1-ethyl-4-oxo-6-[[3-(trifluoromethyl)pyrazol-1-yl]methyl]pyridine-3-carboxylic acid as a cream solid.

1H NMR (400 MHz, chloroform) δ=7.61 (d, 1H), 7.48 (brd, 2H), 7.44-7.38 (m, 1H), 7.18 (dd, 1H), 7.12 (brd, 2H), 7.07 (d, 1H), 6.54 (d, 1H), 5.33 (s, 2H), 4.27-4.15 (m, 2H), 1.17 (t, 2H)

### Example 11: Synthesis of 6-[[3,5-bis(ethoxycarbonyl)pyrazol-1-yl]methyl]-2-(3,4-dichlorophenyl)-1-ethyl-4-oxo-pyridine-3-carboxylic acid (Compound 112)

### Step 1: Synthesis of 5-bromo-6-(bromomethyl)-2-(3,4-dichlorophenyl)-1-ethyl-4-oxo-1,4-dihydropyridine-3-carboxylic acid

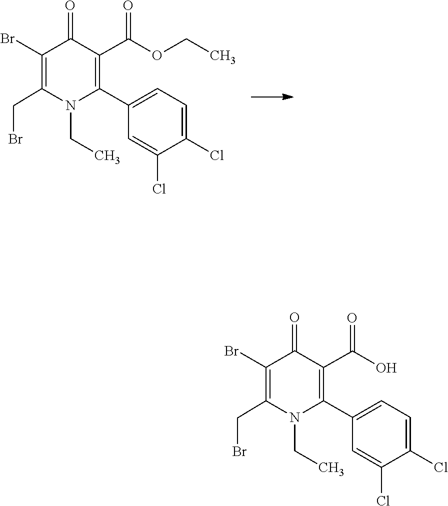 [SMILES: CCOC(=O)c1c(-c2ccc(Cl)c(Cl)c2)n(CC)c(CBr)c(Br)c1=O ; CCn1c(CBr)c(Br)c(=O)c(C(=O)O)c1-c1ccc(Cl)c(Cl)c1]

To a solution of ethyl 5-bromo-6-(bromomethyl)-2-(3,4-dichlorophenyl)-1-ethyl-4-oxo-pyridine-3-carboxylate (5.00 g, 9.77 mmol) in a mixture of tetrahydrofuran (50 mL) and water (10 mL) was added scandium(III)trifluoromethanesulfonate (6.25 g, 12.7 mmol) portion-wise. The reaction mixture was heated with stirring at 50° C. for 12 hours. The cooled reaction mixture was diluted with ethyl acetate (30 mL) and washed sequentially with water and brine then dried over anhydrous magnesium sulfate, filtered and evaporated to dryness under reduced pressure. The crude residue was purified by flash chromatography on silica gel using dichloromethane as eluent to give 5-bromo-6-(bromomethyl)-2-(3,4-dichlorophenyl)-1-ethyl-4-oxo-pyridine-3-carboxylic acid as a white solid.

1H NMR (500 MHz, chloroform) δ=7.60 (d, 1H), 7.36 (d, 1H), 7.12 (dd, 1H), 4.96 (s, 2H), 4.18-4.09 (m, 2H), 1.22 (t, 3H)

### Step 2: Synthesis of 6-((3,5-bis(ethoxycarbonyl)-1H-pyrazol-1-yl) methyl)-5-bromo-2-(3,4-dichlorophenyl)-1-ethyl-4-oxo-1,4-dihydropyridine-3-carboxylic acid

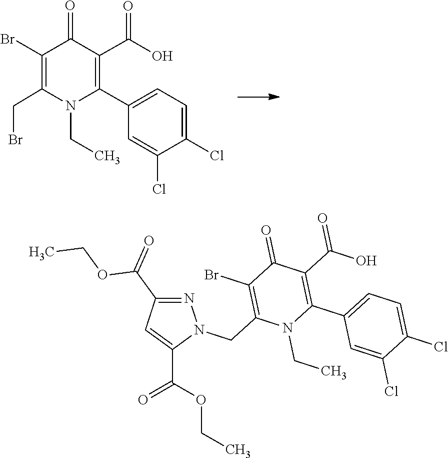 [SMILES: CCn1c(CBr)c(Br)c(=O)c(C(=O)O)c1-c1ccc(Cl)c(Cl)c1 ; CCOC(=O)c1cc(C(=O)OCC)n(Cc2c(Br)c(=O)c(C(=O)O)c(-c3ccc(Cl)c(Cl)c3)n2CC)n1]

To a solution of diethyl 1H-pyrazole-3,5-dicarboxylate (158 mg, 0.74 mmol) in anhydrous tetrahydrofuran (3 mL) and under an atmosphere of nitrogen was added sodium hydride (50 mg). After minutes, a solution of 5-bromo-6-(bromomethyl)-2-(3,4-dichlorophenyl)-1-ethyl-4-oxo-1,4-dihydropyridine-3-carboxylic acid (300 mg, 0.62 mmol) in anhydrous tetrahydrofuran (3 mL) was added drop-wise. The resultant reaction mixture was stirred at room temperature for 1 hour. The reaction mixture was quenched by addition of water and extracted into ethyl acetate (×3). The combined organic extracts were washed with brine, dried over anhydrous sodium sulfate, filtered and evaporated to dryness under reduced pressure. The crude residue was purified by flash chromatography on silica gel using a gradient of 0 to 5% methanol in dichloromethane as eluent to give 6-((3,5-bis(ethoxycarbonyl)-1H-pyrazol-1-yl) methyl)-5-bromo-2-(3,4-dichlorophenyl)-1-ethyl-4-oxo-1,4-dihydropyridine-3-carboxylic acid as a white solid.

1H NMR (400 MHz, DMSO-d6) δ=14.06 (s, 1H), 7.84 (d, 1H), 7.77 (d, 1H), 7.45 (dd, 1H), 7.36 (s, 1H), 6.23 (s, 2H), 4.39 (q, 2H), 4.28 (q, 2H), 3.95 (dd, 2H), 1.35 (t, 3H), 1.29 (t, 3H), 1.03 (t, 3H)

### Step 3: Synthesis of 6-[[3,5-bis(ethoxycarbonyl)pyrazol-1-yl]methyl]-2-(3,4-dichlorophenyl)-1-ethyl-4-oxo-pyridine-3-carboxylic acid

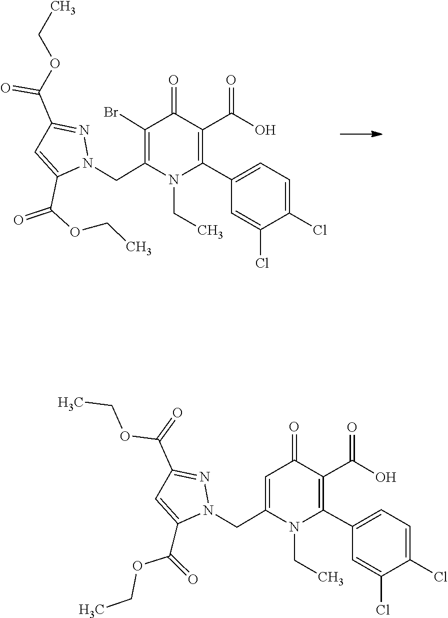 [SMILES: CCOC(=O)c1cc(C(=O)OCC)n(Cc2c(Br)c(=O)c(C(=O)O)c(-c3ccc(Cl)c(Cl)c3)n2CC)n1 ; CCOC(=O)c1cc(C(=O)OCC)n(Cc2cc(=O)c(C(=O)O)c(-c3ccc(Cl)c(Cl)c3)n2CC)n1]

To a solution of 6-((3,5-bis(ethoxycarbonyl)-1H-pyrazol-1-yl) methyl)-5-bromo-2-(3,4-dichlorophenyl)-1-ethyl-4-oxo-1,4-dihydropyridine-3-carboxylic acid 9 (250 mg, 0.41 mmol) in methanol (10 mL) was added 10% palladium on carbon (100 mg) at room temperature and the mixture was stirred under an atmosphere of hydrogen for 12 hours, The reaction mixture was concentrated through diatomaceous earth and the filtrate was evaporated to dryness under reduced pressure. The crude residue was purified by preparative HPLC to give 6-[[3,5-bis(ethoxycarbonyl)pyrazol-1-yl]methyl]-2-(3,4-dichlorophenyl)-1-ethyl-4-oxo-pyridine-3-carboxylic acid as white solid.

1H NMR (400 MHz, DMSO-d6) δ=16.22 (s, 1H), 7.83-7.79 (m, 2H), 7.45 (m, 2H), 6.10 (s, 2H), 5.52 (s, 1H), 4.33-4.29 (m, 4H), 3.86 (q, 2H), 1.32-1.28 (m, 6H), 1.17 (t, 3H)

### Example 12: Synthesis of 2-(3,4-dichlorophenyl)-1-ethyl-6-[[5-hydroxy-3-(trifluoromethyl)pyrazol-1-yl]methyl]-4-oxo-pyridine-3-carboxylic acid (Compound 147)

### Step 1: Synthesis of ethyl 5-bromo-2-(3,4-dichlorophenyl)-1-ethyl-6-(hydrazinomethyl)-4-oxo-pyridine-3-carboxylate

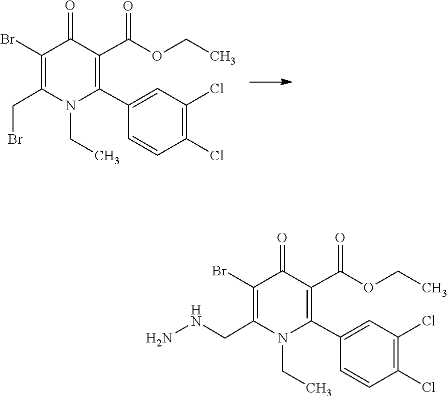 [SMILES: CCOC(=O)c1c(-c2ccc(Cl)c(Cl)c2)n(CC)c(CBr)c(Br)c1=O ; CCOC(=O)c1c(-c2ccc(Cl)c(Cl)c2)n(CC)c(CNN)c(Br)c1=O]

To a solution of hydrazine monohydrate (0.211 g, 4.3 mmol) in acetonitrile (20 mL) and at room temperature was added ethyl 5-bromo-6-(bromomethyl)-2-(3,4-dichlorophenyl)-1-ethyl-4-oxo-pyridine-3-carboxylate (2.0 g, 3.9 mmol). The reaction mixture was stirred at room temperature for 2 hours. On completion of reaction, the reaction was evaporated to dryness under reduced pressure to give ethyl 5-bromo-2-(3,4-dichlorophenyl)-1-ethyl-6-(hydrazinomethyl)-4-oxo-pyridine-3-carboxylate as light yellow solid.

### Step 2: Synthesis of ethyl 5-bromo-2-(3,4-dichlorophenyl)-1-ethyl-6-[[5-hydroxy-3-(trifluoromethyl)pyrazol-1-yl]methyl]-4-oxo-pyridine-3-carboxylate

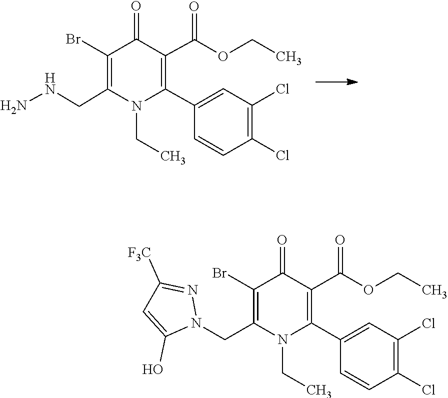 [SMILES: CCOC(=O)c1c(-c2ccc(Cl)c(Cl)c2)n(CC)c(CNN)c(Br)c1=O ; CCOC(=O)c1c(-c2ccc(Cl)c(Cl)c2)n(CC)c(Cn2nc(C(F)(F)F)cc2O)c(Br)c1=O]

To a stirred solution of ethyl 5-bromo-2-(3,4-dichlorophenyl)-1-ethyl-6-(hydrazinomethyl)-4-oxo-pyridine-3-carboxylate (1.4 g, 3.02 mmol) in toluene (3 mL) was added methyl 4,4,4-trifluoro-3-oxo-butanoate (0.514 g, 3.02 mmol) and p-toluenesulfonic acid monohydrate (29 mg, 0.15 mmol). The reaction mixture was heated with stirring at reflux for 8 hours. The cooled reaction mixture was filtered, washing with dichloromethane to give ethyl 5-bromo-2-(3,4-dichlorophenyl)-1-ethyl-6-((5-hydroxy-3-(trifluoromethyl)-1H-pyrazol-1-yl)meyl)-4-oxo-1,4-dihydropyridine-3-carboxylate as a white solid.

### Step 3: Synthesis of 5-bromo-2-(3,4-dichlorophenyl)-1-ethyl-6-[[5-hydroxy-3-(trifluoromethyl)pyrazol-1-yl]methyl]-4-oxo-pyridine-3-carboxylic acid

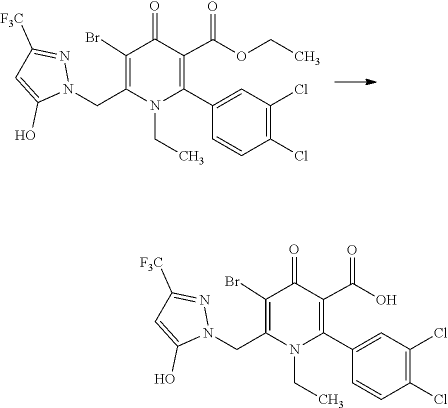 [SMILES: CCOC(=O)c1c(-c2ccc(Cl)c(Cl)c2)n(CC)c(Cn2nc(C(F)(F)F)cc2O)c(Br)c1=O ; CCn1c(Cn2nc(C(F)(F)F)cc2O)c(Br)c(=O)c(C(=O)O)c1-c1ccc(Cl)c(Cl)c1]

To a stirred mixture of ethyl 5-bromo-2-(3,4-dichlorophenyl)-1-ethyl-6-((5-hydroxy-3-(trifluoromethyl) 1H-pyrazol-1-yl)meyl)-4-oxo-1,4-dihydropyridine-3-carboxylate (200 mg, 0.34 mmol) in a mixture of tetrahydrofuran (1 mL) and water (1 mL) was added lithium hydroxide (25 mg, 1.03 mmol). The reaction mixture was heated with stirring at 65° C. for 18 hours. The cooled reaction mixture was evaporated to dryness under reduced pressure and the residue was suspended in water which was acidified by addition of 2 M aqueous hydrogen chloride solution. The product was extracted into ethyl acetate and evaporated to dryness under reduced pressure to 5-bromo-2-(3,4-dichlorophenyl)-1-ethyl-6-[[5-hydroxy-3-(trifluoromethyl)pyrazol-1-yl]methyl]-4-oxo-pyridine-3-carboxylic acid as a light yellow solid.

### Step 4: Synthesis of 2-(3,4-dichlorophenyl)-1-ethyl-6-[[5-hydroxy-3-(trifluoromethyl)pyrazol-1-yl]methyl]-4-oxo-pyridine-3-carboxylic acid

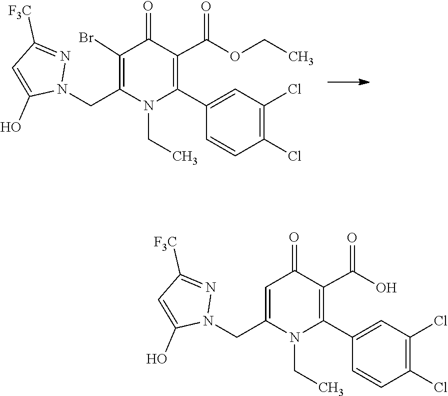 [SMILES: CCOC(=O)c1c(-c2ccc(Cl)c(Cl)c2)n(CC)c(Cn2nc(C(F)(F)F)cc2O)c(Br)c1=O ; CCn1c(Cn2nc(C(F)(F)F)cc2O)cc(=O)c(C(=O)O)c1-c1ccc(Cl)c(Cl)c1]

To a solution of 5-bromo-2-(3,4-dichlorophenyl)-1-ethyl-6-[[5-hydroxy-3-(trifluoromethyl)pyrazol-1-yl]methyl]-4-oxo-pyridine-3-carboxylic acid (140 mg, 0.252 mmol) in acetonitrile (3 mL) was added ammonium formate (79.5 mg, 1.26 mmol) and bis(triphenylphosphine)palladium(II)chloride (17.7 mg, 0.025 mmol). The reaction mixture was heated under microwave irradiation at 120° C. for 1 hour. The cooled reaction mixture was diluted with acetonitrile and filtered through diatomaceous earth, washing with dichloromethane. The filtrate was evaporated to dryness under reduced pressure. The crude residue was purified by flash chromatography on reverse-phase C-18 silica gel using a gradient of 5 to 95% acetonitrile (+0.1% formic acid) in water (+0.1% formic acid) as eluent to give 2-(3,4-dichlorophenyl)-1-ethyl-6-[[5-hydroxy-3-(trifluoromethyl)pyrazol-1-yl]methyl]-4-oxo-pyridine-3-carboxylic acid as a white powder.

1H NMR (400 MHz, DMSO-d6) δ=12.53 (s, 1H), 7.82 (d, 1H), 7.79 (d, 1H), 7.45 (d, 1H), 5.94-5.89 (m, 2H), 5.48 (s, 2H), 3.88 (q, 2H), 1.06 (t, 3H)

### Example 13: Synthesis of 2-(3,4-dichlorophenyl)-1-ethyl-6-[[5-(2-methoxyethoxy)-3-(trifluoromethyl)pyrazol-1-yl]methyl]-4-oxo-pyridine-3-carboxylic acid (Compound 144)

### Step 1: Synthesis of ethyl 2-(3,4-dichlorophenyl)-1-ethyl-6-[[5-(2-methoxyethoxy)-3-(trifluoromethyl) pyrazol-1-yl]methyl]-4-oxo-pyridine-3-carboxylate

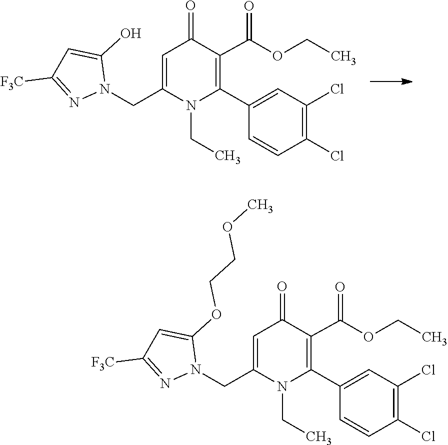 [SMILES: CCOC(=O)c1c(-c2ccc(Cl)c(Cl)c2)n(CC)c(Cn2nc(C(F)(F)F)cc2O)cc1=O ; CCOC(=O)c1c(-c2ccc(Cl)c(Cl)c2)n(CC)c(Cn2nc(C(F)(F)F)cc2OCCOC)cc1=O]

To a stirred solution of ethyl 2-(3,4-dichlorophenyl)-1-ethyl-6-[[5-hydroxy-3-(trifluoromethyl) pyrazol-1-yl]methyl]-4-oxo-pyridine-3-carboxylate (200 mg, 0.40 mmol) in N,N-dimethylformamide (2 mL) was added 1-iodo-2-methoxy-ethane (0.148 g, 0.793 mmol) and potassium carbonate (0.164 g, 1.19 mmol). The reaction mixture was heated with stirring at 50° C. for 1 hour. The cooled reaction mixture was diluted with water and extracted into ethyl acetate. The combined organic phases were washed with brine, dried over anhydrous sodium sulfate, filtered and evaporated to dryness under reduced pressure to give crude ethyl 2-(3,4-dichlorophenyl)-1-ethyl-6-[[5-(2-methoxyethoxy)-3-(trifluoromethyl)pyrazol-1-yl]methyl]-4-oxo-pyridine-3-carboxylate which was directly used for next step without further purification.

### Step 2: Synthesis of 2-(3,4-dichlorophenyl)-1-ethyl-6-[[5-(2-methoxyethoxy)-3-(trifluoromethyl)pyrazol-1-yl]methyl]-4-oxo-pyridine-3-carboxylic acid

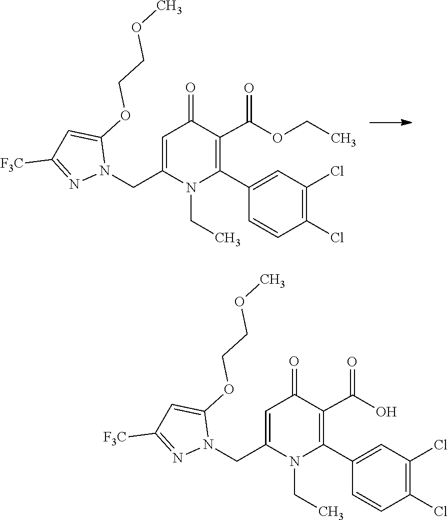

To a stirred solution of ethyl 2-(3,4-dichlorophenyl)-1-ethyl-6-[[5-(2-methoxyethoxy)-3-(trifluoromethyl) pyrazol-1-yl]methyl]-4-oxo-pyridine-3-carboxylate (200 mg, 0.356 mmol) in a mixture of tetrahydrofuran (1 mL) and water (1 mL) at room temperature was added lithium hydroxide monohydrate (0.045 g, 1.07 mmol). The reaction mixture was heated with stirring at 50° C. for 12 hours. The cooled reaction mixture was diluted with dichloromethane and water and acidified to pH 3 by addition of 1 M aqueous hydrogen chloride solution. The phases were separated and the organic phase was dried over anhydrous sodium sulfate, filtered and evaporated to dryness under reduced pressure. The crude residue was purified by preparative-HPLC to give 2-(3,4-dichlorophenyl)-1-ethyl-6-[[5-(2-methoxyethoxy)-3-(trifluoromethyl) pyrazol-1-yl]methyl]-4-oxo-pyridine-3-carboxylic acid as a white solid.

1H NMR (400 MHz, DMSO-d6) δ=7.87-7.70 (m, 2H), 7.44 (dd, 1H), 6.48 (s, 1H), 6.01 (s, 1H), 5.51 (s, 2H), 4.46-4.30 (m, 2H), 3.88 (q, 2H), 3.68-3.61 (m, 2H), 3.25 (s, 3H), 1.05 (t, 3H)

### Example 14: Synthesis of 2-(3,4-dichlorophenyl)-6-[[5-(difluoromethoxy)-3-(trifluoromethyl)pyrazol-1-yl]methyl]-1-ethyl-4-oxo-pyridine-3-carboxylic acid (Compound 151)

### Step 1: Synthesis of ethyl 2-(3,4-dichlorophenyl)-6-[[5-(difluoromethoxy)-3-(trifluoromethyl)pyrazol-1-yl]methyl]-1-ethyl-4-oxo-pyridine-3-carboxylate

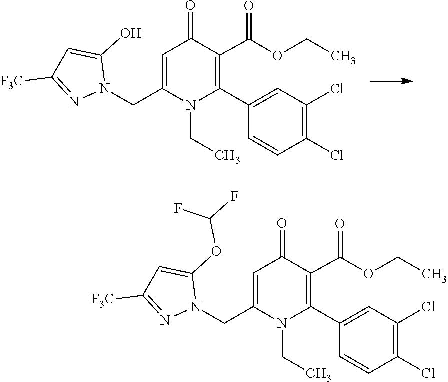

To a stirred mixture of ethyl 2-(3,4-dichlorophenyl)-1-ethyl-6-[[5-hydroxy-3-(trifluoromethyl)pyrazol-1-yl]methyl]-4-oxo-pyridine-3-carboxylate (200 mg, 0.397 mmol) and sodium;2-chloro-2,2-difluoro-acetate (181 mg, 1.2 mmol) in N,N-dimethylformamide (5 mL) was added cesium carbonate (388 mg, 1.2 mmol). The reaction mixture was stirred at room temperature for 12 hours. The reaction mixture was evaporated to dryness under reduced pressure and the residue was diluted with water and extracted into ethyl acetate (×2). The combined organic extracts were evaporated to dryness under reduced pressure and the resultant residue was purified by flash chromatography on silica gel using a gradient of 0 to 3% methanol in dichloromethane as eluent to give ethyl 2-(3,4-dichlorophenyl)-6-[[5-(difluoromethoxy)-3-(trifluoromethyl)pyrazol-1-yl]methyl]-1-ethyl-4-oxo-pyridine-3-carboxylate as a white solid.

### Step 2: Synthesis of 2-(3,4-dichlorophenyl)-6-[[5-(difluoromethoxy)-3-(trifluoromethyl)pyrazol-1-yl]methyl]-1-ethyl-4-oxo-pyridine-3-carboxylic acid

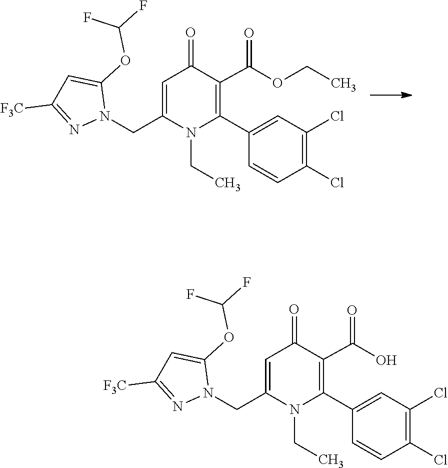 [SMILES: CCOC(=O)c1c(-c2ccc(Cl)c(Cl)c2)n(CC)c(Cn2nc(C(F)(F)F)cc2OC(F)F)cc1=O ; CCn1c(Cn2nc(C(F)(F)F)cc2OC(F)F)cc(=O)c(C(=O)O)c1-c1ccc(Cl)c(Cl)c1]

To a stirred solution of ethyl 2-(3,4-dichlorophenyl)-6-[[5-(difluoromethoxy)-3-(trifluoromethyl)pyrazol-1-yl]methyl]-1-ethyl-4-oxo-pyridine-3-carboxylate (82 mg, 0.148 mmol) in a mixture of tetrahydrofuran (5 mL) and water (1 mL) was added scandium(III) trifluoromethanesulfonate (109 mg, 0.22 mmol) in one portion. The reaction mixture was heated with stirring at 50° C. for 24 hours under an atmosphere of nitrogen. The cooled reaction mixture was evaporated to remove tetrahydrofuran and the residue was extracted into ethyl acetate (×3). The combined organic extracts were evaporated to dryness under reduced pressure. The crude residue was purified by preparative-HPLC to give 2-(3,4-dichlorophenyl)-6-((5-(difluoromethoxy)-3-(trifluoromethyl)-1H-pyrazol-1-yl)methyl)-1-ethyl-4-oxo-1,4-dihydropyridine-3-carboxylic acid as a white solid. 1H NMR (400 MHz, DMSO-d6) δ=16.12 (s, 1H), 7.80 (m, 2H), 7.62-7.26 (m, 1H), 7.47-7.41 (m, 1H), 6.84 (s, 1H), 5.92 (s, 1H), 5.69 (s, 2H), 3.87 (d, 2H), 1.06 (t, 3H)

### Example 15: Synthesis of 6-[(3-acetamidopyrazol-1-yl)methyl]-2-(3,4-dichlorophenyl)-1-ethyl-4-oxo-pyridine-3-carboxylic acid (Compound 154)

### Step 1: Synthesis of ethyl 6-[(3-acetamidopyrazol-1-yl)methyl]-5-bromo-2-(3,4-dichlorophenyl)-1-ethyl-4-oxo-pyridine-3-carboxylate

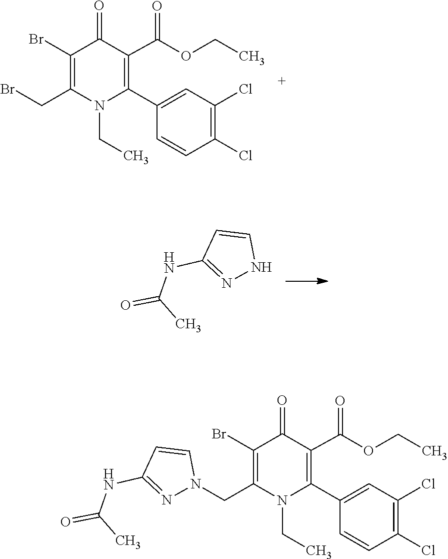

To a stirred solution of N-(1H-pyrazol-3-yl)acetamide (1.47 g, 11.7 mmol) in anhydrous tetrahydrofuran (50 mL) and at 0° C. was added portion-wise over 5 minutes sodium hydride (0.508 g, 12.7 mmol). The reaction mixture was stirred at this temperature for 0.5 hours. A solution of ethyl 5-bromo-6-(bromomethyl)-2-(3,4-dichlorophenyl)-1-ethyl-4-oxo-pyridine-3-carboxylate (5.0 g, 9.8 mmol) in tetrahydrofuran was added by dropwise over 5 minutes. The reaction was stirred at 0° C. for 0.5 hours before being allowed to warm to room temperature and stirred for a further 0.5 hours. The reaction mixture was diluted with water (!00 mL) at 0° C. and extracted into dichloromethane (×3). The combined organic extracts were washed sequentially with water (×3) and brine, dried over anhydrous sodium sulfate, filtered and evaporated to dryness under reduced pressure. The crude residue was purified by flash chromatography on silica gel using a gradient of 10 to 50% ethyl acetate in petroleum ether as eluent to give ethyl 6-[(3-acetamidopyrazol-1-yl)methyl]-5-bromo-2-(3,4-dichlorophenyl)-1-ethyl-4-oxo-pyridine-3-carboxylate.

1H NMR (400 MHz, DMSO) δ=10.46 (s, 1H), 7.89-7.75 (m, 3H), 7.46 (dd, 1H), 6.56 (d, 1H), 5.67 (s, 2H), 4.04-3.78 (m, 4H), 1.97 (s, 3H), 0.98-0.80 (m, 6H)

### Step 2: Synthesis of ethyl 6-[(3-acetamidopyrazol-1-yl)methyl]-2-(3,4-dichlorophenyl)-1-ethyl-4-oxo-pyridine-3-carboxylate

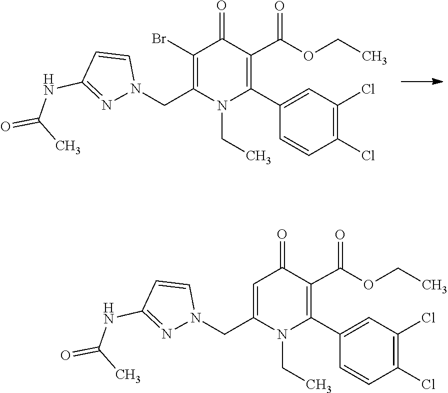 [SMILES: CCOC(=O)c1c(-c2ccc(Cl)c(Cl)c2)n(CC)c(Cn2ccc(NC(C)=O)n2)c(Br)c1=O ; CCOC(=O)c1c(-c2ccc(Cl)c(Cl)c2)n(CC)c(Cn2ccc(NC(C)=O)n2)cc1=O]

To a solution of ethyl 6-[(3-acetamidopyrazol-1-yl)methyl]-5-bromo-2-(3,4-dichlorophenyl)-1-ethyl-4-oxo-pyridine-3-carboxylate (3.00 g, 5.4 mmol) in a mixture of methanol (25 mL) and acetonitrile (25 mL) was added 10% palladium on carbon (0.50 g), The reaction mixture was stirred under a balloon pressure of hydrogen at room temperature for 12 hours. The reaction mixture was filtered through diatomaceous earth and the filtrate was evaporated to dryness under reduced pressure. The crude residue was purified by flash chromatography on reverse-phase C-18 silica gel using a gradient of 5 to 95% acetonitrile (+0.1% formic acid) in water (+0.1% formic acid) as eluent to give ethyl 6-[(3-acetamidopyrazol-1-yl)methyl]-2-(3,4-dichlorophenyl)-1-ethyl-4-oxo-pyridine-3-carboxylate as light yellow solid.

### Step 3: Synthesis of 6-[(3-acetamidopyrazol-1-yl)methyl]-2-(3,4-dichlorophenyl)-1-ethyl-4-oxo-pyridine-3-carboxylic acid

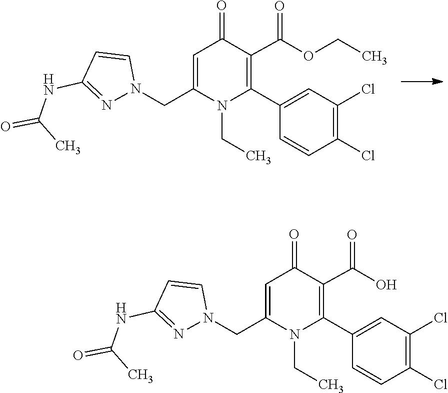 [SMILES: CCOC(=O)c1c(-c2ccc(Cl)c(Cl)c2)n(CC)c(Cn2ccc(NC(C)=O)n2)cc1=O ; CCn1c(Cn2ccc(NC(C)=O)n2)cc(=O)c(C(=O)O)c1-c1ccc(Cl)c(Cl)c1]

To a stirred solution of ethyl 6-[(3-acetamidopyrazol-1-yl)methyl]-2-(3,4-dichlorophenyl)-1-ethyl-4-oxo-pyridine-3-carboxylate (200 mg, 0.42 mmol) in a mixture of tetrahydrofuran (1.5 mL) and water (1.5 mL) and at room temperature was added sodium hydroxide (0.067 g, 1.68 mmol). The reaction mixture was heated with stirring at 50° C. for 2 hours. The cooled reaction mixture was acidified to pH 4 by addition of 1 M aqueous hydrogen chloride solution and extracted into dichloromethane (×3). The combined organic extracts were dried over anhydrous sodium sulfate, filtered and evaporated to dryness under reduced pressure. The crude residue was purified by preparative-HPLC to give 6-[(3-acetamidopyrazol-1-yl)methyl]-2-(3,4-dichlorophenyl)-1-ethyl-4-oxo-pyridine-3-carboxylic acid as a white solid.

1H NMR (400 MHz, DMSO-d6) δ=10.50 (s, 1H), 7.85 (d, 1H), 7.81-7.74 (m, 2H), 7.42 (dd, 1H), 6.63 (d, 1H), 6.12 (s, 1H), 5.56 (s, 2H), 3.88 (d, 2H), 2.00 (s, 3H), 1.03 (t, 3H)

### Example 16: Synthesis of 6-[(3-aminopyrazol-1-yl)methyl]-2-(3,4-dichlorophenyl)-1-ethyl-4-oxo-pyridine-3-carboxylic acid (Compound 156)

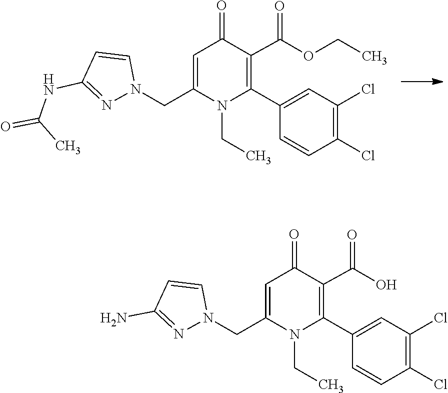 [SMILES: CCOC(=O)c1c(-c2ccc(Cl)c(Cl)c2)n(CC)c(Cn2ccc(NC(C)=O)n2)cc1=O ; CCn1c(Cn2ccc(N)n2)cc(=O)c(C(=O)O)c1-c1ccc(Cl)c(Cl)c1]

To a stirred solution of ethyl 6-[(3-acetamidopyrazol-1-yl)methyl]-2-(3,4-dichlorophenyl)-1-ethyl-4-oxo-pyridine-3-carboxylate (0.200 g, 0.42 mmol) in a mixture of tetrahydrofuran (1.5 mL) and water (1.5 mL) was added sodium hydroxide (0.067 g, 1.68 mmol). The reaction mixture was heated with stirring at 80° C. for 2 hours. The cooled reaction mixture was acidified to pH 4 by addition of 1 M aqueous hydrogen chloride solution and extracted into dichloromethane (×3). The combined organic extracts were dried over anhydrous sodium sulfate, filtered and evaporated to dryness under reduced pressure. The crude residue was purified by preparative-HPLC to give 6-[(3-aminopyrazol-1-yl)methyl]-2-(3,4-dichlorophenyl)-1-ethyl-4-oxo-pyridine-3-carboxylic acid as a white solid.

1H NMR (400 MHz, DMSO-d6) δ=7.82-7.74 (m, 2H), 7.57 (d, 1H), 7.42 (dd, 1H), 6.05 (s, 1H), 5.57 (d, 1H), 5.37 (s, 2H), 4.82 (s, 2H), 3.87 (d, 2H), 1.06 (t, 3H)

### Example 17: Synthesis of 6-[[3-[[(E)-but-2-enoyl]amino]pyrazol-1-yl]methyl]-2-(3,4-dichlorophenyl)-1-ethyl-4-oxo-pyridine-3-carboxylic acid (Compound 155) G-8C

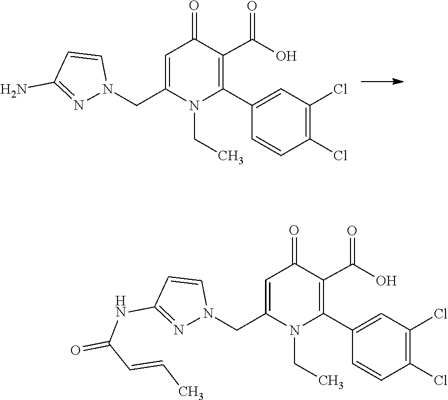 [SMILES: CCn1c(Cn2ccc(N)n2)cc(=O)c(C(=O)O)c1-c1ccc(Cl)c(Cl)c1 ; C/C=C/C(=O)Nc1ccn(Cc2cc(=O)c(C(=O)O)c(-c3ccc(Cl)c(Cl)c3)n2CC)n1]

To a stirred mixture of (E)-but-2-enoic acid (63 mg, 0.74 mmol) in anhydrous N,N-dimethylformamide (5 mL) was added [dimethylamino(triazolo[4,5-b]pyridin-3-yloxy)methylene]-dimethyl-ammonium;hexafluorophosphate (HATU, 280 mg, 0.74 mmol) and triethylamine (150 mg, 1.5 mmol). The reaction mixture was stirred at room temperature for 0.5 hours before addition of 6-((3-amino-1H-pyrazol-1-yl)methyl)-2-(3,4-dichlorophenyl)-1-ethyl-4-oxo-1,4-dihydropyridine-3-carboxylic acid (200 mg, 0.49 mmol). The reaction mixture was stirred at room temperature for 2 hours before being quenched into water and extracted into ethyl acetate (×3). The combined organic extracts were dried over anhydrous sodium sulfate, filtered and evaporated to dryness under reduced pressure. The crude residue was purified by preparative-HPLC to give 6-[[3-[[(E)-but-2-enoyl]amino]pyrazol-1-yl]methyl]-2-(3,4-dichlorophenyl)-1-ethyl-4-oxo-pyridine-3-carboxylic acid as a white solid.

1H NMR (400 MHz, DMSO-d6) δ=10.57 (d, 1H), 7.87 (s, 1H), 7.80-7.76 (m, 2H), 7.42 (d, 1H), 6.86-6.73 (m, 1H), 6.71 (s, 1H), 6.16 (d, 2H), 5.57 (s, 2H), 3.89 (d, 2H), 1.84 (d, 3H), 1.02 (t, 3H)

### Example 18: Synthesis of 2-(3,4-dichlorophenyl)-1-ethyl-6-[(3-hydroxypyrazol-1-yl)methyl]-4-oxo-pyridine-3-carboxylic acid (Compound 167)

### Step 1: Synthesis of ethyl 6-((3-(benzyloxy)-1H-pyrazol-1-yl)methyl)-5-bromo-2-(3,4-dichlorophenyl)-1-ethyl-4-oxo-1,4-dihydropyridine-3-carboxylate

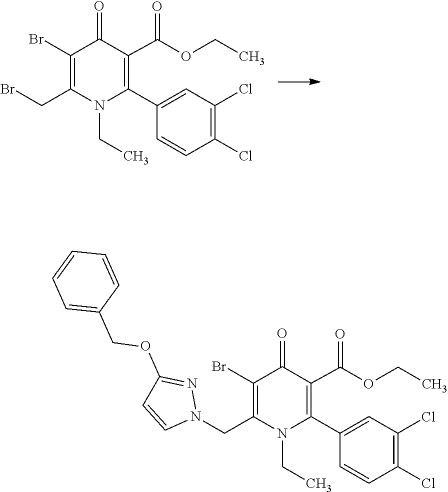 [SMILES: CCOC(=O)c1c(-c2ccc(Cl)c(Cl)c2)n(CC)c(CBr)c(Br)c1=O ; CCOC(=O)c1c(-c2ccc(Cl)c(Cl)c2)n(CC)c(Cn2ccc(OCc3ccccc3)n2)c(Br)c1=O]

To a solution of 1-[3-(benzyloxy)-1H-pyrazol-1-yl]ethanone (680 mg, 3.90 mmol) in anhydrous tetrahydrofuran (10 mL) under an atmosphere of nitrogen was added sodium hydride (230 mg, 10 mmol). The reaction mixture was stirred at room temperature for 0.5 hours followed by dropwise addition of a solution of ethyl 5-bromo-6-(bromomethyl)-2-(3,4-dichlorophenyl)-1-ethyl-4-oxo-pyridine-3-carboxylate (2.0 g, 3.9 mmol) in anhydrous tetrahydrofuran (10 mL). The reaction mixture was stirred at room temperature for 1 hour. The reaction mixture was diluted with water (10 mL) and extracted into ethyl acetate (×3). The combined organic extracts were washed with brine, dried over anhydrous sodium sulfate, filtered and evaporated to dryness under reduced pressure to give ethyl 6-[(3-benzyloxypyrazol-1-yl)methyl]-5-bromo-2-(3,4-dichlorophenyl)-1-ethyl-4-oxo-pyridine-3-carboxylate as a white solid.

### Step 2: Synthesis of ethyl 2-(3,4-dichlorophenyl)-1-ethyl-6-((3-hydroxy-1H-pyrazol-1-yl) methyl)-4-oxo-1,4-dihydropyridine-3-carboxylate

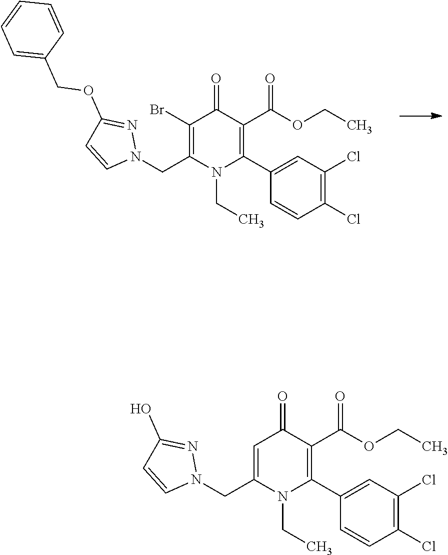 [SMILES: CCOC(=O)c1c(-c2ccc(Cl)c(Cl)c2)n(CC)c(Cn2ccc(OCc3ccccc3)n2)c(Br)c1=O ; CCOC(=O)c1c(-c2ccc(Cl)c(Cl)c2)n(CC)c(Cn2ccc(O)n2)cc1=O]

To a stirred solution of ethyl 6-((3-(benzyloxy)-1H-pyrazol-1-yl)methyl)-5-bromo-2-(3,4-dichlorophenyl)-1-ethyl-4-oxo-1,4-dihydropyridine-3-carboxylate (1.0 g, 1.65 mmol) in tetrahydrofuran (10 mL) was added 10% palladium on carbon (1 g). The reaction mixture was stirred under balloon pressure of hydrogen at room temperature for 6 hours. The reaction mixture was filtered through diatomaceous earth and the filtrate was evaporated to dryness under reduced pressure. The crude residue was purified by flash chromatography on silica gel using a gradient of 0 to 5% methanol in dichloromethane as eluent to give ethyl 2-(3,4-dichlorophenyl)-1-ethyl-6-((3-hydroxy-1H-pyrazol-1-yl)methyl)-4-oxo-1,4-dihydropyridne-3-carboxylate as a white powder.

### Step 3: Synthesis of 2-(3,4-dichlorophenyl)-1-ethyl-6-[(3-hydroxypyrazol-1-yl)methyl]-4-oxo-pyridine-3-carboxylic acid

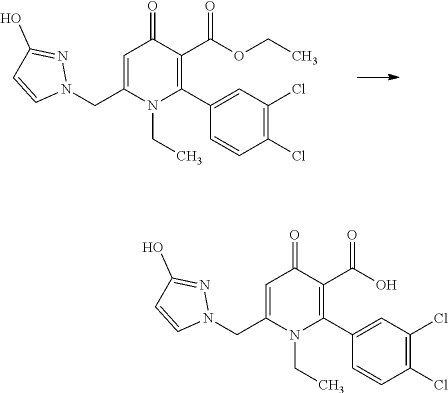 [SMILES: CCOC(=O)c1c(-c2ccc(Cl)c(Cl)c2)n(CC)c(Cn2ccc(O)n2)cc1=O ; CCn1c(Cn2ccc(O)n2)cc(=O)c(C(=O)O)c1-c1ccc(Cl)c(Cl)c1]

To a stirred mixture of ethyl 2-(3,4-dichlorophenyl)-1-ethyl-6-((3-hydroxy-1H-pyrazol-1-yl) methyl)-4-oxo-1,4-dihydropyridine-3-carboxylate (230 mg, 0.527 mmol) in a mixture of tetrahydrofuran (2.5 mL), methanol (2.5 mL) and water (2.5 mL) was added lithium hydroxide (50 mg, 2.09 mmol) in one portion. The reaction mixture was heated with stirring at 50° C. for 18 hours. The cooled reaction mixture was evaporated to dryness under reduced pressure then diluted with water (5 mL) and acidified to pH 1 by addition of 2 M aqueous hydrogen chloride solution. The precipitated solid was filtered, washed with water and then purified by flash chromatography on reverse-phase C-18 silica gel using a gradient of 5 to 100% acetonitrile (+0.1% formic acid) in water (+0.1% formic acid) as eluent to give 2-(3,4-dichlorophenyl)-1-ethyl-6-((3-hydroxy-1H-pyrazol-1-yl) methyl)-4-oxo-1,4-dihydropyridine-3-carboxylic acid as a white powder.

1H NMR (400 MHz, DMSO-d6) δ=9.94 (s, 1H), 7.82-7.73 (m, 2H), 7.66 (d, 1H), 7.45-7.40 (m, 1H), 6.08 (d, 1H), 5.64 (d, 1H), 5.41 (s, 2H), 3.88 (q, 2H), 1.05 (t, 3H)

### Example 19: Synthesis of 2-(3,4-dichlorophenyl)-1-ethyl-6-[[3-(2-methoxyethoxy)pyrazol-1-yl]methyl]-4-oxo-pyridine-3-carboxylic acid (Compound 161)

### Step 1: Synthesis of ethyl 2-(3,4-dichlorophenyl)-1-ethyl-6-[[3-(2-methoxyethoxy)pyrazol-1-yl]methyl]-4-oxo-pyridine-3-carboxylate

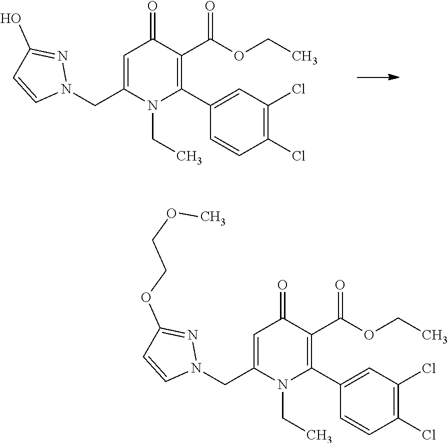 [SMILES: CCOC(=O)c1c(-c2ccc(Cl)c(Cl)c2)n(CC)c(Cn2ccc(O)n2)cc1=O ; CCOC(=O)c1c(-c2ccc(Cl)c(Cl)c2)n(CC)c(Cn2ccc(OCCOC)n2)cc1=O]

To a stirred mixture of ethyl 2-(3,4-dichlorophenyl)-1-ethyl-6-[(3-hydroxypyrazol-1-yl)methyl]-4-oxo-pyridine-3-carboxylate (150 mg, 0.344 mmol) and 1-iodo-2-methoxy-ethane (192 mg, 1.03 mmol) in N,N-dimethylformamide (3 mL) was added potassium carbonate (190 mg, 1.38 mmol). The reaction mixture was stirred at room temperature for 24 hours. The reaction mixture was evaporated to dryness under reduced pressure and the residue was diluted with water and extracted into ethyl acetate (×3). The combined organic extracts were evaporated to dryness under reduced pressure and the crude residue was purified by flash chromatography on silica gel using a gradient of 0 to 3% methanol in dichloromethane as eluent to give ethyl 2-(3,4-dichlorophenyl)-1-ethyl-6-[[3-(2-methoxyethoxy)pyrazol-1-yl]methyl]-4-oxo-pyridine-3-carboxylate as a white solid.

### Step 2: Synthesis of 2-(3,4-dichlorophenyl)-1-ethyl-6-[[3-(2-methoxyethoxy)pyrazol-1-yl]methyl]-4-oxo-pyridine-3-carboxylic acid

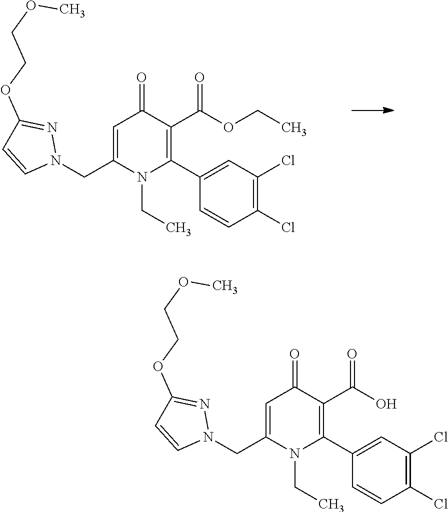 [SMILES: CCOC(=O)c1c(-c2ccc(Cl)c(Cl)c2)n(CC)c(Cn2ccc(OCCOC)n2)cc1=O ; CCn1c(Cn2ccc(OCCOC)n2)cc(=O)c(C(=O)O)c1-c1ccc(Cl)c(Cl)c1]

To a stirred solution of ethyl 2-(3,4-dichlorophenyl)-1-ethyl-6-[[3-(2-methoxyethoxy)pyrazol-1-yl]methyl]-4-oxo-pyridine-3-carboxylate (120 mg, 0.243 mmol) in a mixture of tetrahydrofuran (2 mL) and water (2 mL) was added lithium hydroxide (23 mg, 0.97 mmol) in one portion. The reaction mixture was stirred at room temperature for 24 hours. The tetrahydrofuran was removed under reduced pressure and the aqueous residue was washed with ethyl acetate (×2). The aqueous phase was acidified to pH 2 by addition of 1 M aqueous hydrogen chloride solution and then extracted into ethyl acetate (×3). The combined organic extracts were evaporated to dryness under reduced pressure and the crude residue was purified by preparative-HPLC to give 2-(3,4-dichlorophenyl)-1-ethyl-6-[[3-(2-methoxyethoxy)pyrazol-1-yl]methyl]-4-oxo-pyridine-3-carboxylic acid as a white solid.

1H NMR (400 MHz, DMSO-d6) δ=7.87-7.68 (m, 3H), 7.43 (dd, 1H), 5.95 (s, 1H), 5.88 (d, 1H), 5.50 (s, 2H), 4.23-4.12 (m, 2H), 3.88 (q, 2H), 3.67-3.54 (m, 2H), 3.29 (s, 3H), 1.08 (t, 3H)

### Example 20: Synthesis of 2-(3,4-dichlorophenyl)-1-ethyl-4-oxo-6-[1-[3-(trifluoromethyl)pyrazol-1-yl]ethyl]pyridine-3-carboxylic acid (Compound 172)

### Step 1: Synthesis of 5-(1-hydroxypropylidene)-2,2-dimethyl-1,3-dioxane-4,6-dione

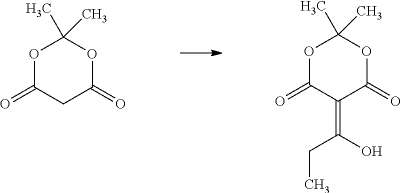

To a stirred solution of 2,2-dimethyl-1,3-dioxane-4,6-dione (10.0 g, 69.4 mmol) in dichloromethane (100 mL) at 0° C. was added pyridine (11.2 mL, 139 mmol) and propanoyl chloride (6.42 g, 69.4 mmol). The reaction mixture was stirred at 0° C. for 1 hour and then at room temperature for 1 hour. The reaction mixture was poured onto ice water, acidified by addition of 2 M aqueous hydrogen chloride solution and extracted into dichloromethane (×2). The combined organic extracts were dried over anhydrous sodium sulfate, filtered and evaporated to dryness under reduced pressure to give 5-(1-hydroxypropylidene)-2,2-dimethyl-1,3-dioxane-4,6-dione as a yellow solid.

1H NMR (400 MHz, chloroform) δ=15.4 (brs, 1H), 3.11 (q, 2H), 1.74 (s, 6H), 1.26 (t, 3H)

### Step 2: Synthesis of 6-ethyl-2,2-dimethyl-1,3-dioxin-4-one

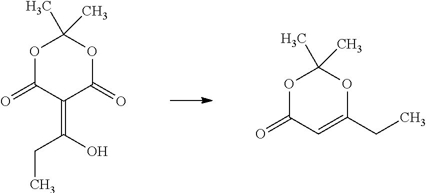

To a stirred solution of 5-(1-hydroxypropylidene)-2,2-dimethyl-1,3-dioxane-4,6-dione (1.0 g, 5.0 mmol) in toluene (10 mL) at room temperature was added acetone (0.145 g, 2.5 mmol). The reaction mixture was heated with stirring at 120° C. for 1 hour. The cooled reaction mixture was poured onto ice and extracted into ethyl acetate (×2). The combined organic extracts were dried over anhydrous sodium sulfate, filtered and evaporated to dryness under reduced pressure. The crude residue was purified by flash chromatography on silica gel using a gradient of 0 to 50% ethyl acetate in hexanes as eluent to give 6-ethyl-2,2-dimethyl-1,3-dioxin-4-one as a colourless liquid.

1H NMR (400 MHz, chloroform) δ=5.22 (s, 1H), 2.25 (q, 2H), 1.68 (s, 6H), 1.13 (t, 3H)

### Step 3: Synthesis of 6-(1-bromoethyl)-2,2-dimethyl-1,3-dioxin-4-one

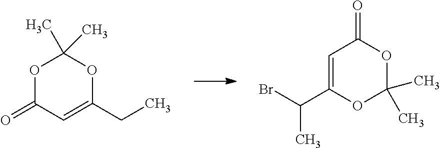

To a stirred solution of 6-ethyl-2,2-dimethyl-1,3-dioxin-4-one (2.0 g, 11.5 mmol) in dichloromethane (20 mL) was added 1-bromopyrrolidine-2,5-dione (2.26 g, 12.7 mmol) and 2-[(E)-(1-cyano-1-methyl-ethyl)azo]-2-methyl-propanenitrile (0.189 g, 1.15 mmol) at room temperature. The reaction mixture was irradiated in the Penn reactor (λ=420 nm) with stirring at room temperature for 16 hours. The reaction mixture was diluted with dichloromethane and a saturated aqueous solution of sodium thiosulfate was added. The phases were separated, and the aqueous phase was extracted into dichloromethane (×2). The combined organic extracts were dried over anhydrous sodium sulfate, filtered and evaporated to dryness under reduced pressure. The crude residue was purified by flash chromatography on silica gel using a gradient of 0 to 50% ethyl acetate in hexanes as eluent to give 6-(1-bromoethyl)-2,2-dimethyl-1,3-dioxin-4-one as a colourless liquid.

1H NMR (400 MHz, chloroform) δ=5.48 (s, 1H), 4.49 (q, 1H), 1.84 (d, 3H), 1.72 (d, 6H).

### Step 4: Synthesis of ethyl 6-(1-bromoethyl)-2-(3,4-dichlorophenyl)-1-ethyl-4-oxo-pyridine-3-carboxylate

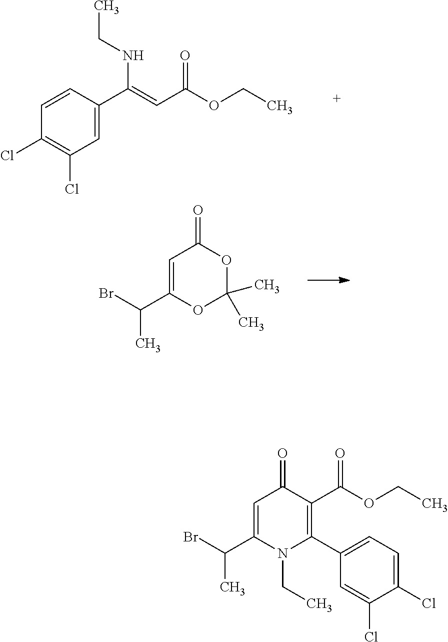 [SMILES: CCN/C(=C\C(=O)OCC)c1ccc(Cl)c(Cl)c1 ; CC(Br)C1=CC(=O)OC(C)(C)O1 ; CCOC(=O)c1c(-c2ccc(Cl)c(Cl)c2)n(CC)c(C(C)Br)cc1=O]

A stirred solution of ethyl (Z)-3-(3,4-dichlorophenyl)-3-(ethylamino)prop-2-enoate (4.0 g, 14 mmol) and 6-(1-bromoethyl)-2,2-dimethyl-1,3-dioxin-4-one (4.9 g, 21 mmol) in xylene (40 mL) was heated with stirring at 120° C. for 0.5 hours. The cooled reaction mixture was evaporated to dryness under reduced pressure and the residue was washed with methyl tert-butyl ether to give ethyl 6-(1-bromoethyl)-2-(3,4-dichlorophenyl)-1-ethyl-4-oxo-pyridine-3-carboxylate as light yellow solid.

1H NMR (400 MHz, DMSO-d6) δ=7.94 (d, 0.5H) 7.85 (d, 0.5H), 7.82 (d, 1H), 7.55-7.47 (m, 1H), 6.90 (d, 1H), 5.61-5.54 (m, 1H), 3.99-3.82 (m, 4H), 2.03 (q, 3H), 1.13 (t, 3H), 0.83 (t, 3H)

### Step 5: Synthesis of ethyl 2-(3,4-dichlorophenyl)-1-ethyl-4-oxo-6-[1-[3-(trifluoromethyl)pyrazol-1-yl]ethyl]pyridine-3-carboxylate

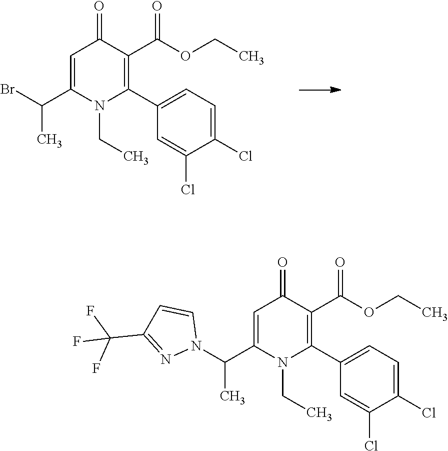 [SMILES: CCOC(=O)c1c(-c2ccc(Cl)c(Cl)c2)n(CC)c(C(C)Br)cc1=O ; CCOC(=O)c1c(-c2ccc(Cl)c(Cl)c2)n(CC)c(C(C)n2ccc(C(F)(F)F)n2)cc1=O]

To a stirred solution of ethyl 6-(1-bromoethyl)-2-(3,4-dichlorophenyl)-1-ethyl-4-oxo-pyridine-3-carboxylate (100 mg, 0.223 mmol) in N,N-dimethylformamide (10 mL) was added 3-(trifluoromethyl)-1H-pyrazole (28 mg, 0.27 mmol) and potassium carbonate (93 mg, 0.67 mmol). The reaction mixture was stirred at room temperature for 6 hours. The reaction mixture was diluted with water (50 mL) extracted into ethyl acetate (×2). The combined organic extracts were dried over anhydrous sodium sulfate, filtered and evaporated to dryness under reduced pressure. The crude residue was purified by flash chromatography on silica gel using a gradient of 10 to 50% ethyl acetate in hexanes as eluent to give ethyl 2-(3,4-dichlorophenyl)-1-ethyl-4-oxo-6-(1-(3-(trifluoromethyl)-1H-pyrazol-1-yl)ethyl)-1,4-dihydropyridine-3-carboxylate as white solid.

### Step 6: Synthesis of 2-(3,4-dichlorophenyl)-1-ethyl-4-oxo-6-[1-[3-(trifluoromethyl)pyrazol-1-yl]ethyl]pyridine-3-carboxylic acid

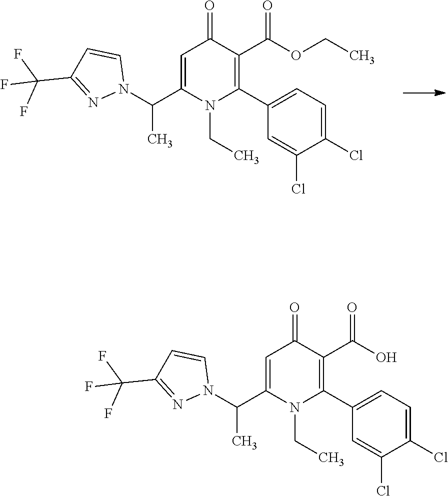 [SMILES: CCOC(=O)c1c(-c2ccc(Cl)c(Cl)c2)n(CC)c(C(C)n2ccc(C(F)(F)F)n2)cc1=O ; CCn1c(C(C)n2ccc(C(F)(F)F)n2)cc(=O)c(C(=O)O)c1-c1ccc(Cl)c(Cl)c1]

To a stirred solution of ethyl 2-(3,4-dichlorophenyl)-1-ethyl-4-oxo-6-[1-[3-(trifluoromethyl)pyrazol-1-yl]ethyl]pyridine-3-carboxylate (180 mg, 0.358 mmol) in a mixture of tetrahydrofuran (8 mL) and water (2 mL) at room temperature was added lithium hydroxide hydrate (30 mg, 0.72 mmol). The reaction mixture was heated with stirring at 80° C. for 6 hours. The cooled reaction mixture was acidified by addition of 2 M aqueous hydrogen chloride solution and extracted into ethyl acetate. The combined organic extract was evaporated to dryness under reduced pressure and the crude residue was purified by flash chromatography on reverse-phase C-18 silica gel using a gradient of 0 to 100% acetonitrile (+0.1% formic acid) in water (+0.1% formic acid) as eluent to give 2-(3,4-dichlorophenyl)-1-ethyl-4-oxo-6-[1-[3-(trifluoromethyl)pyrazol-1-yl]ethyl]pyridine-3-carboxylic acid as a white solid.

1H NMR (400 MHz, DMSO-d6) δ=8.38 (s, 1H), 7.87-7.75 (m, 2H), 7.52-7.44 (m, 1H), 6.93 (s, 1H), 6.63 (s, 0.5H), 6.54 (s, 0.5H), 6.18-6.13 (m, 1H), 4.00-3.85 (m, 2H), 1.93 (d, 3H), 1.14 (t, 3H)

### Example 21: Synthesis of 2-(3,4-dichlorophenyl)-1-ethyl-6-[[3-[(E)-methoxyiminomethyl]pyrazol-1-yl]methyl]-4-oxo-pyridine-3-carboxylic acid (Compound 204)

### Step 1: Synthesis of ethyl 2-(3,4-dichlorophenyl)-1-ethyl-6-[(3-formylpyrazol-1-yl)methyl]-4-oxo-pyridine-3-carboxylate

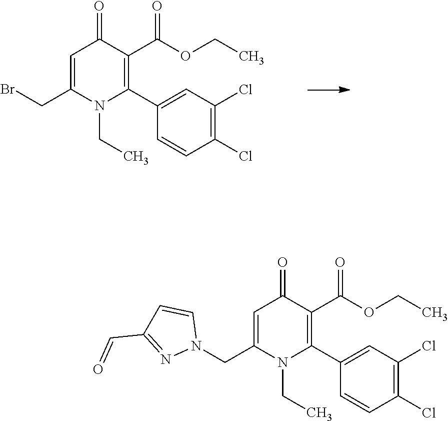 [SMILES: CCOC(=O)c1c(-c2ccc(Cl)c(Cl)c2)n(CC)c(CBr)cc1=O ; CCOC(=O)c1c(-c2ccc(Cl)c(Cl)c2)n(CC)c(Cn2ccc(C=O)n2)cc1=O]

To a stirred solution of ethyl 6-(bromomethyl)-2-(3,4-dichlorophenyl)-1-ethyl-4-oxo-pyridine-3-carboxylate (380 mg, 0.83 mmol) in acetonitrile (15 mL) at room temperature was added 1H-pyrazole-3-carbaldehyde (88 mg, 0.92 mmol) and potassium carbonate (230 mg, 1.67 mmol). The reaction mixture was stirred at room temperature for 24 hours. The reaction mixture was diluted with water and extracted into ethyl acetate (×2). The combined organic extracts were dried over anhydrous sodium sulfate, filtered and evaporated to dryness under reduced pressure to give crude ethyl 2-(3,4-dichlorophenyl)-1-ethyl-6-[(3-formylpyrazol-1-yl)methyl]-4-oxo-pyridine-3-carboxylate which was used in subsequent step without any purification.

### Step 2: Synthesis of ethyl 2-(3,4-dichlorophenyl)-1-ethyl-6-[[3-[(E)-methoxyiminomethyl]pyrazol-1-yl]methyl]-4-oxo-pyridine-3-carboxylate

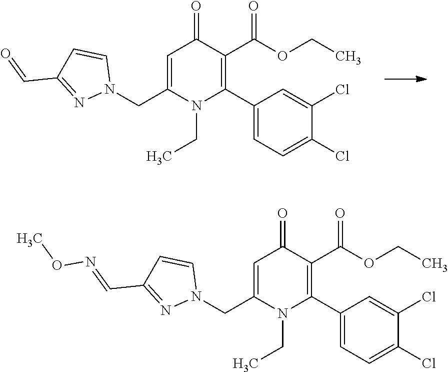 [SMILES: CCOC(=O)c1c(-c2ccc(Cl)c(Cl)c2)n(CC)c(Cn2ccc(C=O)n2)cc1=O ; CCOC(=O)c1c(-c2ccc(Cl)c(Cl)c2)n(CC)c(Cn2ccc(/C=N/OC)n2)cc1=O]

To a stirred solution of ethyl 2-(3,4-dichlorophenyl)-1-ethyl-6-[(3-formylpyrazol-1-yl)methyl]-4-oxo-pyridine-3-carboxylate (140 mg, 0.312 mmol) in a mixture of methanol (5 mL) and water (1.5 mL) at 0° C. was added sodium acetate (0.038 g, 0.47 mmol) and O-methylhydroxylamine;hydrochloride (0.052 g, 0.63 mmol). The reaction mixture was stirred at room temperature for 5 minutes and then heated with stirring at 80° C. for 6 hours. The reaction mixture was quenched by addition of water and extracted into ethyl acetate (×3). The combined organic extracts were washed sequentially with water and brine, dried over anhydrous sodium sulfate, filtered and evaporated to dryness under reduced pressure. The crude residue was purified by flash chromatography on silica gel using a gradient of 0 to 50% ethyl acetate in hexanes as eluent to give ethyl 2-(3,4-dichlorophenyl)-1-ethyl-6-[[3-[(E)-methoxyiminomethyl]pyrazol-1-yl]methyl]-4-oxo-pyridine-3-carboxylate as a colourless liquid.

### Step 3: Synthesis of 2-(3,4-dichlorophenyl)-1-ethyl-6-[[3-[(E)-methoxyiminomethyl]pyrazol-1-yl]methyl]-4-oxo-pyridine-3-carboxylic acid

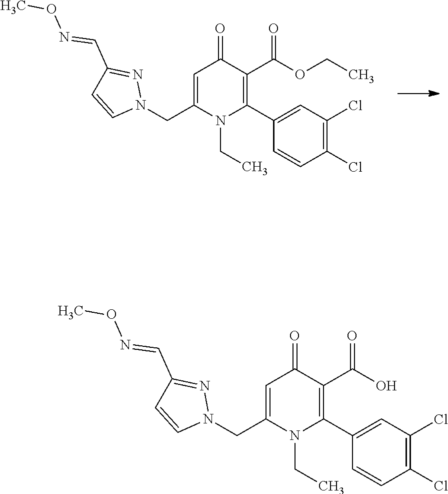 [SMILES: CCOC(=O)c1c(-c2ccc(Cl)c(Cl)c2)n(CC)c(Cn2ccc(/C=N/OC)n2)cc1=O ; CCn1c(Cn2ccc(/C=N/OC)n2)cc(=O)c(C(=O)O)c1-c1ccc(Cl)c(Cl)c1]

To a stirred solution of ethyl 2-(3,4-dichlorophenyl)-1-ethyl-6-[[3-[(E)-methoxyiminomethyl]pyrazol-1-yl]methyl]-4-oxo-pyridine-3-carboxylate (110 mg, 0.22 mmol) in a mixture of tetrahydrofuran (8 mL) and water (2 mL) at room temperature was added lithium hydroxide (28 mg, 0.66 mmol). The reaction mixture was heated with stirring at 80° C. for 16 hours. The reaction mixture was diluted with 1 M aqueous hydrogen chloride solution and extracted into ethyl acetate. The combined organic extracts were washed sequentially with water and brine, dried over anhydrous sodium sulfate, filtered and evaporated to dryness under reduced pressure. The crude residue was purified by flash chromatography on reverse-phase C-18 silica gel using water as eluent to give 2-(3,4-dichlorophenyl)-1-ethyl-6-[[3-[(E)-methoxyiminomethyl]pyrazol-1-yl]methyl]-4-oxo-pyridine-3-carboxylic acid as a white solid.

1H NMR (400 MHz, DMSO-d6) δ=8.16 (s, 1H), 8.03 (d, 1H), 7.79 (d, 1H), 7.77 (d, 1H), 7.42 (dd, 1H), 6.68 (d, 1H), 6.00 (s, 1H), 5.71 (s, 2H), 3.91-3.88 (m, 2H), 3.88 (s, 3H), 1.03 (t, 3H)

### Example 22: Synthesis of 2-(3,4-dichlorophenyl)-1-ethyl-4-oxo-6-[[3-(2,2,2-trifluoroethoxy)pyrazol-1-yl]methyl]pyridine-3-carboxylic acid (Compound 186)

### Step 1: Synthesis of ethyl 2-(3,4-dichlorophenyl)-6-[[3-(difluoromethyl)pyrazol-1-yl]methyl]-1-ethyl-4-oxo-pyridine-3-carboxylate

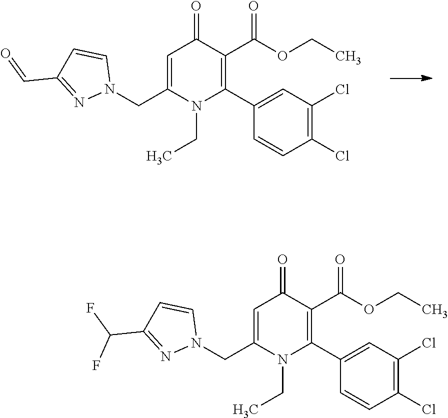 [SMILES: CCOC(=O)c1c(-c2ccc(Cl)c(Cl)c2)n(CC)c(Cn2ccc(C=O)n2)cc1=O ; CCOC(=O)c1c(-c2ccc(Cl)c(Cl)c2)n(CC)c(Cn2ccc(C(F)F)n2)cc1=O]

To a stirred solution of ethyl 2-(3,4-dichlorophenyl)-1-ethyl-6-[(3-formylpyrazol-1-yl)methyl]-4-oxo-pyridine-3-carboxylate (250 mg, 0.50 mmol) in dichloromethane (15 mL) at −20° C. was added N-ethyl-N-(trifluoro-λ⁴-sulfanyl)ethanamine (0.20 mL, 1.51 mmol). The reaction mixture was stirred at room temperature for 5 hours. The reaction mixture was diluted with water and extracted into dichloromethane (×3). The combined organic extracts were dried over anhydrous sodium sulfate, filtered and evaporated to dryness under reduced pressure. The crude residue was purified by flash chromatography on silica gel using a gradient of 10 to 100% ethyl acetate in hexanes as eluent to give ethyl 2-(3,4-dichlorophenyl)-6-[[3-(difluoromethyl)pyrazol-1-yl]methyl]-1-ethyl-4-oxo-pyridine-3-carboxylate as a light yellow solid.

1H NMR (400 MHz, DMSO-d6) δ=8.09 (s, 1H), 7.84-7.79 (m, 2H), 7.48 (d, 1H), 7.18-6.91 (s, 1H), 6.77 (s, 1H), 5.67 (s, 1H), 5.60 (s, 2H), 3.86-3.76 (m, 4H), 0.97 (t, 3H), 0.82 (t, 3H).

### Step 2: Synthesis of 2-(3,4-dichlorophenyl)-1-ethyl-4-oxo-6-[[3-(2,2,2-trifluoroethoxy)pyrazol-1-yl]methyl]pyridine-3-carboxylic acid

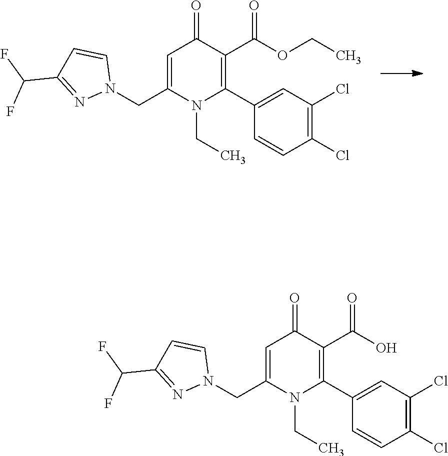 [SMILES: CCOC(=O)c1c(-c2ccc(Cl)c(Cl)c2)n(CC)c(Cn2ccc(C(F)F)n2)cc1=O ; CCn1c(Cn2ccc(C(F)F)n2)cc(=O)c(C(=O)O)c1-c1ccc(Cl)c(Cl)c1]

To a stirred solution of ethyl 2-(3,4-dichlorophenyl)-6-[[3-(difluoromethyl)pyrazol-1-yl]methyl]-1-ethyl-4-oxo-pyridine-3-carboxylate (160 mg, 0.34 mmol) in a mixture of tetrahydrofuran (5 mL) and water (2 mL) at room temperature was added lithium hydroxide;hydrate (29 mg, 0.68 mmol). The reaction mixture was heated with stirring at 80° C. for 16 hours. The cooled reaction mixture was acidified by addition of 2 M aqueous hydrogen chloride solution and extracted into ethyl acetate. The combined organic extract was evaporated to dryness under reduced pressure and the crude residue was purified by flash chromatography on reverse-phase C-18 silica gel using a gradient of 0 to 100% acetonitrile (+0.1% formic acid) in water (+0.1% formic acid) as eluent to give 2-(3,4-dichlorophenyl)-6-[[3-(difluoromethyl)pyrazol-1-yl]methyl]-1-ethyl-4-oxo-pyridine-3-carboxylic acid as a white solid.

1H NMR (400 MHz, DMSO-d6) δ=8.12 (d, 1H), 7.79 (t, 2H), 7.43 (dd, 1H), 7.19-6.92 (m, 1H), 6.71 (s, 1H), 5.99 (s, 1H), 5.75 (s, 2H), 3.91-3.86 (m, 2H), 1.03 (t, 3H)

Throughout this description, temperatures are given in degrees Celsius (° C.) and “m.p.” means melting point. LC/MS means Liquid Chromatography Mass Spectrometry and the description of the apparatus and the methods are as follows:

Method A: Spectra were recorded on a Mass Spectrometer from Waters Aquity UPLC-MS using a Sample Organizer with Sample Manager FTN, H-class QSM, Column Manager, 2×Column Manager Aux, photodiode array, ELSD and a QDA SQD 2. SQD Mass Spectrometer-ionization method: electrospray (ESI), Polarity: positive ions, Capillary (kV) 3.00, Cone (V) 30.00, Extractor (V) 2.00, Source Temperature (° C.) 150, Desolvation Temperature (° C.) 350, Cone Gas Flow (L/Hr) 0, Desolvation Gas Flow (L/Hr) 650). Instrument equipped with a Waters HSS T3 C18 column (column length 50 mm, internal diameter of column 2.3 mm, particle size 1.8 micron). Gradient elution 5-100% MeCN in water over 3.3 mins at 0.6 ml/min. MeCN and water both containing 0.05% v/v TFA.

Method B: Spectra were recorded on a Mass Spectrometer from Waters (QDa Single Quadrupole mass spectrometer) equipped with an electrospray source (polarity: positive and negative ions, Probe Temperature: 600° C., Cone: 15 V, Source Temperature: 120° C., ESI Capillary: 0.8 kV, Mass range: positive 130 to 700 Da, negative 130 to 700 Da, data: centroid) and an Acquity UPLC from Waters using a 2777 Sample Manager, H-class BSM, Column Manager UBM, Photodiode Array Detector UPD and Corona Veo RS Charged Aerosol Detector. Column: Phenomenex Kinetex 2.6 um C18 100 Å 50×4.6 mm P.N. 00B-4462-E0. Temp: 40° C., DAD wavelength range: 205 to 310 nm, Solvent gradient: Solvent A: H₂O with 0.1% formic acid, Solvent B: CH₃CN with 0.1% formic acid, gradient: 0 min 30% B, 70% A; 1.2 min 90% B, 10% A; 1.7 min 90% B, 10% A; 1.8 min 30% B, 70% A; 2.0 min 30% B, 70% A. Flow 2.0 ml/min. Injection volume 2 ul. Total run time 2.0 min

TABLE 2
¹H NMR and LC/MS for selected compounds according to the invention.

| | Compound | | |
|---|---|---|---|
| Compound No. | Name | Structure & ¹H NMR Data | LC/MS |
| 1 | ethyl 2-(3,4- dichlorophenyl)-1- ethyl-4-oxo-6-[[3- (trifluoromethyl) pyrazol-1- yl]methyl]pyridine- 3-carboxylate | 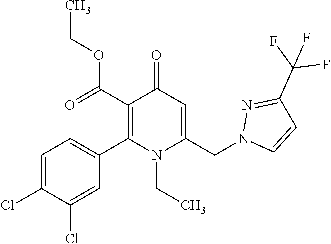 [SMILES: CCOC(=O)c1c(-c2ccc(Cl)c(Cl)c2)n(CC)c(Cn2ccc(C(F)(F)F)n2)cc1=O] | |
| | | 1H NMR (400 MHz, chloroform) δ = 7.65-7.62 (m, 1H), | |
| | | 7.57 (d, 1H), 7.51 (d, 1H), 7.26-7.23 (m, 1H), 6.65 (d, | |
| | | 1H), 6.34 (s, 1H), 5.35 (s, 2H), 4.09-3.97 (m, 2H), 3.87 | |
| | | (q, 2H), 1.04-0.95 (m, 6H) | |
| 2 | 2-(3,4- dichlorophenyl)-1- ethyl-4-oxo-6-[[3- (trifluoromethyl) pyrazol-1- yl]methyl]pyridine- 3-carboxylic acid | 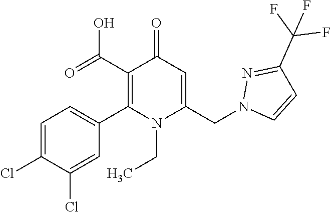 [SMILES: CCn1c(Cn2ccc(C(F)(F)F)n2)cc(=O)c(C(=O)O)c1-c1ccc(Cl)c(Cl)c1] | |
| | | 1H NMR (400 MHz, chloroform) δ = 7.73-7.67 (m, 1H), | |
| | | 7.61 (d, 1H), 7.42 (d, 1H), 7.16 (dd, 1H), 6.67 (d, 1H), | |
| | | 6.23 (s, 1H), 5.47 (s, 2H), 4.04-3.90 (m, 2H), 1.24- | |
| | | 1.17 (m, 3H) | |
| 3 | ethyl 6-[(3- chloropyrazol-1- yl)methyl]-2-(3,4- dichlorophenyl)-1- ethyl-4-oxo- pyridine-3- carboxylate | 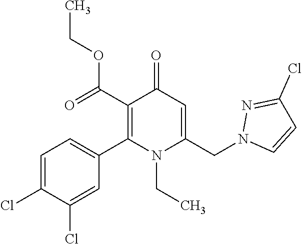 [SMILES: CCOC(=O)c1c(-c2ccc(Cl)c(Cl)c2)n(CC)c(Cn2ccc(Cl)n2)cc1=O] | |
| | | 1H NMR (400 MHz, chloroform) δ = 7.59 (d, 1H), 7.52 | |
| | | (dd, 2H), 7.26-7.24 (m, 1H), 6.74 (s, 1H), 6.32 (d, 1H), | |
| | | 5.29 (s, 2H), 4.08-3.97 (m, 4H), 1.09 (t, 3H), 1.00 (t, | |
| | | 3H) | |
| 4 | 6-[(3-chloropyrazol- 1-yl)methyl]-2-(3,4- dichlorophenyl)-1- ethyl-4-oxo- pyridine-3- carboxylic acid | 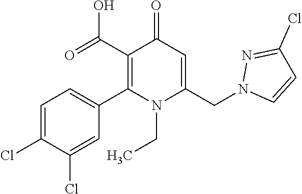 [SMILES: CCn1c(Cn2ccc(Cl)n2)cc(=O)c(C(=O)O)c1-c1ccc(Cl)c(Cl)c1] | |
| | | 1H NMR (400 MHz, chloroform) δ = 7.61 (d, 1H), 7.51 | |
| | | (d, 1H), 7.38 (d, 1H), 7.13 (dd, 1H), 6.39 (s, 1H), 6.35 (d, | |
| | | 1H), 5.34 (s, 2H), 3.97 (q, 2H), 1.20 (t, 3H) | |
| 5 | 6-[(3-chloro-1,2,4- triazol-1-yl)methyl]- 2-(3,4- dichlorophenyl)-1- ethyl-4-oxo- pyridine-3- carboxylic acid | 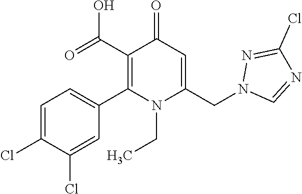 [SMILES: CCn1c(Cn2cnc(Cl)n2)cc(=O)c(C(=O)O)c1-c1ccc(Cl)c(Cl)c1] | |
| | | 1H NMR (400 MHz, methanol) δ = 8.67 (s, 1H), 7.72 (d, | |
| | | 1H), 7.68 (d, 1H), 7.40-7.33 (m, 1H), 6.49 (s, 1H), 5.74 | |
| | | (s, 2H), 4.05 (q, 2H), 1.21 (t, 3H) | |
| 6 | 2-(3,4- dichlorophenyl)-1- ethyl-4-oxo-6-[[4- (trifluoromethyl) pyrazol-1- yl]methyl]pyridine- 3-carboxylic acid | 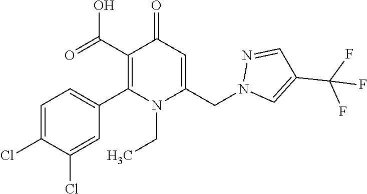 [SMILES: CCn1c(Cn2cc(C(F)(F)F)cn2)cc(=O)c(C(=O)O)c1-c1ccc(Cl)c(Cl)c1] | |
| 7 | 2-(3,4- dichlorophenyl)-1- ethyl-4-oxo-6-[[3- (trifluoromethyl)- 1,2,4-triazol-1- yl]methyl]pyridine- 3-carboxylic acid | 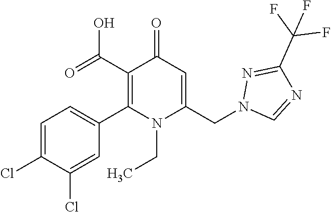 [SMILES: CCn1c(Cn2cnc(C(F)(F)F)n2)cc(=O)c(C(=O)O)c1-c1ccc(Cl)c(Cl)c1] | |
| | | 1H NMR (400 MHz, DMSO-d6) δ 9.11 (s, 1H), 7.81 (d, | |
| | | 1H), 7.79 (d, 1H), 7.44 (dd, 1H), 6.40 (s, 1H), 5.90 (s, | |
| | | 2H), 3.89 (q, 2H), 1.03 (t, 3H) | |
| 8 | ethyl 2-(3,4- dichlorophenyl)-1- ethyl-6-[(4- fluoropyrazol-1- yl)methyl]-4-oxo- pyridine-3- carboxylate | 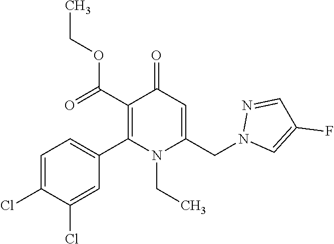 [SMILES: CCOC(=O)c1c(-c2ccc(Cl)c(Cl)c2)n(CC)c(Cn2cc(F)cn2)cc1=O] | |
| | | 1H NMR (400 MHz, chloroform) δ = 7.57 (d, 1H), 7.51 | |
| | | (d, 1H), 7.45-7.38 (m, 2H), 7.25 (d, 1H), 6.37 (s, 1H), | |
| | | 5.20 (s, 2H), 4.03 (dd, 2H), 3.85 (brd, 2H), 1.04-0.96 | |
| | | (m, 6H) | |
| 9 | 2-(3,4- dichlorophenyl)-1- ethyl-6-[(4- fluoropyrazol-1- yl)methyl]-4-oxo- pyridine-3- carboxylic acid | 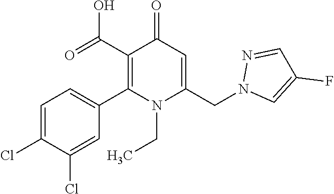 [SMILES: CCn1c(Cn2cc(F)cn2)cc(=O)c(C(=O)O)c1-c1ccc(Cl)c(Cl)c1] | |
| | | 1H NMR (400 MHz, DMSO-d6) δ = 8.13 (brd, 1H), 7.80 | |
| | | (brs, 1H), 7.78 (brs, 1H), 7.72 (brd, 1H), 7.44 (brd, 1H), | |
| | | 6.06 (s, 1H), 5.61 (s, 2H), 3.88 (brd, 2H), 1.05 (brt, 3H) | |
| 10 | 2-(3,4- dichlorophenyl)-1- ethyl-4-oxo-6-[[4- (trifluoromethyl) imidazol-1- yl]methyl]pyridine 3-carboxylic acid | 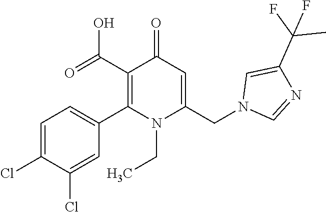 [SMILES: CCn1c(Cn2cnc(C(C)(F)F)c2)cc(=O)c(C(=O)O)c1-c1ccc(Cl)c(Cl)c1] | |
| | | 1H NMR (400 MHz, methanol) δ = 8.07-8.00 (m, 1H), | |
| | | 7.89-7.84 (m, 1H), 7.77-7.71 (m, 1H), 7.68-7.65 (m, | |
| | | 1H), 7.39-7.33 (m, 1H), 6.10 (s, 1H), 5.67 (s, 2H), 4.02- | |
| | | 3.94 (m, 2H), 1.22 (t, 3H) | |
| 11 | ethyl 6-[(3-chloro- 1,2,4-triazol-1- yl)methyl]-2-(3,4- dichlorophenyl)-1- ethyl-4-oxo-pyridine- 3-carboxamide | 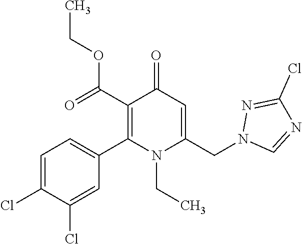 [SMILES: CCOC(=O)c1c(-c2ccc(Cl)c(Cl)c2)n(CC)c(Cn2cnc(Cl)n2)cc1=O] | |
| | | 1H NMR (400 MHz, chloroform) δ = 8.29 (s, 1H), 7.62 (d, | |
| | | 1H), 7.56 (d, 1H), 7.31 (dd, 1H), 6.69 (s, 1H), 5.39 (s, 2H), | |
| | | 4.08-3.98 (m, 4H), 1.18 (t, 3H), 0.99 (t, 3H) | |
| 12 | ethyl 2-(3,4- dichlorophenyl)-1- ethyl-4-oxo-6-[[3- (trifluoromethyl)-1,2,4- triazol-1- yl]methane]pyridine-3- carboxylate | 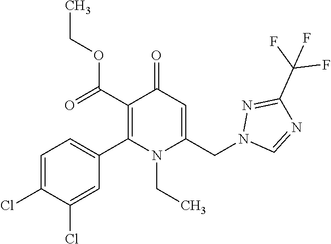 [SMILES: CCOC(=O)c1c(-c2ccc(Cl)c(Cl)c2)n(CC)c(Cn2cnc(C(F)(F)F)n2)cc1=O] | |
| | | 1H NMR (400 MHz, chloroform) δ = 8.55 (s, 1H), 7.60 (d, | |
| | | 1H), 7.56 (d, 1H), 7.31 (dd, 1H), 6.31 (s, 1H), 5.47 (d, 2H), | |
| | | 4.08-3.96 (m, 2H), 3.92 (q, 2H), 1.14 (t, 3H), 0.98 (t, 3H) | |
| 13a | 2-(3,4- dichlorophenyl)-1- ethyl-6-[[5-methyl-3- (trifluoromethyl)pyrazol- 1-yl]methyl]-4-oxo- pyridine-3-carboxylic acid | 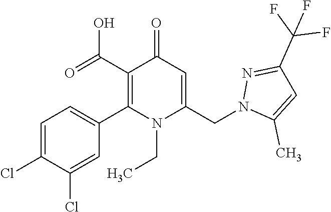 [SMILES: CCn1c(Cn2nc(C(F)(F)F)cc2C)cc(=O)c(C(=O)O)c1-c1ccc(Cl)c(Cl)c1] | Rₜ = 2.27 min (A); MS: m/z = 474.18 (M + 1) Mixture with 13b |
| 13b | 2-(3,4- dichlorophenyl)-1- ethyl-6-[[3-methyl-5- (trifluoromethyl)pyrazol- 1-yl]methyl]-4-oxo- pyridine-3-carboxylic acid | 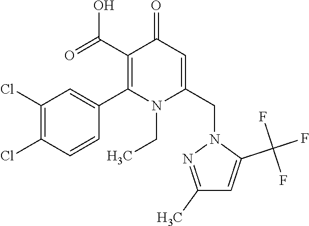 [SMILES: CCn1c(Cn2nc(C)cc2C(F)(F)F)cc(=O)c(C(=O)O)c1-c1ccc(Cl)c(Cl)c1] | Rₜ = 2.27 min (A); MS: m/z = 474.18 (M + 1) Mixture with 13a |
| 14 | 6-[(4-chloropyrazol-1- yl)methyl]-2-(3,4- dichlorophenyl)-1- ethyl-4-oxo-pyridine- 3-carboxylic acid | 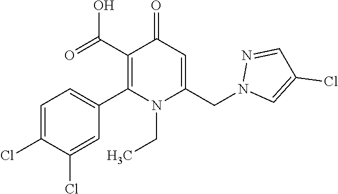 [SMILES: CCn1c(Cn2cc(Cl)cn2)cc(=O)c(C(=O)O)c1-c1ccc(Cl)c(Cl)c1] | |
| | | 1H NMR (400 MHz, chloroform) δ = 7.63-7.58 (m, 2H), | |
| | | 7.52 (s, 1H), 7.40 (d, 1H), 7.17 (dd, 1H), 6.31 (s, 1H), 5.36 | |
| | | (s, 2H), 3.95 (q, 2H), 1.27-1.14 (m, 3H) | |
| 15 | 6-[(3-chloro-5-methyl- pyrazol-1-yl)methyl]- 2-(3,4- dichlorophenyl)-1- ethyl-4-oxo-pyridine- 3-carboxylic acid | 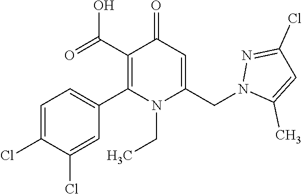 [SMILES: CCn1c(Cn2nc(Cl)cc2C)cc(=O)c(C(=O)O)c1-c1ccc(Cl)c(Cl)c1] | Rₜ = 0.93 min (B); MS: m/z = 442.19 (M + 1) Mixture with 17 |
| 16 | 2-(3,4- dichlorophenyl)-1- ethyl-4-oxo-6-[[5- (trifluoromethyl) imidazol-1- yl]methyl]pyridine-3- carboxylic acid | 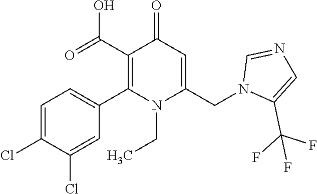 [SMILES: CCn1c(Cn2cncc2C(F)(F)F)cc(=O)c(C(=O)O)c1-c1ccc(Cl)c(Cl)c1] | |
| | | 1H NMR (400 MHz, chloroform) δ = 7.78 (s, 1H), 7.66- | |
| | | 7.60 (m, 2H), 7.41 (d, 1H), 7.17 (dd, 1H), 5.99 (s, 1H), 5.37 | |
| | | (s, 2H), 3.84 (q, 2H), 1.33-1.17 (m, 3H) | |
| 17 | 6-[(5-chloro-3-methyl- pyrazol-1-yl)methyl]-2-(3,4- dichlorophenyl)-1- ethyl-4-oxo-pyridine- 3-carboxylic acid | 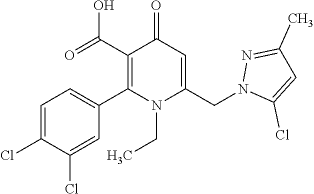 [SMILES: CCn1c(Cn2nc(C)cc2Cl)cc(=O)c(C(=O)O)c1-c1ccc(Cl)c(Cl)c1] | Rₜ = 0.93 min (B); MS: m/z = 442.19 (M + 1) Mixture with 17 |
| 18 | 5-bromo-2-(3,4- dichlorophenyl)-1- ethyl-4-oxo-6-[[3- (trifluoromethyl)pyrazol- 1-yl]methyl]pyridine-3- carboxylic acid | 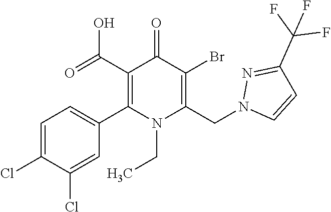 [SMILES: CCn1c(Cn2ccc(C(F)(F)F)n2)c(Br)c(=O)c(C(=O)O)c1-c1ccc(Cl)c(Cl)c1] | |
| | | 1H NMR (400 MHz, chloroform) δ = 7.84 (d, 1H), 7.62 (d, | |
| | | 1H), 7.36 (d, 1H), 7.11 (dd, 1H), 6.64 (d, 1H), 5.86 (s, 2H), | |
| | | 4.44 (brs, 2H), 1.28-1.15 (m, 3H) | |
| 19 | 2-(4-chlorophenyl)-1- ethyl-4-oxo-6-[[3- (trifluoromethyl)pyrazol- 1-yl]methyl] pyridine-3- carboxylic acid | 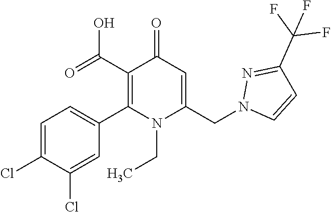 [SMILES: CCn1c(Cn2ccc(C(F)(F)F)n2)cc(=O)c(C(=O)O)c1-c1ccc(Cl)c(Cl)c1] | |
| | | 1H NMR (400 MHz, chloroform) δ = 7.71 (d, 1H), 7.51- | |
| | | 7.45 (m, 2H), 7.26-7.20 (m, 2H), 6.63 (d, 1H), 6.11 (s, 1H), | |
| | | 5.48 (s, 2H), 3.93 (q, 2H), 1.16 (t, 3H) | |
| 20 | 2-(3,4- dichlorophenyl)-1- ethyl-5-methyl-4-oxo- 6-[[3- (trifluoromethyl)pyrazol- 1-yl]methyl]pyridine-3- carboxylic acid | 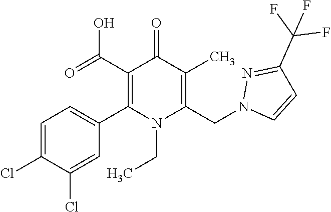 [SMILES: CCn1c(Cn2ccc(C(F)(F)F)n2)c(C)c(=O)c(C(=O)O)c1-c1ccc(Cl)c(Cl)c1] | |
| | | 1H NMR (400 MHz, methanol) δ = 7.64 (d, 1H), 7.61 (d, | |
| | | 1H), 7.40 (d, 1H), 7.14 (dd, 1H), 6.65 (d, 1H), 5.61 (s, 2H), | |
| | | 4.19-4.08 (m, 2H), 2.36 (s, 3H), 1.14 (t, 3H) | |
| 21 | 5-(4-chlorophenyl)-2- (3,4-dichlorophenyl)- 1-ethyl-4-oxo-6-[[3- (trifluoromethyl)pyrazol- 1-yl]methyl]pyridine-3- carboxylic acid | 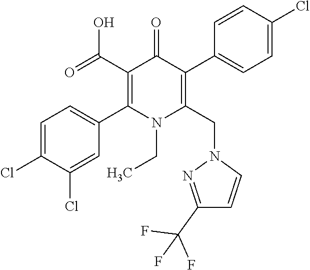 [SMILES: CCn1c(Cn2ccc(C(F)(F)F)n2)c(-c2ccc(Cl)cc2)c(=O)c(C(=O)O)c1-c1ccc(Cl)c(Cl)c1] | |
| | | 1H NMR (400 MHz, chloroform) δ = 7.61 (d, 1H), 7.48 (brd, | |
| | | 2H), 7.44-7.38 (m, 1H), 7.18 (dd, 1H), 7.12 (brd, 2H), | |
| | | 7.07 (d, 1H), 6.54 (d, 1H), 5.33 (s, 2H), 4.27-4.15 (m, 2H), | |
| | | 1.17 (t, 2H) | |
| 22 | methyl 2-(4- chlorophenyl)-1-ethyl- 4-oxo-6-[[3- (trifluoromethyl) pyrazol- 1-yl]methyl]pyridine-3- carboxylate | 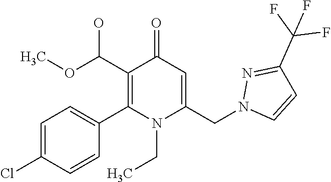 [SMILES: CCn1c(Cn2ccc(C(F)(F)F)n2)cc(=O)c(C(O)OC)c1-c1ccc(Cl)cc1] | |
| | | 1H NMR (400 MHz, chloroform + 2 drops MeOD) δ = 7.76 | |
| | | (s, 1H), 7.48 (d, J = 8.3 Hz, 2H), 7.36-7.30 (m, 2H), 6.65 | |
| | | (d, J = 2.1 Hz, 1H), 6.40 (s, 1H), 5.45 (s, 2H), 3.95 (q, J = | |
| | | 7.1 Hz, 2H), 3.51 (s, 3H), 1.02 (t, J = 7.1 Hz, 3H) | |
| 23 | ethyl 5-bromo-2-(3,4- dichlorophenyl)-1- ethyl-4-oxo-6-[[3- (trifluoromethyl)pyrazol- 1-yl]methyl]pyridine-3- carboxylate | 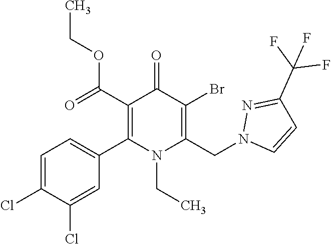 [SMILES: CCOC(=O)c1c(-c2ccc(Cl)c(Cl)c2)n(CC)c(Cn2ccc(C(F)(F)F)n2)c(Br)c1=O] | |
| | | 1H NMR (400 MHz, chloroform) δ = 7.80 (dd, 1H), 7.58 (d, | |
| | | 1H), 7.50 (d, 1H), 7.23 (dd, 1H), 6.64-6.61 (m, 1H), 5.81 | |
| | | (s, 2H), 4.23 (brd, 2H), 4.11-3.99 (m, 2H), 1.09-1.01 (m, | |
| | | 6H) | |
| 24 | ethyl 5-bromo-2-(3,4- dichlorophenyl)-1- ethyl-6-(imidazol-1- ylmethyl)-4-oxo- pyridine-3-carboxylate | 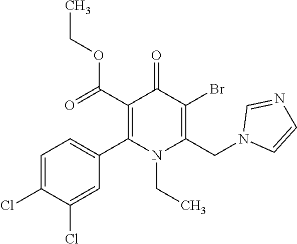 [SMILES: CCOC(=O)c1c(-c2ccc(Cl)c(Cl)c2)n(CC)c(Cn2ccnc2)c(Br)c1=O] | |
| | | 1H NMR (400 MHz, chloroform) δ = 7.71-7.60 (m, 1H), | |
| | | 7.57 (d, 1H), 7.48 (d, 1H), 7.22 (dd, 1H), 7.19 (brd, 1H), | |
| | | 7.13-6.98 (m, 1H), 5.60 (s, 2H), 4.14-3.99 (m, 2H), 3.79 | |
| | | (q, 2H), 1.04 (t, 3H), 0.99 (t, 3H) | |
| 25 | 2-(3-chlorophenyl)-1- ethyl-4-oxo-6-[[3- (trifluoromethyl)pyrazol- 1-yl]methyl]pyridine-3- carboxylic acid | 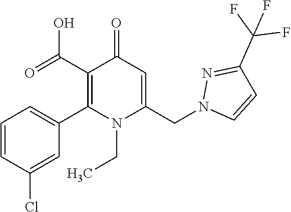 [SMILES: CCn1c(Cn2ccc(C(F)(F)F)n2)cc(=O)c(C(=O)O)c1-c1cccc(Cl)c1] | |
| | | 1H NMR (400 MHz, chloroform) δ = 7.70 (d, 1H), 7.56- | |
| | | 7.44 (m, 2H), 7.30 (t, 1H), 7.19 (d, 1H), 6.67 (d, 1H), 6.25 | |
| | | (s, 1H), 5.48 (s, 2H), 4.01-3.90 (m, 2H), 1.19 (t, 3H) | |
| 26 | ethyl 2-(3- chlorophenyl)-1-ethyl- 4-oxo-6-[[3- (trifluoromethyl) pyrazol-1- yl]methyl]pyridine-3- carboxylate | 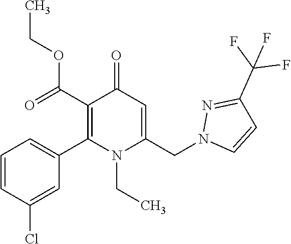 [SMILES: CCOC(=O)c1c(-c2cccc(Cl)c2)n(CC)c(Cn2ccc(C(F)(F)F)n2)cc1=O] | |
| | | 1H NMR (400 MHz, chloroform) δ = 7.59 (d, 1H), 7.51- | |
| | | 7.46 (m, 1H), 7.45-7.37 (m, 2H), 7.28 (t, 1H), 6.66 (d, | |
| | | 1H), 6.42 (s, 1H), 5.34 (s, 2H), 4.00 (dq, 2H), 3.86 (q, 2H), | |
| | | 1.00 (t, 3H), 0.93 (t, 3H) | |
| 27 | ethyl 5-bromo-6-[(3- chloropyrazo1-1- yl)methyl]-2-(3,4- dichlorophenyl)-1- ethyl-4-oxo-pyridine- 3-carboxylate | 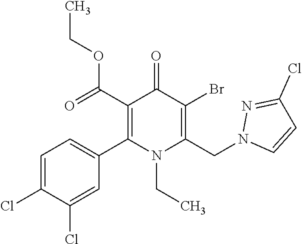 [SMILES: CCOC(=O)c1c(-c2ccc(Cl)c(Cl)c2)n(CC)c(Cn2ccc(Cl)n2)c(Br)c1=O] | |
| | | 1H NMR (400 MHz, chloroform-d) δ = 7.66 (d, J = 2.3 Hz, | |
| | | 1H), 7.60-7.56 (m, 1H), 7.51-7.50 (m, 1H), 7.25-7.23 | |
| | | (m, 1H), 6.30-6.25 (m, 1H), 5.68 (s, 2H), 4.24 (q, J = 6.9 | |
| | | Hz, 2H), 4.12-3.99 (m, 2H), 1.10 (t, J = 7.1 Hz, 3H), 1.07- | |
| | | 1.01 (m, 3H) | |
| 28 | 2-(3-chloro-4-fluoro- phenyl)-6-[(3- chloropyrazol-1- yl)methyl]-1-ethyl-4- oxo-pyridine-3- carboxylic acid | 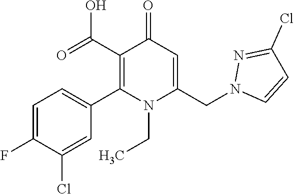 [SMILES: CCn1c(Cn2ccc(Cl)n2)cc(=O)c(C(=O)O)c1-c1ccc(F)c(Cl)c1] | |
| | | 1H NMR (400 MHz, DMSO-d6) δ = 8.06 (d, 1H), 7.77- | |
| | | 7.75 (dd, 1H), 7.58-7.54 (t, 1H), 7.47-7.43 (m, 1H), 6.52 | |
| | | (d, 1H), 6.08 (s, 1H), 5.65 (s, 2H), 3.90-3.85 (q, 2H), 1.03 (t, | |
| | | 3H) | |
| 29 | 6-[(3-chloropyrazol-1- yl)methyl]-2-(3,4- difluorophenyl)-1- ethyl-4-oxo-pyridine- 3-carboxylic acid | 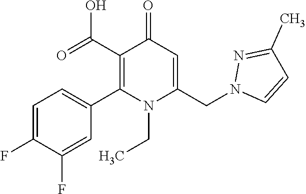 [SMILES: CCn1c(Cn2ccc(C)n2)cc(=O)c(C(=O)O)c1-c1ccc(F)c(F)c1] | |
| | | 1H NMR (400 MHz, DMSO-d6) δ = 8.05 (d, 1H), 7.67- | |
| | | 7.54 (m, 2H), 7.28-7.27 (m, 1H), 6.52 (d, 1H), 6.08 (s, | |
| | | 1H), 5.65 (s, 2H), 3.91-3.86 (m, 2H), 1.04 (t, 3H) | |
| 30 | 2-(4-chloro-3-fluoro- phenyl)-1-ethyl-4-oxo- 6-[[3- (trifluoromethyl) pyrazol-1- yl]methyl]pyridine-3- carboxylic acid | 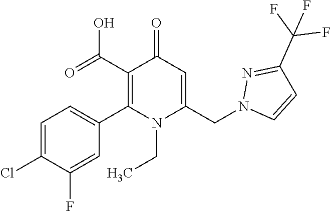 [SMILES: CCn1c(Cn2ccc(C(F)(F)F)n2)cc(=O)c(C(=O)O)c1-c1ccc(Cl)c(F)c1] | |
| | | 1H NMR (400 MHz, DMSO-d6) δ = 8.24 (s, 1H), 7.75 (t, | |
| | | 1H), 7.65 (d, 1H), 7.29 (d, 1H), 6.92 (s, 1H), 5.99 (s, 1H), | |
| | | 5.85 (s, 2H), 3.89 (s, 2H), 1.13 (t, 3H) | |
| 31 | 2-(3,4-difluorophenyl)- 1-ethyl-4-oxo-6-[[3- (trifluoromethyl) pyrazol-1- yl]methyl]pyridine-3- carboxylic acid | 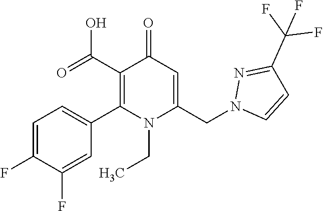 [SMILES: CCn1c(Cn2ccc(C(F)(F)F)n2)cc(=O)c(C(=O)O)c1-c1ccc(F)c(F)c1] | |
| | | 1H NMR (400 MHz, DMSO-d6) δ = 8.24 (d, 1H), 7.66- | |
| | | 7.55 (m, 2H), 7.29-7.27 (m, 1H), 6.94 (d, 1H), 5.98 (s, | |
| | | 1H), 5.82 (s, 2H), 3.91-3.86 (m, 2H), 1.03 (t, 3H) | |
| 32 | 2-(3-chloro-4-fluoro- phenyl)-1-ethyl-4-oxo- 6-[[3- (trifluoromethyl) pyrazol-1- yl]methyl]pyridine-3- carboxylic acid | 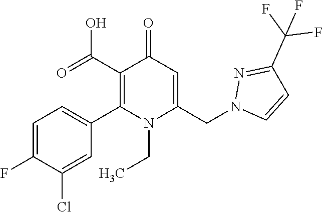 [SMILES: CCn1c(Cn2ccc(C(F)(F)F)n2)cc(=O)c(C(=O)O)c1-c1ccc(F)c(Cl)c1] | |
| | | 1H NMR (400 MHz, DMSO-d6) δ = 8.24-8.23 (m, 1H), | |
| | | 7.76-7.74 (m, 1H), 7.62-7.52 (m, 1H), 7.52-7.43 (m, | |
| | | 1H), 6.92 (d, 1H), 5.99 (s, 1H), 5.82 (s, 2H), 3.89-3.87 (m, | |
| | | 2H), 1.02 (t, 3H) | |
| 33 | 2-(3,4- dichlorophenyl)-1- methyl-4-oxo-6-[[3- (trifluoromethyl) pyrazol-1- yl]methyl]pyridine-3- carboxylic acid | 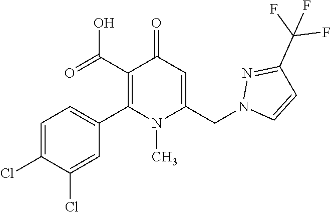 [SMILES: Cn1c(Cn2ccc(C(F)(F)F)n2)cc(=O)c(C(=O)O)c1-c1ccc(Cl)c(Cl)c1] | |
| | | 1H NMR (400 MHz, chloroform) δ = 7.68-7.64 (m, 1H), | |
| | | 7.61 (d, 1H), 7.35 (d, 1H), 7.08 (dd, 1H), 6.68 (d, 1H), 6.29 | |
| | | (s, 1H), 5.44 (s, 2H), 3.41 (s, 3H) | |
| 34 | 6-[(3-chloropyrazol-1- yl)methyl]-2-(3,4- dichlorophenyl)-1- methyl-4-oxo- pyridine-3-carboxylic acid | 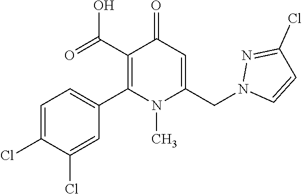 [SMILES: Cn1c(Cn2ccc(Cl)n2)cc(=O)c(C(=O)O)c1-c1ccc(Cl)c(Cl)c1] | |
| | | 1H NMR (400 MHz, chloroform) δ = 7.61 (d, 1H), 7.52 (d, | |
| | | 1H), 7.35 (d, 1H), 7.09 (dd, 1H), 6.33 (d, 1H), 6.29 (s, 1H), | |
| | | 5.32 (s, 2H), 3.40 (s, 3H) | |
| 35 | 2-(3,4- dichlorophenyl)-1- ethyl-6-[(3- methylsulfanylpyrazol- 1-yl)methyl]-4-oxo- pyridine-3-carboxylic acid | 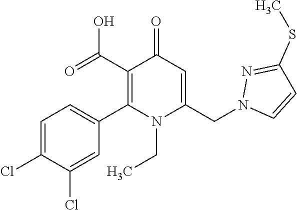 [SMILES: CCn1c(Cn2ccc(SC)n2)cc(=O)c(C(=O)O)c1-c1ccc(Cl)c(Cl)c1] | |
| | | 1H NMR (500 MHz, DMSO-d6) δ = 7.81 (d, 1H), 7.78 (d, | |
| | | 1H), 7.48 (d, 1H), 7.45 (dd, 1H), 5.86 (d, 1H), 5.82 (s, 1H), | |
| | | 5.39 (s, 2H), 3.92 (s, 3H), 3.89 (q, 2H), 1.07 (t, 3H) | |
| 36 | 2-(3,4- dichlorophenyl)-1- ethyl-6-[(3- fluoropyrazol-1- yl)methyl]-4-oxo- pyridine-3-carboxylic acid | 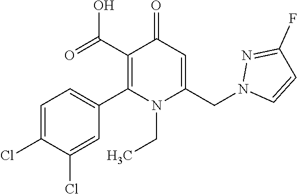 [SMILES: CCn1c(Cn2ccc(F)n2)cc(=O)c(C(=O)O)c1-c1ccc(Cl)c(Cl)c1] | |
| | | 1H NMR (500 MHz, DMSO-d6) δ = 7.82-7.77 (m, 2H), | |
| | | 7.65-7.60 (m, 1H), 7.47-7.42 (m, 1H), 6.18 (dd, 1H), | |
| | | 5.96 (s, 1H), 5.62 (s, 2H), 3.95-3.85 (m, 2H), 1.07 (t, 3H) | |
| 37 | 2-(3,4- dichlorophenyl)-6-[[3- (difluoromethoxy) pyrazol-1-yl]methyl]-1- ethyl-4-oxo-pyridine- 3-carboxylic acid | 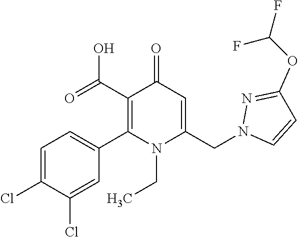 [SMILES: CCn1c(Cn2ccc(OC(F)F)n2)cc(=O)c(C(=O)O)c1-c1ccc(Cl)c(Cl)c1] | |
| | | 1H NMR (500 MHz, DMSO-d6) δ = 7.82-7.77 (m, 2H), | |
| | | 7.64 (d, 1H), 7.45 (dd, 1H), 7.35 (s, 1H), 6.22-6.19 (m, | |
| | | 1H), 5.86 (s, 1H), 5.54 (s, 2H), 3.89 (q, 2H), 1.07 (t, 3H) | |
| 38 | 2-(3,4- dichlorophenyl)-1- ethyl-6-[(3- methoxypyrazol-1- yl)methyl]-4-oxo- pyridine-3-carboxylic acid | 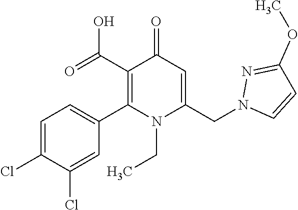 [SMILES: CCn1c(Cn2ccc(OC)n2)cc(=O)c(C(=O)O)c1-c1ccc(Cl)c(Cl)c1] | Rₜ = 1.90 min (A); MS: m/z = 422.14 (M + 1) |
| 39 | 2-(3,4- dichlorophenyl)-6- [(3,5-dimethylpyrazol- 1-yl)methyl]-1-ethyl-4- oxo-pyridine-3- carboxylic acid | 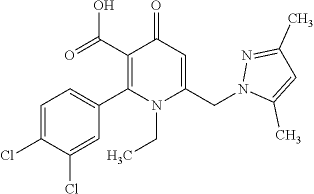 [SMILES: CCn1c(Cn2nc(C)cc2C)cc(=O)c(C(=O)O)c1-c1ccc(Cl)c(Cl)c1] | Rₜ = 1.94 min (A); MS: m/z = 420.18 (M + 1) |
| 40 | 2-(3,4- dichlorophenyl)-1- ethyl-6-[(4- methoxycarbonyl- pyrazol-1-yl)methyl]-4- oxo-pyridine-3- carboxylic acid | 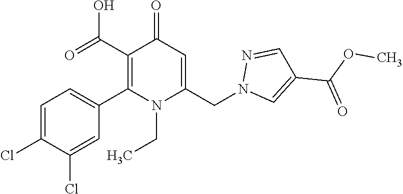 [SMILES: CCn1c(Cn2cc(C(=O)OC)cn2)cc(=O)c(C(=O)O)c1-c1ccc(Cl)c(Cl)c1] | |
| | | 1H NMR (400 MHz, DMSO-d6) δ = 8.66 (s, 1H), 8.07 (s, | |
| | | 1H), 7.86-7.73 (m, 2H), 7.44 (dd, 1H), 6.18 (s, 1H), 5.73 | |
| | | (s, 2H), 3.94-3.89 (m, 2H), 3.78 (s, 3H), 1.05 (t, 3H) | |
| 41 | ethyl 6-[(5-chloro- 1,2,4-triazol-1- yl)methyl]-2-(3,4- dichlorophenyl)-1- ethyl-4-oxo-pyridine- 3-carboxylate | 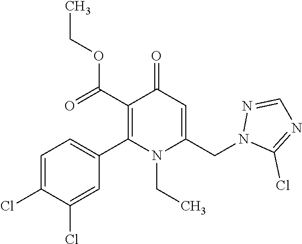 [SMILES: CCOC(=O)c1c(-c2ccc(Cl)c(Cl)c2)n(CC)c(Cn2ncnc2Cl)cc1=O] | |
| | | 1H NMR (400 MHz, chloroform) δ = 7.95 (s, 1H), 7.59 (d, | |
| | | 1H), 7.54 (d, 1H), 7.28-7.26 (m, 1H), 6.33 (s, 1H), 5.32 (s, | |
| | | 2H), 4.08-3.93 (m, 4H), 1.17 (t, 3H), 0.98 (t, 3H) | |
| 42 | ethyl 2-(3,4- dichlorophenyl)-1- ethyl-6-[(4- formylpyrazol-1- yl)methyl]-4-oxo- pyridine-3-carboxylate | 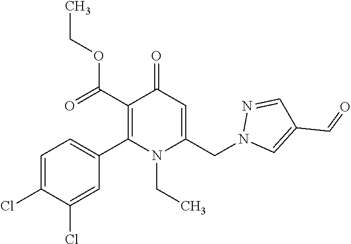 [SMILES: CCOC(=O)c1c(-c2ccc(Cl)c(Cl)c2)n(CC)c(Cn2cc(C=O)cn2)cc1=O] | |
| | | 1H NMR (400 MHz, chloroform) δ = 9.92 (s, 1H), 8.15 (s, | |
| | | 1H), 8.05 (s, 1H), 7.59 (d, 1H), 7.54 (d, 1H), 7.27-7.25 (m, | |
| | | 1H), 6.39 (s, 1H), 5.37 (s, 2H), 4.09-4.00 (m, 2H), 3.90 (q, | |
| | | 2H), 1.09 (t, 3H), 1.00 (t, 3H) | |
| 43 | ethyl 2-(3,4- dichlorophenyl)-1- ethyl-6-[(3-nitro-1,2,4- triazol-1-yl)methyl]-4- oxo-pyridine-3- carboxylate | 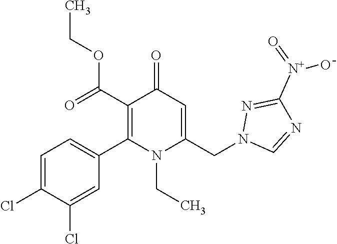 | |
| | | 1H NMR (400 MHz, DMSO-d6) δ = 9.10 (s, 1H), 7.85 (d, | |
| | | 1H), 7.82 (d, 1H), 7.49 (dd, 1H), 6.17 (s, 1H), 5.78 (s, 2H), | |
| | | 3.91-3.76 (m, 4H), 1.03 (t, 3H), 0.82 (t, 3H) | |
| 44 | 2-(3,4- dichlorophenyl)-1- ethyl-6-[(3-methoxy- 1,2,4-triazol-1- yl)methyl]-4-oxo- pyridine-3-carboxylic acid | 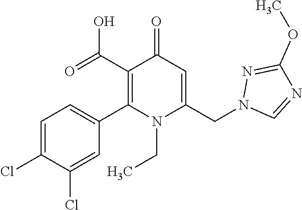 [SMILES: CCn1c(Cn2cnc(OC)n2)cc(=O)c(C(=O)O)c1-c1ccc(Cl)c(Cl)c1] | |
| | | 1H NMR (400 MHz, DMSO-d6) δ = 8.48 (s, 1H), 7.84- | |
| | | 7.80 (m, 1H), 7.78 (d, 1H), 7.44 (dd, 1H), 6.27 (s, 1H), 5.62 | |
| | | (s, 2H), 3.92-3.86 (m, 5H), 1.07 (t, 3H) | |
| 45 | 2-(3,4- dichlorophenyl)-1- ethyl-4-oxo-6-(triazol- 1-ylmethyl)pyridine-3- carboxylic acid | 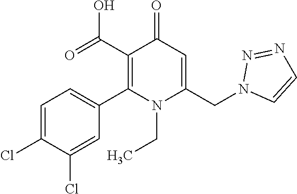 [SMILES: CCn1c(Cn2ccnn2)cc(=O)c(C(=O)O)c1-c1ccc(Cl)c(Cl)c1] | |
| | | 1H NMR (400 MHz, DMSO-d6) δ = 8.13 (brd, 1H), 7.80 | |
| | | (brs, 1H), 7.78 (brs, 1H), 7.72 (brd, 1H), 7.44 (brd, 1H), | |
| | | 6.06 (s, 1H), 5.61 (s, 2H), 3.88 (brd, 2H), 1.05 (brt, 3H) | |
| 46 | ethyl 6-[(3- cyanopyrazol-1- yl)methyl]-2-(3,4- dichlorophenyl)-1- ethyl-4-oxo-pyridine- 3-carboxylate | 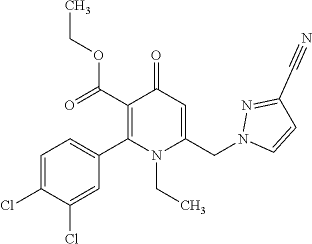 [SMILES: CCOC(=O)c1c(-c2ccc(Cl)c(Cl)c2)n(CC)c(Cn2ccc(C#N)n2)cc1=O] | |
| | | 1H NMR (400 MHz, chloroform) δ = 7.76 (d, 1H), 7.58 (d, | |
| | | 1H), 7.54 (d, 1H), 7.31-7.25 (m, 1H), 6.79 (d, 1H), 6.28 (s, | |
| | | 1H), 5.39 (s, 2H), 4.07-3.95 (m, 2H), 3.95-3.85 (m, 2H), | |
| | | 1.07 (t, 3H), 0.98 (t, 3H) | |
| 47 | ethyl 6-[(5- cyanopyrazol-1- yl)methyl]-2-(3,4- dichlorophenyl)-1- ethyl-4-oxo-pyridine- 3-carboxylate | 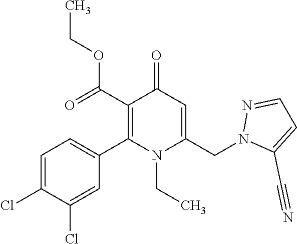 [SMILES: CCOC(=O)c1c(-c2ccc(Cl)c(Cl)c2)n(CC)c(Cn2nccc2C#N)cc1=O] | |
| | | 1H NMR (400 MHz, chloroform) δ = 7.74 (d, 1H), 7.61 (d, | |
| | | 1H), 7.57 (d, 1H), 7.32-7.29 (m, 1H), 6.96 (d, 1H), 6.06 (s, | |
| | | 1H), 5.50 (s, 2H), 4.05 (dd, 2H), 3.93 (d, 2H), 1.22 (t, 3H), | |
| | | 0.99 (t, 3H) | |
| 48 | 2-(3,4- dichlorophenyl)-1- ethyl-4-oxo-6-(triazol- 2-ylmethyl)pyridine-3- carboxylic acid | 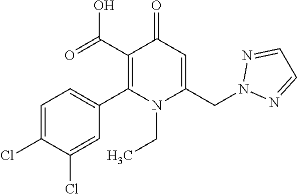 [SMILES: CCn1c(Cn2nccn2)cc(=O)c(C(=O)O)c1-c1ccc(Cl)c(Cl)c1] | |
| | | 1H NMR (400 MHz, DMSO-d6) δ = 8.13 (brd, 1H), 7.80 | |
| | | (brs, 1H), 7.78 (brs, 1H), 7.72 (brd, 1H), 7.44 (brd, 1H), | |
| | | 6.06 (s, 1H), 5.61 (s, 2H), 3.88 (brd, 2H), 1.05 (brt, 3H) | |
| 49 | 2-(3,4- dichlorophenyl)-1- ethyl-4-oxo-6-(1,2,4- triazol-1- ylmethyl)pyridine-3- carboxylic acid | 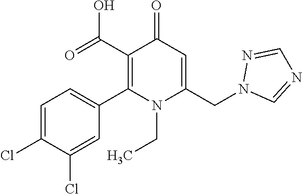 [SMILES: CCn1c(Cn2cncn2)cc(=O)c(C(=O)O)c1-c1ccc(Cl)c(Cl)c1] | |
| | | 1H NMR (400 MHz, DMSO-d6) δ = 8.80 (s, 1H), 8.19 (s, | |
| | | 1H), 7.81 (d, 1H), 7.79 (d, 1H), 7.44 (dd, 1H), 6.20 (s, 1H), | |
| | | 5.79 (s, 2H), 3.92 (brd, 2H), 1.06 (t, 3H) | |
| 50 | 2-(3,4- dichlorophenyl)-1- ethyl-6-[(4- nitroimidazol-1- yl)methyl]-4-oxo- pyridine-3-carboxylic acid | 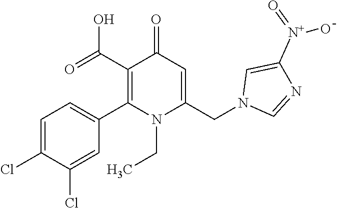 | |
| | | 1H NMR (400 MHz, DMSO-d6) δ = 8.57-8.53 (m, 1H), | |
| | | 8.08-8.04 (m, 1H), 7.82-7.79 (m, 2H), 7.47-7.42 (m, | |
| | | 1H), 6.36-6.33 (m, 1H), 5.67-5.64 (m, 2H), 3.88-3.82 | |
| | | (m, 2H), 1.10-1.06 (m, 3H) | |
| 51 | ethyl 2-(3,4- dichlorophenyl)-1- ethyl-6-[(4- nitroimidazol-1- yl)methyl]-4-oxo- pyridine-3-carboxylate | 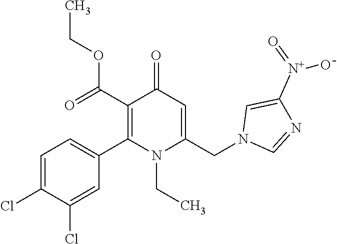 | |
| | | 1H NMR (400 MHz, DMSO-d6) δ = 8.34-8.21 (m, 1H), | |
| | | 8.16-8.07 (m, 1H), 7.89-7.80 (m, 2H), 7.53-7.44 (m, | |
| | | 1H), 5.79-5.70 (m, 1H), 5.55-5.44 (m, 2H), 3.94-3.82 | |
| | | (m, 2H), 3.78-3.67 (m, 2H), 1.05-0.95 (m, 3H), 0.85- | |
| | | 0.77 (m, 3H) | |
| 52 | ethyl 6-[(6- chloropyrazolo[4,3- c]pyridin-1-yl)methyl]- 2-(3,4- dichlorophenyl)-1- ethyl-4-oxo-pyridine- 3-carboxylate | 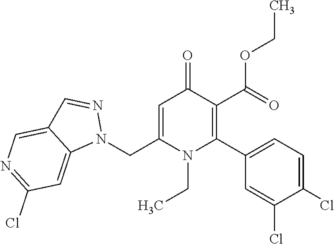 [SMILES: CCOC(=O)c1c(-c2ccc(Cl)c(Cl)c2)n(CC)c(Cn2ncc3cnc(Cl)cc32)cc1=O] | |
| | | 1H NMR (400 MHz, DMSO-d6) δ = 9.04 (s, 1H), 8.56 (s, | |
| | | 1H), 8.03 (s, 1H), 7.79-7.79 (m, 2H), 7.49 (d, 1H), 5.88 | |
| | | (brs, 2H), 5.41 (s, 1H), 3.96-3.87 (m, 4H), 1.08 (t, 3H), | |
| | | 0.80 (t, 3H) | |
| 53 | ethyl 6-[(4- cyanoimidazol-1- yl)methyl]-2-(3,4- dichlorophenyl)-1- ethyl-4-oxo-pyridine- 3-carboxylate | 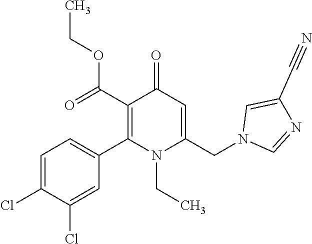 [SMILES: CCOC(=O)c1c(-c2ccc(Cl)c(Cl)c2)n(CC)c(Cn2cnc(C#N)c2)cc1=O] | |
| | | 1H NMR (400 MHz, DMSO-d6) δ = 8.34-8.21 (m, 1H), | |
| | | 8.16-8.07 (m, 1H), 7.89-7.80 (m, 2H), 7.53-7.44 (m, | |
| | | 1H), 5.79-5.70 (m, 1H), 5.55-5.44 (m, 2H), 3.94-3.82 | |
| | | (m, 2H), 3.78-3.67 (m, 2H), 1.05-0.95 (m, 3H), 0.85- | |
| | | 0.77 (m, 3H) | |
| 54 | ethyl 6-[(3-cyano- 1,2,4-triazol-1- yl)methyl]-2-(3,4- dichlorophenyl)-1- ethyl-4-oxo-pyridine- 3-carboxylate | 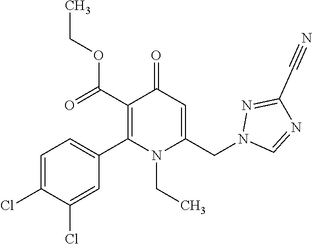 [SMILES: CCOC(=O)c1c(-c2ccc(Cl)c(Cl)c2)n(CC)c(Cn2cnc(C#N)n2)cc1=O] | |
| | | 1H NMR (400 MHz, DMSO-d6) δ = 9.19-9.12 (m, 1H), | |
| | | 7.90-7.79 (m, 2H), 7.51-7.43 (m, 1H), 6.13-6.07 (m, | |
| | | 1H), 5.79-5.70 (m, 2H), 3.91-3.73 (m, 4H), 1.04-0.96 | |
| | | (m, 3H), 0.86-0.78 (m, 3H) | |
| 55 | 6-(benzotriazol-2- ylmethyl)-2-(3,4- dichlorophenyl)-1- ethyl-4-oxo-pyridine- 3-carboxylic acid | 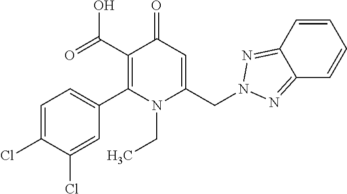 [SMILES: CCn1c(Cn2nc3ccccc3n2)cc(=O)c(C(=O)O)c1-c1ccc(Cl)c(Cl)c1] | |
| | | 1H NMR (400 MHz, DMSO-d6) δ = 8.03-7.97 (m, 2H), | |
| | | 7.81 (d, 1H), 7.76 (d, 1H), 7.55-7.48 (m, 2H), 7.44 (dd, | |
| | | 1H), 6.50 (s, 1H), 6.34 (s, 2H), 3.96 (q, 2H), 0.96 (t, 3H) | |
| 56 | ethyl 2-(3,4- dichlorophenyl)-1- ethyl-6-[(5-methyl-4- nitro-pyrazol-1- yl)methyl]-4-oxo- pyridine-3-carboxylate | 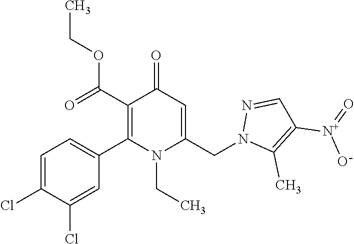 | |
| | | 1H NMR (400 MHz, DMSO-d6) δ = 8.42 (s, 1H), 7.86 (d, | |
| | | 1H), 7.83 (d, 1H), 7.49 (dd, 1H), 5.63 (s, 2H), 5.47 (s, 1H), | |
| | | 3.96-3.73 (m, 4H), 2.65 (s, 3H), 1.09 (t, 3H), 0.81 (t, 3H) | |
| 57 | ethyl 2-(3,4- dichlorophenyl)-1- ethyl-6-[(5-ethyl-1,2,4- triazol-1-yl)methyl]-4- oxo-pyridine-3- carboxylate | 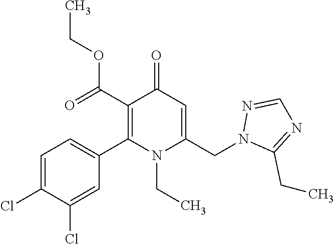 [SMILES: CCOC(=O)c1c(-c2ccc(Cl)c(Cl)c2)n(CC)c(Cn2ncnc2CC)cc1=O] | |
| | | 1H NMR (400 MHz, methanol) δ = 8.02 (s, 1H), 7.77-7.70 | |
| | | (m, 2H), 7.47 (dd, 1H), 5.79 (s, 1H), 5.62 (s, 2H), 4.05- | |
| | | 3.87 (m, 4H), 2.88 (q, 2H), 1.36 (t, 3H), 1.18 (t, 3H), 0.94 | |
| | | (t, 3H) | |
| 58 | ethyl 2-(3,4- dichlorophenyl)-1- ethyl-4-oxo-6-(triazol- 2-ylmethyl)pyridine-3- carboxylate | 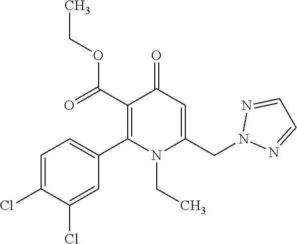 [SMILES: CCOC(=O)c1c(-c2ccc(Cl)c(Cl)c2)n(CC)c(Cn2nccn2)cc1=O] | |
| | | 1H NMR (400 MHz, chloroform) δ = 7.73 (s, 2H), 7.58 (d, | |
| | | 1H), 7.53 (d, 1H), 7.26 (dd, 1H), 6.45 (s, 1H), 5.60 (s, 2H), | |
| | | 4.09-3.99 (m, 2H), 3.92 (q, 2H), 1.09 (t, 3H), 1.00 (t, 3H) | |
| 59 | ethyl 2-(3,4- dichlorophenyl)-1- ethyl-4-oxo-6-(triazol- 1-ylmethyl)pyridine-3- carboxylate | 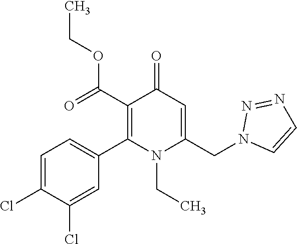 [SMILES: CCOC(=O)c1c(-c2ccc(Cl)c(Cl)c2)n(CC)c(Cn2ccnn2)cc1=O] | |
| | | 1H NMR (400 MHz, chloroform) δ = 7.83 (d, 1H), 7.75 (d, | |
| | | 1H), 7.57 (d, 1H), 7.49 (d, 1H), 7.22 (dd, 1H), 6.55 (s, 1H), | |
| | | 5.57 (s, 2H), 4.12-3.96 (m, 2H), 3.95-3.85 (m, 2H), 1.07- | |
| | | 0.98 (m, 6H) | |
| 60 | ethyl 2-(3,4- dichlorophenyl)-1- ethyl-4-oxo-6-(1,2,4- triazol-1- ylmethyl)pyridine-3- carboxylate | 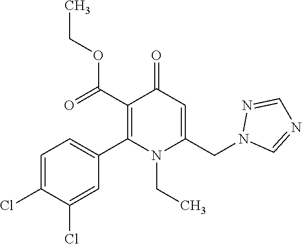 [SMILES: CCOC(=O)c1c(-c2ccc(Cl)c(Cl)c2)n(CC)c(Cn2cncn2)cc1=O] | |
| | | 1H NMR (400 MHz, DMSO-d6) δ = 8.78 (s, 1H), 8.15 (s, | |
| | | 1H), 7.86 (d, 1H), 7.81 (d, 1H), 7.48 (dd, 1H), 5.81 (s, 1H), | |
| | | 5.64 (s, 2H), 3.92-3.76 (m, 4H), 1.00 (t, 3H), 0.82 (t, 3H) | |
| 61 | tert-butyl 2-(3,4- dichlorophenyl)-1- ethyl-6-[(4- methoxycarbonyl- pyrazol-1-yl)methyl]- 4-oxo-pyridine-3- carboxylate | 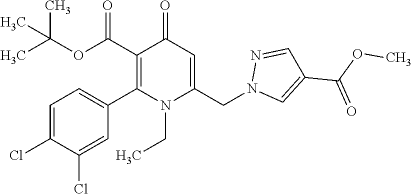 [SMILES: CCn1c(Cn2cc(C(=O)OC)cn2)cc(=O)c(C(=O)OC(C)(C)C)c1-c1ccc(Cl)c(Cl)c1] | |
| | | 1H NMR (400 MHz, chloroform) δ = 8.16-8.12 (m, 1H), | |
| | | 7.99-7.97 (m, 1H), 7.64-7.59 (m, 1H), 7.58-7.55 (m, | |
| | | 1H), 7.33-7.29 (m, 1H), 6.85-6.44 (m, 1H), 5.56-5.27 | |
| | | (m, 2H), 4.01-3.93 (m, 2H), 3.89-3.86 (m, 3H), 1.26- | |
| | | 1.20 (m, 9H), 1.14-1.05 (m, 3H) | |
| 62 | ethyl 2-(3,4- dichlorophenyl)-1- ethyl-6-[(5- nitroimidazol-1- yl)methyl]-4-oxo- pyridine-3-carboxylate | 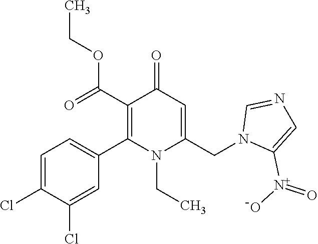 | |
| | | 1H NMR (400 MHz, chloroform) δ = 8.17 (brs, 1H), 7.91 | |
| | | (brs, 1H), 7.65-7.57 (m, 2H), 7.33 (dd, 1H), 5.65 (s, 1H), | |
| | | 5.60 (s, 2H), 3.88-3.78 (m, 2H), 4.10-3.99 (m, 2H), 1.30 | |
| | | (t, 3H), 1.01 (t, 3H) | |
| 63 | ethyl 2-(3,4- dichlorophenyl)-1- ethyl-6-[(3-methyl-4- nitro-pyrazol-1- yl)methyl]-4-oxo- pyridine-3-carboxylate | 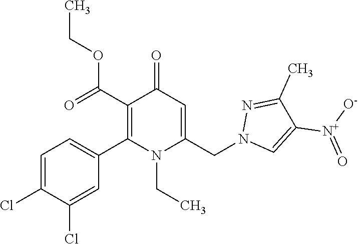 [SMILES: CCOC(=O)c1c(-c2ccc(Cl)c(Cl)c2)n(CC)c(Cn2cc([N+](=O)[O-])c(C)n2)cc1=O] | |
| | | 1H NMR (400 MHz, DMSO-d6) δ = 9.06-8.96 (m, 1H), | |
| | | 7.86-7.77 (m, 2H), 7.52-7.43 (m, 1H), 6.04-5.99 (m, | |
| | | 1H), 5.59-5.51 (m, 2H), 3.92-3.81 (m, 4H), 2.48-2.45 | |
| | | (m, 3H), 1.06-0.97 (m, 3H), 0.83-0.77 (m, 3H) | |
| 64 | 2-(3,4- dichlorophenyl)-1- ethyl-6-[(3-methyl-4- nitro-pyrazol-1- yl)methyl]-4-oxo- pyridine-3-carboxylic acid | 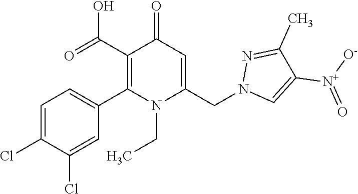 [SMILES: CCn1c(Cn2cc([N+](=O)[O-])c(C)n2)cc(=O)c(C(=O)O)c1-c1ccc(Cl)c(Cl)c1] | |
| | | 1H NMR (400 MHz, DMSO-d6) δ = 9.06-8.96 (m, 1H), | |
| | | 7.86-7.77 (m, 2H), 7.52-7.43 (m, 1H), 6.04-5.99 (m, | |
| | | 1H), 5.59-5.51 (m, 2H), 3.92-3.81 (m, 4H), 2.48-2.45 | |
| | | (m, 3H), 1.06-0.97 (m, 3H), 0.83-0.77 (m, 3H) | |
| 65 | ethyl-6-[(4- chloroimidazo-1- yl)methyl]-2-(3,4- dichlorophenyl)-1- ethyl-4-oxo-pyridine- 3-carboxylate | 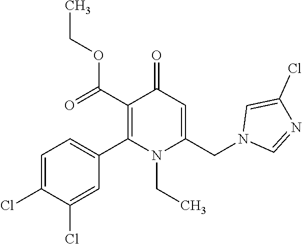 [SMILES: CCOC(=O)c1c(-c2ccc(Cl)c(Cl)c2)n(CC)c(Cn2cnc(Cl)c2)cc1=O] | |
| | | 1H NMR (400 MHz, chloroform) δ = 7.61 (d, 1H), 7.55- | |
| | | 7.52 (m, 1H), 7.46 (d, 1H), 7.28-7.25 (m, 1H), 6.91 (d, | |
| | | 1H), 6.37 (s, 1H), 5.08 (s, 2H), 4.06 (dd, 2H), 3.69 (d, 2H), | |
| | | 1.10 (t, 3H), 1.01 (t, 3H) | |
| 66 | ethyl 6-[(5- chloroimidazol-1- yl)methyl]-2-(3,4- dichlorophenyl)-1- ethyl-4-oxo-pyridine- 3-carboxylate | 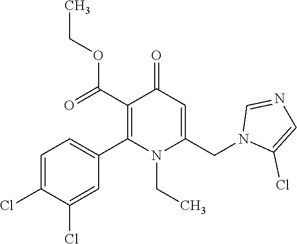 [SMILES: CCOC(=O)c1c(-c2ccc(Cl)c(Cl)c2)n(CC)c(Cn2cncc2Cl)cc1=O] | |
| | | 1H NMR (400 MHz, chloroform) δ = 7.63-7.54 (m, 3H), | |
| | | 7.30 (s, 1H), 7.28-7.25 (m, 1H), 6.07 (s, 1H), 5.11 (s, 2H), | |
| | | 4.06 (dd, 2H), 3.74 (d, 2H), 1.19 (t, 3H), 1.01 (t, 3H) | |
| 67 | ethyl 6-[(5- chloropyrazolo-1- yl)methyl]-2-(3,4- dichlorophenyl)-1- ethyl-4-oxo-pyridine- 3-carboxylate | 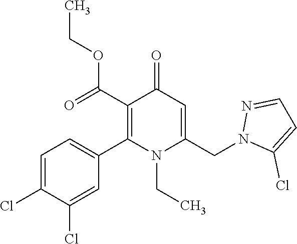 [SMILES: CCOC(=O)c1c(-c2ccc(Cl)c(Cl)c2)n(CC)c(Cn2nccc2Cl)cc1=O] | |
| | | 1H NMR (400 MHz, chloroform) δ = 7.61-7.56 (m, 2H), | |
| | | 7.54 (d, 1H), 7.29 (d, 1H), 6.35 (d, 2H), 5.37 (s, 2H), 4.09- | |
| | | 3.97 (m, 4H), 1.17 (t, 3H), 0.98 (t, 3H) | |
| 68 | ethyl 6-[(4,5- dichloroimidazol-1- yl)methyl]-2-(3,4- dichlorophenyl)-1- ethyl-4-oxo-pyridine- 3-carboxylate | 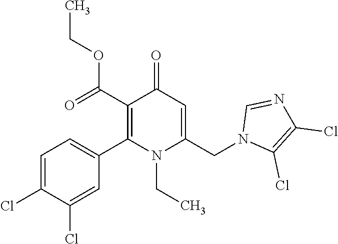 [SMILES: CCOC(=O)c1c(-c2ccc(Cl)c(Cl)c2)n(CC)c(Cn2cnc(Cl)c2Cl)cc1=O] | |
| | | 1H NMR (400 MHz, chloroform) δ = 7.68-7.54 (m, 2H), | |
| | | 7.52-7.44 (m, 1H), 7.37-7.27 (m, 1H), 5.81 (s, 1H), 5.18 | |
| | | (s, 2H), 4.05-3.92 (m, 2H), 3.77 (q, 2H), 1.19 (t, 3H), 0.96 | |
| | | (t, 3H) | |
| 69 | 2-(3,4- dichlorophenyl)-1- ethyl-6-[(5-methyl-4- nitro-pyrazol-1- yl)methyl]-4-oxo- pyridine-3-carboxylic acid | 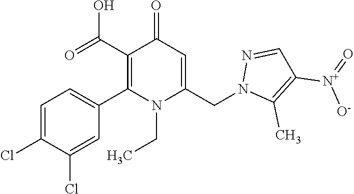 [SMILES: CCn1c(Cn2ncc([N+](=O)[O-])c2C)cc(=O)c(C(=O)O)c1-c1ccc(Cl)c(Cl)c1] | |
| | | 1H NMR (500 MHz, chloroform) δ = 8.19 (s, 1H), 7.61 (d, J = | |
| | | 8.2 Hz, 1H), 7.42-7.35 (m, 1H), 7.17-7.11 (m, 1H), | |
| | | 6.16 (s, 1H), 5.40 (s, 2H), 4.08-3.92 (m, 1H), 4.00 (br d, J = | |
| | | 7.2 Hz, 1H), 2.71 (s, 3H), 1.27 (t, J = 7.1 Hz, 3H) | |
| 70 | 2-(3,4- dichlorophenyl)-1- ethyl-6-[(5-ethyl-1,2,4- triazol-1-yl)methyl]-4- oxo-pyridine-3- carboxylic acid | 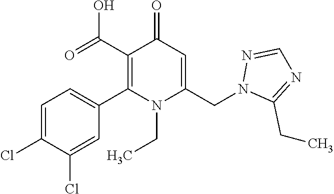 [SMILES: CCc1ncnn1Cc1cc(=O)c(C(=O)O)c(-c2ccc(Cl)c(Cl)c2)n1CC] | |
| | | 1H NMR (500 MHz, chloroform) δ = 7.91 (s, 1H), 7.61 (brd, | |
| | | 1H), 7.38 (s, 1H), 7.14 (dd, 1H), 6.29 (s, 1H), 5.36 (s, 2H), | |
| | | 4.03 (q, 2H), 2.79 (q, 2H), 1.42 (t, 3H), 1.30-1.17 (m, 3H) | |
| 71 | tert-butyl 2-(3,4- dichlorophenyl)-1- ethyl-6-[(4- methoxycarbonyl- imidazol-1-yl)methyl]-4- oxo-pyridine-3- carboxylate | 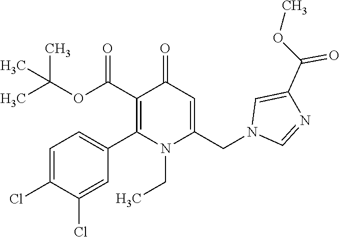 [SMILES: CCn1c(Cn2cnc(C(=O)OC)c2)cc(=O)c(C(=O)OC(C)(C)C)c1-c1ccc(Cl)c(Cl)c1] | |
| | | 1H NMR (400 MHz, chloroform) δ = 7.69-7.67 (m, 1H), | |
| | | 7.66-7.63 (m, 1H), 7.62-7.59 (m, 1H), 7.54 (d, 1H), 7.28- | |
| | | 7.26 (m, 1H), 6.28 (s, 1H), 5.16 (s, 2H), 3.93 (s, 3H), | |
| | | 3.66 (q, 2H), 1.23 (s, 9H), 1.06 (t, 3H) | |
| 72 | ethyl 2-(3,4- dichlorophenyl)-1- ethyl-6-[(3- iodopyrazol-1- yl)methyl]-4-oxo- pyridine-3-carboxylate | 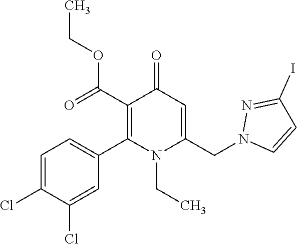 [SMILES: CCOC(=O)c1c(-c2ccc(Cl)c(Cl)c2)n(CC)c(Cn2ccc(I)n2)cc1=O] | |
| | | 1H NMR (400 MHz, chloroform) δ = 7.59-7.57 (m, 1H), | |
| | | 7.50 (d, 1H), 7.35 (d, 1H), 7.25-7.23 (m, 1H), 6.55 (d, | |
| | | 1H), 6.40 (s, 1H), 5.30 (s, 2H), 4.08-3.98 (m, 2H), 3.86 (q, | |
| | | 2H), 1.06-0.97 (m, 6H) | |
| 73 | ethyl 2-(3,4- dichlorophenyl)-1- ethyl-6-[(4- fluoroimidazol-1- yl)methyl]-4-oxo- pyridine-3-carboxylate | 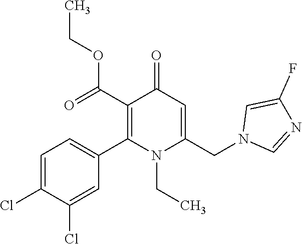 [SMILES: CCOC(=O)c1c(-c2ccc(Cl)c(Cl)c2)n(CC)c(Cn2cnc(F)c2)cc1=O] | |
| | | 1H NMR (400 MHz, chloroform) δ = 7.59 (d, 1H), 7.53- | |
| | | 7.50 (m, 1H), 7.25-7.22 (m, 1H), 7.20 (s, 1H), 6.52 (dd, | |
| | | 1H), 6.35 (s, 1H), 5.04 (s, 2H), 4.10-3.98 (m, 2H), 3.69 | |
| | | (q, 2H), 1.08 (t, 3H), 1.00 (t, 3H) | |
| 74 | 6-[(4-chloroimidazol- 1-yl)methyl]-2-(3,4- dichlorophenyl)-1- ethyl-4-oxo-pyridine- 3-carboxylic acid | 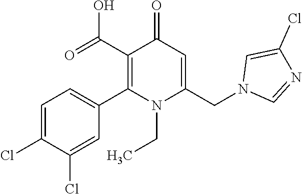 [SMILES: CCn1c(Cn2cnc(Cl)c2)cc(=O)c(C(=O)O)c1-c1ccc(Cl)c(Cl)c1] | |
| | | 1H NMR (400 MHz, DMSO-d6) δ = 7.85 (d, 1H), 7.79 (dd, | |
| | | 2H), 7.45-7.40 (m, 2H), 6.08 (s, 1H), 5.56 (s, 2H), 3.83 (q, | |
| | | 2H), 1.05 (t, 3H) | |
| 75 | 6-[(6- chloropyrazolo[4,3- c]pyridin-1-yl)methyl]- 2-(3,4- dichlorophenyl)-1- ethyl-4-oxo-pyridine- 3-carboxylic acid | 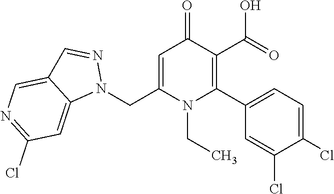 [SMILES: CCn1c(Cn2ncc3cnc(Cl)cc32)cc(=O)c(C(=O)O)c1-c1ccc(Cl)c(Cl)c1] | |
| | | 1H NMR (400 MHz, DMSO-d6) δ = 9.05 (d, 1H), 8.59 (d, | |
| | | 1H), 8.03 (s, 1H), 7.87-7.72 (m, 2H), 7.45 (dd, 1H), 6.02 | |
| | | (s, 2H), 5.76 (s, 1H), 4.02-3.94 (m, 2H), 1.14 (t, 3H) | |
| 76 | 2-(3,4- dichlorophenyl)-1- ethyl-6-[(4- methoxycarbonyl- imidazol-1-yl)methyl]-4- oxo-pyridine-3- carboxylic acid | 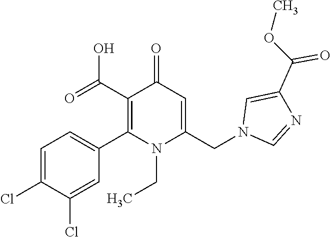 [SMILES: CCn1c(Cn2cnc(C(=O)OC)c2)cc(=O)c(C(=O)O)c1-c1ccc(Cl)c(Cl)c1] | |
| | | 1H NMR (400 MHz, DMSO-d6) δ = 8.09 (s, 1H), 8.00 (s, | |
| | | 1H), 7.82-7.76 (m, 2H), 7.42 (dd, 1H), 6.10 (s, 1H), 5.61 | |
| | | (s, 2H), 3.88-3.80 (m, 2H), 3.78 (s, 3H), 1.05 (t, 3H) | |
| 77 | 2-(3,4- dichlorophenyl)-1- ethyl-6-[(4- fluoroimidazol-1- yl)methyl]-4-oxo- pyridine-3-carboxylic acid | 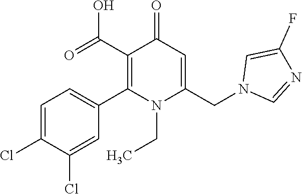 [SMILES: CCn1c(Cn2cnc(F)c2)cc(=O)c(C(=O)O)c1-c1ccc(Cl)c(Cl)c1] | |
| | | 1H NMR (400 MHz, DMSO-d6) δ = 7.83-7.73 (m, 2H), | |
| | | 7.58 (s, 1H), 7.42 (dd, 1H), 7.02 (dd, 1H), 6.09 (s, 1H), | |
| | | 5.51 (s, 2H), 3.83 (q, 2H), 1.05 (t, 3H) | |
| 78 | 2-(3,4- dichlorophenyl)-1- ethyl-6-[(3- iodopyrazol-1- yl)methyl]-4-oxo- pyridine-3-carboxylic acid | 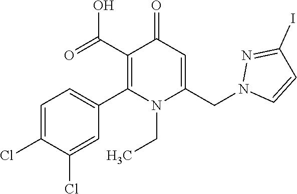 [SMILES: CCn1c(Cn2ccc(I)n2)cc(=O)c(C(=O)O)c1-c1ccc(Cl)c(Cl)c1] | |
| | | 1H NMR (400 MHz, DMSO-d6) δ = 7.91 (d, 1H), 7.83- | |
| | | 7.75 (m, 2H), 7.43 (dd, 1H), 6.67 (d, 1H), 5.99 (s, 1H), 5.71 | |
| | | (s, 2H), 3.87 (q, 2H), 1.04 (t, 3H) | |
| 79 | 6-[(5-chloroimidazol- 1-yl)methyl]-2-(3,4- dichlorophenyl)-1- ethyl-4-oxo-pyridine- 3-carboxylic acid | 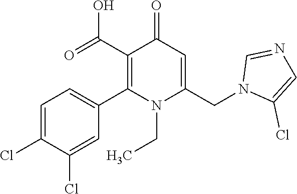 [SMILES: CCn1c(Cn2cncc2Cl)cc(=O)c(C(=O)O)c1-c1ccc(Cl)c(Cl)c1] | |
| | | 1H NMR (400 MHz, chloroform) δ = 7.63 (d, 2H), 7.39 (d, | |
| | | 1H), 7.19-7.11 (m, 2H), 6.11 (s, 1H), 5.23 (s, 2H), 3.87 | |
| | | (brd, 2H), 1.29 (brt, 3H) | |
| 80 | 6-[(5-chloropyrazol-1- yl)methyl]-2-(3,4- dichlorophenyl)-1- ethyl-4-oxo-pyridine- 3-carboxylic acid | 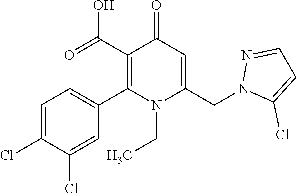 [SMILES: CCn1c(Cn2nccc2Cl)cc(=O)c(C(=O)O)c1-c1ccc(Cl)c(Cl)c1] | |
| | | 1H NMR (400 MHz, chloroform) δ = 7.63 (d, 1H), 7.61 (d, | |
| | | 1H), 7.37 (d, 1H), 7.13 (dd, 1H), 6.38 (d, 1H), 6.27 (s, 1H), | |
| | | 5.43 (s, 2H), 4.00 (q, 2H), 1.24 (t, 3H) | |
| 81 | ethyl 6-[(3- chloropyrazol-1- yl)methyl]-2-(3,4- dichlorophenyl)-1- methyl-4-oxo- pyridine-3-carboxylate | 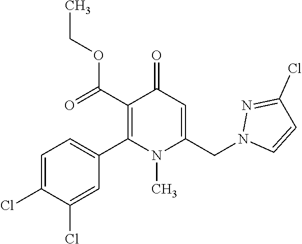 [SMILES: CCOC(=O)c1c(-c2ccc(Cl)c(Cl)c2)n(C)c(Cn2ccc(Cl)n2)cc1=O] | |
| | | 1H NMR (400 MHz, chloroform) δ = 7.63-7.57 (m, 2H), | |
| | | 7.55 (d, 1H), 7.31 (dd, 1H), 6.35 (s, 1H), 6.29 (d, 1H), 5.34 | |
| | | (s, 2H), 4.03 (dq, 2H), 3.44 (s, 3H), 0.99 (t, 3H) | |
| 82 | ethyl 2-(3,4- dichlorophenyl)-1- methyl-4-oxo-6-[[3- (trifluoromethyl) pyrazol-1- yl]methyl]pyridine-3- carboxylate | 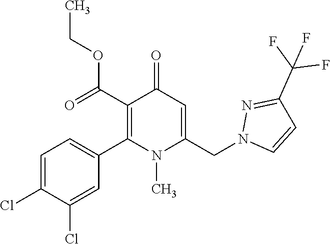 [SMILES: CCOC(=O)c1c(-c2ccc(Cl)c(Cl)c2)n(C)c(Cn2ccc(C(F)(F)F)n2)cc1=O] | |
| | | 1H NMR (400 MHz, chloroform) δ = 7.70 (s, 1H), 7.63- | |
| | | 7.57 (m, 1H), 7.51 (d, 1H), 7.26-7.24 (m, 1H), 6.64 (d, | |
| | | 1H), 6.44 (s, 1H), 5.43 (s, 2H), 4.04 (dq, 2H), 3.46-3.39 | |
| | | (m, 3H), 1.00 (t, 3H) | |
| 83 | 6-[(3-cyanopyrazol-1- yl)methyl]-2-(3,4- dichlorophenyl)-1- ethyl-4-oxo-pyridine- 3-carboxylic acid | 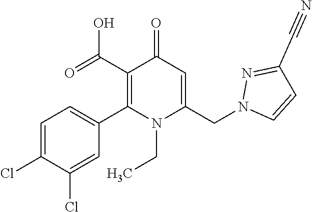 [SMILES: CCn1c(Cn2ccc(C#N)n2)cc(=O)c(C(=O)O)c1-c1ccc(Cl)c(Cl)c1] | |
| | | 1H NMR (400 MHz, chloroform) δ = 7.76-7.66 (m, 1H), | |
| | | 7.62 (brd, 1H), 7.41 (brs, 1H), 7.19-7.12 (m, 1H), 6.83 (s, | |
| | | 1H), 6.35 (brs, 1H), 5.72-5.00 (m, 2H), 4.06-3.93 (m, | |
| | | 2H), 1.21 (brm, 3H) | |
| 84 | 6-[(3-chloro-4-methyl- imidazol-1-yl)methyl]- 2-(3,4- dichlorophenyl)-1- ethyl-4-oxo-pyridine- 3-carboxylic acid | 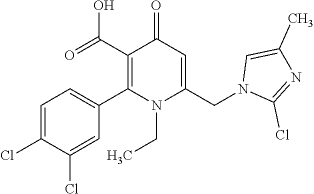 [SMILES: CCn1c(Cn2cc(C)nc2Cl)cc(=O)c(C(=O)O)c1-c1ccc(Cl)c(Cl)c1] | |
| | | 1H NMR (500 MHz, DMSO-d6) δ = 7.82 (d, 1H), 7.79 (d, | |
| | | 1H), 7.46 (dd, 1H), 7.16 (s, 1H), 5.73 (s, 1H), 5.45 (s, 2H), | |
| | | 3.86 (brq, 2H), 2.15 (s, 3H), 1.14 (t, 3H) | |
| 85 | 2-(3,4- dichlorophenyl)-6-[[5- (difluoromethoxy) pyrazol-1-yl]methyl]-1- ethyl-4-oxo-pyridine- 3-carboxylic acid | 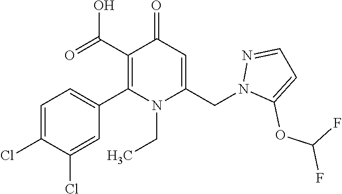 [SMILES: CCn1c(Cn2nccc2OC(F)F)cc(=O)c(C(=O)O)c1-c1ccc(Cl)c(Cl)c1] | Rₜ = 2.03 min (A); MS: m/z = 458.16 (M + 1) |
| 86 | 2-(3,4- dichlorophenyl)-1- ethyl-6-[(5- methylsulfanylpyrazol- 1-yl)methyl]-4-oxo- pyridine-3-carboxylic acid | 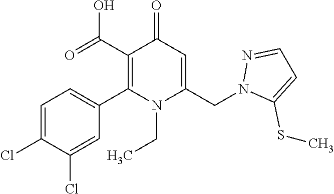 [SMILES: CCn1c(Cn2nccc2SC)cc(=O)c(C(=O)O)c1-c1ccc(Cl)c(Cl)c1] | Rₜ = 2.05 min (A); MS: m/z = 438.12 (M + 1) |
| 87 | 2-(3,4- dichlorophenyl)-6-[[3- (difluoromethyl)-6,7- dihydro-4H- pyrano[4,3-c]pyrazol- 1-yl]methyl]-1-ethyl-4- oxo-pyridine-3- carboxylic acid | 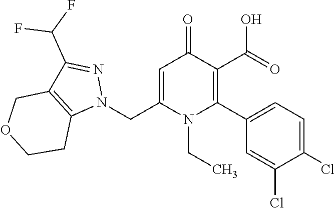 [SMILES: CCn1c(Cn2nc(C(F)F)c3c2CCOC3)cc(=O)c(C(=O)O)c1-c1ccc(Cl)c(Cl)c1] | Rₜ = 2.06 min (A); MS: m/z = 498.15 (M + 1) |
| 88 | 6-[(4-chloro-5-methyl- pyrazol-1-yl)methyl]- 2-(3,4- dichlorophenyl)-1- ethyl-4-oxo-pyridine- 3-carboxylic acid | 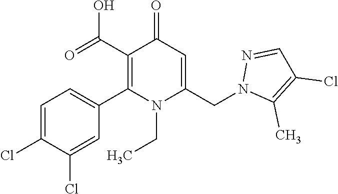 [SMILES: CCn1c(Cn2ncc(Cl)c2C)cc(=O)c(C(=O)O)c1-c1ccc(Cl)c(Cl)c1] | Rₜ = 2.13 min (A); MS: m/z = 440.14 (M + 1) |
| 89 | 6-[(4-chloro-3-methyl- pyrazol-1-yl)methyl]- 2-(3,4- dichlorophenyl)-1- ethyl-4-oxo-pyridine- 3-carboxylic acid | 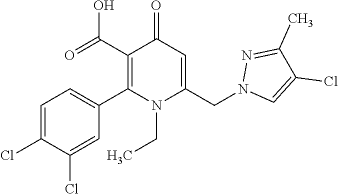 [SMILES: CCn1c(Cn2cc(Cl)c(C)n2)cc(=O)c(C(=O)O)c1-c1ccc(Cl)c(Cl)c1] | Rₜ = 2.16 min (A); MS: m/z = 440.13 (M + 1) |
| 90 | 2-(3,4- dichlorophenyl)-1- ethyl-6-[(4- methylsulfonylpyrazol- 1-yl)methyl]-4-oxo- pyridine-3-carboxylic acid | 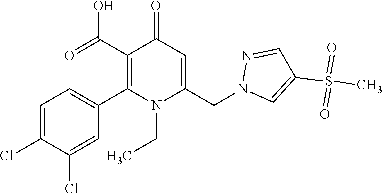 [SMILES: CCn1c(Cn2cc(S(C)(=O)=O)cn2)cc(=O)c(C(=O)O)c1-c1ccc(Cl)c(Cl)c1] | Rₜ = 1.73 min (A); MS: m/z = 470.14 (M + 1) |
| 91 | 2-(3,4- dichlorophenyl)-1- ethyl-6-[(4- methylpyrazol-1- yl)methyl]-4-oxo- pyridine-3-carboxylic acid | 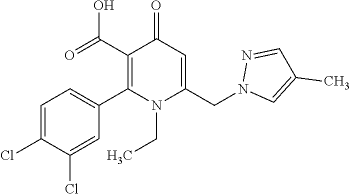 [SMILES: CCn1c(Cn2cc(C)cn2)cc(=O)c(C(=O)O)c1-c1ccc(Cl)c(Cl)c1] | Rₜ = 1.94 min (A); MS: m/z = 406.12 (M + 1) |
| 92 | 2-(3,4- dichlorophenyl)-6- (4,6-dihydrofuro[3,4- c]pyrazol-2-ylmethyl)- 1-ethyl-4-oxo- pyridine-3-carboxylic acid | 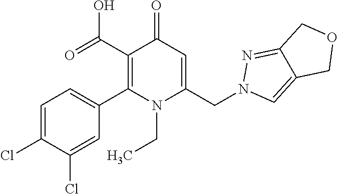 [SMILES: CCn1c(Cn2cc3c(n2)COC3)cc(=O)c(C(=O)O)c1-c1ccc(Cl)c(Cl)c1] | Rₜ = 1.75 min (A); MS: m/z = 434.19 (M + 1) |
| 93 | 6-[(4-chloro-5- cyclopropyl-pyrazol-1- yl)methyl]-2-(3,4- dichlorophenyl)-1- ethyl-4-oxo-pyridine- 3-carboxylic acid | 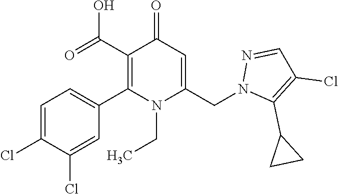 [SMILES: CCn1c(Cn2ncc(Cl)c2C2CC2)cc(=O)c(C(=O)O)c1-c1ccc(Cl)c(Cl)c1] | Rₜ = 2.30 min (A); MS: m/z = 466.11 (M + 1) |
| 94 | 6-[(4-chloro-3- cyclopropyl-pyrazol-1- yl)methyl]-2-(3,4- dichlorophenyl)-1- ethyl-4-oxo-pyridine- 3-carboxylic acid | 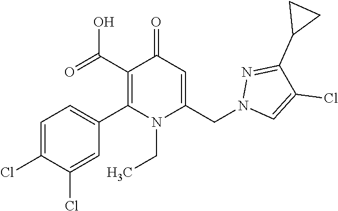 [SMILES: CCn1c(Cn2cc(Cl)c(C3CC3)n2)cc(=O)c(C(=O)O)c1-c1ccc(Cl)c(Cl)c1] | Rₜ = 2.36 min (A); MS: m/z = 466.15 (M + 1) |
| 95 | 6-[(2- cyclopropylimidazol- 1-yl)methyl]-2-(3,4- dichlorophenyl)-1- ethyl-4-oxo-pyridine- 3-carboxylic acid | 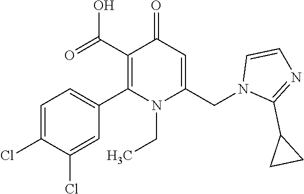 [SMILES: CCn1c(Cn2ccnc2C2CC2)cc(=O)c(C(=O)O)c1-c1ccc(Cl)c(Cl)c1] | Rₜ = 1.46 min (A); MS: m/z = 432.24 (M + 1) |
| 96 | 2-(3,4- dichlorophenyl)-1- ethyl-6-[[4-methyl-3- (trifluoromethyl) pyrazol-1-yl]methyl]-4-oxo- pyridine-3-carboxylic acid | 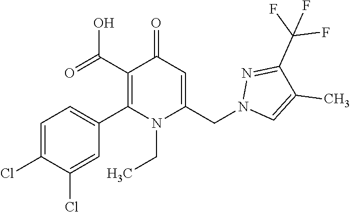 [SMILES: CCn1c(Cn2cc(C)c(C(F)(F)F)n2)cc(=O)c(C(=O)O)c1-c1ccc(Cl)c(Cl)c1] | Rₜ = 2.33 min (A); MS: m/z = 474.20 (M + 1) |
| 97 | 2-(3,4- dichlorophenyl)-6-[[3- (difluoromethyl)-6,7- dihydro-4H- pyrano[4,3-c]pyrazol- 2-yl]methyl]-1-ethyl-4- oxo-pyridine-3- carboxylic acid | 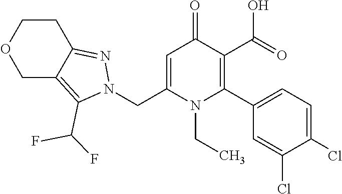 [SMILES: CCn1c(Cn2nc3c(c2C(F)F)COCC3)cc(=O)c(C(=O)O)c1-c1ccc(Cl)c(Cl)c1] | Rₜ = 2.02 min (A); MS: m/z = 498.20 (M + 1) |
| 98 | 6-[(5-chloroindazol-1- yl)methyl]-2-(3,4- dichlorophenyl)-1- ethyl-4-oxo-pyridine- 3-carboxylic acid | 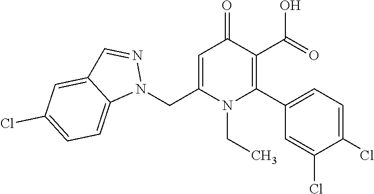 [SMILES: CCn1c(Cn2ncc3cc(Cl)ccc32)cc(=O)c(C(=O)O)c1-c1ccc(Cl)c(Cl)c1] | Rₜ = 2.34 min (A); MS: m/z = 476.12 (M + 1) |
| 99 | 6-[(5-chloroindazol-2- yl)methyl]-2-(3,4- dichlorophenyl)-1- ethyl-4-oxo-pyridine- 3-carboxylic acid | 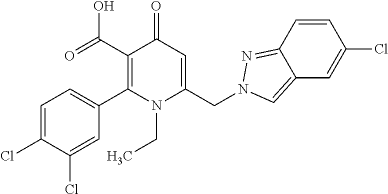 [SMILES: CCn1c(Cn2cc3cc(Cl)ccc3n2)cc(=O)c(C(=O)O)c1-c1ccc(Cl)c(Cl)c1] | Rₜ = 2.30 min (A); MS: m/z = 476.15 (M + 1) |
| 100 | 6-[(4-acetyl-3-methyl- pyrazol-1-yl)methyl]- 2-(3,4- dichlorophenyl)-1- ethyl-4-oxo-pyridine- 3-carboxylic acid | 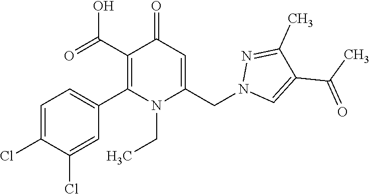 [SMILES: CCn1c(Cn2cc(C(C)=O)c(C)n2)cc(=O)c(C(=O)O)c1-c1ccc(Cl)c(Cl)c1] | Rₜ = 1.84 min (A); MS: m/z = 448.18 (M + 1) |
| 101 | 2-(3,4- dichlorophenyl)-1- ethyl-4-oxo-6- (4,5,6,7- tetrahydroindazol-2- ylmethyl)pyridine-3- carboxylic acid | 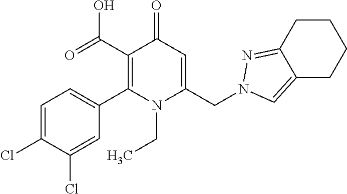 [SMILES: CCn1c(Cn2cc3c(n2)CCCC3)cc(=O)c(C(=O)O)c1-c1ccc(Cl)c(Cl)c1] | Rₜ = 2.16 min (A); MS: m/z = 446.19 (M + 1) |
| 102 | 2-(3,4- dichlorophenyl)-1- ethyl-6-[(5- fluoroindazol-1- yl)methyl]-4-oxo- pyridine-3-carboxylic acid | 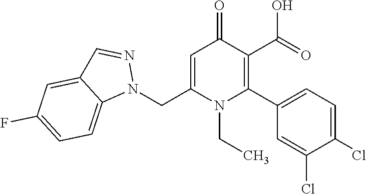 [SMILES: CCn1c(Cn2ncc3cc(F)ccc32)cc(=O)c(C(=O)O)c1-c1ccc(Cl)c(Cl)c1] | Rₜ = 2.21 min (A); MS: m/z = 460.20 (M + 1) |
| 103 | 2-(3,4- dichlorophenyl)-1- ethyl-6-[[2-methyl-4- (trifluoromethyl) imidazol-1-yl] methyl]-4-oxo- pyridine-3-carboxylic acid | 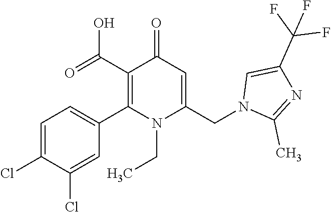 [SMILES: CCn1c(Cn2cc(C(F)(F)F)nc2C)cc(=O)c(C(=O)O)c1-c1ccc(Cl)c(Cl)c1] | Rₜ = 1.94 min (A); MS: m/z = 474.18 (M + 1) |
| 104 | 6-[[3-chloro-5- (trifluoromethyl) pyrazol-1- yl]methyl]-2-(3,4- dichlorophenyl)-1- ethyl-4-oxo-pyridine- 3-carboxylic acid | 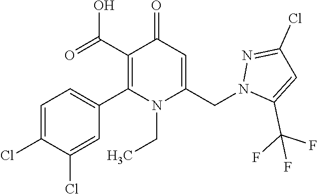 [SMILES: CCn1c(Cn2nc(Cl)cc2C(F)(F)F)cc(=O)c(C(=O)O)c1-c1ccc(Cl)c(Cl)c1] | Rₜ = 2.36 min (A); MS: m/z = 494.09 (M + 1) |
| 105 | 2-(3,4- dichlorophenyl)-1- ethyl-6-[indazol-1- ylmethyl)-4-oxo- pyridine-3-carboxylic acid | 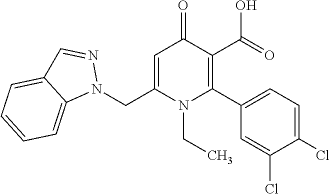 [SMILES: CCn1c(Cn2ncc3ccccc32)cc(=O)c(C(=O)O)c1-c1ccc(Cl)c(Cl)c1] | Rₜ = 2.17 min (A); MS: m/z = 442.15 (M + 1) |
| 106 | 2-(3,4- dichlorophenyl)-1- ethyl-6-(indazol-2- ylmethyl)-4-oxo- pyridine-3-carboxylic acid | 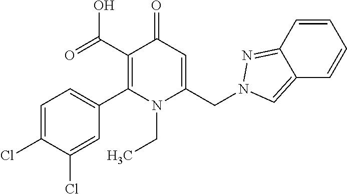 [SMILES: CCn1c(Cn2cc3ccccc3n2)cc(=O)c(C(=O)O)c1-c1ccc(Cl)c(Cl)c1] | Rₜ = 2.09 min (A); MS: m/z = 442.21 (M + 1) |
| 107 | 2-(3,4- dichlorophenyl)-1- ethyl-4-oxo-6-[[3- (trifluoromethyl)-6,7- dihydro-4H- pyrano[4,3-c]pyrazol- 1-yl]methyl]pyridine-3- carboxylic acid | 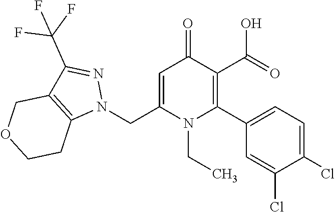 [SMILES: CCn1c(Cn2nc(C(F)(F)F)c3c2CCOC3)cc(=O)c(C(=O)O)c1-c1ccc(Cl)c(Cl)c1] | Rₜ = 2.23 min (A); MS: m/z = 516.15 (M + 1) |
| 108 | 2-(3,4- dichlorophenyl)-1- ethyl-6-[[5-methyl-3- (trifluoromethyl)-1,2,4- triazol-1-yl]methyl]-4- oxo-pyridine-3- carboxylic acid | 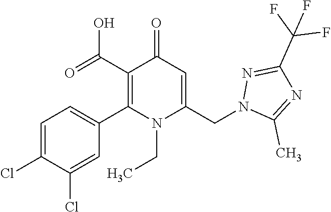 [SMILES: CCn1c(Cn2nc(C(F)(F)F)nc2C)cc(=O)c(C(=O)O)c1-c1ccc(Cl)c(Cl)c1] | Rₜ = 2.07 min (A); MS: m/z = 475.18 (M + 1) |
| 109 | 2-(3,4- dichlorophenyl)-6-[[4- (difluoromethyl) pyrazol-1-yl]methyl]-1- ethyl-4-oxo-pyridine- 3-carboxylic acid | 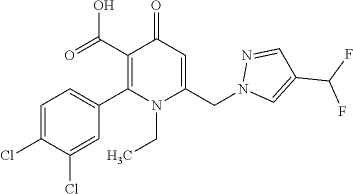 [SMILES: CCn1c(Cn2cc(C(F)F)cn2)cc(=O)c(C(=O)O)c1-c1ccc(Cl)c(Cl)c1] | Rₜ = 2.01 min (A); MS: m/z = 442.17 (M + 1) |
| 110 | 2-(3,4- dichlorophenyl)-6-[(2- ethoxycarbonylimidazol- 1-yl)methyl]-1- ethyl-4-oxo-pyridine- 3-carboxylic acid | 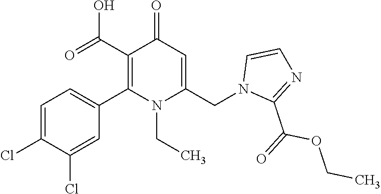 [SMILES: CCOC(=O)c1nccn1Cc1cc(=O)c(C(=O)O)c(-c2ccc(Cl)c(Cl)c2)n1CC] | |
| | | 1H NMR (400 MHz, DMSO-d6) δ = 7.83 (d, 1H), 7.80 (d, | |
| | | 1H), 7.65 (s, 1H), 7.45 (dd, 1H), 7.32 (s, 1H), 5.89 (s, 2H), | |
| | | 5.48 (s, 1H), 4.27 (q, 2H), 3.87 (q, 2H), 1.27 (t, 3H), 1.19 (t, | |
| | | 3H) | |
| 111 | 6-[[4,5- bis(methoxycarbonyl) imidazol-1-yl]methyl]- 2-(3,4- dichlorophenyl)-1- ethyl-4-oxo-pyridine- 3-carboxylic acid | 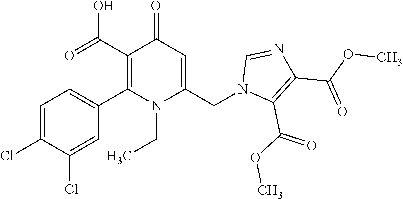 [SMILES: CCn1c(Cn2cnc(C(=O)OC)c2C(=O)OC)cc(=O)c(C(=O)O)c1-c1ccc(Cl)c(Cl)c1] | |
| | | 1H NMR (400 MHz, DMSO-d6) δ = 8.17 (s, 1H), 7.82 (dd, | |
| | | 2H), 7.46 (dd, 1H), 5.78 (s, 2H), 5.65 (s, 1H), 3.90-3.82 | |
| | | (m, 5H), 3.79 (s, 3H), 1.16 (t, 3H) | |
| 112 | 6-[[3,5- bis(ethoxycarbonyl) pyrazol-1-yl]methyl]-2- (3,4-dichlorophenyl)- 1-ethyl-4-oxo- pyridine-3-carboxylic acid | 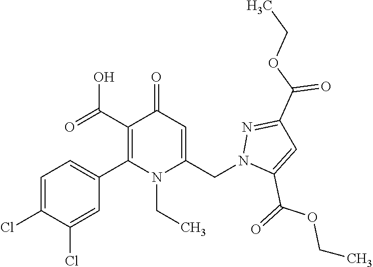 [SMILES: CCOC(=O)c1cc(C(=O)OCC)n(Cc2cc(=O)c(C(=O)O)c(-c3ccc(Cl)c(Cl)c3)n2CC)n1] | |
| | | 1H NMR (400 MHz, DMSO-d6) δ = 16.22 (s, 1H), 7.83- | |
| | | 7.79 (m, 2H), 7.45 (m, 2H), 6.10 (s, 2H), 5.52 (s, 1H), 4.33- | |
| | | 4.29 (m, 4H), 3.86 (q, 2H), 1.32-1.28 (m, 6H), 1.17 (t, | |
| | | 3H) | |
| 113 | 2-(3,4- dichlorophenyl)-6- [(4,5-dicyanoimidazol- 1-yl)methyl]-1-ethyl-4- oxo-pyridine-3- carboxylic acid | 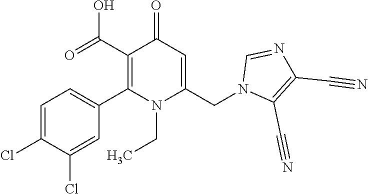 [SMILES: CCn1c(Cn2cnc(C#N)c2C#N)cc(=O)c(C(=O)O)c1-c1ccc(Cl)c(Cl)c1] | |
| | | 1H NMR (400 MHz, DMSO-d6) δ = 15.91 (s, 1H), 8.48 | |
| | | (s, 1H), 7.82 (dd, 2H), 7.47 (dd, 1H), 6.37 (s, 1H), 5.80 (s, | |
| | | 2H), 3.83 (q, 2H), 1.11 (t, 3H) | |
| 114 | 6-[[4,5- bis(ethoxycarbonyl) imidazol-1-yl]methyl]-2- (3,4-dichlorophenyl)- 1-ethyl-4-oxo- pyridine-3-carboxylic acid | 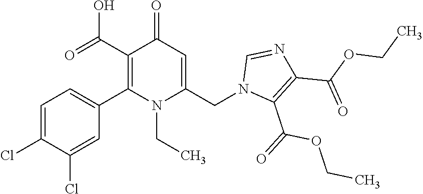 [SMILES: CCOC(=O)c1ncn(Cc2cc(=O)c(C(=O)O)c(-c3ccc(Cl)c(Cl)c3)n2CC)c1C(=O)OCC] | |
| | | 1H NMR (400 MHz, DMSO-d6) δ = 16.13 (s, 1H), 8.16 (s, | |
| | | 1H), 7.80 (d, 2H), 7.44 (d, 1H), 5.78 (s, 2H), 5.65 (s, 1H), | |
| | | 4.37-4.15 (m, 4H), 3.85 (d, 2H), 1.30 (t, 3H), 1.21 (t, | |
| | | 3H), 1.16 (t, 3H) | |
| 115 | 6-[[3,5- bis(methoxycarbonyl) pyrazol-1-yl]methyl]- 2-(3,4- dichlorophenyl)-1- ethyl-4-oxo-pyridine- 3-carboxylic acid | 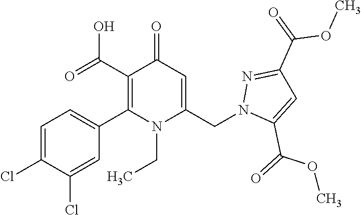 [SMILES: CCn1c(Cn2nc(C(=O)OC)cc2C(=O)OC)cc(=O)c(C(=O)O)c1-c1ccc(Cl)c(Cl)c1] | |
| | | 1H NMR (400 MHz, DMSO-d6) δ = 16.23 (brs, 1H), 7.86- | |
| | | 7.77 (m, 2H), 7.50-7.44 (m, 2H), 6.09 (s, 2H), 5.54 (s, | |
| | | 1H), 3.88-3.84 (m, 8H), 1.18 (t, 3H) | |
| 116 | 2-(4-chloro-3- methylsulfonyl- phenyl)-1-ethyl-4-oxo- 6-[[3- (trifluoromethyl) pyrazol-1- yl]methyl]pyridine-3- carboxylic acid | 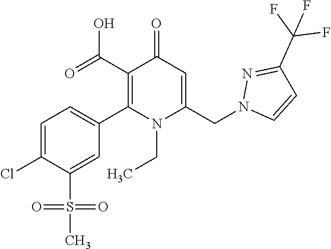 [SMILES: CCn1c(Cn2ccc(C(F)(F)F)n2)cc(=O)c(C(=O)O)c1-c1ccc(Cl)c(S(C)(=O)=O)c1] | |
| | | 1H NMR (400 MHz, DMSO-d6) δ = 8.23 (t, 1H), 8.07 (d, | |
| | | 1H), 7.89 (d, 1H), 7.84-7.82 (dd, 1H), 6.94 (d, 1H), 6.01 | |
| | | (s, 1H), 5.82 (s, 2H), 3.93-3.81 (m, 2H), 3.41 (s, 3H), 1.01 | |
| | | (t, 3H) | |
| 117 | 2-(3-chloro-4-cyano- phenyl)-1-methyl-4- oxo-6-[[3- (trifluoromethyl) pyrazol-1- yl]methyl]pyridine-3- carboxylic acid | 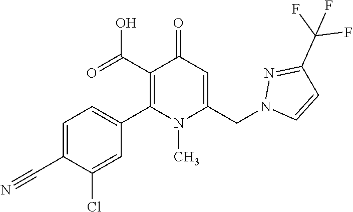 [SMILES: Cn1c(Cn2ccc(C(F)(F)F)n2)cc(=O)c(C(=O)O)c1-c1ccc(C#N)c(Cl)c1] | |
| | | 1H NMR (400 MHz, DMSO-d6) δ = 8.24 (d, 1H), 8.14 (d, | |
| | | 1H), 7.94 (d, 1H), 7.66-7.64 (dd, 1H), 6.94 (d, 1H), 6.04 | |
| | | (s, 1H), 5.83 (s, 2H), 3.89-3.83 (q, 2H), 1.03 (t, 3H) | |
| 118 | 2-(4-chloro-3-fluoro- phenyl)-6-[(3- chloropyrazol-1- yl)methyl]-1-ethyl-4- oxo-pyridine-3- carboxylic acid | 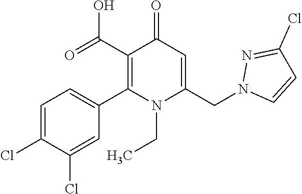 [SMILES: CCn1c(Cn2ccc(Cl)n2)cc(=O)c(C(=O)O)c1-c1ccc(Cl)c(Cl)c1] | |
| | | 1H NMR (400 MHz, DMSO-d6) δ = 8.06 (d, 1H), 7.77- | |
| | | 7.75 (dd, 1H), 7.58-7.54 (m, 1H), 7.47-7.43 (m, 1H), | |
| | | 6.52 (d, 1H), 6.08 (s, 1H), 5.65 (s, 2H), 3.90-3.85 (q, 2H), | |
| | | 1.03 (t, 3H) | |
| 119 | 2-(3-chloro-4-cyano- phenyl)-6-[(3- chloropyrazol-1- yl)methyl]-1-ethyl-4- oxo-pyridine-3- carboxylic acid | 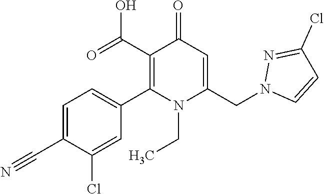 [SMILES: CCn1c(Cn2ccc(Cl)n2)cc(=O)c(C(=O)O)c1-c1ccc(C#N)c(Cl)c1] | |
| | | 1H NMR (400 MHz, DMSO-d6) δ = 8.14 (d, 1H), 8.08 (d, | |
| | | 1H), 7.97 (d, 1H), 7.67-7.64 (dd, 1H), 6.54 (d, 1H), 6.13 | |
| | | (s, 1H), 5.67 (s, 2H), 3.88-3.83 (q, 2H), 1.03 (t, 3H) | |
| 120 | 2-(4-chloro-3- methylsulfonyl- phenyl)-6-[(3- chloropyrazol-1- yl)methyl]-1-ethyl-4- oxo-pyridine-3- carboxylic acid | 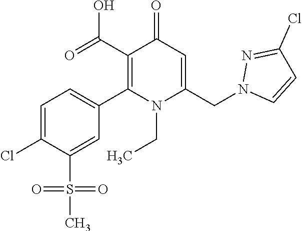 [SMILES: CCn1c(Cn2ccc(Cl)n2)cc(=O)c(C(=O)O)c1-c1ccc(Cl)c(S(C)(=O)=O)c1] | |
| | | 1H NMR (400 MHz, DMSO-d6) δ = 8.07 (d, 2H), 7.90- | |
| | | 7.88 (t, 1H), 7.84-7.82 (dd, 1H), 6.53 (d, 1H), 6.10 (s, 1H), | |
| | | 5.66 (s, 2H), 3.91-3.82 (m, 2H), 3.41 (s, 3H), 1.02 (t, 3H) | |
| 121 | ethyl 2-(3,4- dichlorophenyl)-6-[[3- (difluoromethyl)-1,2,4- triazol-4-yl]methyl]-1- ethyl-4-oxo-pyridine- 3-carboxylate | 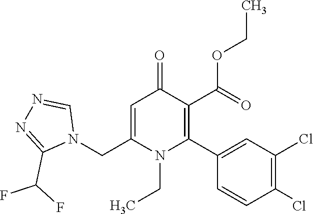 [SMILES: CCOC(=O)c1c(-c2ccc(Cl)c(Cl)c2)n(CC)c(Cn2cnnc2C(F)F)cc1=O] | Rₜ = 1.95 min (A); MS: m/z = 471.17 (M + 1) |
| 122 | 2-(3,4- dichlorophenyl)-6-[(5- ethoxycarbonyl-4- methyl-pyrazol-1- yl)methyl]-1-ethyl-4- oxo-pyridine-3- carboxylic acid | 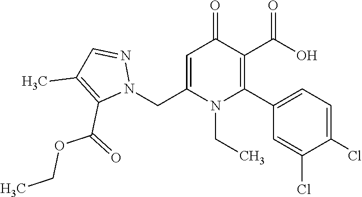 [SMILES: CCOC(=O)c1c(C)cnn1Cc1cc(=O)c(C(=O)O)c(-c2ccc(Cl)c(Cl)c2)n1CC] | |
| | | 1H NMR (400 MHz, chloroform) δ = 7.60 (d, 1H), 7.50 (s, | |
| | | 1H), 7.38 (s, 1H), 7.14 (dd, 1H), 5.85 (s, 2H), 5.85 (s, 1H), | |
| | | 4.34 (q, 2H), 3.93 (q, 2H), 2.33 (s, 3H), 1.39 (t, 3H), 1.32 (t, | |
| | | 3H) | |
| 123 | 2-(3,4- dichlorophenyl)-6-[(3- ethoxycarbonyl-4- methyl-pyrazol-1- yl)methyl]-1-ethyl-4- oxo-pyridine-3- carboxylic acid | 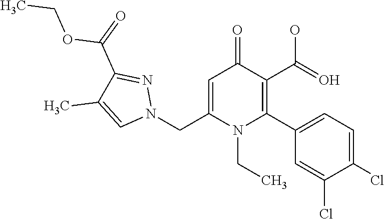 [SMILES: CCOC(=O)c1nn(Cc2cc(=O)c(C(=O)O)c(-c3ccc(Cl)c(Cl)c3)n2CC)cc1C] | |
| | | 1H NMR (400 MHz, DMSO-d6) δ = 16.35 (brs, 1H), 7.90 | |
| | | (s, 1H), 7.80 (d, 1H), 7.78 (d, 1H), 7.43 (d, 1H), 5.95 (s, | |
| | | 1H), 5.73 (s, 2H), 4.26 (q, 2H), 3.87 (q, 2H), 2.25 (s, 3H), | |
| | | 1.29 (t, 3H), 1.06 (t, 3H) | |
| 124 | 2-(3,4- dichlorophenyl)-1- ethyl-6-[(5-methoxy- 1,2,4-triazol-1- yl)methyl]-4-oxo- pyridine-3-carboxylic acid | 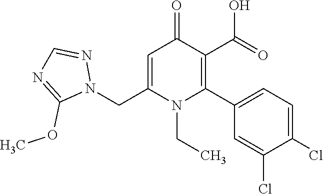 [SMILES: CCn1c(Cn2ncnc2OC)cc(=O)c(C(=O)O)c1-c1ccc(Cl)c(Cl)c1] | |
| | | 1H NMR (400 MHz, chloroform) δ = 7.65-7.59 (m, 2H), | |
| | | 7.36 (d, 1H), 7.12 (dd, 1H), 6.64 (s, 1H), 5.19 (s, 2H), 4.18 | |
| | | (s, 3H), 4.06 (q, 2H), 1.26-1.18 (m, 3H) | |
| 125 | 2-(3,4- dichlorophenyl)-1- ethyl-6-(imidazo[4,5- b]pyridin-1-ylmethyl)- 4-oxo-pyridine-3- carboxylic acid | 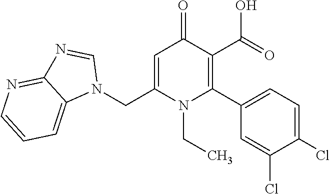 [SMILES: CCn1c(Cn2cnc3ncccc32)cc(=O)c(C(=O)O)c1-c1ccc(Cl)c(Cl)c1] | Rₜ = 1.49 min (A); MS: m/z = 443.33 (M + 1) |
| 126 | 6-[(4,5- dichloroimidazol-1- yl)methyl]-2-(3,4- dichlorophenyl)-1- ethyl-4-oxo-pyridine- 3-carboxylic acid | 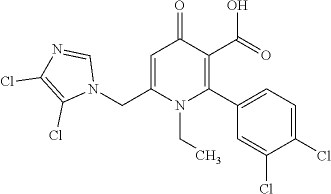 [SMILES: CCn1c(Cn2cnc(Cl)c2Cl)cc(=O)c(C(=O)O)c1-c1ccc(Cl)c(Cl)c1] | |
| | | 1H NMR (400 MHz, DMSO-d6) δ = 8.03 (s, | |
| | | 1H), 7.79 (dd, 2H), 7.47 (dd, 1H), 5.76 (s, 1H), 5.58 (s, | |
| | | 2H), 3.86 (q, 2H), 1.14 (t, 3H) | |
| 127 | 2-(3,4- dichlorophenyl)-1- ethyl-6-[(5-methoxy-3- methyl-pyrazol-1- yl)methyl]-4-oxo- pyridine-3-carboxylic acid | 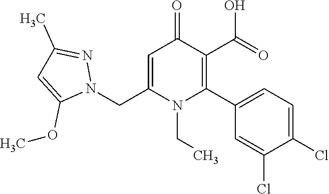 [SMILES: CCn1c(Cn2nc(C)cc2OC)cc(=O)c(C(=O)O)c1-c1ccc(Cl)c(Cl)c1] | |
| | | 1H NMR (400 MHz, DMSO-d6) δ = 7.81 (d, 1H), 7.78 (d, | |
| | | 1H), 7.44 (dd, 1H), 5.84 (s, 1H), 5.68 (s, 1H), 5.29 (s, | |
| | | 2H), 3.88 (s, 3H), 3.86 (m, 2H), 2.14 (s, 3H), 1.06 (t, 3H) | |
| 128 | 2-(4-chloro-3-cyano- phenyl)-1-ethyl-4-oxo- 6-[[3- (trifluoromethyl) pyrazol-1- yl]methyl]pyridine-3- carboxylic acid | 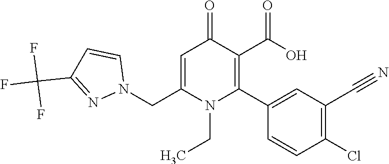 [SMILES: CCn1c(Cn2ccc(C(F)(F)F)n2)cc(=O)c(C(=O)O)c1-c1ccc(Cl)c(C#N)c1] | |
| | | 1H NMR (400 MHz, DMSO-d6) δ = 8.25 (dd, 1H), 8.16 (d, | |
| | | 1H), 7.91 (d, 1H), 7.83-7.80 (m, 1H), 6.94 (d, 1H), 6.05 (s, | |
| | | 1H), 5.84 (s, 2H), 3.90-3.86 (q, 2H), 1.02 (t, 3H) | |
| 129 | 2-(3-chloro-4- methylsulfonyl- phenyl)-1-ethyl-4-oxo- 6-[[3- (trifluoromethyl) pyrazol-1- yl]methyl]pyridine-3- carboxylic acid | 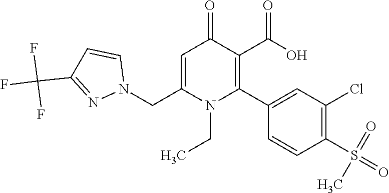 [SMILES: CCn1c(Cn2ccc(C(F)(F)F)n2)cc(=O)c(C(=O)O)c1-c1ccc(S(C)(=O)=O)c(Cl)c1] | |
| | | 1H NMR (400 MHz, DMSO-d6) δ = 8.25 (s, 1H), 8.15 (d, | |
| | | 1H), 7.94 (d, 1H), 7.70-7.68 (dd, 1H), 6.95 (s, 1H), 6.02 | |
| | | (s, 1H), 5.84 (s, 2H), 3.87-3.85 (q, 2H), 3.45 (s, 3H), 1.05 | |
| | | (t, 3H) | |
| 130 | 2-(3-chloro-4- methylsulfonyl- phenyl)-6-[(3- chloropyrazol-1- yl)methyl]-1-ethyl-4- oxo-pyridine-3- carboxylic acid | 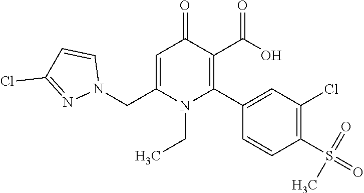 [SMILES: CCn1c(Cn2ccc(Cl)n2)cc(=O)c(C(=O)O)c1-c1ccc(S(C)(=O)=O)c(Cl)c1] | |
| | | 1H NMR (400 MHz, DMSO-d6) δ = 8.15 (d, 1H), 8.08 (d, | |
| | | 1H), 7.95 (d, 1H), 7.70-7.68 (dd, 1H), 6.54 (d, 1H), 6.11 | |
| | | (s, 1H), 5.67 (s, 2H), 3.86 (q, 2H), 3.45 (s, 3H), 1.05 (t, 3H) | |
| 131 | 2-(4-chloro-3-cyano- phenyl)-6-[(3- chloropyrazol-1- yl)methyl]-1-ethyl-4- oxo-pyridine-3- carboxamide acid | 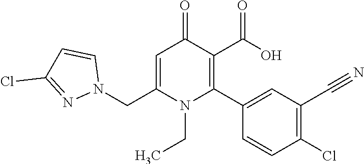 [SMILES: CCn1c(Cn2ccc(Cl)n2)cc(=O)c(C(=O)O)c1-c1ccc(Cl)c(C#N)c1] | |
| | | 1H NMR (400 MHz, DMSO-d6) δ = 8.17 (d, 1H), 8.08 (d, | |
| | | 1H), 7.93-7.90 (m, 1H), 7.83-7.81 (m, 1H), 6.54 (d, 1H), | |
| | | 6.14 (d, 1H), 5.68 (s, 2H), 3.90 (q, 2H), 1.02 (t, 3H) | |
| 132 | 2-(3,4- dichlorophenyl)-6- [(3,5-dicyanopyrazol- 1-yl)methyl]-1-ethyl-4- oxo-pyridine-3- carboxylic acid | 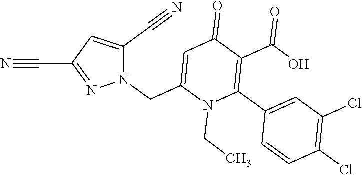 [SMILES: CCn1c(Cn2nc(C#N)cc2C#N)cc(=O)c(C(=O)O)c1-c1ccc(Cl)c(Cl)c1] | |
| | | 1H NMR (400 MHz, DMSO-d6) δ = 15.92 (brs, 1H), 8.13 | |
| | | (s, 1H), 7.80-7.79 (m, 2H), 7.47-7.44 (m, 1H), 6.25 (s, | |
| | | 1H), 6.02 (s, 2H), 3.86 (q, 2H), 1.08 (t, 3H) | |
| 133 | 2-(4-amino-3-chloro- phenyl)-1-ethyl-4-oxo- 6-[[3- (trifluoromethyl) pyrazol-1- yl]methyl]pyridine-3- carboxylic acid | 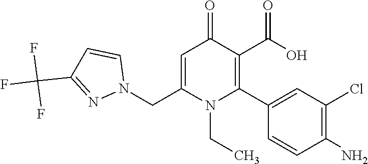 [SMILES: CCn1c(Cn2ccc(C(F)(F)F)n2)cc(=O)c(C(=O)O)c1-c1ccc(N)c(Cl)c1] | |
| | | 1H NMR (400 MHz, DMSO-d6) δ = 8.22 (d, 1H), 7.20 (d, | |
| | | 1H), 6.98-6.96 (dd, 1H), 6.92 (d, 1H), 6.84 (d, 1H), 5.85 | |
| | | (s, 1H), 5.76 (s, 2H), 5.76 (s, 2H), 3.93-3.88 (q, 2H), | |
| | | 1.00 (t, 3H) | |
| 134 | 2-(3,4- dichlorophenyl)-6- [(3,5-dichloropyrazol- 1-yl)methyl]-1-ethyl-4- oxo-pyridine-3- carboxylic acid | 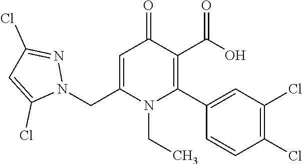 [SMILES: CCn1c(Cn2nc(Cl)cc2Cl)cc(=O)c(C(=O)O)c1-c1ccc(Cl)c(Cl)c1] | |
| | | 1H NMR (400 MHz, DMSO-d6) δ = 7.81 (dd, 2H), 7.46 (dd, | |
| | | 1H), 6.90 (s, 1H), 5.81 (s, 1H), 5.69 (s, 2H), 3.87 (q, 2H), | |
| | | 1.09 (t, 3H) | |
| 135 | 6-[[3,5- bis(trifluoromethyl) pyrazol-1-yl]methyl]-2- (3,4-dichlorophenyl)- 1-ethyl-4-oxo- pyridine-3-carboxylic acid | 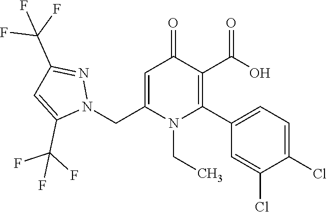 [SMILES: CCn1c(Cn2nc(C(F)(F)F)cc2C(F)(F)F)cc(=O)c(C(=O)O)c1-c1ccc(Cl)c(Cl)c1] | |
| | | 1H NMR (500 MHz, DMSO-d6) δ = 7.83 (s, 1H), 7.72 (d, | |
| | | 1H), 7.64 (d, 1H), 7.36 (dd, 1H), 5.83 (s, 2H), 5.34 (s, 1H), | |
| | | 3.76 (q, 2H), 0.99 (t, 3H) | |
| 136 | methyl 5-bromo-2- (3,4-difluorophenyl)-1- ethyl-4-oxo-6-[[3- (trifluoromethyl) pyrazol-1- yl]methyl]pyridine-3- carboxylate | 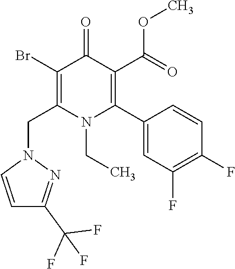 [SMILES: CCn1c(Cn2ccc(C(F)(F)F)n2)c(Br)c(=O)c(C(=O)OC)c1-c1ccc(F)c(F)c1] | |
| | | 1H NMR (400 MHz, DMSO-d6) δ = 8.22 (s, 1H), 7.77- | |
| | | 7.72 (m, 1H), 7.66-7.59 (m, 1H), 7.36-7.34 (m, 1H), 6.86 | |
| | | (d, 1H), 5.86 (s, 2H), 4.02-3.97 (q, 2H), 3.41 (s, 3H), 0.91 | |
| | | (t, 3H) | |
| 137 | methyl 2-(3-chloro-4- fluoro-phenyl)-6-[(3- chloropyrazol-1- yl)methyl]-1-ethyl-4- oxo-pyridine-3- carboxylate | 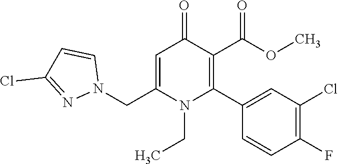 [SMILES: CCn1c(Cn2ccc(Cl)n2)cc(=O)c(C(=O)OC)c1-c1ccc(F)c(Cl)c1] | |
| | | 1H NMR (400 MHz, DMSO-d6) δ = 8.02 (d, 1H), 7.82- | |
| | | 7.79 (dd, 1H), 7.57 (t, 1H), 7.51-7.47 (m, 1H), 6.49 (d, | |
| | | 1H), 5.72 (s, 1H), 5.49 (s, 2H), 3.78 (q, 2H), 3.36 (s, 3H), | |
| | | 0.97 (t, 3H) | |
| 138 | methyl 5-bromo-2-(3- chloro-4-nitro-phenyl)- 6-[(3-chloropyrazol-1- yl)methyl]-1-ethyl-4- oxo-pyridine-3- carboxylate | 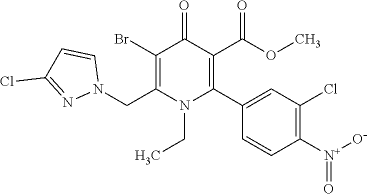 | |
| | | 1H NMR (400 MHz, DMSO-d6) δ = 8.23 (d, 1H), 8.10 (d, | |
| | | 1H), 8.05 (d, 1H), 7.78-7.76 (m, 1H), 6.44 (d, 1H), 5.72 (s, | |
| | | 2H), 4.05-3.99 (m, 2H), 3.42 (s, 3H), 0.96 (t, 3H) | |
| 139 | methyl 5-bromo-2-(3- chloro-4-nitro-phenyl)- 1-ethyl-4-oxo-6-[[3- (trifluoromethyl) pyrazol-1- yl]methyl]pyridine-3- carboxylate | 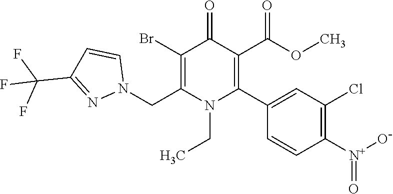 | |
| | | 1H NMR (400 MHz, DMSO-d6) δ = 8.23 (d, 1H), 8.20 (t, | |
| | | 1H), 8.08 (d, 1H), 7.78-7.75 (dd, 1H), 6.86 (d, 1H), 5.86 | |
| | | (s, 2H), 3.99 (q, 2H), 3.43 (s, 3H), 0.93 (t, 3H) | |
| 140 | methyl 5-bromo-2-(4- chloro-3-fluoro- phenyl)-6-[(3- chloropyrazol-1- yl)methyl]-1-ethyl-4- oxo-pyridine-3- carboxylate | 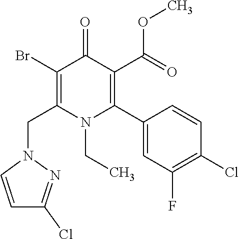 [SMILES: CCn1c(Cn2ccc(Cl)n2)c(Br)c(=O)c(C(=O)OC)c1-c1ccc(Cl)c(F)c1] | |
| | | 1H NMR (400 MHz, chloroform) δ = 8.36 (d, 1H), 8.21 (d, | |
| | | 1H), 7.95 (dd, 1H), 7.83 (dd, 1H), 6.83 (d, 1H), 5.85 (s, | |
| | | 2H), 4.05-3.95 (m, 2H), 3.42 (s, 3H), 0.92 (t, 3H) | |
| 141 | methyl 2-(4-chloro-3- fluoro-phenyl)-1-ethyl- 5-iodo-4-oxo-6-[[3- (trifluoromethyl) pyrazol-1- yl]methyl]pyridine-3- carboxylate | 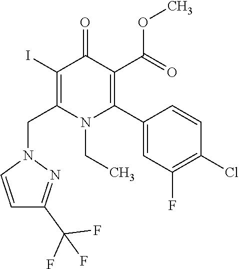 [SMILES: CCn1c(Cn2ccc(C(F)(F)F)n2)c(I)c(=O)c(C(=O)OC)c1-c1ccc(Cl)c(F)c1] | |
| | | 1H NMR (400 MHz, chloroform) δ = 7.89 (d, 1H), 7.56 (t, | |
| | | 1H), 7.19 (dd, 1H), 7.11 (dd, 1H), 6.61 (s, 1H), 5.91 (s, | |
| | | 2H), 4.22 (q, 2H), 3.59 (s, 3H), 1.07 (t, 3H) | |
| 142 | methyl 2-(4-chloro-3- fluoro-phenyl)-1-ethyl- 4-oxo-6-[[3- (trifluoromethyl) pyrazol-1- yl]methyl]pyridine-3- carboxylate | 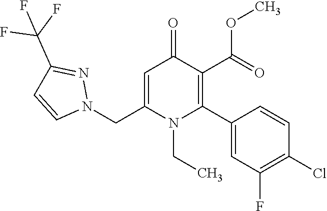 [SMILES: CCn1c(Cn2ccc(C(F)(F)F)n2)cc(=O)c(C(=O)OC)c1-c1ccc(Cl)c(F)c1] | |
| | | 1H NMR (400 MHz, chloroform) δ = 7.68-7.42 (m, 2H), | |
| | | 7.21-7.08 (m, 2H), 6.65 (d, 1H), 6.45 (s, 1H), 5.39 (s, 2H), | |
| | | 3.85 (m, 2H), 3.51 (s, 3H), 1.01 (t, 3H) | |
| 143 | 2-(3,4- dichlorophenyl)-1- ethyl-6-[[5-(2- methoxyethoxy)-3- methyl-pyrazol-1- yl]methyl]-4-oxo- pyridine-3-carboxylic acid | 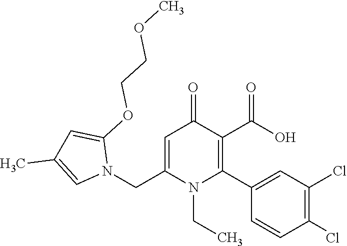 [SMILES: CCn1c(Cn2cc(C)cc2OCCOC)cc(=O)c(C(=O)O)c1-c1ccc(Cl)c(Cl)c1] | |
| | | 1H NMR (400 MHz, DMSO-d6) δ = 7.82-7.75 (m, 2H), | |
| | | 7.43 (d, 1H), 5.99 (s, 1H), 5.68 (s, 1H), 5.28 (s, 2H), 4.26- | |
| | | 4.16 (m, 2H), 3.87 (m, 2H), 3.65-3.56 (m, 2H), 3.24 (s, | |
| | | 3H), 2.13 (s, 3H), 1.06 (t, 3H) | |
| 144 | 2-(3,4- dichlorophenyl)-1- ethyl-6-[[5-(2- methoxyethoxy)-3- (trifluoromethyl) pyrazol-1- yl]methyl]-4-oxo- pyridine-3-carboxylic acid | 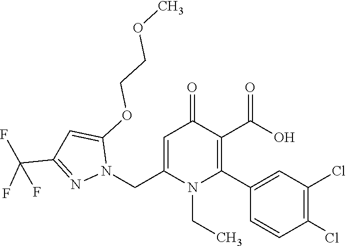 [SMILES: CCn1c(Cn2nc(C(F)(F)F)cc2OCCOC)cc(=O)c(C(=O)O)c1-c1ccc(Cl)c(Cl)c1] | |
| | | 1H NMR (400 MHz, DMSO-d6) δ = 7.87-7.70 (m, 2H), | |
| | | 7.44 (dd, 1H), 6.48 (s, 1H), 6.01 (s, 1H), 5.51 (s, 2H), 4.46- | |
| | | 4.30 (m, 2H), 3.88 (q, 2H), 3.68-3.61 (m, 2H), 3.25 (s, | |
| | | 3H), 1.05 (t, 3H) | |
| 145 | 2-(3,4- dichlorophenyl)-1- ethyl-6-[[5-methoxy-3- (trifluoromethyl)pyrazol- 1-yl]methyl]-4-oxo- pyridine-3-carboxylic acid | 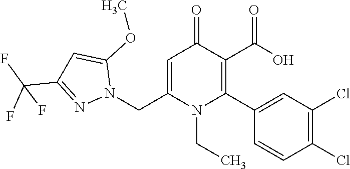 [SMILES: CCn1c(Cn2nc(C(F)(F)F)cc2OC)cc(=O)c(C(=O)O)c1-c1ccc(Cl)c(Cl)c1] | |
| | | 1H NMR (400 MHz, DMSO-d6) δ = 7.87-7.67 (m, 2H), | |
| | | 7.45 (dd, 1H), 6.47 (s, 1H), 5.85 (s, 1H), 5.53 (s, 2H), 4.01 | |
| | | (s, 3H), 3.87 (q, 2H), 1.05 (t, 3H) | |
| 146 | 2-(3,4- dichlorophenyl)-1- ethyl-6-[(5-hydroxy-3- methyl-pyrazol-1- yl)methy]-4-oxo- pyridine-3-carboxylic acid | 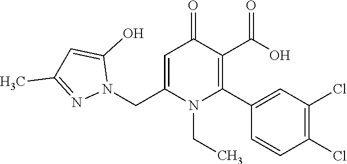 [SMILES: CCn1c(Cn2nc(C)cc2O)cc(=O)c(C(=O)O)c1-c1ccc(Cl)c(Cl)c1] | |
| | | 1H NMR (400 MHz, DMSO-d6) δ = 11.37 (s, 1H), 7.82- | |
| | | 7.77 (m, 2H), 7.46-7.43 (m, 1H), 5.94 (s, 1H), 5.31-4.98 | |
| | | (m, 3H), 3.88 (d, 2H), 2.09 (s, 3H), 1.07 (t, 3H) | |
| 147 | 2-(3,4- dichlorophenyl)-1- ethyl-6-[[5-hydroxy-3- (trifluoromethyl) pyrazol-1-yl]methyl]-4-oxo- pyridine-3-carboxylic acid | 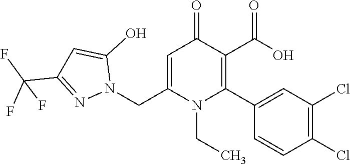 [SMILES: CCn1c(Cn2nc(C(F)(F)F)cc2O)cc(=O)c(C(=O)O)c1-c1ccc(Cl)c(Cl)c1] | |
| | | 1H NMR (400 MHz, DMSO-d6) δ = 12.53 (s, 1H), 7.82 (d, | |
| | | 1H), 7.79 (d, 1H), 7.45 (d, 1H), 5.94-5.89 (m, 2H), 5.48 (s, | |
| | | 2H), 3.88 (q, 2H), 1.06 (t, 3H) | |
| 148 | 2-(3,4- dichlorophenyl)-6-[[5- ethoxy-3- (trifluoromethyl) pyrazol-1- yl]methyl]-1- ethyl-4-oxo-pyridine- 3-carboxylic acid | 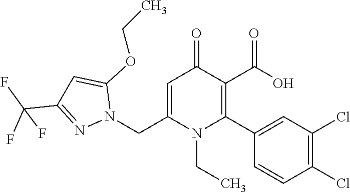 [SMILES: CCOc1cc(C(F)(F)F)nn1Cc1cc(=O)c(C(=O)O)c(-c2ccc(Cl)c(Cl)c2)n1CC] | |
| | | 1H NMR (400 MHz, DMSO-d6) δ = 7.92-7.70 (m, 2H), | |
| | | 7.45 (d, 1H), 6.45 (s, 1H), 5.94 (s, 1H), 5.51 (s, 2H), 4.29 | |
| | | (q, 2H), 3.87 (q, 2H), 1.34 (t, 3H), 1.05 (t, 3H) | |
| 149 | 2-(3,4- dichlorophenyl)-6-[(5- ethoxy-3-methyl- pyrazol-1-yl)methyl]- 1-ethyl-4-oxo- pyridine-3-carboxylic acid | 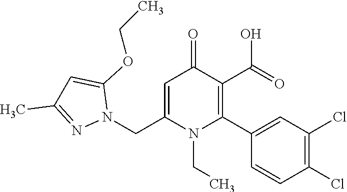 [SMILES: CCOc1cc(C)nn1Cc1cc(=O)c(C(=O)O)c(-c2ccc(Cl)c(Cl)c2)n1CC] | |
| | | 1H NMR (400 MHz, DMSO-d6) δ = 7.84-7.74 (m, 2H), | |
| | | 7.44 (d, 1H), 5.93 (s, 1H), 5.65 (s, 1H), 5.27 (s, 2H), 4.14 | |
| | | (q, 2H), 3.87 (q, 2H), 2.13 (s, 3H), 1.30 (t, 3H), 1.06 (t, 3H) | |
| 150 | 2-(3,4- dichlorophenyl)-6-[[5- (difluoromethoxy)-3- methyl-pyrazol-1- yl]methyl]-1-ethyl-4- oxo-pyridine-3- carboxylic acid | 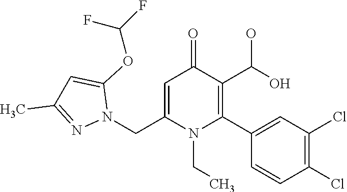 [SMILES: CCn1c(Cn2nc(C)cc2OC(F)F)cc(=O)c(C(O)O)c1-c1ccc(Cl)c(Cl)c1] | |
| | | 1H NMR (400 MHz, DMSO-d6) δ = 7.81 (d, 1H), 7.78 (d, | |
| | | 1H), 7.51-7.13 (m, 2H), 6.02 (s, 1H), 5.87 (s, 1H), 5.44 | |
| | | (s, 2H), 3.87 (q, 2H), 2.19 (s, 3H), 1.07 (t, 3H) | |
| 151 | 2-(3,4- dichlorophenyl)-6-[[5- (difluoromethoxy)-3- (trifluoromethyl) pyrazol-1-yl]methyl]-1- ethyl-4-oxo-pyridine- 3-carboxylic acid | 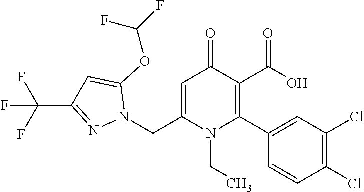 [SMILES: CCn1c(Cn2nc(C(F)(F)F)cc2OC(F)F)cc(=O)c(C(=O)O)c1-c1ccc(Cl)c(Cl)c1] | |
| | | 1H NMR (400 MHz, DMSO-d6) δ = 16.12 (s, 1H), 7.80 (m, | |
| | | 2H), 7.62-7.26 (m, 1H), 7.47-7.41 (m, 1H), 6.84 (s, 1H), | |
| | | 5.92 (s, 1H), 5.69 (s, 2H), 3.87 (d, 2H), 1.06 (t, 3H) | |
| 152 | 6-[(5-tert-butoxy-3- methyl-pyrazol-1-yl) methyl]-2-(3,4- dichlorophenyl)-1-ethyl- 4-oxo-pyridine- 3-carboxylic acid | 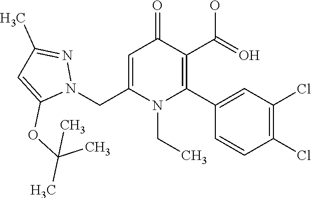 [SMILES: CCn1c(Cn2nc(C)cc2OC(C)(C)C)cc(=O)c(C(=O)O)c1-c1ccc(Cl)c(Cl)c1] | |
| | | 1H NMR (400 MHz, DMSO-d6) δ = 7.80 (m, 2H), 7.44 (dd, | |
| | | 1H), 5.91 (s, 1H), 5.76 (s, 1H), 5.27 (s, 2H), 3.87 (q, 2H), | |
| | | 2.14 (s, 3H), 1.38 (s, 9H), 1.06 (t, 3H) | |
| 153 | (2,3- dichlorophenyl)-1- ethyl-6-[[3-[(2- methoxyacetyl)amino] pyrazol-1-yl]methyl]- 4-oxo-pyridine-3- carboxylic acid | 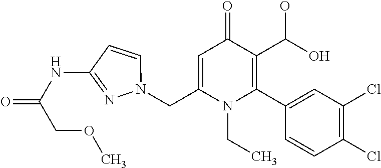 [SMILES: CCn1c(Cn2ccc(NC(=O)COC)n2)cc(=O)c(C(O)O)c1-c1ccc(Cl)c(Cl)c1] | |
| | | 1H NMR (400 MHz, DMSO-d6) δ = 10.28 (s, 1H), 7.89- | |
| | | 7.86 (d, 1H), 7.80-7.77 (m, 2H), 7.44-7.42 (d, 1H), 6.66 | |
| | | (s, 1H), 6.12 (s, 1H), 5.58 (s, 2H), 4.40 (s, 2H), 3.90 (dd, | |
| | | 2H), 3.34 (s, 3H), 1.05 (t, 3H) | |
| 154 | 6-[(3- acetamidopyrazol-1- yl)methyl]-2-(3,4- dichlorophenyl)-1- ethyl-4-oxo-pyridine- 3-carboxylic acid | 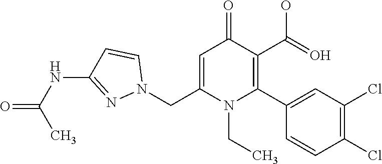 [SMILES: CCn1c(Cn2ccc(NC(C)=O)n2)cc(=O)c(C(=O)O)c1-c1ccc(Cl)c(Cl)c1] | |
| | | 1H NMR (400 MHz, DMSO-d6) δ = 10.50 (s, 1H), 7.85 (d, | |
| | | 1H), 7.81-7.74 (m, 2H), 7.42 (dd, 1H), 6.63 (d, 1H), 6.12 | |
| | | (s, 1H), 5.56 (s, 2H), 3.88 (d, 2H), 2.00 (s, 3H), 1.03 (t, 3H) | |
| 155 | 6-[[3-[[(E)-but-2- enoyl]amino]pyrazol- 1-yl]methyl]-2-(3,4- dichlorophenyl)-1- ethyl-4-oxo-pyridine- 3-carboxylic acid | 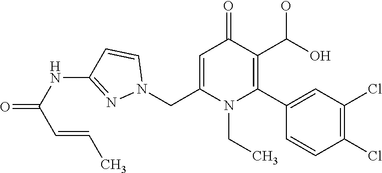 [SMILES: C/C=C/C(=O)Nc1ccn(Cc2cc(=O)c(C(O)O)c(-c3ccc(Cl)c(Cl)c3)n2CC)n1] | |
| | | 1H NMR (400 MHz, DMSO-d6) δ = 10.57 (d, 1H), 7.87 (s, | |
| | | 1H), 7.80-7.76 (m, 2H), 7.42 (d, 1H), 6.86-6.73 (m, 1H), | |
| | | 6.71 (s, 1H), 6.16 (d, 2H), 5.57 (s, 2H), 3.89 (d, 2H), 1.84 | |
| | | (d, 3H), 1.02 (t, 3H) | |
| 156 | 6-[(3-aminopyrazol-1- yl)methyl]-2-(3,4- dichlorophenyl)-1- ethyl-4-oxo-pyridine- 3-carboxylic acid | 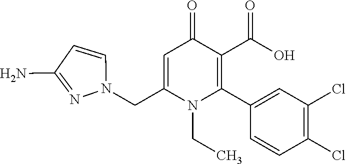 [SMILES: CCn1c(Cn2ccc(N)n2)cc(=O)c(C(=O)O)c1-c1ccc(Cl)c(Cl)c1] | |
| | | 1H NMR (400 MHz, DMSO-d6) δ = 7.82-7.74 (m, 2H), | |
| | | 7.57 (d, 1H), 7.42 (dd, 1H), 6.05 (s, 1H), 5.57 (d, 1H), 5.37 | |
| | | (s, 2H), 4.82 (s, 2H), 3.87 (d, 2H), 1.06 (t, 3H) | |
| 157 | 2-(3,4- dichlorophenyl)-1- ethyl-4-oxo-6-[[3- (prop-2- enoylamino)pyrazol- 1-yl]methyl]pyridine-3- carboxylic acid | 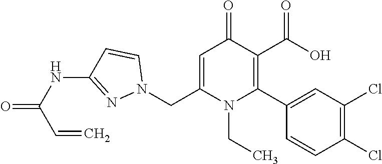 [SMILES: C=CC(=O)Nc1ccn(Cc2cc(=O)c(C(=O)O)c(-c3ccc(Cl)c(Cl)c3)n2CC)n1] | |
| | | 1H NMR (400 MHz, DMSO-d6) δ = 10.79 (s, 1H), 7.90 (d, | |
| | | 1H), 7.78 (s, 1H), 7.65 (s, 1H), 7.42 (dd, 1H), 6.74 (d, 1H), | |
| | | 6.46 (dd, 1H), 6.25 (dd, 1H), 6.19 (s, 1H), 5.80-5.67 (m, | |
| | | 1H), 5.58 (s, 2H), 3.89 (q, 2H), 1.02 (t, 3H) | |
| 158 | 2-(3,4- dichlorophenyl)-1- ethyl-6-[[3-(3- methoxypropanoyl- amino)pyrazol-1- yl]methyl]-4-oxo- pyridine-3-carboxylic acid | 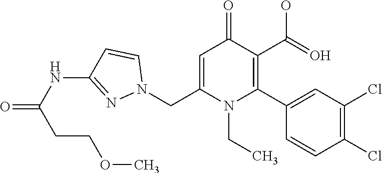 [SMILES: CCn1c(Cn2ccc(NC(=O)CCOC)n2)cc(=O)c(C(=O)O)c1-c1ccc(Cl)c(Cl)c1] | |
| | | 1H NMR (400 MHz, DMSO-d6) δ = 10.53 (s, 1H), 7.86 (d, | |
| | | 1H), 7.80 (d, 1H), 7.78 (d, 1H), 7.43 (dd, 1H), 6.65 (d, 1H), | |
| | | 6.14 (s, 1H), 5.56 (s, 2H), 3.89 (dd, 2H), 3.59 (t, 2H), 3.22 | |
| | | (s, 3H), 2.55-2.50 (m, 2H), 1.04 (t, 3H) | |
| 159 | 6-[[3-(but-3- enoylamino)pyrazol- 1-yl]methyl]-2-(3,4- dichlorophenyl)-1- ethyl-4-oxo-pyridine- 3-carboxylic acid | 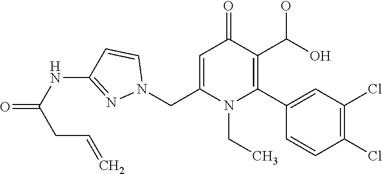 [SMILES: C=CCC(=O)Nc1ccn(Cc2cc(=O)c(C(O)O)c(-c3ccc(Cl)c(Cl)c3)n2CC)n1] | |
| | | 1H NMR (400 MHz, DMSO-d6) δ = 10.57 (s, 1H), 7.87 (d, | |
| | | 1H), 7.79 (d, 1H), 7.77 (d, 1H), 7.42 (dd, 1H), 6.82-6.76 | |
| | | (m, 1H), 6.71 (d, 1H), 6.17 (s, 1H), 6.17 (s, 1H), 6.17-6.13 | |
| | | (m, 1H), 5.57 (s, 2H), 3.88 (dd, 2H), 1.84 (dd, 2H), 1.03 (t, | |
| | | 3H) | |
| 160 | 2-(3,4- dichlorophenyl)-1- ethyl-4-oxo-6-[(3- propoxypyrazol-1- yl)methyl]pyridine-3- carboxylic acid | 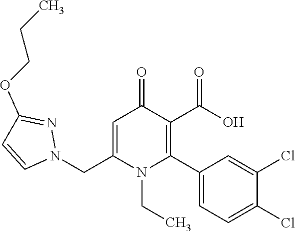 [SMILES: CCCOc1ccn(Cc2cc(=O)c(C(=O)O)c(-c3ccc(Cl)c(Cl)c3)n2CC)n1] | |
| | | 1H NMR (400 MHz, DMSO-d6) δ = 7.82-7.75 (m, 3H), | |
| | | 7.43 (dd, 1H), 5.94 (s, 1H), 5.87 (d, 1H), 5.49 (s, 2H), 4.01 | |
| | | (t, 2H), 3.88 (dd, 2H), 1.76-1.64 (m, 2H), 1.08 (t, 3H), | |
| | | 0.94 (t, 3H) | |
| 161 | 2-(3,4- dichlorophenyl)-1- ethyl-6-[[3-(2- methoxyethoxy) pyrazol-1-yl]methyl]-4-oxo- pyridine-3-carboxylic acid | 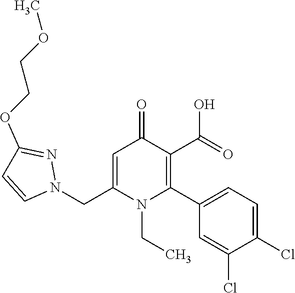 [SMILES: CCn1c(Cn2ccc(OCCOC)n2)cc(=O)c(C(=O)O)c1-c1ccc(Cl)c(Cl)c1] | |
| | | 1H NMR (400 MHz, DMSO-d6) δ = 7.87-7.68 (m, 3H), | |
| | | 7.43 (dd, 1H), 5.95 (s, 1H), 5.88 (d, 1H), 5.50 (s, 2H), 4.23- | |
| | | 4.12 (m, 2H), 3.88 (q, 2H), 3.67-3.54 (m, 2H), 3.29 (s, | |
| | | 3H), 1.08 (t, 3H) | |
| 162 | 2-(3,4- dichlorophenyl)-1- ethyl-6-[[3- (methoxymethyl) pyrazol-1-yl]methyl]-4-oxo- pyridine-3-carboxylic acid | 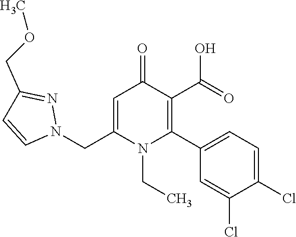 [SMILES: CCn1c(Cn2ccc(COC)n2)cc(=O)c(C(=O)O)c1-c1ccc(Cl)c(Cl)c1] | |
| | | 1H NMR (400 MHz, DMSO-d6) δ = 7.94 (d, 1H), 7.79 (d, | |
| | | 1H), 7.78 (d, 1H), 7.43 (dd, 1H), 6.41 (d, 1H), 5.92 (s, 1H), | |
| | | 5.66 (s, 2H), 4.39 (s, 2H), 3.94-3.85 (m, 2H), 3.28-3.25 | |
| | | (m, 3H), 1.06 (t, 3H) | |
| 163 | 6-[(3-allyloxypyrazol- 1-yl)methyl]-2-(3,4- dichlorophenyl)-1- ethyl-4-oxo-pyridine- 3-carboxylic acid | 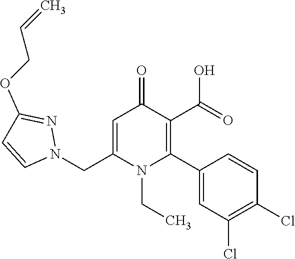 [SMILES: C=CCOc1ccn(Cc2cc(=O)c(C(=O)O)c(-c3ccc(Cl)c(Cl)c3)n2CC)n1] | |
| | | 1H NMR (400 MHz, DMSO-d6) δ = 7.81-7.75 (m, 3H), | |
| | | 7.43 (d, 1H), 6.03 (m, 1H), 5.95 (s, 1H), 5.89 (s, 1H), 5.50 | |
| | | (s, 2H), 5.37 (d, 1H), 5.24 (d, 1H), 4.61 (d, 2H), 3.88 (q, | |
| | | 2H), 1.07 (t, 3H) | |
| 164 | 2-(3,4- dichlorophenyl)-6-[(3- ethoxypyrazol-1- yl)methyl]-1-ethyl-4- oxo-pyridine-3- carboxylic acid | 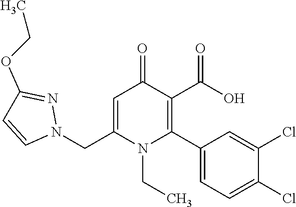 [SMILES: CCOc1ccn(Cc2cc(=O)c(C(=O)O)c(-c3ccc(Cl)c(Cl)c3)n2CC)n1] | |
| | | 1H NMR (500 MHz, DMSO-d6) δ = 7.82-7.75 (m, 3H), | |
| | | 7.43 (dd, 1H), 5.95 (s, 1H), 5.86 (d, 1H), 5.49 (s, 2H), 4.11 | |
| | | (q, J = 7.0 Hz, 2H), 3.93-3.84 (m, 2H), 1.36-1.24 (m, | |
| | | 3H), 1.08 (t, J = 7.2 Hz, 3H) | |
| 165 | 2-(3,4- dichlorophenyl)-6-[[3- (2,2- difluoroethoxy)pyrazol- 1-yl]methyl]-1-ethyl- 4-oxo-pyridine-3- carboxylic acid | 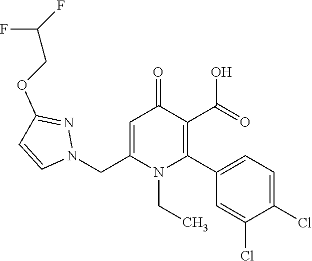 [SMILES: CCn1c(Cn2ccc(OCC(F)F)n2)cc(=O)c(C(=O)O)c1-c1ccc(Cl)c(Cl)c1] | |
| | | 1H NMR (500 MHz, DMSO-d6) δ = 7.81 (brs, 1H), 7.72 (d, | |
| | | 1H), 7.67 (s, 1H), 7.36 (dd, 1H), 6.35 (tt, 1H), 5.93 (d, 1H), | |
| | | 5.80 (s, 1H), 5.40 (s, 2H), 4.37 (dt, 2H), 3.86-3.77 (m, | |
| | | 2H), 0.99 (t, 3H) | |
| 166 | 2-(3,4- dichlorophenyl)-1- ethyl-6-[(3- isopropoxypyrazol-1- yl)methyl]-4-oxo- pyridine-3-carboxylic acid | 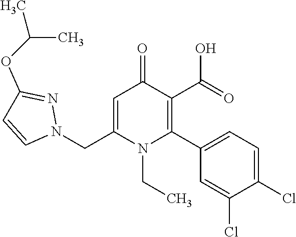 [SMILES: CCn1c(Cn2ccc(OC(C)C)n2)cc(=O)c(C(=O)O)c1-c1ccc(Cl)c(Cl)c1] | |
| | | 1H NMR (500 MHz, DMSO-d6) δ = 7.80-7.73 (m, 3H), | |
| | | 7.41 (dd, 1H), 5.92 (s, 1H), 5.83 (d, 1H), 5.46 (s, 2H), 4.66 | |
| | | (td, 1H), 3.87 (q, 2H), 1.27 (d, 6H), 1.07 (t, 3H) | |
| 167 | 2-(3,4- dichlorophenyl)-1- ethyl-6-[(3- hydroxypyrazol-1- yl)methyl]-4-oxo- pyridine-3-carboxylic acid | 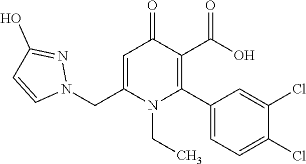 [SMILES: CCn1c(Cn2ccc(O)n2)cc(=O)c(C(=O)O)c1-c1ccc(Cl)c(Cl)c1] | |
| | | 1H NMR (400 MHz, DMSO-d6) δ = 9.94 (s, 1H), 7.82- | |
| | | 7.73 (m, 2H), 7.66 (d, 1H), 7.45-7.40 (m, 1H), 6.08 (d, | |
| | | 1H), 5.64 (d, 1H), 5.41 (s, 2H), 3.88 (q, 2H), 1.05 (t, 3H) | |
| 168 | 6-[[4,5- bis(ethoxycarbonyl) imidazol-1-yl]methyl]-5- bromo-2-(3,4- dichlorophenyl)-1- ethyl-4-oxo-pyridine- 3-carboxylic acid | 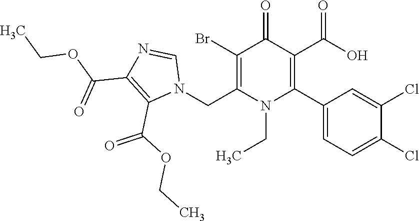 [SMILES: CCOC(=O)c1ncn(Cc2c(Br)c(=O)c(C(=O)O)c(-c3ccc(Cl)c(Cl)c3)n2CC)c1C(=O)OCC] | |
| | | 1H NMR (400 MHz, DMSO-d6) δ = 14.16 (s, 1H), 7.93 (s, | |
| | | 1H), 7.91 (d, 1H), 7.83 (d, 1H), 7.58-7.47 (m 1H), 5.78 | |
| | | (d, 2H), 4.39-4.23 (m, 4H), 3.90 (d, 2H), 1.29 (q, 6H), | |
| | | 1.05 (t, 3H) | |
| 169 | 6-[[5-chloro-3- (trifluoromethyl) pyrazol-1-yl]methyl]-2-(3,4- dichlorophenyl)-1- ethyl-4-oxo-pyridine- 3-carboxylic acid | 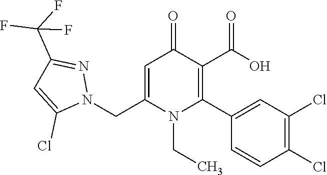 [SMILES: CCn1c(Cn2nc(C(F)(F)F)cc2Cl)cc(=O)c(C(=O)O)c1-c1ccc(Cl)c(Cl)c1] | |
| | | 1H NMR (400 MHz, chloroform) δ = 7.61 (d, 1H), 7.37 | |
| | | (d, 1H), 7.13 (dd, 1H), 6.67 (s, 1H), 6.31 (s, 1H), 5.47 (s, | |
| | | 2H), 4.01 (dq, 2H), 2.24 (t, 3H) | |
| 170 | 2-(3,4- dichlorophenyl)-1-(2- methoxyethyl)-4-oxo- 6-[[3- (trifluoromethyl) pyrazol-1- yl]methyl]pyridine-3- carboxylic acid | 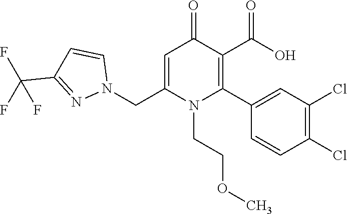 [SMILES: COCCn1c(Cn2ccc(C(F)(F)F)n2)cc(=O)c(C(=O)O)c1-c1ccc(Cl)c(Cl)c1] | |
| | | 1H NMR (400 MHz, DMSO-d6) δ = 15.89 (s, 1H), 8.18 (s, | |
| | | 1H), 7.89-7.76 (m, 2H), 7.42-7.40 (dd, 1H), 6.93 (d, 1H), | |
| | | 5.94 (s, 1H), 5.83 (s, 2H), 4.07 (t, 2H), 3.38 (t, 2H), 2.07 (s, | |
| | | 3H) | |
| 171 | 6-[(3-chloropyrazol-1- yl)methyl]-2-(3,4- dichlorophenyl)-1-(2- methoxyethyl)-4-oxo- pyridine-3-carboxylic acid | 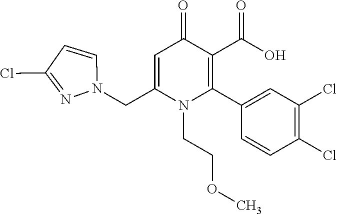 [SMILES: COCCn1c(Cn2ccc(Cl)n2)cc(=O)c(C(=O)O)c1-c1ccc(Cl)c(Cl)c1] | |
| | | 1H NMR (400 MHz, DMSO-d6) δ = 8.00 (d, 1H), 7.79- | |
| | | 7.76 (m, 2H), 7.42-7.39 (dd, 1H), 6.52 (d, 1H), 6.01 (s, | |
| | | 1H), 5.68 (s, 2H), 4.06 (t, 2H), 3.37 (t, 2H), 3.17 (s, 3H) | |
| 172 | 2-(3,4- dichlorophenyl)-1- ethyl-4-oxo-6-[1-[3- (trifluoromethyl) pyrazol-1-yl]ethyl]pyridine- 3-carboxylic acid | 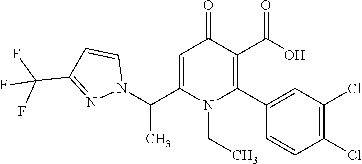 [SMILES: CCn1c(C(C)n2ccc(C(F)(F)F)n2)cc(=O)c(C(=O)O)c1-c1ccc(Cl)c(Cl)c1] | |
| | | 1H NMR (400 MHz, DMSO-d6) δ = 8.38 (s, 1H), 7.87- | |
| | | 7.75 (m, 2H), 7.52-7.44 (m, 1H), 6.93 (s, 1H), 6.63 (s, | |
| | | 0.5H), 6.54 (s, 0.5H), 6.18-6.13 (m, 1H), 4.00-3.85 (m, | |
| | | 2H), 1.93 (d, 3H), 1.14 (t, 3H) | |
| 173 | 2-(3,4- dichlorophenyl)-6-[[3- (ethoxymethyl)pyrazol- 1-yl]methyl]-1-ethyl- 4-oxo-pyridine-3- carboxylic acid | 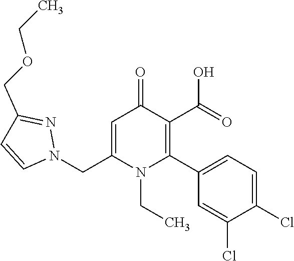 [SMILES: CCOCc1ccn(Cc2cc(=O)c(C(=O)O)c(-c3ccc(Cl)c(Cl)c3)n2CC)n1] | |
| | | 1H NMR (400 MHz, DMSO-d6) δ = 7.92 (d, 1H), 7.83- | |
| | | 7.71 (m, 2H), 7.43 (dd, 1H), 6.40 (d, 1H), 5.91 (s, 1H), 5.65 | |
| | | (s, 2H), 4.42 (s, 2H), 3.89 (d, 2H), 3.47 (q, 2H), 1.12 (t, | |
| | | 3H), 1.05 (t, 3H) | |
| 174 | 6-[(3-carboxypyrazol- 1-yl)methyl]-2-(3,4- dichlorophenyl)-1- ethyl-4-oxo-pyridine- 3-carboxylic acid | 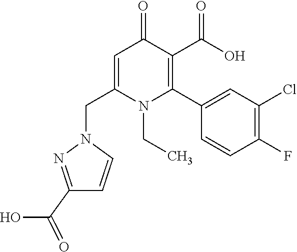 [SMILES: CCn1c(Cn2ccc(C(=O)O)n2)cc(=O)c(C(=O)O)c1-c1ccc(F)c(Cl)c1] | |
| | | 1H NMR (500 MHz, DMSO-d6) δ = 8.08 (d, 1H), 7.82- | |
| | | 7.77 (m, 2H), 7.44 (dd, 1H), 6.86 (d, 1H), 6.01 (s, 1H), 5.78 | |
| | | (s, 2H), 3.92-3.88 (m, 2H), 1.05 (t, 3H) | |
| 175 | 6-[[3- (cyanomethyl)pyrazol- 1-yl]methyl]-2-(3,4- dichlorophenyl)-1- ethyl-4-oxo-pyridine- 3-carboxylic acid | 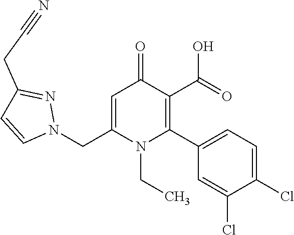 [SMILES: CCn1c(Cn2ccc(CC#N)n2)cc(=O)c(C(=O)O)c1-c1ccc(Cl)c(Cl)c1] | |
| | | 1H NMR (400 MHz, DMSO-d6) δ = 7.98 (d, 1H), 7.79- | |
| | | 7.77 (m, 2H), 7.45-7.42 (dd, 1H), 6.42 (d, 1H), 5.93 (s, | |
| | | 1H), 5.68 (s, 2H), 4.06 (s, 2H), 3.89 (q, 2H), 1.08-1.04 (t, | |
| | | 3H) | |
| 176 | 6-[(5-cyanopyrazol-1- yl)methyl]-2-(3,4- dichlorophenyl)-1- ethyl-4-oxo-pyridine- 3-carboxylic acid | 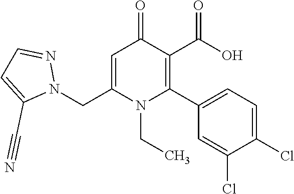 [SMILES: CCn1c(Cn2nccc2C#N)cc(=O)c(C(=O)O)c1-c1ccc(Cl)c(Cl)c1] | |
| | | 1H NMR (400 MHz, DMSO-d6) δ = 16.24 (brs, 1H), 8.29 | |
| | | (d, 1H), 7.90-7.67 (m, 2H), 7.44 (d, 1H), 7.17 (d, 1H), | |
| | | 6.16 (s, 1H), 5.82 (s, 2H), 3.88 (q, 2H), 1.02 (t, 3H) | |
| 177 | 6-[(3-bromopyrazol-1- yl)methyl]-2-(3,4- dichlorophenyl)-1- ethyl-4-oxo-pyridine- 3-carboxylic acid | 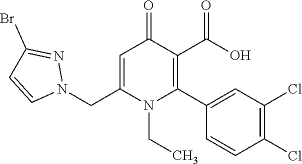 [SMILES: CCn1c(Cn2ccc(Br)n2)cc(=O)c(C(=O)O)c1-c1ccc(Cl)c(Cl)c1] | |
| | | 1H NMR (400 MHz, chloroform) δ = 7.61 (d, 1H), 7.49 (d, | |
| | | 1H), 7.39 (d, 1H), 7.14 (dd, 1H), 6.43 (d, 1H), 6.34 (s, 1H), | |
| | | 5.37 (s, 2H), 3.96 (q, 2H), 1.20 (t, 3H) | |
| 178 | 2-(3,4- dichlorophenyl)-1- ethyl-6-[(3- nitropyrazol-1- yl)methyl]-4-oxo- pyridine-3-carboxylic acid | 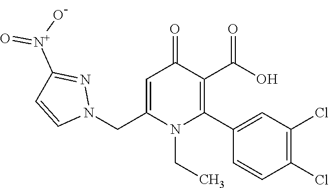 | |
| | | 1H NMR (500 MHz, DMSO-d6) δ = 8.28 (d, 1H), 7.82- | |
| | | 7.77 (m, 2H), 7.44 (dd, 1H), 7.23 (d, 1H), 6.24 (s, 1H), 5.85 | |
| | | (s, 2H), 3.89 (q, 2H), 1.06 (t, 3H) | |
| 179 | 6-[(3- cyclopropylpyrazol-1- yl)methyl]-2-(3,4- dichlorophenyl)-1- ethyl-4-oxo-pyridine- 3-carboxylic acid | 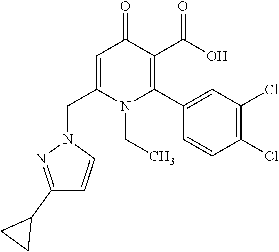 [SMILES: CCn1c(Cn2ccc(C3CC3)n2)cc(=O)c(C(=O)O)c1-c1ccc(Cl)c(Cl)c1] | |
| | | 1H NMR (500 MHz, DMSO-d6) δ = 7.82-7.74 (m, 3H), | |
| | | 7.42 (dd, 1H), 6.12 (d, 1H), 5.87 (s, 1H), 5.56 (s, 2H), 3.88 | |
| | | (brd, 2H), 1.91 (dt, 1H), 1.06 (t, 3H), 0.93-0.84 (m, 2H), | |
| | | 0.69-0.62 (m, 2H) | |
| 180 | 6-[(3-acetylpyrazol-1- yl)methyl]-2-(3,4- dichlorophenyl)-1- ethyl-4-oxo-pyridine- 3-carboxylic acid | 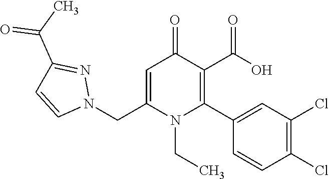 [SMILES: CCn1c(Cn2ccc(C(C)=O)n2)cc(=O)c(C(=O)O)c1-c1ccc(Cl)c(Cl)c1] | |
| | | 1H NMR (500 MHz, DMSO-d6) δ = 16.33 (brs, 1H), 8.11 | |
| | | (d, 1H), 7.81 (d, 1H), 7.79 (d, 1H), 7.44 (dd, 1H), 6.89 (d, | |
| | | 1H), 5.99 (s, 1H), 5.82 (s, 2H), 3.92 (q, 2H), 2.50 (s, 3H), | |
| | | 1.09 (t, 3H) | |
| 181 | 6-[[3-tert-butyl-5- (trifluoromethyl) pyrazol-1-yl]methyl]-2-(3,4- dichlorophenyl)-1- ethyl-4-oxo-pyridine- 3-carboxylic acid | 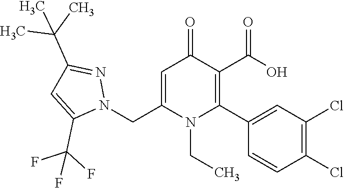 [SMILES: CCn1c(Cn2nc(C(C)(C)C)cc2C(F)(F)F)cc(=O)c(C(=O)O)c1-c1ccc(Cl)c(Cl)c1] | |
| | | 1H NMR (500 MHz, DMSO-d6) δ = 7.71 (d, 1H), 7.63 (d, | |
| | | 1H), 7.35 (dd, 1H), 7.04 (s, 1H), 5.60 (s, 2H), 5.17 (s, 1H), | |
| | | 3.78 (q, 2H), 1.30 (s, 9H), 0.99 (t, 3H) | |
| 182 | 2-(3,4- dichlorophenyl)-1- ethyl-6-[(5-methyl-3- nitro-pyrazol-1- yl)methyl]-4-oxo- pyridine-3-carboxylic acid | 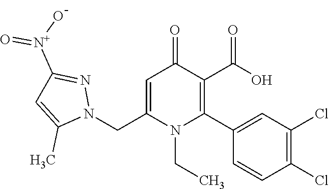 | |
| | | 1H NMR (500 MHz, DMSO-d6) δ = 7.80 (dd, 2H), 7.45 (dd, | |
| | | 1H), 7.07 (s, 1H), 5.80 (s, 2H), 5.76 (s, 1H), 3.95-3.81 (m, | |
| | | 2H), 2.39 (s, 3H), 1.12 (t, 3H) | |
| 183 | 6-[[tert-butyl-3- (trifluoromethyl) pyrazol-1-yl]methyl]-2-(3,4- dichlorophenyl)-1- ethyl-4-oxo-pyridine- 3-carboxylic acid | 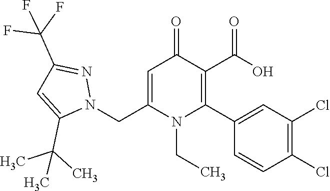 [SMILES: CCn1c(Cn2nc(C(F)(F)F)cc2C(C)(C)C)cc(=O)c(C(=O)O)c1-c1ccc(Cl)c(Cl)c1] | |
| | | 1H NMR (500 MHz, DMSO-d6) δ = 7.82 (d, 1H), 7.80 (d, | |
| | | 1H), 7.46 (d, 1H), 6.75 (s, 1H), 5.80 (s, 2H), 5.37 (s, 1H), | |
| | | 3.93-3.86 (m, 2H), 1.37 (s, 9H), 1.14 (t, 3H) | |
| 184 | 6-[[5-cyclopropyl-3- (trifluoromethyl) pyrazol-1-yl]methyl]-2-(3,4- dichlorophenyl)-1- ethyl-4-oxo-pyridine- 3-carboxylic acid | 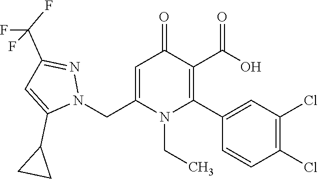 [SMILES: CCn1c(Cn2nc(C(F)(F)F)cc2C2CC2)cc(=O)c(C(=O)O)c1-c1ccc(Cl)c(Cl)c1] | |
| | | 1H NMR (500 MHz, DMSO-d6) δ = 7.71 (d, 1H), 7.64 (d, | |
| | | 1H), 7.40-7.32 (m, 1H), 6.58 (s, 1H), 5.70 (s, 2H), 5.34 (s, | |
| | | 1H), 3.85-3.74 (m, 2H), 2.01-1.92 (m, 1H), 1.06-0.93 | |
| | | (m, 5H), 0.81-0.71 (m, 2H) | |
| 185 | 2-(3,4- dichlorophenyl)-1- ethyl-4-oxo-6-[[3- (2,2,2- trifluoroethoxy)pyrazol- 1-yl]methyl]pyridine- 3-carboxylic acid | 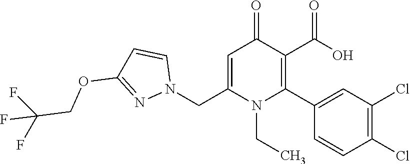 [SMILES: CCn1c(Cn2ccc(OCC(F)(F)F)n2)cc(=O)c(C(=O)O)c1-c1ccc(Cl)c(Cl)c1] | |
| | | 1H NMR (500 MHz, DMSO-d6) δ = 7.86 (d, 1H), 7.80 (d, | |
| | | 1H), 7.78 (d, 1H), 7.43 (dd, 1H), 6.04 (d, 1H), 6.01 (s, 1H), | |
| | | 5.53 (s, 2H), 4.80 (q, 2H), 3.89 (q, 2H), 1.08 (t, 3H) | |
| 186 | 2-(3,4- dichlorophenyl)-6-[[3- (difluoromethyl)pyrazol- 1-yl]methyl]-1- ethyl-4-oxo-pyridine- 3-carboxylic acid | 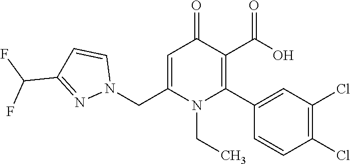 [SMILES: CCn1c(Cn2ccc(C(F)F)n2)cc(=O)c(C(=O)O)c1-c1ccc(Cl)c(Cl)c1] | |
| | | 1H NMR (400 MHz, DMSO-d6) δ = 8.12 (d, 1H), 7.79 (t, | |
| | | 2H), 7.43 (dd, 1H), 7.19-6.92 (m, 1H), 6.71 (s, 1H), 5.99 | |
| | | (s, 1H), 5.75 (s, 2H), 3.91-3.86 (m, 2H), 1.03 (t, 3H) | |
| 187 | 2-(3,4- dichlorophenyl)-6-[[3- (difluoromethyl)-5- methyl-pyrazol-1- yl]methyl]-1-ethyl-4- oxo-pyridine-3- carboxylic acid | 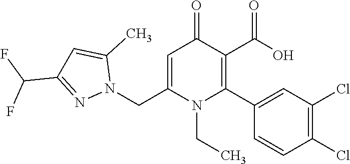 [SMILES: CCn1c(Cn2nc(C(F)F)cc2C)cc(=O)c(C(=O)O)c1-c1ccc(Cl)c(Cl)c1] | |
| | | 1H NMR (400 MHz, DMSO-d6) δ = 7.81-7.77 (m, 2H), | |
| | | 7.4 (dd, 1H), 7.32 (s, 0.2H), 7.13 (s, 0.3H), 6.99 (s, 0.3H), | |
| | | 6.80 (s, 0.2H), 6.64 (s, 0.3H), 6.53 (s, 0.7H), 5.67 (s, 2H), | |
| | | 5.53 (s, 1H), 3.39 (q, 2H), 2.33 (s, 3H), 1.09 (t, 3H) | |
| 188 | 2-(3,4- dichlorophenyl)-6-[[5- (ethoxymethyl)pyrazol- 1-yl]methyl]-1-ethyl- 4-oxo-pyridine-3- carboxylic acid | 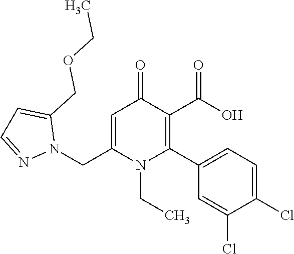 [SMILES: CCOCc1ccnn1Cc1cc(=O)c(C(=O)O)c(-c2ccc(Cl)c(Cl)c2)n1CC] | |
| | | 1H NMR (400 MHz, DMSO-d6) δ = 7.78 (dd, 2H), 7.62 (d, | |
| | | 1H), 7.41 (dd, 1H), 6.46 (d, 1H), 5.65 (d, 3H), 4.56 (s, 2H), | |
| | | 3.89 (d, 2H), 3.40 (dd, 2H), 1.10 (t, 3H), 1.01 (t, 3H) | |
| 189 | 2-(3,4- dichlorophenyl)-1- ethyl-4-oxo-6-[[4- (trifluoromethyl)triazol- 2-yl]methyl]pyridine- 3-carboxylic acid | 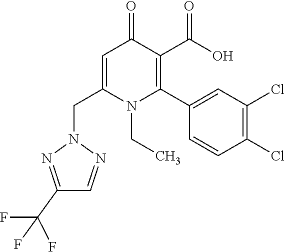 [SMILES: CCn1c(Cn2ncc(C(F)(F)F)n2)cc(=O)c(C(=O)O)c1-c1ccc(Cl)c(Cl)c1] | |
| | | 1H NMR (400 MHz, chloroform) δ = 7.98 (s, 1H), 7.61 (d, | |
| | | 1H), 7.37 (d, 1H), 7.14 (dd, 1H), 6.70 (d, 1H), 5.74 (s, 2H), | |
| | | 4.02 (q, 2H), 1.20 (t, 3H) | |
| 190 | 2-(3,4- dichlorophenyl)-1- ethyl-6-[[5- (methoxymethyl) pyrazol-1-yl]methyl]-4-oxo- pyridine-3-carboxylic acid | 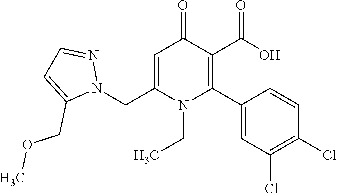 [SMILES: CCn1c(Cn2nccc2COC)cc(=O)c(C(=O)O)c1-c1ccc(Cl)c(Cl)c1] | |
| | | 1H NMR (500 MHz, DMSO-d6) δ = 7.82-7.76 (m, 3H), | |
| | | 7.63 (d, 1H), 7.46-7.41 (m, 1H), 6.48 (d, 1H), 5.64 (s, 2H), | |
| | | 4.52 (s, 2H), 3.88 (q, 2H), 3.22 (s, 3H), 1.10 (t, 3H) | |
| 191 | 2-(3,4- dichlorophenyl)-1- ethyl-4-oxo-6-[[4- (trifluoromethyl)triazol- 1-yl]methyl]pyridine- 3-carboxylic acid | 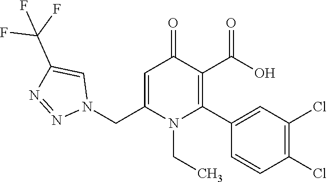 [SMILES: CCn1c(Cn2cc(C(F)(F)F)nn2)cc(=O)c(C(=O)O)c1-c1ccc(Cl)c(Cl)c1] | |
| | | 1H NMR (400 MHz, chloroform) δ = 8.36 (s, 1H), 7.59 (d, | |
| | | 1H), 7.37 (d, 1H), 7.12 (dd, 1H), 6.63 (s, 1H), 5.74 (s, 2H), | |
| | | 4.04 (q, 2H), 1.27-1.12 (m, 3H) | |
| 192 | 2-(3,4- dichlorophenyl)-1- ethyl-4-oxo-6-[[3- (propanoylamino) pyrazol-1- yl]methyl]pyridine-3- carboxylic acid | 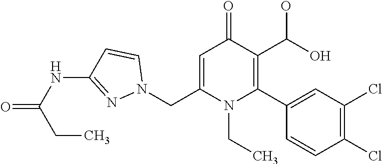 [SMILES: CCC(=O)Nc1ccn(Cc2cc(=O)c(C(O)O)c(-c3ccc(Cl)c(Cl)c3)n2CC)n1] | CHECK SCOPE |
| | | 1H NMR (400 MHz, DMSO-d6) δ = 10.45 (s, 1H), 7.85- | |
| | | 7.77 (m, 3H), 7.42 (d, 1H), 6.65 (s, 1H), 6.12 (s, 1H), 5.56 | |
| | | (s, 2H), 3.88 (d, 2H), 2.28 (d, 2H), 1.09-0.99 (m, 6H) | |
| 193 | 2-(3,4- dichlorophenyl)-1- ethyl-6-[[3-(4- fluorophenyl)pyrazol- 1-yl]methyl]-4-oxo- pyridine-3-carboxylic acid | 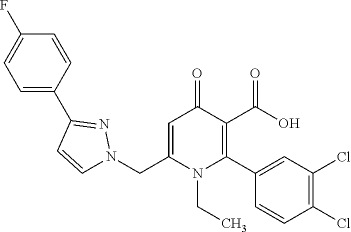 [SMILES: CCn1c(Cn2ccc(-c3ccc(F)cc3)n2)cc(=O)c(C(=O)O)c1-c1ccc(Cl)c(Cl)c1] | |
| | | 1H NMR (500 MHz, DMSO-d6) δ = 16.48 (brs, 1H), 8.05 | |
| | | (d, 1H), 7.88 (dd, 2H), 7.81 (d, 1H), 7.78 (d, 1H), 7.45 (dd, | |
| | | 1H), 7.26 (t, 2H), 6.92 (d, 1H), 6.03 (s, 1H), 5.74 (s, 2H), | |
| | | 3.94 (q, 2H), 1.11 (t, 3H) | |
| 194 | 2-(3,4- dichlorophenyl)-1- ethyl-4-oxo-6-[[3-(2- thienyl)pyrazol-1- yl]methyl]pyridine-3- carboxylic acid | 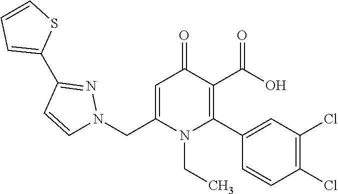 [SMILES: CCn1c(Cn2ccc(-c3cccs3)n2)cc(=O)c(C(=O)O)c1-c1ccc(Cl)c(Cl)c1] | |
| | | 1H NMR (500 MHz, DMSO-d6) δ = 7.97 (d, 1H), 7.70 (d, | |
| | | 1H), 7.64 (d, 1H), 7.51-7.41 (m, 2H), 7.35 (dd, 1H), 7.10 | |
| | | (dd, 1H), 6.76 (d, 1H), 5.70 (s, 1H), 5.57 (s, 2H), 3.87- | |
| | | 3.77 (m, 2H), 0.99 (t, 3H) | |
| 195 | 2-(3,4- dichlorophenyl)-1- ethyl-4-oxo-6-[[3-(4- pyridyl)pyrazol-1- yl]methyl]pyridine-3- carboxylic acid | 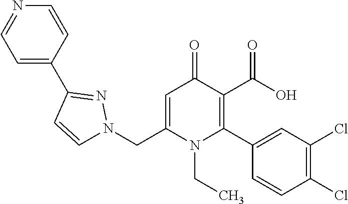 [SMILES: CCn1c(Cn2ccc(-c3ccncc3)n2)cc(=O)c(C(=O)O)c1-c1ccc(Cl)c(Cl)c1] | |
| | | 1H NMR (500 MHz, DMSO-d6) δ = 8.66 (brs, 2H), 8.25 (s, | |
| | | 1H), 7.90 (brs, 2H), 7.86-7.75 (m, 2H), 7.46 (dd, 1H), 7.17 | |
| | | (brs, 1H), 6.04 (s, 1H), 5.87 (s, 2H), 3.97 (q, 2H), 1.10 (t, | |
| | | 3H) | |
| 196 | 2-(3,4- dichlorophenyl)-1- ethyl-4-oxo-6-[[3-(p- tolyl)pyrazol-1- yl]methyl]pyridine-3- carboxylic acid | 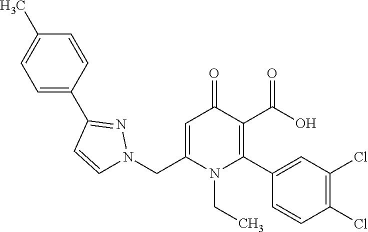 [SMILES: CCn1c(Cn2ccc(-c3ccc(C)cc3)n2)cc(=O)c(C(=O)O)c1-c1ccc(Cl)c(Cl)c1] | |
| | | 1H NMR (500 MHz, DMSO-d6) δ = 8.02 (d, 1H), 7.81 (s, | |
| | | 1H), 7.78 (d, 1H), 7.72 (m, 2H), 7.45 (dd, 1H), 7.23 (m, | |
| | | 2H), 6.87 (d, 1H), 6.01 (s, 1H), 5.73 (s, 2H), 3.94 (q, 2H), | |
| | | 2.33 (s, 3H), 1.11 (t, 3H) | |
| 197 | 2-(3,4- dichlorophenyl)-1- ethyl-6-[[3-(3- nitrophenyl)pyrazol-1- yl]methyl]-4-oxo- pyridine-3-carboxylic acid | 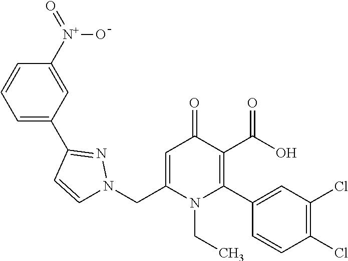 | |
| | | 1H NMR (500 MHz, DMSO-d6) δ = 8.63 (s, 1H), 8.30 (d, | |
| | | 1H), 8.19 (dd, 1H), 8.13 (d, 1H), 7.81 (d, 1H), 7.79 (d, 1H), | |
| | | 7.75 (t, 1H), 7.45 (dd, 1H), 7.15 (d, 1H), 6.01 (s, 1H), 5.80 | |
| | | (s, 2H), 3.94 (q, 2H), 1.11 (t, 3H) | |
| 198 | 2-(3,4- dichlorophenyl)-1- ethyl-4-oxo-6-[[3- phenyl-5- (trifluoromethyl) pyrazol-1- yl]methyl]pyridine-3- carboxylic acid | 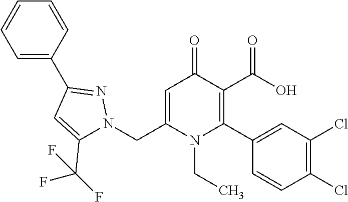 [SMILES: CCn1c(Cn2nc(-c3ccccc3)cc2C(F)(F)F)cc(=O)c(C(=O)O)c1-c1ccc(Cl)c(Cl)c1] | |
| | | 1H NMR (500 MHz, DMSO-d6) δ = 7.92 (d, 2H), 7.72 (d, | |
| | | 1H), 7.69 (s, 1H), 7.66 (d, 1H), 7.48 (t, 2H), 7.43-7.40 (m, | |
| | | 1H), 7.38 (dd, 1H), 5.73 (s, 2H), 5.34 (s, 1H), 3.82 (q, 2H), | |
| | | 1.03 (t, 3H) | |
| 199 | 6-[[3-(4- chlorophenyl)pyrazol- 1-yl]methyl]-2-(3,4- dichlorophenyl)-1- ethyl-4-oxo-pyridine- 3-carboxylic acid | 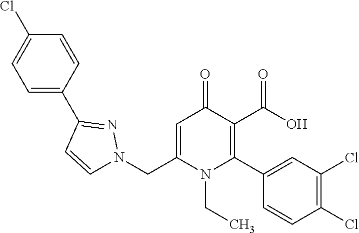 [SMILES: CCn1c(Cn2ccc(-c3ccc(Cl)cc3)n2)cc(=O)c(C(=O)O)c1-c1ccc(Cl)c(Cl)c1] | |
| | | 1H NMR (500 MHz, DMSO-d6) δ = 8.06 (d, 1H), 7.86 (d, | |
| | | 2H), 7.81 (s, 1H), 7.78 (d, 1H), 7.49 (d, 2H), 7.45 (dd, 1H), | |
| | | 6.95 (d, 1H), 6.03 (s, 1H), 5.75 (s, 2H), 3.94 (q, 2H), 1.10 | |
| | | (t, 3H) | |
| 200 | 2-(3,4- dichlorophenyl)-1- ethyl]-6-[[3-(2- furyl)pyrazol-1- yl]methyl]-4-oxo- pyridine-3-carboxylic acid | 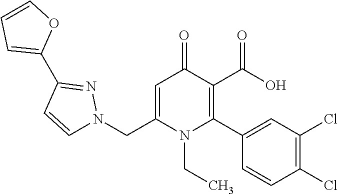 [SMILES: CCn1c(Cn2ccc(-c3ccco3)n2)cc(=O)c(C(=O)O)c1-c1ccc(Cl)c(Cl)c1] | |
| | | 1H NMR (500 MHz, DMSO-d6) δ = 8.01 (brs, 1H), 7.70 (d, | |
| | | 2H), 7.66 (d, 1H), 7.40-7.32 (m, 1H), 6.74 (d, 1H), 6.67 | |
| | | (d, 1H), 6.57 (dd, 1H), 5.76 (s, 1H), 5.60 (brs, 2H), 3.83 | |
| | | (brq, 2H), 0.98 (brt, 3H) | |
| 201 | 2-(3,4- dichlorophenyl)-1- ethyl-4-oxo-6-[(3- thiazol-2-ylpyrazol-1- yl)methyl]pyridine-3- carboxylic acid | 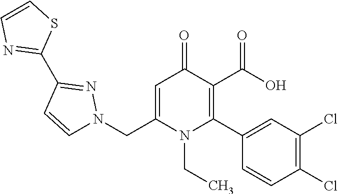 [SMILES: CCn1c(Cn2ccc(-c3nccs3)n2)cc(=O)c(C(=O)O)c1-c1ccc(Cl)c(Cl)c1] | |
| | | 1H NMR (500 MHz, DMSO-d6) δ = 8.15 (s, 1H), 7.91 (d, | |
| | | 1H), 7.81 (d, 1H), 7.79 (d, 1H), 7.74 (d, 1H), 7.45 (dd, 1H), | |
| | | 6.94 (d, 1H), 5.98 (s, 1H), 5.81 (s, 2H), 3.92 (q, 2H), 1.10 | |
| | | (t, 3H) | |
| 202 | 6-[[3-(3- chlorophenyl)pyrazol- 1-yl]methyl]-2-(3,4- dichlorophenyl)-1- ethyl-4-oxo-pyridine- 3-carboxylic acid | 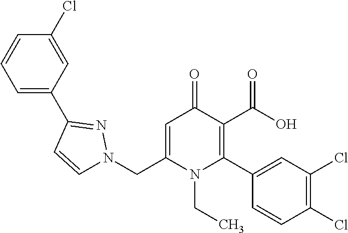 [SMILES: CCn1c(Cn2ccc(-c3cccc(Cl)c3)n2)cc(=O)c(C(=O)O)c1-c1ccc(Cl)c(Cl)c1] | |
| | | 1H NMR (500 MHz, DMSO-d6) δ = 8.03 (brs, 1H), 7.86 (t, | |
| | | 1H), 7.80 (d, 1H), 7.69 (d, 1H), 7.64 (d, 1H), 7.45 (t, 1H), | |
| | | 7.41-7.33 (m, 2H), 6.96 (d, 1H), 5.76 (s, 1H), 5.60 (brs, | |
| | | 2H), 3.85 (brq, 2H), 0.98 (brt, 3H) | |
| 203 | 2-(3,4- dichlorophenyl)-1- ethyl-4-oxo-6-[(3- pyrazin-2-ylpyrazol-1- yl)methyl]pyridine-3- carboxylic acid | 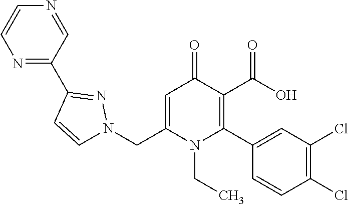 [SMILES: CCn1c(Cn2ccc(-c3cnccn3)n2)cc(=O)c(C(=O)O)c1-c1ccc(Cl)c(Cl)c1] | |
| | | 1H NMR (500 MHz, DMSO-d6) δ = 9.15 (d, 1H), 8.70- | |
| | | 8.67 (m, 1H), 8.60 (d, 1H), 8.16 (d, 1H), 7.83-7.77 (m, | |
| | | 2H), 7.46 (dd, 1H), 7.09 (d, 1H), 6.08 (s, 1H), 5.83 (s, 2H), | |
| | | 3.97 (q, 2H), 1.12 (t, 3H) | |
| 204 | 2-(3,4- dichlorophenyl)-1- ethyl-6-[[3-[(E)- methoxyiminomethyl] pyrazol-1-yl]methyl]- 4-oxo-pyridine-3- carboxylic acid | 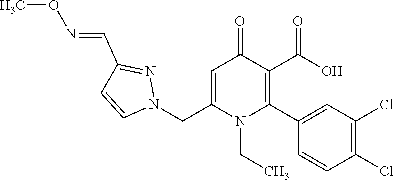 [SMILES: CCn1c(Cn2ccc(/C=N/OC)n2)cc(=O)c(C(=O)O)c1-c1ccc(Cl)c(Cl)c1] | |
| | | 1H NMR (400 MHz, DMSO-d6) δ = 8.16 (s, 1H), 8.03 (d, | |
| | | 1H), 7.79 (d, 1H), 7.77 (d, 1H), 7.42 (dd, 1H), 6.68 (d, 1H), | |
| | | 6.00 (s, 1H), 5.71 (s, 2H), 3.91-3.88 (m, 2H), 3.88 (s, | |
| | | 3H), 1.03 (t, 3H) | |
| 205 | 6-[(5-carboxypyrazol- 1-yl)methyl]-2-(3,4- dichlorophenyl)-1- ethyl-4-oxo-pyridine- 3-carboxylic acid | 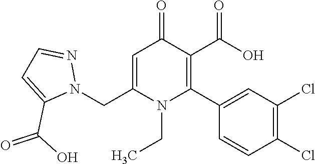 [SMILES: CCn1c(Cn2nccc2C(=O)O)cc(=O)c(C(=O)O)c1-c1ccc(Cl)c(Cl)c1] | |
| | | 1H NMR (500 MHz, DMSO-d6) δ = 16.23 (brs, 1H), 7.84- | |
| | | 7.76 (m, 3H), 7.46 (dd, 1H), 7.05 (d, 1H), 6.01 (s, 2H), 5.47 | |
| | | (s, 1H), 3.90 (q, 2H), 1.16 (t, 3H) | |
| 206 | 2-(3,4- dichlorophenyl)-1- ethyl-5-fluoro-4-oxo- 6-[[3- (trifluoromethyl) pyrazol-1- yl]methyl]pyridine-3- carboxylic acid | 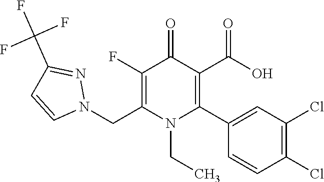 [SMILES: CCn1c(Cn2ccc(C(F)(F)F)n2)c(F)c(=O)c(C(=O)O)c1-c1ccc(Cl)c(Cl)c1] | |
| | | 1H NMR (400 MHz, DMSO-d6) δ = 8.24 (d, 1H), 7.81 (dd, | |
| | | 2H), 7.46 (dd, 1H), 6.86 (d, 1H), 5.80 (d, 2H), 3.96 (q, 2H), | |
| | | 0.59 (t, 3H) | |
| 207 | ethyl 2-(3,4- dichlorophenyl)-1- ethyl-6-[(4- methyltriazol-2- yl)methyl]-4-oxo- pyridine-3-carboxylate | 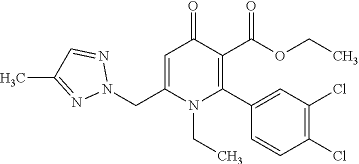 [SMILES: CCOC(=O)c1c(-c2ccc(Cl)c(Cl)c2)n(CC)c(Cn2ncc(C)n2)cc1=O] | |
| | | 1H NMR (400 MHz, chloroform) δ = 7.62-7.57 (m, 2H), | |
| | | 7.45 (s, 1H), 7.36-7.31 (m, 1H), 6.51 (s, 1H), 5.60 (s, 2H), | |
| | | 4.06-3.94 (m, 4H), 2.33 (s, 3H), 1.10 (t, 3H), 0.97 (t, 3H) | |

### BIOLOGICAL EXAMPLES

Seeds of a variety of test species are sown in standard soil in pots (Setaria faberi (SETFA), Echinochloa crus-galli (ECHCG), Zea mays (ZEAMX), Ipomoea hederacea (IPOHE), Amaranthus palmeri (AMAPA), Amaranthus retoflexus (AMARE)). After 8 days cultivation under controlled conditions in a glasshouse (at 24° C./16° C., day/night; 14 hours light; 65% humidity), the plants are sprayed with an aqueous spray solution derived from the formulation of the technical active ingredient in acetone/water (50:50) solution containing 0.5% Tween 20 (polyoxyethelyene sorbitan monolaurate, CAS RN 9005-64-5). Compounds are applied at 1000 g/ha unless otherwise stated. The test plants are then grown in a glasshouse under controlled conditions in a glasshouse (at 24° C./16° C., day/night; 14 hours light; 65% humidity) and watered twice daily. After 13 days the test is evaluated for the percentage damage caused to the plant. The biological activities are shown in the following table on a five-point scale (5=81-100%; 4=61-80%; 3=41-60%; 2=21-40%; 1=0-20%; -=not tested).

TABLE B1
Pre-emergence Test

| Com- | | | | | | |
|---|---|---|---|---|---|---|
| pound | | | | | | |
| No. | SETFA | ECHCG | ZEAMX | IPOHE | AMAPA | AMARE |
| 1 | 4 | 3 | 0 | 0 | 0 | 0 |
| 1* | 4 | 2 | 0 | 0 | 0 | 0 |
| 2 | 5 | 5 | 4 | 3 | 3 | 3 |
| 2* | 5 | 5 | 4 | 4 | 0 | 1 |
| 3 | 5 | 4 | 0 | 0 | 0 | 1 |
| 3* | 1 | 1 | 0 | 0 | 0 | 0 |
| 4 | 5 | 5 | 3 | 3 | 1 | 2 |
| 4* | 5 | 5 | 1 | 2 | 0 | 0 |
| 5 | 3 | 4 | 0 | 0 | 2 | 0 |
| 5* | 2 | 3 | 0 | 0 | 0 | 0 |
| 6 | 4 | 3 | 0 | 2 | — | 0 |
| 6* | 2 | 3 | 0 | 1 | 1 | 0 |
| 7 | 3 | 3 | 1 | 0 | 0 | 0 |
| 8 | 1 | 3 | 0 | 1 | 0 | 0 |
| 9 | 5 | 5 | 0 | 0 | 2 | 1 |
| 9* | 5 | 5 | 0 | 1 | 1 | 0 |
| 11 | 3 | 3 | 2 | 4 | 4 | 3 |
| 11* | 3 | 3 | 0 | 2 | 4 | 2 |
| 12 | 0 | 0 | 0 | 0 | 0 | 0 |
| 13a | 4 | 3 | 0 | 0 | 0 | 0 |
| 13a* | 2 | 2 | 0 | 0 | 1 | 0 |
| 14 | 5 | 5 | 0 | 0 | 1 | 1 |
| 14* | 3 | 4 | 0 | 0 | 0 | 0 |
| 15 | 4 | 4 | 2 | 3 | 5 | 2 |
| 16 | 1 | 4 | 0 | 0 | 5 | 4 |
| 16* | 0 | 1 | 0 | 0 | 3 | 1 |
| 17 | 4 | 4 | 2 | 3 | 5 | 2 |
| 18 | 2 | 1 | 0 | 0 | 0 | 0 |
| 19 | 5 | 5 | 3 | 3 | 1 | 1 |
| 19* | 5 | 2 | 0 | 0 | 0 | 0 |
| 20 | 2 | 4 | 1 | 0 | 0 | 0 |
| 21* | 0 | 0 | 0 | 1 | 0 | 0 |
| 22 | 4 | 3 | 0 | 0 | 0 | 0 |
| 22* | 4 | 1 | 0 | 0 | 0 | 0 |
| 25 | 5 | 5 | 1 | 1 | 2 | 2 |
| 26 | 3 | 2 | 1 | 0 | 0 | 0 |
| 27 | 0 | 0 | 0 | 0 | 0 | 1 |
| 28 | 5 | 5 | 0 | 1 | 0 | 0 |
| 29 | 5 | 4 | 0 | 0 | 1 | 1 |
| 30 | 5 | 5 | 5 | 4 | 1 | 0 |
| 31 | 5 | 5 | 0 | 1 | 0 | 0 |
| 32 | 5 | 5 | 4 | 3 | 4 | 3 |
| 33 | 5 | 5 | 0 | 0 | 3 | 0 |
| 34 | 5 | 5 | 0 | 0 | 1 | 0 |
| 35 | 4 | 5 | 5 | 4 | 2 | 1 |
| 36* | 4 | 3 | 0 | 0 | 2 | 1 |
| 37 | 5 | 5 | 5 | 4 | 2 | 1 |
| 38 | 5 | 5 | 5 | 4 | 3 | 2 |
| 39 | 3 | 4 | 0 | 2 | 1 | 1 |
| 40 | 1 | 0 | 0 | 2 | 3 | 1 |
| 44 | 1 | 2 | 0 | 1 | 3 | 2 |
| 45 | 1 | 4 | 0 | 0 | 1 | 0 |
| 48 | 5 | 5 | 0 | 0 | 1 | 0 |
| 49 | 0 | 1 | 0 | 0 | 1 | 2 |
| 50 | 0 | 1 | 0 | 0 | 1 | 1 |
| 52 | 0 | — | 0 | 0 | 1 | 1 |
| 61 | 1 | — | 0 | 0 | 1 | 1 |
| 64* | 2 | 0 | 0 | 3 | 1 | 0 |
| 75* | 1 | 1 | 0 | 0 | 1 | 1 |
| 78 | 5 | 5 | 3 | 4 | 2 | 2 |
| 78* | 5 | 5 | 4 | 4 | 3 | 2 |
| 79* | 1 | 1 | 0 | 0 | 1 | 0 |
| 80 | 5 | 5 | 0 | 3 | 1 | 0 |
| 80* | 4 | 3 | 0 | 2 | 1 | 0 |
| 84 | 0 | 0 | 0 | 0 | 0 | 0 |
| 85* | 3 | 2 | 0 | 1 | 0 | 0 |
| 88 | 2 | 3 | 1 | 2 | — | — |
| 89 | 4 | 5 | 1 | 4 | 3 | 2 |
| 89* | 3 | 4 | 0 | 3 | 0 | 0 |
| 91 | 0 | 0 | 0 | 0 | 1 | 1 |
| 92 | 4 | 4 | 0 | 2 | 2 | 0 |
| 92* | 3 | 3 | 0 | 3 | 1 | 0 |
| 93* | 0 | 0 | 0 | 0 | 2 | 1 |
| 94 | 1 | 2 | 0 | 1 | 2 | 1 |
| 94* | 1 | 2 | 0 | 0 | 1 | 0 |
| 96 | 3 | 3 | 0 | 1 | 1 | 1 |
| 98* | 0 | 0 | 0 | 0 | 1 | 0 |
| 100 | 1 | 0 | 0 | 2 | 2 | 2 |
| 101 | 1 | 0 | 1 | 2 | 2 | 1 |
| 102 | 1 | 1 | 0 | 3 | 1 | 1 |
| 103 | 1 | 1 | 0 | 0 | 2 | 1 |
| 104 | 5 | 5 | 5 | 4 | 1 | 1 |
| 104* | 5 | 5 | 2 | 1 | 1 | 1 |
| 108 | 0 | 0 | 0 | 0 | 0 | 0 |
| 109 | 5 | 5 | 0 | 4 | 0 | 0 |
| 109* | 2 | 3 | 0 | 1 | 0 | 0 |
| 112 | 0 | 0 | 0 | 0 | 1 | 1 |
| 115 | 1 | 0 | 0 | 0 | 1 | 0 |
| 117 | 5 | 4 | 1 | 2 | 2 | 2 |
| 117* | 4 | 3 | 0 | 0 | 1 | 0 |
| 118 | 4 | 4 | 0 | 2 | 0 | 1 |
| 118* | 5 | 4 | 0 | 1 | 0 | 1 |
| 119 | 4 | 3 | 0 | 0 | 1 | 0 |
| 119* | 3 | 1 | 0 | 0 | 1 | 0 |
| 122* | 1 | 0 | 0 | 0 | 1 | 0 |
| 123 | 1 | 0 | 0 | 0 | 1 | 0 |
| 124* | 0 | 0 | 0 | 0 | 1 | 1 |
| 125* | 1 | 0 | 0 | 0 | 1 | 0 |
| 127 | 5 | 3 | 4 | 2 | 1 | 2 |
| 127* | 3 | 1 | 0 | 0 | 1 | 1 |
| 128 | 3 | 1 | 0 | 1 | 1 | 1 |
| 130 | 0 | 0 | 0 | 0 | 1 | 1 |
| 134 | 5 | 5 | 4 | 4 | 4 | 2 |
| 134* | 5 | 4 | 0 | 1 | 2 | 1 |
| 135 | 0 | 1 | 1 | 2 | 0 | 1 |
| 143 | 0 | 0 | 0 | 0 | 1 | 1 |
| 144 | 5 | 1 | 0 | 1 | 0 | 0 |
| 145 | 5 | 4 | 0 | 3 | 0 | 1 |
| 145* | 5 | 2 | 0 | 0 | 0 | 0 |
| 146 | 1 | 1 | 0 | 0 | 0 | 0 |
| 147 | 1 | 0 | 0 | 0 | 1 | 0 |
| 148 | 5 | 2 | 0 | 2 | 1 | 0 |
| 148* | 2 | 0 | 0 | 0 | 0 | 0 |
| 149 | 4 | 1 | 0 | 0 | 3 | 1 |
| 150 | 5 | 5 | 3 | 4 | 2 | 1 |
| 150* | 5 | 4 | 1 | 1 | 1 | 0 |
| 151 | 5 | 5 | 2 | 4 | 1 | 0 |
| 151* | 5 | 5 | 2 | 2 | 0 | 0 |
| 160 | 5 | 5 | 3 | 2 | 0 | 1 |
| 160* | 2 | 2 | 0 | 2 | 0 | 0 |
| 161 | 4 | 3 | 0 | 3 | 1 | 0 |
| 161* | 1 | 0 | 0 | 0 | 0 | 0 |
| 162 | — | 5 | 4 | 4 | 1 | 3 |
| 162* | — | 3 | 1 | 1 | 0 | 1 |
| 163 | 3 | 4 | 2 | 3 | 0 | 0 |
| 163* | 0 | 1 | 0 | 1 | 0 | 0 |
| 164 | — | 5 | 0 | 4 | 1 | 1 |
| 164* | — | 4 | 1 | 4 | 0 | 1 |
| 165 | — | 5 | 4 | 4 | 2 | 1 |
| 165* | — | 4 | 1 | 3 | 0 | 0 |
| 166 | — | 5 | 5 | 4 | 1 | 1 |
| 166* | — | 5 | 1 | 2 | 0 | 0 |
| 169* | 5 | 4 | 0 | 1 | 1 | 1 |
| 170 | 4 | 3 | 0 | 2 | 0 | 0 |
| 171 | 4 | 1 | 1 | 0 | 0 | 1 |
| 173 | 4 | 4 | 0 | 3 | 0 | 0 |
| 175 | 3 | 1 | 0 | 1 | 0 | 0 |
| 176 | 4 | 4 | 0 | 1 | 2 | 0 |
| 176* | 4 | 2 | 0 | 0 | 1 | 0 |
| 177 | — | 5 | 0 | 2 | 1 | 1 |
| 177* | — | 4 | 0 | 1 | 1 | 0 |
| 178 | — | 2 | 0 | 0 | 0 | 0 |
| 178* | — | 1 | 0 | 0 | 0 | 0 |
| 179 | — | 5 | 5 | 4 | 2 | 4 |
| 179* | — | 1 | 0 | 0 | 0 | — |
| 180 | — | 1 | 0 | 0 | 1 | 2 |
| 185 | — | 5 | 3 | 3 | 2 | 3 |
| 185* | — | 1 | 0 | 1 | 1 | 1 |
| 186 | 5 | 5 | 3 | 4 | 1 | 2 |
| 186* | 5 | 4 | 0 | 2 | 0 | 0 |
| 187 | — | 1 | 1 | 3 | 0 | 0 |
| 189* | — | 5 | 4 | 3 | 2 | 2 |
| 190 | — | 4 | 2 | 3 | 1 | 1 |
| 190* | — | 1 | 1 | 1 | 0 | 0 |
| 193 | — | 0 | 0 | 1 | 0 | 0 |
| 194 | — | 2 | 1 | 2 | 0 | 0 |
| 194* | — | 1 | 0 | 1 | 0 | 0 |
| 198 | — | 1 | 1 | 0 | 1 | 1 |
| 201 | — | 0 | 0 | 1 | 0 | 0 |
| 203 | — | 2 | 1 | 0 | 1 | 1 |
| 204 | — | 4 | 2 | 4 | 1 | 1 |
| 204* | — | 2 | 1 | 2 | 1 | 0 |

*Compounds are applied at 250 g/Ha

TABLE B2
Post-emergence Test

| Com- | | | | | | |
|---|---|---|---|---|---|---|
| pound | | | | | | |
| No. | SETFA | ECHCG | ZEAMX | IPOHE | AMAPA | AMARE |
| 1 | 4 | 4 | 3 | 1 | 1 | 1 |
| 1* | 3 | 3 | 1 | 2 | 0 | 0 |
| 2 | 5 | 4 | 4 | 4 | 4 | 4 |
| 2* | 4 | 4 | 4 | 3 | 3 | 3 |
| 3 | 3 | 3 | 2 | 2 | 2 | 1 |
| 3* | 2 | 1 | 0 | 4 | 1 | 1 |
| 4 | 4 | 4 | 3 | 4 | 4 | 2 |
| 4* | 4 | 4 | 2 | — | 3 | 3 |
| 5 | 4 | 1 | 2 | 1 | 1 | 1 |
| 6 | 4 | 4 | 4 | 1 | 2 | 0 |
| 6* | 0 | 3 | 0 | 1 | 0 | 1 |
| 7 | 4 | 2 | 1 | 1 | 1 | 1 |
| 8 | 4 | 1 | 1 | 1 | 1 | 1 |
| 9 | 3 | 4 | 2 | 1 | 1 | 0 |
| 9* | 4 | 3 | 0 | 3 | 0 | 0 |
| 10 | 0 | 0 | 1 | 1 | 0 | 0 |
| 11 | 1 | 1 | 1 | 1 | 1 | 1 |
| 12 | 1 | 1 | 1 | 2 | 2 | 1 |
| 13a | 4 | 4 | 3 | 2 | 2 | 0 |
| 13a* | 3 | 2 | 0 | 3 | 0 | 1 |
| 14 | 4 | 4 | 1 | 3 | 3 | 1 |
| 14* | 2 | 1 | 1 | 3 | 1 | 1 |
| 15 | 4 | 4 | 3 | 5 | 5 | 3 |
| 15* | 3 | 4 | 2 | 3 | 5 | 5 |
| 16 | 5 | 4 | 4 | 5 | 5 | 1 |
| 16* | 2 | 2 | 0 | 2 | 4 | 4 |
| 17 | 4 | 4 | 3 | 5 | 5 | 3 |
| 17* | 3 | 4 | 2 | 3 | 5 | 5 |
| 18 | 3 | 3 | 2 | 1 | 2 | 1 |
| 18* | 1 | 1 | 1 | 1 | 0 | 1 |
| 19 | 4 | 4 | 3 | 2 | 1 | 4 |
| 19* | 3 | 2 | 2 | 4 | 0 | 0 |
| 20 | 4 | 3 | 1 | 0 | 0 | 1 |
| 21 | 0 | 0 | 1 | 3 | 2 | 1 |
| 22 | 3 | 3 | 1 | 2 | 1 | 1 |
| 22* | 3 | 0 | 0 | 1 | 1 | 1 |
| 23 | 2 | 2 | 1 | 2 | 2 | 1 |
| 24 | 1 | 1 | 3 | 1 | 1 | 1 |
| 24* | 1 | 1 | 1 | 2 | 1 | 1 |
| 25 | 4 | 4 | 3 | 4 | 2 | 2 |
| 25* | 4 | 3 | 0 | 4 | 2 | 2 |
| 26 | 1 | 1 | 2 | 3 | 2 | 1 |
| 26* | 1 | 1 | 1 | 1 | 1 | 1 |
| 27 | 0 | 0 | 0 | 0 | 0 | 0 |
| 28 | 4 | 4 | 4 | 1 | 2 | 0 |
| 28* | 4 | 3 | 0 | 4 | 1 | 1 |
| 29 | 4 | 4 | 4 | 1 | 1 | 2 |
| 29* | 4 | 3 | 0 | 4 | 0 | 1 |
| 30 | 5 | 4 | 4 | 2 | 2 | 4 |
| 30* | 4 | 3 | 3 | 4 | 0 | 1 |
| 31 | 4 | 4 | 3 | 2 | 2 | 2 |
| 31* | 4 | 2 | 1 | 4 | 0 | 1 |
| 32 | 4 | 4 | 3 | 2 | 2 | 4 |
| 32* | 4 | 3 | 1 | 4 | 2 | 1 |
| 33 | 4 | 4 | 2 | 3 | 2 | 3 |
| 33* | 3 | 3 | 1 | 4 | 1 | 1 |
| 34 | 4 | 4 | 2 | 4 | 3 | 1 |
| 34* | 3 | 2 | 1 | 4 | 1 | 1 |
| 35 | 4 | 4 | 3 | 2 | 1 | 3 |
| 35* | 4 | 3 | 2 | 4 | 1 | — |
| 36* | 4 | 3 | 2 | 4 | 1 | 2 |
| 37 | 5 | 4 | 4 | 2 | 4 | 1 |
| 37* | 4 | 4 | 1 | 4 | 2 | 2 |
| 38 | 4 | 4 | 4 | 3 | 4 | 4 |
| 38* | 4 | 3 | 2 | 4 | 1 | 2 |
| 39 | 4 | 4 | 4 | 2 | 2 | 2 |
| 39* | 1 | 3 | 0 | 2 | 1 | 2 |
| 40 | 1 | 0 | 2 | 2 | 1 | 0 |
| 40* | 0 | 1 | 0 | 0 | 0 | 1 |
| 43 | 1 | 1 | 1 | 0 | 1 | 0 |
| 43* | 0 | 1 | 0 | 0 | 1 | 1 |
| 44 | 1 | 1 | 1 | 2 | 3 | 0 |
| 45 | 1 | 1 | 0 | 0 | 1 | 0 |
| 48 | 4 | 5 | 3 | 2 | 3 | 0 |
| 48* | 4 | 3 | 0 | 4 | 1 | 1 |
| 49 | 2 | 2 | 0 | 0 | 1 | 0 |
| 49* | 1 | 0 | 0 | 1 | 0 | 0 |
| 50 | 2 | 1 | 1 | 2 | 3 | 0 |
| 51 | 1 | 1 | 0 | 1 | 1 | 0 |
| 52 | 1 | 1 | 0 | 2 | 2 | 0 |
| 54 | 1 | 1 | 1 | 0 | 1 | 1 |
| 55 | 1 | 2 | 1 | 3 | 3 | 0 |
| 55* | 0 | 2 | 0 | 0 | 1 | 1 |
| 58 | 1 | 1 | 0 | 0 | 0 | 1 |
| 60 | 1 | 2 | 0 | 1 | 0 | 1 |
| 61 | 1 | 0 | 0 | 0 | 0 | 1 |
| 64* | 2 | 3 | 1 | 1 | 2 | 3 |
| 67* | 0 | 0 | 0 | 0 | 1 | 1 |
| 68 | 1 | 1 | 0 | 1 | 1 | 1 |
| 68* | 1 | 0 | 1 | 1 | 1 | 1 |
| 69 | 3 | 1 | 2 | 1 | 1 | 1 |
| 70 | 2 | 1 | 0 | 1 | 1 | 1 |
| 74 | 0 | 0 | 1 | 0 | 0 | 0 |
| 75* | 0 | 1 | 0 | 0 | 1 | 1 |
| 76 | 1 | 0 | 1 | 0 | 1 | 1 |
| 77 | 0 | 1 | 1 | 0 | 1 | 1 |
| 78 | 4 | 4 | 3 | 4 | 2 | 3 |
| 78* | 4 | 3 | 1 | 4 | 2 | 1 |
| 79* | 0 | 1 | 1 | 1 | 0 | 1 |
| 80 | 4 | 4 | 3 | 3 | 2 | 2 |
| 80* | 3 | 3 | 1 | 4 | 2 | 2 |
| 84 | 0 | 0 | 1 | 0 | 0 | 0 |
| 84* | 0 | 1 | 0 | 0 | 0 | 0 |
| 85* | 4 | 2 | 0 | 4 | 0 | 0 |
| 87 | 1 | 1 | 1 | 1 | 2 | 1 |
| 87* | 0 | 1 | 1 | 0 | 0 | 0 |
| 88 | 4 | 2 | 3 | 2 | 2 | 1 |
| 88* | 0 | 3 | 0 | 2 | 1 | 1 |
| 89 | 4 | 4 | 3 | 3 | 3 | 1 |
| 89* | 2 | 4 | 0 | — | 1 | 1 |
| 90 | 0 | 1 | 0 | 0 | 1 | 1 |
| 91 | 1 | 1 | 1 | 3 | 4 | 2 |
| 91* | 0 | 3 | 1 | 0 | 1 | 1 |
| 92 | 3 | 4 | 2 | 3 | 2 | 2 |
| 92* | 3 | 3 | 2 | 3 | 2 | 2 |
| 93* | 1 | 2 | 1 | 1 | 1 | 1 |
| 94 | 4 | 1 | 0 | 3 | 3 | 3 |
| 94* | 0 | 4 | 0 | 0 | 2 | 3 |
| 95 | 1 | 1 | 1 | 1 | 1 | 0 |
| 96 | 3 | 2 | 4 | 0 | 0 | 1 |
| 96* | 0 | 2 | 0 | 0 | 0 | 0 |
| 97* | 0 | 1 | 1 | 0 | 1 | 1 |
| 98* | 1 | 2 | 1 | 1 | 0 | 1 |
| 99 | 0 | 0 | 1 | 1 | 0 | 0 |
| 99* | 0 | 1 | 0 | 0 | 0 | 0 |
| 100 | 2 | 2 | 1 | 3 | 2 | 2 |
| 100* | 0 | 3 | 0 | 0 | 0 | 0 |
| 101 | 0 | 0 | 0 | 3 | 0 | 0 |
| 101* | 0 | 1 | 0 | 0 | 0 | 0 |
| 102 | 2 | 3 | 0 | 3 | 4 | 4 |
| 102* | 1 | 3 | 1 | 1 | 2 | 1 |
| 103 | 1 | 1 | 1 | 0 | 1 | 1 |
| 104 | 4 | 4 | 4 | 3 | 4 | 4 |
| 104* | 4 | 4 | 4 | 4 | 3 | 3 |
| 105 | 2 | 3 | 1 | 4 | 2 | 2 |
| 105* | 0 | 3 | 0 | 0 | 0 | 0 |
| 106 | 1 | 1 | 0 | 3 | 1 | 1 |
| 106* | 0 | 2 | 0 | 0 | 0 | 0 |
| 107 | 1 | 1 | 0 | 2 | 1 | 1 |
| 107* | 0 | 2 | 0 | 0 | 0 | 0 |
| 108 | 2 | 2 | 1 | 0 | 0 | 0 |
| 108* | 1 | 1 | 0 | 0 | 0 | 0 |
| 109 | 4 | 4 | 3 | 3 | 3 | 1 |
| 109* | 1 | 3 | 1 | 2 | 0 | 0 |
| 110 | 1 | 1 | 0 | 1 | 0 | 0 |
| 111 | 1 | 2 | 0 | 0 | 1 | 1 |
| 112 | 1 | 2 | 0 | 2 | 1 | 1 |
| 113 | 1 | 1 | 1 | 0 | 1 | 1 |
| 115 | 0 | 0 | 1 | 0 | 2 | 0 |
| 115* | 0 | 0 | 0 | 0 | 1 | 1 |
| 116 | 1 | 1 | 1 | 1 | 1 | 1 |
| 117 | 4 | 4 | 4 | 3 | 2 | 1 |
| 117* | 3 | 3 | 1 | 3 | 0 | 0 |
| 118 | 4 | 4 | 2 | 4 | — | 2 |
| 118* | 4 | 3 | 2 | 4 | 2 | 2 |
| 119 | 4 | 4 | 0 | 2 | — | 0 |
| 119* | 1 | 1 | 0 | 1 | 0 | 1 |
| 123 | 1 | 1 | 1 | 2 | — | 1 |
| 123* | 0 | 2 | 0 | 0 | 0 | 0 |
| 124* | 2 | 4 | 1 | 0 | 0 | 1 |
| 125* | 1 | 1 | 1 | 1 | 0 | 1 |
| 126 | 0 | 0 | 1 | 0 | 1 | 0 |
| 126* | 0 | 1 | 0 | 0 | 0 | 0 |
| 127 | 3 | 4 | 3 | 3 | 3 | 2 |
| 127* | 3 | 2 | 1 | 1 | 0 | 0 |
| 128 | 3 | 4 | 1 | 1 | 1 | 1 |
| 128* | 0 | 1 | 0 | 1 | 0 | 0 |
| 129 | 0 | 1 | 1 | 0 | 0 | 0 |
| 130 | 1 | 0 | 0 | 1 | 2 | 1 |
| 131 | 2 | — | 0 | 1 | 0 | 0 |
| 132 | 1 | 1 | 1 | 0 | — | 0 |
| 133 | 0 | 0 | 0 | 1 | 1 | 1 |
| 133* | 1 | 0 | 0 | 0 | 1 | 1 |
| 134 | 4 | 4 | 3 | 3 | 4 | 4 |
| 134* | 4 | 3 | 2 | 4 | 1 | 2 |
| 135 | 4 | — | 2 | 3 | 1 | 2 |
| 135* | — | 1 | 1 | 2 | 1 | 1 |
| 143 | 2 | 2 | 1 | 2 | 1 | 1 |
| 144 | 3 | 3 | 3 | 2 | 1 | 2 |
| 144* | 2 | 2 | 0 | 1 | 0 | 1 |
| 145 | 4 | 4 | 3 | 3 | 3 | 3 |
| 145* | 3 | 3 | 1 | 3 | 0 | 1 |
| 146 | 1 | 0 | 0 | 0 | 0 | 0 |
| 147 | 0 | 0 | 0 | 1 | 0 | 1 |
| 148 | 4 | 4 | 2 | 2 | 3 | 3 |
| 148* | 3 | 2 | 1 | 2 | 1 | 1 |
| 149 | 3 | 4 | 2 | 2 | 1 | 2 |
| 149* | 1 | 1 | 0 | 1 | 0 | 0 |
| 150 | 4 | 4 | 3 | 2 | 1 | 2 |
| 150* | 4 | 2 | 1 | 4 | 0 | 0 |
| 151 | 4 | 4 | 3 | 4 | 2 | 3 |
| 151* | 4 | 3 | 1 | 4 | 1 | 1 |
| 152 | 0 | 1 | 1 | 0 | 1 | 1 |
| 153 | 0 | 0 | 1 | 2 | 0 | 1 |
| 154 | 0 | 0 | 1 | 0 | 0 | 0 |
| 154* | 0 | 0 | 1 | 0 | 0 | 0 |
| 155 | 0 | 1 | 0 | 0 | 1 | 0 |
| 155* | 1 | 1 | 0 | 1 | 1 | 1 |
| 160 | 4 | 4 | 4 | 3 | 1 | 1 |
| 160* | 2 | 2 | 1 | 3 | 0 | 0 |
| 161 | 3 | 3 | 1 | 3 | 1 | 0 |
| 161* | 2 | 2 | 1 | 1 | 0 | 1 |
| 162 | 5 | — | 3 | 4 | 2 | 2 |
| 162* | — | 3 | 1 | 2 | 1 | 1 |
| 163 | 4 | 3 | 1 | 2 | 1 | 1 |
| 164 | 5 | — | 4 | 4 | 3 | 3 |
| 164* | — | 4 | 4 | 3 | 1 | 1 |
| 165 | 4 | — | 3 | 4 | 2 | 3 |
| 165* | — | 3 | 2 | 4 | 1 | 1 |
| 166 | 5 | — | 4 | 4 | 1 | 1 |
| 166* | — | 4 | 2 | 2 | 1 | 0 |
| 167 | 0 | 0 | 0 | 0 | 1 | 0 |
| 169* | 4 | 3 | 4 | 4 | 3 | 3 |
| 170 | 4 | 4 | 1 | 3 | 1 | 1 |
| 170* | 3 | 3 | 0 | 3 | 0 | 0 |
| 171 | 3 | 3 | 1 | 1 | 0 | 0 |
| 171* | 0 | 1 | 1 | 1 | 0 | 0 |
| 172 | 1 | — | 1 | 1 | 2 | 2 |
| 172* | — | 0 | 0 | 0 | 1 | 1 |
| 173 | 4 | 4 | 1 | 2 | 1 | 1 |
| 173* | 1 | 2 | 0 | 1 | 0 | 0 |
| 175 | 3 | 2 | 1 | 2 | 0 | 0 |
| 175* | 0 | 1 | 0 | 1 | 0 | 0 |
| 176 | 4 | 4 | 1 | 3 | — | 3 |
| 176* | 2 | 2 | 0 | 2 | 0 | 0 |
| 177 | 4 | — | 1 | 3 | 2 | 3 |
| 177* | — | 3 | 1 | 4 | 2 | 1 |
| 178 | 2 | — | 0 | 0 | 1 | 1 |
| 178* | — | 1 | 0 | 1 | 1 | 0 |
| 179 | 4 | — | 4 | 4 | 3 | 3 |
| 179* | — | 3 | 3 | 4 | 1 | 0 |
| 180 | 1 | — | 1 | 1 | 0 | 1 |
| 180* | — | 1 | 0 | 0 | 0 | 0 |
| 181 | 0 | — | 0 | 1 | 1 | 1 |
| 181* | — | 1 | 0 | 0 | 0 | 0 |
| 184 | 4 | — | 3 | 4 | 3 | 3 |
| 184* | — | 3 | 1 | 1 | 1 | 1 |
| 185 | 4 | — | 4 | 3 | 3 | 3 |
| 185* | — | 3 | 1 | 3 | 1 | 1 |
| 186 | 4 | 4 | 1 | 4 | 2 | 3 |
| 186* | 4 | 4 | 1 | 4 | 2 | 1 |
| 187 | 1 | — | 1 | 4 | 1 | 1 |
| 187* | — | 2 | 0 | 1 | 0 | 0 |
| 188 | 0 | 0 | 0 | 1 | 1 | 0 |
| 189* | — | 4 | 4 | 4 | 5 | 4 |
| 190 | 4 | — | 3 | 3 | 1 | 2 |
| 190* | — | 3 | 0 | 1 | 1 | 1 |
| 192 | 0 | 0 | 0 | 0 | 1 | 1 |
| 193 | 0 | — | 1 | 2 | 1 | 1 |
| 193* | — | 3 | 0 | 0 | 0 | 0 |
| 194 | 3 | — | 1 | 2 | 1 | 2 |
| 194* | — | 2 | 1 | 1 | 0 | 0 |
| 195 | 2 | — | 1 | 1 | 0 | 1 |
| 195* | — | 1 | 0 | 0 | 0 | 0 |
| 196 | 0 | — | 1 | 0 | 0 | 0 |
| 196* | — | 1 | 0 | 0 | 0 | 0 |
| 198 | 2 | — | 2 | 2 | 3 | 4 |
| 198* | — | 2 | 1 | 1 | 1 | 1 |
| 199 | 0 | — | 0 | 2 | 0 | 0 |
| 199* | — | 1 | 0 | 0 | 0 | 0 |
| 200 | 1 | — | 1 | 1 | 0 | 0 |
| 200* | — | 1 | 1 | 0 | 0 | 0 |
| 201 | 2 | — | 1 | 2 | 1 | 2 |
| 201* | — | 1 | 0 | 0 | 0 | 0 |
| 202 | 1 | — | 1 | 1 | 0 | 0 |
| 203 | 1 | — | 1 | 1 | 0 | 0 |
| 203* | — | 1 | 1 | 2 | 1 | 1 |
| 204 | 4 | — | 3 | 3 | 1 | 2 |
| 204* | — | 3 | 2 | 2 | 1 | 1 |

*Compounds are applied at 250 g/Ha

1. A compound of Formula (I): 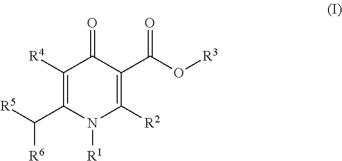
wherein
R¹ is C₁-C₆alkyl, C₁-C₆alkoxy, C₂-C₆alkenyl, C₂-C₆alkynyl, or C₁-C₆alkoxyC₁-C₆alkyl;
R² is phenyl or heteroaryl, wherein the heteroaryl moiety is a 5- or 6-membered aromatic ring which comprises 1, 2, 3 or 4 heteroatoms individually selected from N, O and S, and wherein each phenyl and heteroaryl moiety may be optionally substituted with 1, 2, 3, or 4 groups, which may be the same or different, represented by R⁷;
R³ is hydrogen or C₁-C₆alkyl;
R⁴ is hydrogen, halogen, C₁-C₆alkyl, C₁-C₆haloalkyl, or phenyl, and wherein the phenyl moieties may each be optionally substituted with 1, 2, 3, or 4 groups, which may be the same or different, represented by R⁸;
R⁵ is heteroaryl wherein the heteroaryl moiety is a 5-membered aromatic monocyclic ring comprising 1, 2, or 3 nitrogen atoms, and wherein the heteroaryl moieties are attached to the rest of the molecule through a nitrogen atom in the heteroaryl ring, and wherein the heteroaryl moieties may each be optionally substituted with 1, 2, 3, or 4 groups, which may be the same or different, represented by R⁹;
R⁶ is hydrogen, C₁-C₃alkyl, or C₁-C₆alkoxy;
R⁷ is cyano, nitro, amino, halogen, C₁-C₆alkyl, C₁-C₆alkoxy, C₁-C₆haloalkyl, C₁-C₆haloalkoxy, C₁-C₆alkoxyC₁-C₆alkyl, C₁-C₆alkylsulfanyl, C₁-C₆alkylsulfinyl, C₁-C₆alkylsulfonyl, C₁-C₆alkylsulfonamido, C₁-C₆alkylcarbonyl, C₁-C₆alkoxycarbonyl, C₁-C₆alkylaminocarbonyl, C₃-C₆cycloalkyl, C₃-C₆cycloalkylaminocarbonyl, or N,N-di(C₁-C₄alkyl)aminocarbonyl;
R⁸ is halogen, C₁-C₃alkyl, or C₁-C₃alkoxy;
R⁹ is formyl, acetyl, cyano, amino, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkyl, cyanoC₁-C₆alkoxy, C₁-C₆alkoxyC₁-C₆alkyl, C₁-C₆alkoxyC₁-C₆alkoxy, C₁-C₆alkoxycarbonylC₁-C₆alkoxy, C₁-C₆alkoxycarbonyl, C₁-C₆alkylsulfanyl, C₁-C₆alkylsulfinyl, C₁-C₆alkylsulfonyl, C₁-C₆alkylsulfonamido, hydroxycarbonyl, C₁-C₆alkylcarbonyl, C₁-C₆alkylaminocarbonyl, C₁-C₆alkylcarbonylamino, C₁-C₆alkylamino, C₁-C₆alkoxyC₁-C₆alkylaminocarbonyl, C₁-C₆alkoxyiminoC₁-C₆alkyl, C₁-C₆alkoxyC₁-C₆alkylcarbonylamino, C₂-C₆alkenylcarbonylamino, C₃-C₆cycloalkyl, C₃-C₆cycloalkylaminocarbonyl, N,N-di(C₁-C₄alkyl)amino, N,N-di(C₁-C₄alkyl)aminocarbonyl, phenyl, benzyloxy, or heteroaryl, wherein the heteroaryl moiety is a 5- or 6-membered aromatic ring which comprises 1, 2, or 3 heteroatoms individually selected from N, O and S, and wherein the phenyl and heteroaryl moieties may be optionally substituted with 1, 2, 3 or 4 groups, which may be the same or different, represented by R¹⁰; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, 3 or 4 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, 3 or 4 heteroatoms individually selected from N, O and S, and wherein the heteroaryl moiety may be optionally substituted with 1, 2, 3 or 4 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, 3 or 4 groups, which may be the same or different, represented by R¹⁰;
R¹⁰ is halogen, nitro, C₁-C₃alkyl, or C₁-C₃alkoxy;
or a salt or an N-oxide thereof.

2. The compound according to claim 1, wherein R² is phenyl or heteroaryl, wherein the heteroaryl moiety is a 5- or 6-membered aromatic ring which comprises 1 or 2 heteroatoms individually selected from N and O, and wherein each phenyl and heteroaryl moiety may be optionally substituted with 1, 2, or 3 groups, which may be the same or different, represented by R⁷.

3. The compound according to claim 1, wherein R² is phenyl optionally substituted with 1 or 2 groups, which may be the same or different, represented by R⁷.

4. The compound according to claim 1, wherein R³ is hydrogen or C₁-C₄alkyl.

5. The compound according to claim 1, wherein R⁴ is hydrogen, halogen, C₁-C₃alkyl, or phenyl, wherein the phenyl moieties may be optionally substituted with a single R⁸ group.

6. The compound according to claim 1, wherein R⁵ is heteroaryl wherein the heteroaryl moiety is a 5-membered aromatic monocyclic ring comprising 1, 2, or 3 nitrogen atoms, and wherein the heteroaryl moieties are attached to the rest of the molecule through a nitrogen atom in the heteroaryl ring, and wherein the heteroaryl moieties may each be optionally substituted with 1, 2, or 3 groups, which may be the same or different, represented by R⁹.

7. The compound according to claim 1, wherein R⁵ is heteroaryl wherein the heteroaryl moiety is a 5-membered aromatic monocyclic ring comprising 2 or 3 nitrogen atoms, and wherein the heteroaryl moieties are attached to the rest of the molecule through a nitrogen atom in the heteroaryl ring, and wherein the heteroaryl moieties may each be optionally substituted with 1, 2, or 3 groups, which may be the same or different, represented by R⁹.

8. The compound according to claim 1 wherein R⁹ is formyl, acetyl, cyano, amino, nitro, hydroxy, halogen, C₁-C₆alkyl, C₁-C₆alkoxy, C₁-C₆haloalkyl, C₁-C₆haloalkoxy, C₂-C₆alkenyloxy, cyanoC₁-C₆alkyl, C₁-C₆alkoxyC₁-C₆alkyl, C₁-C₆alkoxyC₁-C₆alkoxy, C₁-C₆alkoxycarbonyl, C₁-C₆alkylsulfanyl, C₁-C₆alkylsulfonyl, hydroxycarbonyl, C₁-C₆alkylcarbonyl, C₁-C₆alkylcarbonylamino, C₁-C₆alkoxyiminoC₁-C₆alkyl, C₁-C₆alkoxyC₁-C₆alkylcarbonylamino, C₂-C₆alkenylcarbonylamino, C₃-C₆cycloalkyl, phenyl, or heteroaryl, wherein the heteroaryl moiety is a 5- or 6-membered aromatic ring which comprises 1, 2, or 3 heteroatoms individually selected from N, O and S, and wherein the phenyl and heteroaryl moieties may be optionally substituted with 1, 2, or 3 groups, which may be the same or different, represented by R¹⁰; or
any two adjacent R⁹ groups together with the carbon atoms to which they are attached, may form a C₃-C₆cycloalkyl ring or phenyl ring, wherein the C₃-C₆cycloalkyl and phenyl moieties may be optionally substituted with 1, 2, or 3 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1, 2, or 3 heteroatoms individually selected from N, O and S, and wherein the heteroaryl moiety may be optionally substituted with 1, 2, or 3 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1, 2, or 3 groups, which may be the same or different, represented by R¹⁰.

9. The compound according to claim 1, wherein R⁹ is acetyl, cyano, nitro, hydroxy, halogen, C₁-C₄alkyl, C₁-C₄alkoxy, C₁-C₄haloalkyl, C₁-C₄haloalkoxy, C₂-C₄alkenyloxy, cyanoC₁-C₄alkyl, C₁-C₄alkoxyC₁-C₄alkyl, C₁-C₄alkoxyC₁-C₄alkoxy, C₁-C₄alkoxycarbonyl, C₁-C₄alkylsulfanyl, C₁-C₄alkylsulfonyl, C₁-C₄alkylcarbonyl, C₁-C₄alkylcarbonylamino, C₁-C₄alkoxyiminoC₁-C₄alkyl, C₁-C₄alkoxyC₁-C₄alkylcarbonylamino, C₂-C₄alkenylcarbonylamino, C₃-C₄cycloalkyl, phenyl, or heteroaryl, wherein the heteroaryl moiety is a 5- or 6-membered aromatic ring which comprises 1 or 2 heteroatoms individually selected from N, O and S, and wherein the phenyl and heteroaryl moieties may be optionally substituted with 1 or 2 groups, which may be the same or different, represented by R¹⁰; or
any two adjacent R⁹ groups together with the carbon atoms to which they are attached, may form a C₅-C₆cycloalkyl ring or phenyl ring, wherein the C₅-C₆cycloalkyl and phenyl moieties may be optionally substituted with 1 or 2 groups, which may be the same or different, represented by R¹⁰; or
any two adjacent R⁹ groups together with the carbon atoms to which they are attached, may form a 5- or 6-membered heteroaryl ring, wherein the heteroaryl moiety is a 5- or 6-membered aromatic ring which comprises 1 or 2 heteroatoms individually selected from N, O and S, and wherein the heteroaryl moiety may be optionally substituted with 1 or 2 groups, which may be the same or different, represented by R¹⁰; or
any two adjacent R⁹ groups together with the carbon atoms to which they are attached, may form a 5- or 6-membered heterocyclyl ring, wherein the heterocyclyl moiety is a 5- or 6-membered comprising 1 or 2 heteroatoms selected from O and N, and wherein the heterocyclyl ring may be optionally substituted with 1 or 2 groups, which may be the same or different, represented by R¹⁰.

10. The compound according to claim 1, wherein R¹⁰ is halogen.

11. A herbicidal composition comprising a compound according to claim 1 and an agriculturally acceptable formulation adjuvant.

12. A herbicidal composition according to claim 11, further comprising at least one additional pesticide.

13. A herbicidal composition according to claim 12, wherein the additional pesticide is a herbicide or herbicide safener.

14. A method of controlling unwanted plant growth, comprising applying a compound of Formula (I) as defined in claim 1 to the unwanted plants or to the locus thereof.

15. Use of a compound of Formula (I) according to claim 1 as a herbicide.
